(19) United States
(12) Patent Application Publication
(10) Pub. No.: US 2024/0112752 A1
(43) Pub. Date: Apr. 4, 2024

(54) METHODS AND SYSTEMS FOR ANNOTATING GENOMIC DATA

(71) Applicant: Martingale Labs, Inc., Brooklyn, NY (US)

(72) Inventors: Tejal Patwardhan, Brooklyn, NY (US); Katy Shi, Brooklyn, NY (US)

(21) Appl. No.: 18/372,389

(22) Filed: Sep. 25, 2023

Related U.S. Application Data
(60) Provisional application No. 63/410,086, filed on Sep. 26, 2022.
(60) Provisional application No. 63/419,601, filed on Oct. 26, 2022.
(60) Provisional application No. 63/436,849, filed on Jan. 3, 2023.

(51) Int. Cl. G16B 20/20; G16B 40/20; G16H 50/20; G16H 50/30

(52) U.S. Cl. CPC G16B 20/20; G16B 40/20; G16H 50/20; G16H 50/30

(57) ABSTRACT

In variants, the method can include receiving a subject's unannotated genomic data, optionally generating annotated variant loci, and optionally determining a risk score for the subject. The method can function to: provide genomic data analysis to a user; predict disease risk; and/or provide recommendations for screenings, treatment, and/or lifestyle changes.

### CROSS-REFERENCE TO RELATED APPLICATIONS

This application claims the benefit of U.S. Provisional Application No. 63/410,086 filed 26 Sep. 2022, U.S. Provisional Application No. 63/419,601 filed 26 Oct. 2022, and U.S. Provisional Application No. 63/436,849 filed 3 Jan. 2023, each of which is incorporated in its entirety by this reference.

### TECHNICAL FIELD

The subject matter disclosed herein is generally directed to converting genomic data to an optimized format and annotating the genomic data with their biological response to diseases and therapeutics. In particular embodiments, the system annotates individual genomic data with clinical records and studies from disparate sources to the user workstation real-time and on-demand.

### BACKGROUND

Every year, millions of patients take genetic tests to screen for hereditary disease risk. Currently, the majority of clinical genetic tests offered to patients involve analyzing coding regions of the genome (including very large gene panels or sometimes exomes) to find highly penetrant coding variants that influence disease risk; whole genome sequencing is rare. Clinicians, including medical geneticists and genetic counselors, use these test results to make a variety of recommendations to patients, such as follow up screenings, therapeutics management, and behavioral changes. In the example of breast cancer, some changes recommended after a pathogenic variant is found might look like: adjusting screenings (e.g., starting mammograms 10 years earlier, alternating with MRIs), therapeutics management (e.g., recommending birth control or preventative surgery), and lifestyle changes (e.g., limiting dairy intake). These measures could help catch disease early or prevent progression to a life-threatening state of disease. Moreover, many types of procedures are not covered by insurance in various countries unless genetic tests come back positive for a pathogenic variant.

Citation or identification of any document in this application is not an admission that such a document is available as prior art to the present invention.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—A schematic representation of a variant of the method.

FIG. 2—A schematic representation of a first example of the system, including communications and processing architecture.

FIG. 3—A schematic representation of a second example of the system, including computing machine and modules, in accordance with certain examples of the technology disclosed herein.

FIG. 4A—Illustrative example of segmenting annotation data.

FIG. 4B—Illustrative example of annotating genomic data.

FIG. 5—Example of determining search regions.

FIG. 6A-6B—Examples of segmenting annotation data across multiple chromosomes.

FIG. 7—Example of annotating genomic data, including matching a variable value set for a variant locus across multiple subsets of annotation data.

FIG. 8—Example of annotating genomic data using a genome annotation network.

FIG. 9—Examples of using annotated genomic data.

FIG. 10A-10C—Examples of aggregating annotations.

FIG. 11—Example of filtering annotations.

FIG. 12—Example of training a risk model.

FIG. 13—Example of determining a risk score.

FIG. 14—Examples of using a risk score.

FIG. 15A and FIG. 15B—Illustrative examples of determining a functional group contribution to the risk score.

FIG. 16—Example output displaying annotated variant loci of a subject.

FIG. 17A-17E—Example Process diagram of embodiments described herein.

FIG. 18—Example output displaying annotated variant loci of a subject.

FIG. 19—Example output of polygenic risk score.

FIG. 20—Example input of unannotated genomic data.

FIG. 21—Example output of annotated variant loci of a subject.

FIG. 22—Example user interface to upload unannotated genomic data.

FIG. 23—Example user interface authorizing execution of methods described herein.

FIG. 24—Another example input of unannotated genomic data.

FIG. 25A-25C—Example layouts of user interface for methods described herein.

FIG. 26—Example user interface to upload unannotated genomic data.

FIG. 27—Example of successful upload of a subject's unannotated genomic data.

FIG. 28—Example load screen after successful upload of a subject's unannotated genomic data.

FIG. 29A-29C—Examples of displaying the subject's annotated variant loci and providing a disease diagnosis or prognosis if the polygenic risk score is above a threshold value.

FIG. 30A-30B—Examples of displaying the subject's annotated variant loci.

FIG. 31A-31B—Examples of displaying the subject's annotated variant loci.

FIG. 32—Example of displaying the subject's annotated variant loci as a downloadable file.

FIG. 33—Example of loci, identified variant loci data, a variable value set, annotation data, and an annotation associated with the variable value set.

FIG. 34A-34D—Examples of displaying genomic data analysis for a subject.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

In variants, the method can include receiving a subject's unannotated genomic data, optionally generating annotated variant loci, and optionally determining a risk score for the subject. The method can function to: provide genomic data analysis (e.g., annotated variant loci, disease risk, etc.) to a user; predict disease risk (e.g., risk of developing polygenic conditions, such as heritable cancers, cardiovascular conditions, immune conditions, etc.); and/or provide recommendations for screenings, treatment, and/or lifestyle changes.

There are currently ˜5000 certified US genetic counselors that help recommend and interpret genetic tests (www.gao.gov/assets/gao-20-593.pdf), with projected 100% growth over the next 10 years (www.nsgc.org/Portals/0/Executive %20Summary %202021%20FINAL %2005-03-21.pdf). Of genetic counselors in the NSGC database (www.nsgc.org/), ˜62% cover complex polygenic conditions (e.g., most prenatal specialists would not benefit from our tests, but cancer and cardiovascular specialists would). These genetic counselors each see approximately 10 new patients weekly. This translates to ˜1.55 million eligible new patients per year in the US that take these genetic tests.

The current pain point is that for polygenic conditions, only ˜10-20% of tests come back positive, and counselors suspect many more should be positive (e.g., every woman in someone's family has breast cancer, but the coding genetic test says “negative.”) When patients get false negative results, it can mean life-threatening conditions are caught too late. Moreover, current coding tests are often less accurate in individuals of non-European ancestry.

The methods and systems described herein could improve the accuracy and ethnic inclusivity of clinical genetic tests. Likewise, the methods and systems described herein make possible personalized medical treatments such as patient (e.g., a subject or individual) specific disease prevention or drug regimen. By receiving a patient's genomic data, aggregating (i.e., acquire, organize, categorize) annotated genomic data from a plurality of resources; standardizing the data, and generating annotated variant loci of the patient's genomic data, these methods and systems can significantly improve medical diagnostics including the speed and accuracy of diagnosis. Furthermore, with continuously produced evidence, including improved evidence, the automatic updating of the annotation data will provide the most up-to-date results for patients using these methods and systems.

In one aspect, technologies herein provide methods to first receive a subject's unannotated genomic data by one or more computing systems. The unannotated genomic data is then converted into a standardized file format based, at least in part, on identified variant loci in the unannotated genomic data. The identified variant loci data is then matched to annotation data from a plurality of data sources comprising different data types to generate annotated variant loci. The subject's annotated variant loci are then displayed.

In one aspect, technology herein includes designing genomic data annotation to operate on user computing devices. The application may be a downloadable application or application programming interface for use on a computing device that annotates genomic data. The data may include unannotated genomic data. The unannotated genomic data may include variant loci of a subject.

In another aspect, the technology includes applications and systems to annotate genomic data. For example, applications may be provided to individual users capable of communicating through wireless means.

In another aspect, technologies herein provide methods to determine disease risk or prognosis in a subject. In another aspect, technologies herein provide methods to treating or modifying a treatment plan.

In one aspect, disclosed herein a computer-implemented method for annotating genomic data, comprising: receiving, by one or more computing systems, a subject's unannotated genomic data; converting, by one or more computing systems, the unannotated genomic data into a standardized file format based, at least in part, on identified variant loci in the unannotated genomic data; generating, by the one or more computing systems, annotated variant loci by matching annotation data from a plurality of data sources comprising different data types with the corresponding identified variant loci; and displaying, by the one or more computing systems, the subject's annotated variant loci.

In an example embodiment, the standardized file format includes values for a set of match-optimized variables for each identified variant locus and configured to optimize search of the annotations from the plurality of data sources. In an example embodiment, the match-optimized variables include one or more variable selected from chromosome number, overall chromosome location, variant start position, variant stop position, variant identification number, variant type, reference allele(s), present allele(s), reference assembly number. In an example embodiment, the standardized file format includes pre-segmenting the unannotated genomic data into subsets.

In an example embodiment, the annotation data from the plurality of data sources is parsed into a matching structure to optimize a search speed of the annotation data. In an example embodiment, the matching structure includes pre-segmenting annotation data into subsets. In an example embodiment, each subset is independently stored to allow parallel searching of multiple subsets. In an example embodiment, each subset corresponds to a chromosome number. In an example embodiment, the annotation data is first pre-segmented into subsets corresponding to a chromosome number, the annotation data in each subset corresponding to a chromosome number is then further pre-segmented into additional subsets.

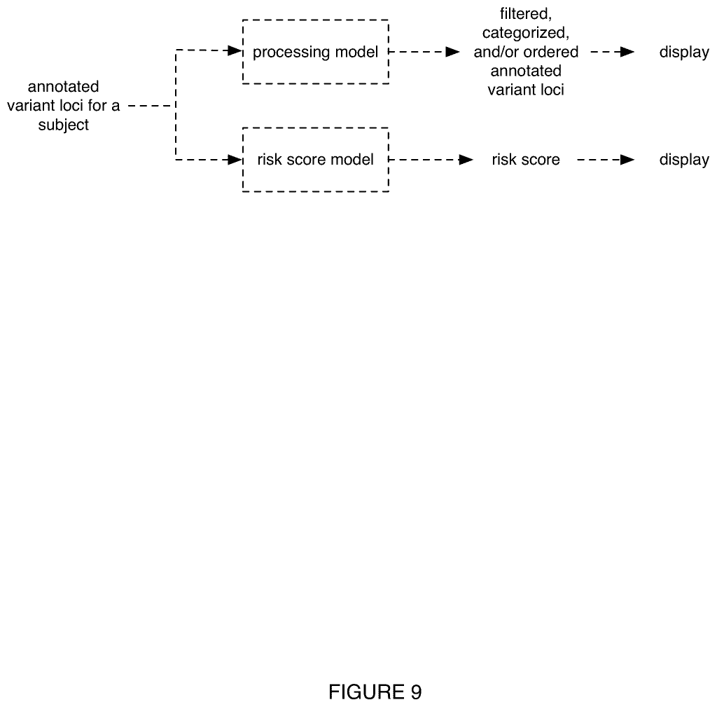

In an example embodiment, the annotation data in each subset is stored in a multi-dimensional array data structure. In an example embodiment, displaying annotated variant loci further includes; filtering annotation data associated with each variant based on a weight metric; and/or categorizing each annotation by annotation type. An example is shown in FIG. 9. In an example embodiment, the weight metric is computed based on number of published annotations, whether the annotation data is clinical grade, whether these annotations are based on expert panel review presence and number of conflicting annotation. In an example embodiment, the method further includes identifying conflicting annotations and selecting the annotation with a higher weight metric.

In an example embodiment, the annotation type includes risk variant type, protective variant type, drug responsiveness, metabolic effects, or any combination thereof. In an example embodiment, displaying the annotated variant loci includes generating a graphical user interface configured to facilitate ease of interpretation and visualization of data. In an example embodiment, the GUI associates a set of visual elements with each identified variant locus, each visual element representing an annotation and grouped by annotation type. In an example embodiment, the visual element further includes one or more links to additional information about the annotation. In an example embodiment, the identified variants and associated visual elements are displayed in ranked order, based at least in part, on the weight metric.

In an example embodiment, the plurality of annotation sources include genotype information, phenotype information, evidence levels, drug efficacy data, drug toxicity data metabolic data, or any combination thereof. In an example embodiment, genotype information includes non-coding DNA variant information. In an example embodiment, a connection to non-coding variants to coding genes or disease states is determined from genome-wide association studies (GWAS), CRISPR-based functional screens, or by activity-by-contact models. In an example embodiment, multiple non-coding variants mapping to the same gene or disease state are ranked based on predictive weight, and wherein the predictive weight is determined by a weighing algorithm or a supervised learning model.

In an example embodiment, the method further includes providing, by the one or more computer systems and based on the identified annotated variant loci; i) a recommendation for further clinical testing ii) a disease risk prognosis; iii) a disease diagnosis; iv) a recommended therapeutic regimen or modification to an existing therapeutic regimen; or a combination thereof. In an example embodiment, the recommended therapeutic regimen or modification to an existing modification includes recommend therapeutic agents and a dosage recommendation.

In one aspect, disclosed herein a method of determining disease risk or prognosis in a subject comprising; receiving genomic data from a subject; identifying disease-specific variant loci in the genomic data; matching annotation data from a plurality of data sources comprising different data types with the corresponding identified disease-specific variant loci; converting the annotation data into a polygenic risk score using a weighting algorithm; and providing a disease diagnosis or prognosis if the polygenic risk score is above a threshold value. In an example embodiment, the annotation data is matched using the method of those described above and herein. In an example embodiment, the annotation data includes disease-specific non-coding DNA variants.

In one aspect, disclosed herein a method of treating or modifying a treatment plan comprising: obtaining genomic data from a subject to be treated or currently undergoing a treatment; identifying therapeutic agent-specific variant loci in the genomic data; matching annotation data from a plurality of data sources with the corresponding identified drug-specific variant loci; providing a therapeutic regimen for the subject based on the annotation data, the therapeutic regimen providing one or more therapeutic agent to be administering and a recommend dose and/or schedule for the one or more therapeutic agents. In an example embodiment, the annotation data is matched using the method of any one of those described above and herein. In an example embodiment, the annotations are ranked using a weighting algorithm and only those annotations meeting a defined threshold are used to determine the therapeutic regimen. In an example embodiment, the therapeutic agent-specific variant includes therapeutic-specific non-coding DNA variants.

In one aspect, disclosed herein a system to annotate genomic data, comprising: a storage device; and a processor communicatively coupled to the storage device, wherein the processor executes application code instructions that are stored in the storage device to cause the system to: a) receive, by one or more computing systems, a subject's unannotated genomic data; b) converting, by one or more computing systems, the unannotated genomic data into a standardized file format based, at least in part, on identified variant loci in the unannotated genomic data; d) generate by the one or more computing systems, annotated variant loci by matching annotation data from a plurality of data sources comprising different data types with the corresponding identified variant loci; and e) display the subject's annotated variant loci to a device associated with a user.

In an example embodiment, the standardized file format includes values for a set of match-optimized variables for each identified variant locus and configured to optimize search of the annotations from the plurality of data sources. In an example embodiment, the match-optimized variables include one or more variable selected from chromosome number, overall chromosome location, variant start position, variant stop position, variant identification number, variant type, reference allele(s), present allele(s), reference assembly number. In an example embodiment, the standardized file format includes pre-segmenting the unannotated genomic data into subsets.

In an example embodiment, the annotation data from the plurality of data sources is parsed into a matching structure to optimize a search speed of the annotation data. In an example embodiment, the matching structure includes pre-segmenting annotation data into subsets. In an example embodiment, each subset is independently stored to allow parallel searching of multiple subsets. In an example embodiment, each subset corresponds to a chromosome number. In an example embodiment, the annotation data is first pre-segmented into subsets corresponding to a chromosome number, the annotation data in each subset corresponding to a chromosome number is then further pre-segmented into additional subsets.

In an example embodiment, the annotation data in each subset is stored in a multi-dimensional array data structure. In an example embodiment, displaying annotated variant loci further includes; filtering annotation data associated with each variant based on a weight metric; and/or categorizing each annotation by annotation type. In an example embodiment, the weight metric is computed based on number of published annotations, annotation data is clinical grade, whether these annotations are based on expert panel review presence and number of conflicting annotation. In an example embodiment, the system further includes identifying conflicting annotations and selecting the annotation with a higher weight metric.

In an example embodiment, the annotation type includes risk variant type, protective variant type, drug responsiveness, metabolic effects, or any combination thereof. In an example embodiment, displaying the annotated variant loci includes generating a graphical user interface configured to facilitate ease of interpretation and visualization of data. In an example embodiment, the GUI associates a set of visual elements with each identified variant locus, each visual element representing an annotation and grouped by annotation type. In an example embodiment, the visual element further includes one or more links to additional information about the annotation. In an example embodiment, the identified variants and associated visual elements are displayed in ranked order, based at least in part, on the weight metric.

In an example embodiment, the plurality of annotation sources include genotype information, phenotype information, evidence levels, drug efficacy data, drug toxicity data metabolic data, or any combination thereof. In an example embodiment, genotype information includes non-coding DNA variant information. In an example embodiment, a connection to non-coding variants to coding genes or disease states is determined from genome-wide association studies (GWAS), CRISPR-based functional screens, or by activity-by-contact models. In an example embodiment, multiple non-coding variants mapping to the same gene or disease state are ranked based on predictive weight, and wherein the predictive weight is determined by a weighing algorithm or a supervised learning model.

In an example embodiment, the system further includes providing, by the one or more computer systems and based on the identified annotated variant loci; i) a recommendation for further clinical testing ii) a disease risk prognosis; iii) a disease diagnosis; iv) a recommended therapeutic regimen or modification to an existing therapeutic regimen; or a combination thereof. In an example embodiment, the recommended therapeutic regimen or modification to an existing modification includes recommend therapeutic agents and a dosage recommendation.

In one aspect, disclosed herein a computer program product comprising: a non-transitory computer-readable storage device having computer-executable program instructions embodied thereon that when executed by a computer cause the computer to annotate genomic data, the computer-executable program instructions comprising: a) computer-executable program instructions to receive, with one or more computing systems, a subject's unannotated genomic data; b) computer-executable program instructions to convert the unannotated genomic data into a standardized file format based, at least in part, on identified variant loci in the unannotated genomic data; c) computer-executable program instructions to generate annotated variant loci by matching annotation data from a plurality of data sources comprising different data types with the corresponding identified variant loci; and d) computer-executable program instructions to display the subject's annotated variant loci.

In an example embodiment, the standardized file format includes values for a set of match-optimized variables for each identified variant locus and configured to optimize search of the annotations from the plurality of data sources. In an example embodiment, the match-optimized variables include one or more variable selected from chromosome number, overall chromosome location, variant start position, variant stop position, variant identification number, variant type, reference allele(s), present allele(s), reference assembly number. In an example embodiment, the standardized file format includes pre-segmenting the unannotated genomic data into subsets.

In an example embodiment, the annotation data from the plurality of data sources is parsed into a matching structure to optimize a search speed of the annotation data. In an example embodiment, the matching structure includes pre-segmenting annotation data into subsets. In an example embodiment, each subset is independently stored to allow parallel searching of multiple subsets. In an example embodiment, each subset corresponds to a chromosome number. In an example embodiment, the annotation data is first pre-segmented into subsets corresponding to a chromosome number, the annotation data in each subset corresponding to a chromosome number is then further pre-segmented into additional subsets.

In an example embodiment, the annotation data in each subset is stored in a multi-dimensional array data structure. In an example embodiment, displaying annotated variant loci further includes; filtering annotation data associated with each variant based on a weight metric; and/or categorizing each annotation by annotation type. In an example embodiment, the weight metric is computed based on number of published annotations, annotation data is clinical grade, whether these annotations are based on expert panel review presence and number of conflicting annotation. In an example embodiment, the product further includes identifying conflicting annotations and selecting the annotation with a higher weight metric.

In an example embodiment, the annotation type includes risk variant type, protective variant type, drug responsiveness, metabolic effects, or any combination thereof. In an example embodiment, displaying the annotated variant loci includes generating a graphical user interface configured to facilitate ease of interpretation and visualization of data. In an example embodiment, the GUI associates a set of visual elements with each identified variant locus, each visual element representing an annotation and grouped by annotation type. In an example embodiment, the visual element further includes one or more links to additional information about the annotation. In an example embodiment, the identified variants and associated visual elements are displayed in ranked order, based at least in part, on the weight metric.

In an example embodiment, the plurality of annotation sources include genotype information, phenotype information, evidence levels, drug efficacy data, drug toxicity data metabolic data, or any combination thereof. In an example embodiment, genotype information includes non-coding DNA variant information. In an example embodiment, a connection to non-coding variants to coding genes or disease states is determined from genome-wide association studies (GWAS), CRISPR-based functional screens, or by activity-by-contact models. In an example embodiment, multiple non-coding variants mapping to the same gene or disease state are ranked based on predictive weight, and wherein the predictive weight is determined by a weighing algorithm or a supervised learning model.

In an example embodiment, the product further includes providing, by the one or more computer systems and based on the identified annotated variant loci; i) a recommendation for further clinical testing ii) a disease risk prognosis; iii) a disease diagnosis; iv) a recommended therapeutic regimen or modification to an existing therapeutic regimen; or a combination thereof. In an example embodiment, the recommended therapeutic regimen or modification to an existing modification includes recommend therapeutic agents and a dosage recommendation.

These and other aspects, objects, features, and advantages of the example embodiments will become apparent to those having ordinary skill in the art upon consideration of the following detailed description of example embodiments.

Standard techniques related to making and using aspects of the invention may or may not be described in detail herein. Various aspects of computing systems and specific computer programs to implement the various technical features described herein are well known.

### EXAMPLES

In a first example, the method can include: receiving a subject's unannotated genomic data, converting the unannotated genomic data into a standardized format, generating annotated variant loci based on annotation data from a plurality of data sources, and optionally displaying the subject's annotated variant loci. Converting the unannotated genomic data into a standardized format can include determining a variable value set for each identified variant locus (e.g., wherein a variant locus can be a locus corresponding to a genetic variant) in the unannotated genomic data, wherein each variable value set can include values for one or more variables (e.g., match optimized variables). Examples of variables can include: chromosome number, overall chromosome location, variant start position, variant stop position, variant identification number, variant type, reference allele(s), present allele(s), reference assembly number, and/or any other genomic information. The annotation data can include annotations mapped to variable value sets (e.g., variable value sets for coding and/or non-coding DNA variants). In a specific example, when multiple annotations correspond to the same variable value set, a weighted aggregation can be performed across the multiple annotations (e.g., filtering out annotations with weights below a threshold, ranking annotations according to their respective weights, selecting the annotation with the highest weight between two conflicting annotations, etc.). In an example, the annotation data can be segmented into subsets (e.g., segments) of annotation data, each subset corresponding to a search region spanning a set of loci. Generating annotated variant loci can include, for each identified variant locus (corresponding to a variable value set) in the unannotated genomic data: selecting a search region from the set of search regions (e.g., selecting the search region containing the variant locus, selecting a search region adjacent to the search region containing the variant locus, etc.), and searching within the subset of annotation data corresponding to the selected search region to identify annotations associated with a matching variable value set. The identified variant locus can be annotated with one or more identified annotations. In an illustrative example, for a subject with a variant (‘variant A’) at locus 15 of chromosome 1, the variant A can be represented by a corresponding variable value set. The variable value set can then be compared to variable value sets in a subset of the annotation data (e.g., in a subset corresponding to loci 1-10, in a subset corresponding to loci 11-20, etc.) to identify annotations associated with variant A at locus 15. Illustrative examples of annotations for variant A at locus 15 can include: variant A at locus 15 is associated with an increased risk of breast cancer; any variant at locus 15 is associated with an increased risk of breast cancer; variants found in loci 1-20 are associated with increased risk of breast cancer; locus 15 is associated with the inflammation functional category; patients with variant A at locus 15 and a phenotype (e.g., cystic fibrosis) may respond to a specific treatment (e.g., ivacaftor); and/or any other annotation.

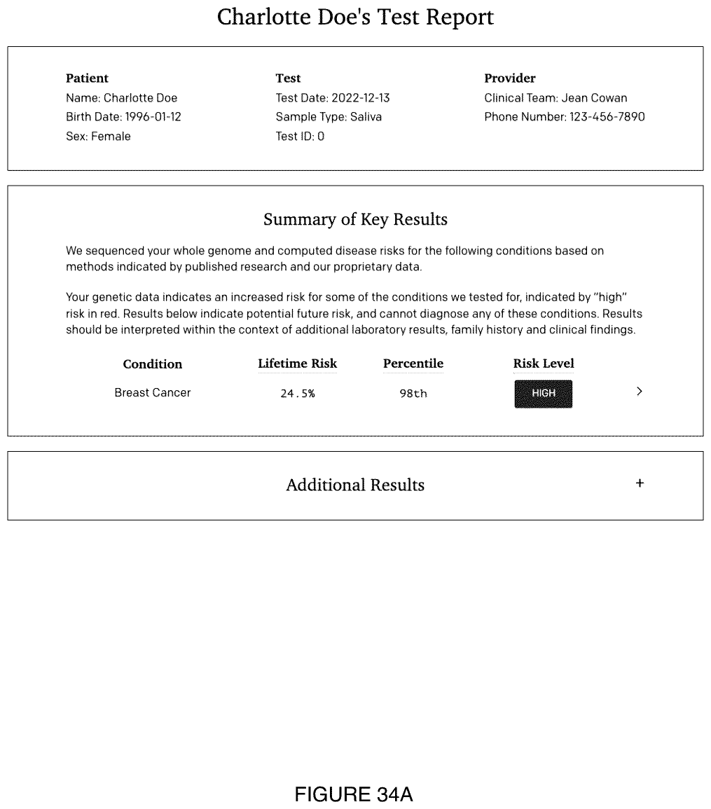

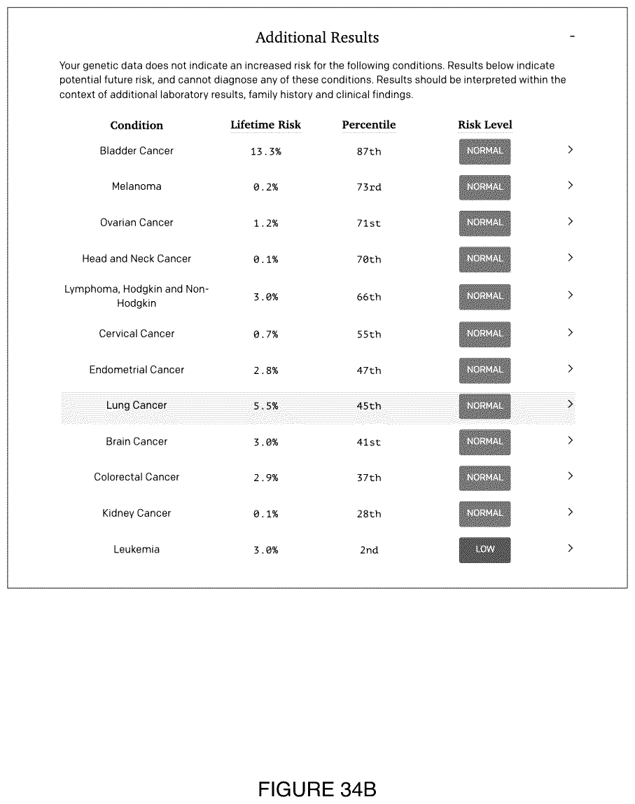

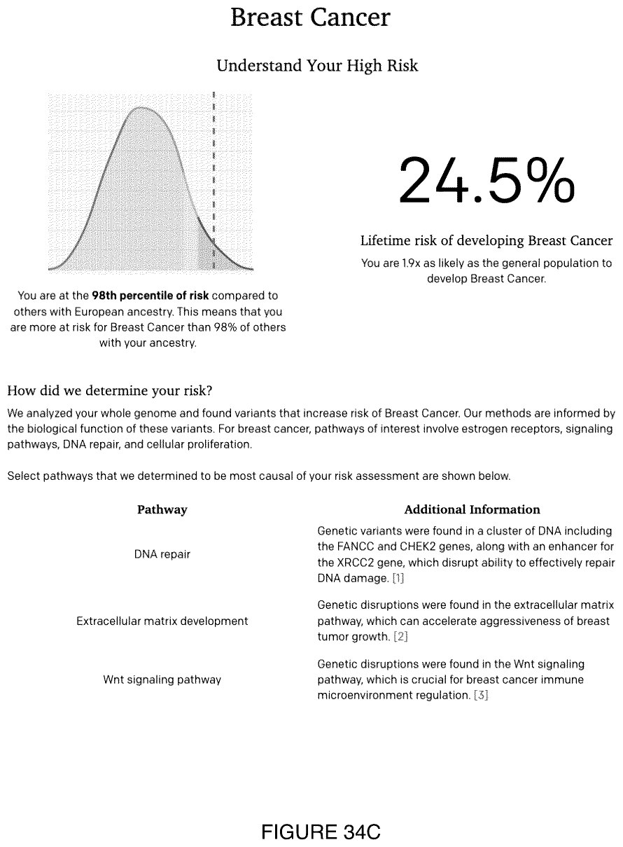

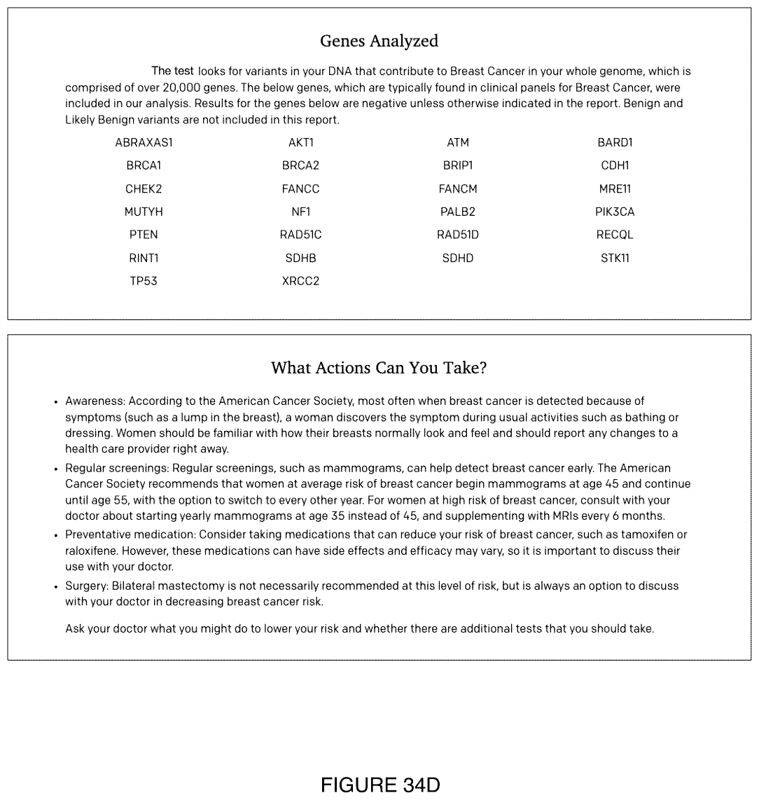

In a second example, the method can include: receiving a subject's unannotated genomic data, optionally generating annotated variant loci, determining a risk score for the subject (e.g., using a risk model), and optionally analyzing the risk score. In a first specific example, the risk score can be a genomic risk score for a disease of interest determined using a (trained) genomic risk model, based on unannotated and/or annotated identified variant loci in the genomic data for the subject. The identified variant loci can correspond to coding loci and/or noncoding loci. In a second specific example, the risk score can be a composite risk score for the disease of interest determined using a (trained) composite risk model, based on the genomic risk score and clinical features (e.g., demographic data, family history, clinical results, etc.) for the subject. The risk score (e.g., the genomic risk score and/or the composite risk score) can optionally be used to determine: treatment recommendations, a lifetime risk score, a percentile risk for the subject relative to a reference population, and/or any other information. The risk model (e.g., genomic risk model) can be trained using population genomic data labeled with a disease label (e.g., the risk model can be trained to predict, based on training genomic data, a risk score corresponding to the disease label for the training genomic data). Training the risk model can include: segmenting a set of loci into a set of functional groups based on functional data (e.g., annotation data from a plurality of data sources), wherein each functional group corresponds to a disease pathway; and training the risk model using a set of priors associated with the functional groups. The set of loci can include coding loci and/or non-coding loci. In an example, non-coding loci can be greater than a threshold percentage of the set of loci (e.g., greater than 20%, greater than 50%, greater than 60%, greater than 70%, greater than 80%, greater than 90%, etc.). In an example, the set of priors can include an initial weight corresponding to each functional group (e.g., weight corresponding to each locus within the functional group), wherein the initial weight for each functional group is determined based on the respective disease pathway (e.g., whether the disease pathway that the functional group is associated with is relevant to the disease of interest). Training the risk model can include updating the initial weights (e.g., individually updating weights for each locus and/or updating a weight corresponding to all loci within a functional group). In a specific example, analyzing the risk score for the subject can include: determining a contribution to the risk score due to a functional group and/or due to one or more variant loci, and optionally determining a subset of functional groups (and corresponding disease pathways) and/or variant loci with the highest contribution to the risk score. Risk analysis results can optionally be displayed (e.g., examples shown in FIG. 34A-34D).

### Technical Advantages

Variants of the technology can confer one or more advantages over conventional technologies.

In an example, variants of the technology can train a functionally informed whole-genome predictive machine learning (ML) model for disease risk. This new polygenic prediction model can combine the power of a Bayesian supervised learning method that leverages trait-specific functional prior annotations, LDpred-funct, and an enhancer-gene connection framework, called activity-by-contact (ABC)-the first and most accurate method to comprehensively map the function of non-coding regions across the genome, creating a robust prior set for the Bayesian supervised learning model. Preliminary work using Genome-in-a-Bottle samples and in silico sequences showed that this ML model successfully incorporates the 90% of common variants in the non-coding genome using new functional annotations. In a specific example, the method can include incorporating functional information into the development of risk scores rather than developing them purely based on associations in GWAS. This approach not only improves ethnic generalizability of risk scores, it also improves the interpretability of results. Developing such functionally-informed methods requires data on genomic function across the whole genome; the lack of data on the non-coding genome previously was an obstacle. While once thought of as “junk DNA,” the non-coding genome plays a key functional role in disease by regulating the expression of coding elements. Recent evidence has shown that over 90% of variants causal for common diseases, including cardiovascular disease and cancers, lie in non-coding regions of the genome. Indeed, most causal variants in GWAS, which are key to developing polygenic risk scores, do not directly alter protein-coding sequences and instead occur in non-coding gene regulatory elements such as enhancers; enhancers control how genes are expressed in specific cell types and harbor most genetic variants that influence risk for common diseases. Therefore, there has been an unmet need for systematic functional mapping of the non-coding genome in use for polygenic predictive risk models.

However, further advantages can be provided by the system and method disclosed herein.

### Example System Architectures

Turning now to the drawings, in which like numerals represent like (but not necessarily identical) elements throughout the figures, example embodiments are described in detail.

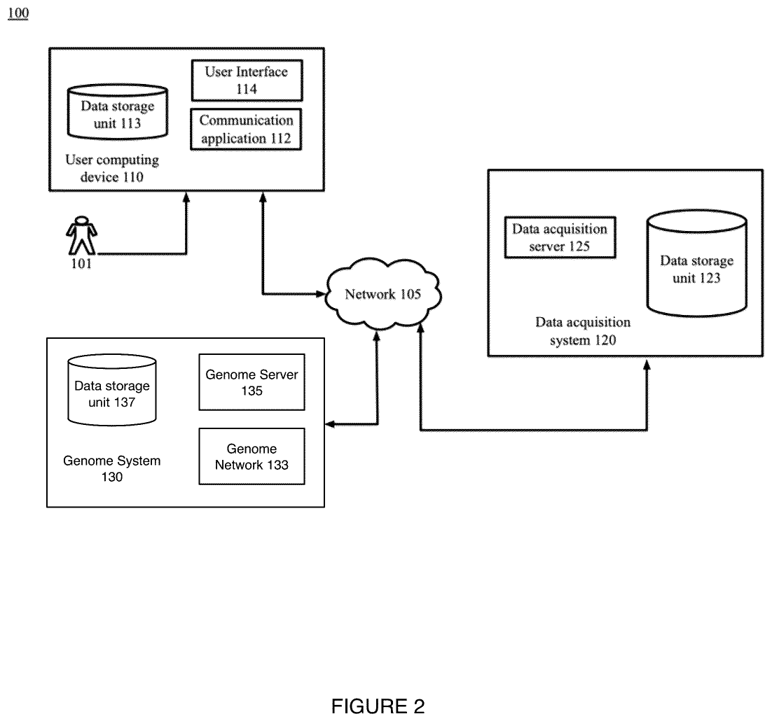

FIG. 2 is a block diagram depicting a system 100 to annotate genomic data and/or determine risk scores. In one example embodiment, a user 101 associated with a user computing device 110 must install an application, and or make a feature selection to obtain the benefits of the techniques described herein. In example embodiments, the user 101 is the subject associated with the genomic data. In example embodiments, the user 101 is not the subject associated with the genomic data but provides the subject's unannotated genomic data for receiving by the genome system 130.

As depicted in FIG. 2, the system 100 includes network computing devices/systems 110, 120, and 130 that are configured to communicate with one another via one or more networks 105 or via any suitable communication technology.

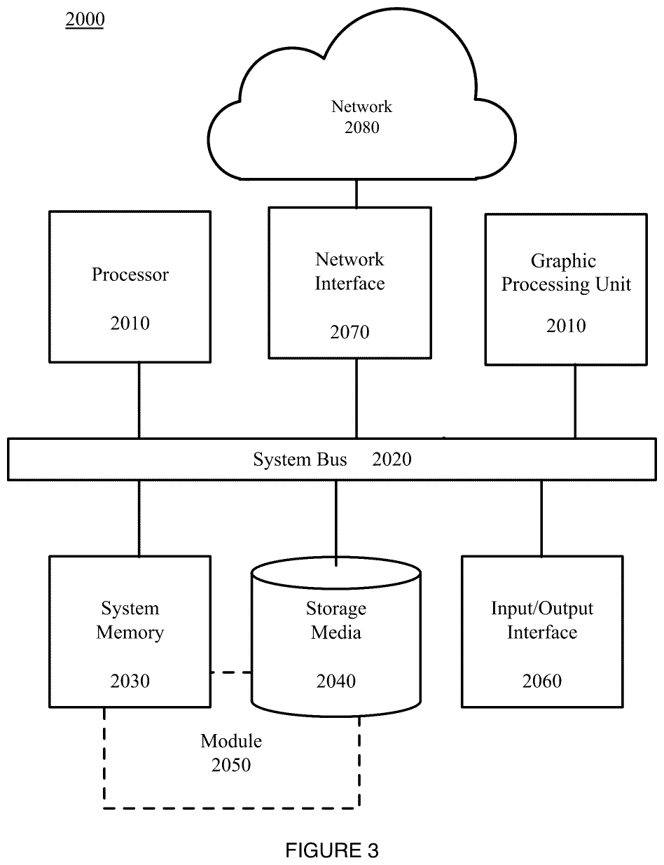

Each network 105 includes a wired or wireless telecommunication means by which network devices/systems (including devices 110, 120, and 130) can exchange data. For example, each network 105 can include any of those described herein such as the network 2080 described in FIG. 3 or any combination thereof or any other appropriate architecture or system that facilitates the communication of signals and data. Throughout the discussion of example embodiments, it should be understood that the terms “data” and “information” are used interchangeably herein to refer to text, images, audio, video, or any other form of information that can exist in a computer-based environment. The communication technology utilized by the devices/systems 110, 120, and 130 may be similar networks to network 105 or an alternative communication technology.

Each network computing device/system 110, 120, and 130 includes a computing device having a communication module capable of transmitting and receiving data over the network 105 or a similar network. For example, each network device/system 110, 120, and 130 can include any computing machine 2000 described herein and found in FIG. 3 or any other wired or wireless, processor-driven device. In the example embodiment depicted in FIG. 2, the network devices/systems 110, 120, and 130 are operated by user 101, data acquisition system operators, and genome system operators, respectively.

The user computing device 110 includes a user interface 114. The user interface 114 may be used to display a graphical user interface and other information to the user 101 to allow the user 101 to interact with the data acquisition system 120, the genome system 130, and others. The user interface 114 receives user input for data acquisition and/or genome annotation and displays results to user 101. In another example embodiment, the user interface 114 may be provided with a graphical user interface by the data acquisition system 120 and or the genome system 130. The user interface 114 may be accessed by the processor of the user computing device 110. The user interface may display 114 may display a webpage associated with the data acquisition system 120 and/or the genome system 130. The user interface 114 may be used to provide input, configuration data, and other display direction by the webpage of the data acquisition system 120 and/or the genome system 130. In another example embodiment, the user interface 114 may be managed by the data acquisition system 120, the genome system 130, or others. In another example embodiment, the user interface 114 may be managed by the user computing device 110 and be prepared and displayed to the user 101 based on the operations of the user computing device 110.

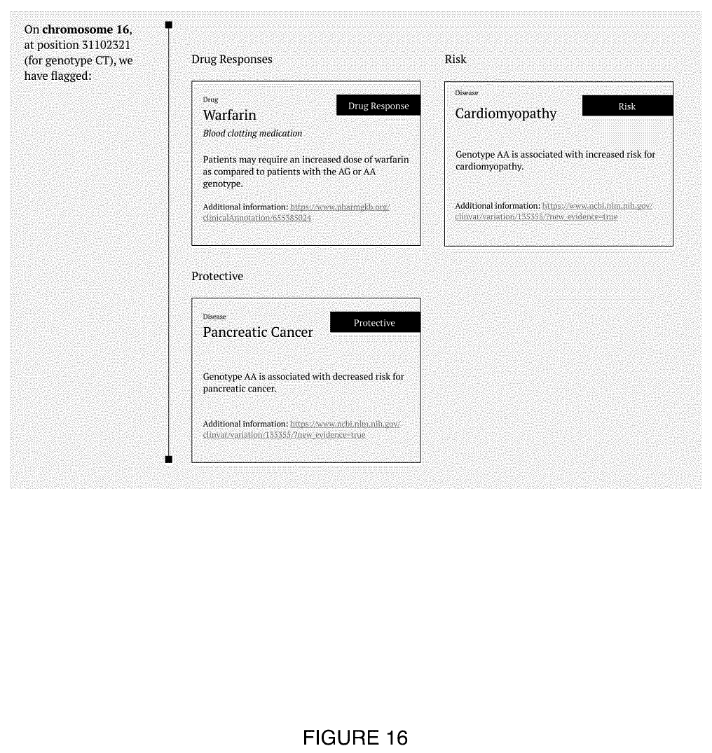

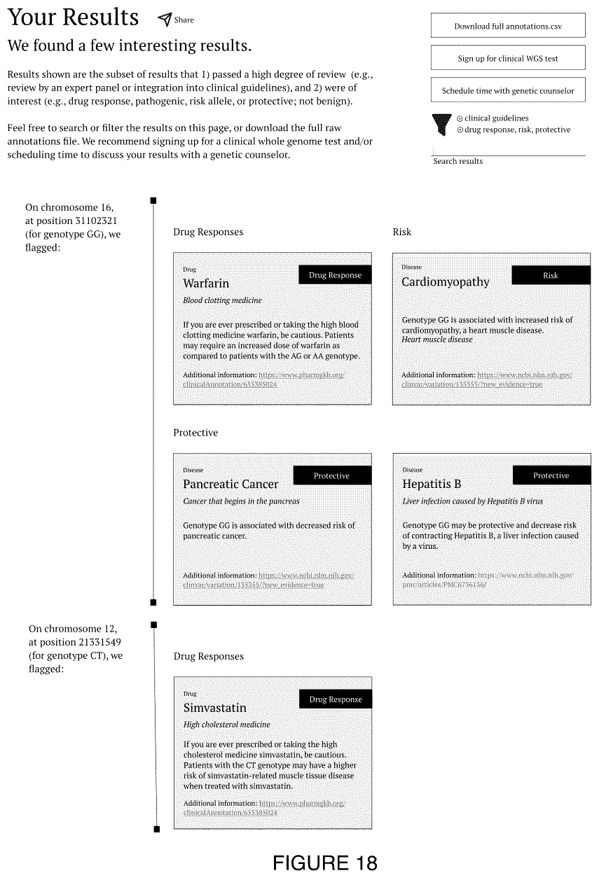

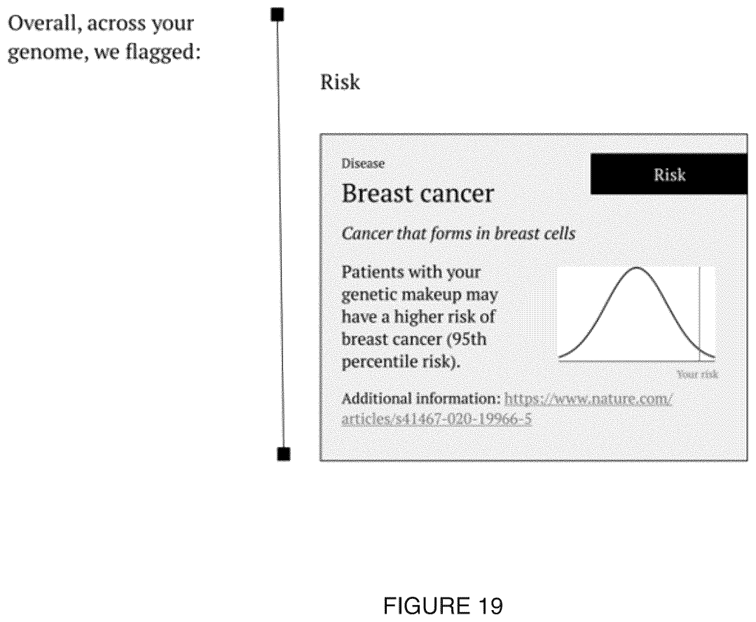

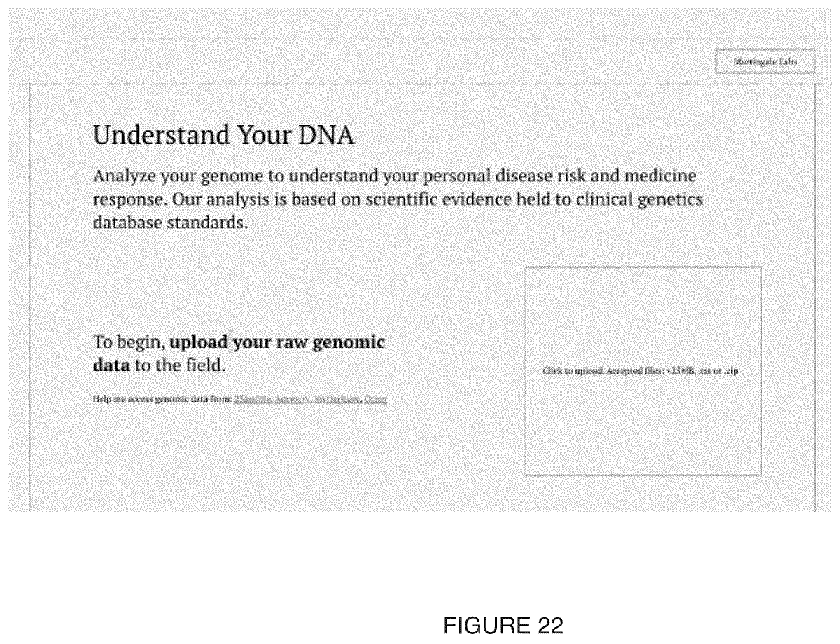

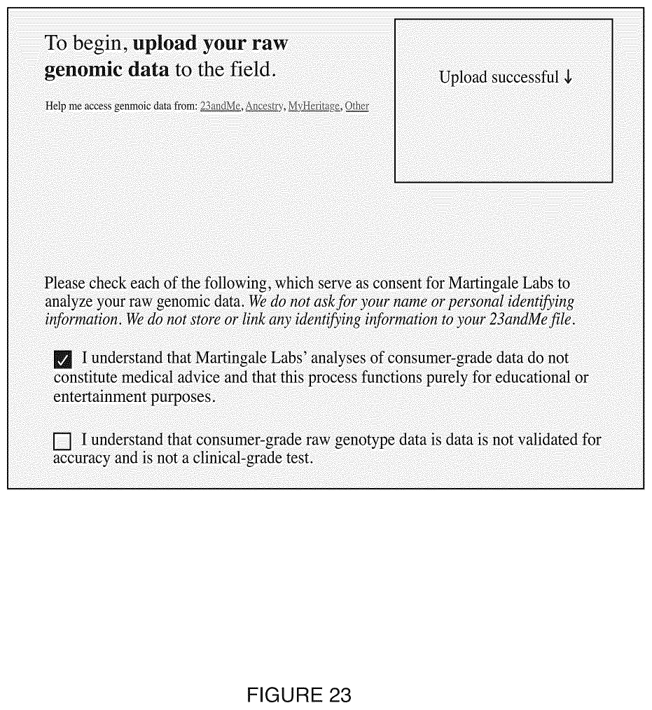

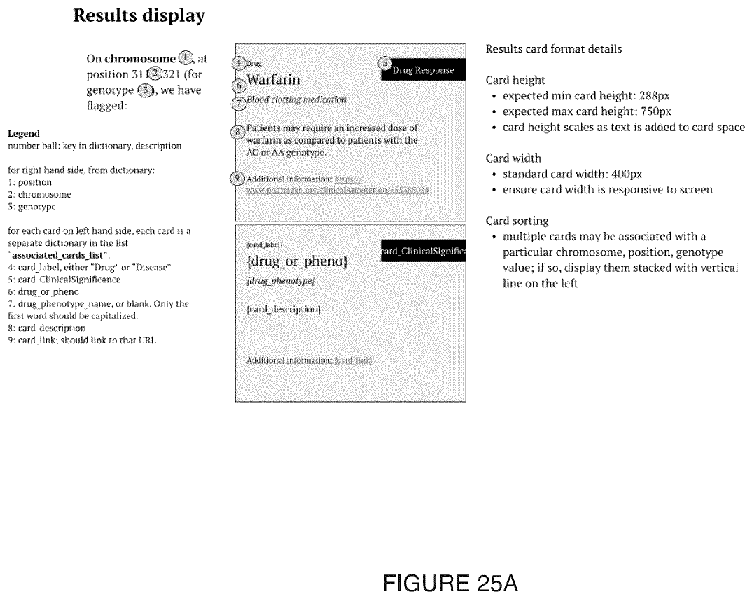

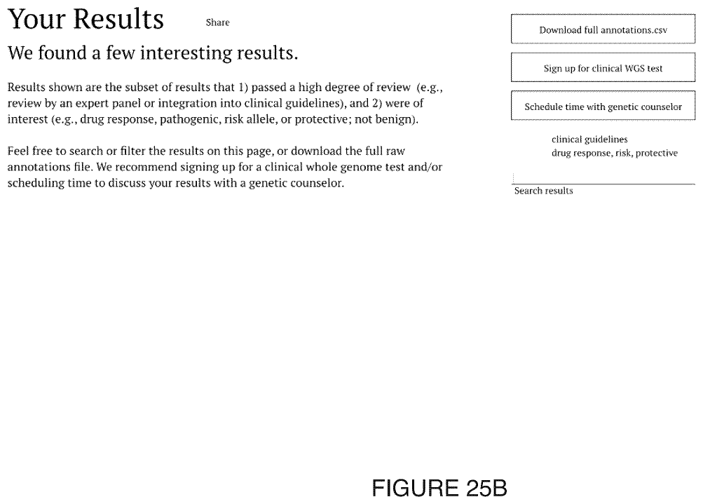

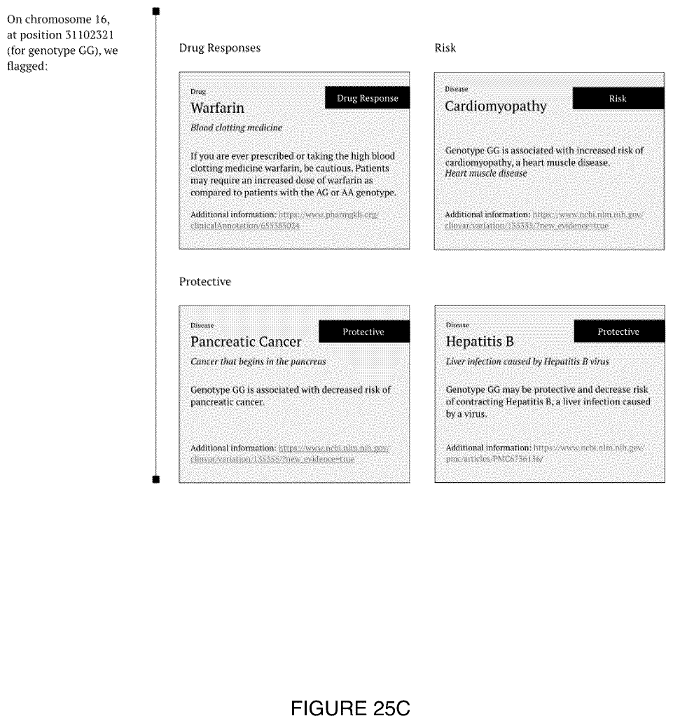

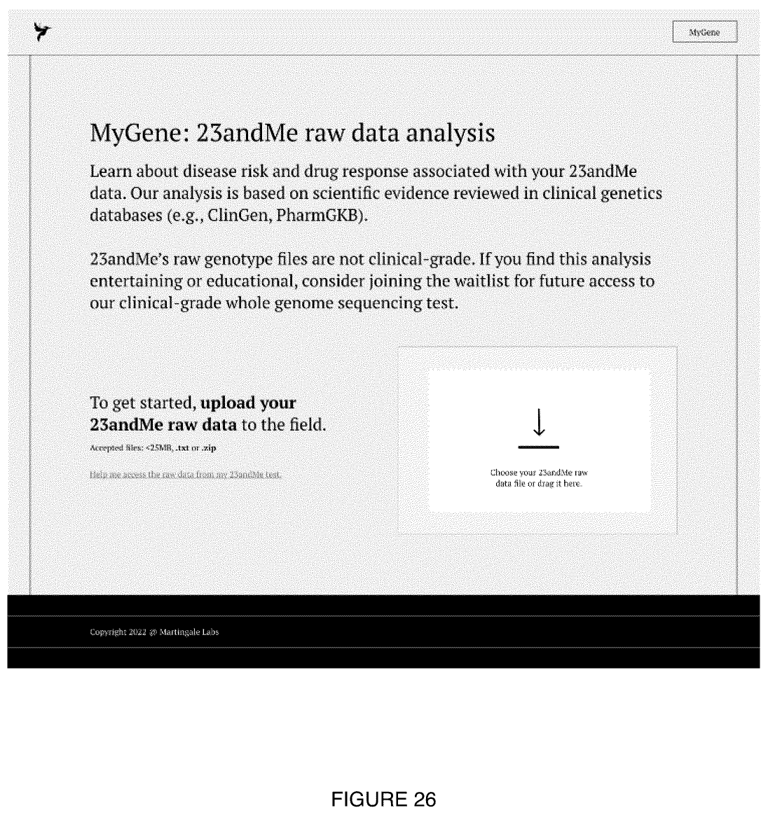

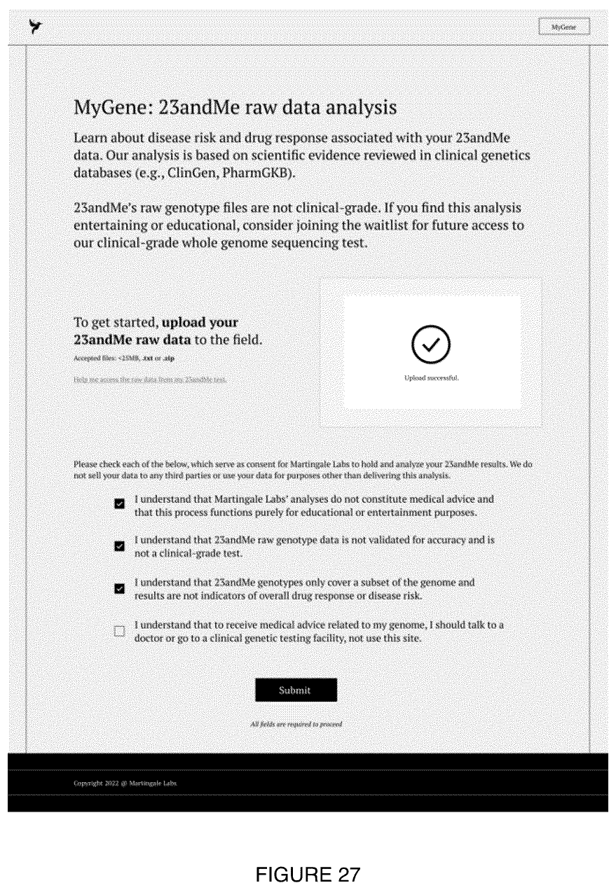

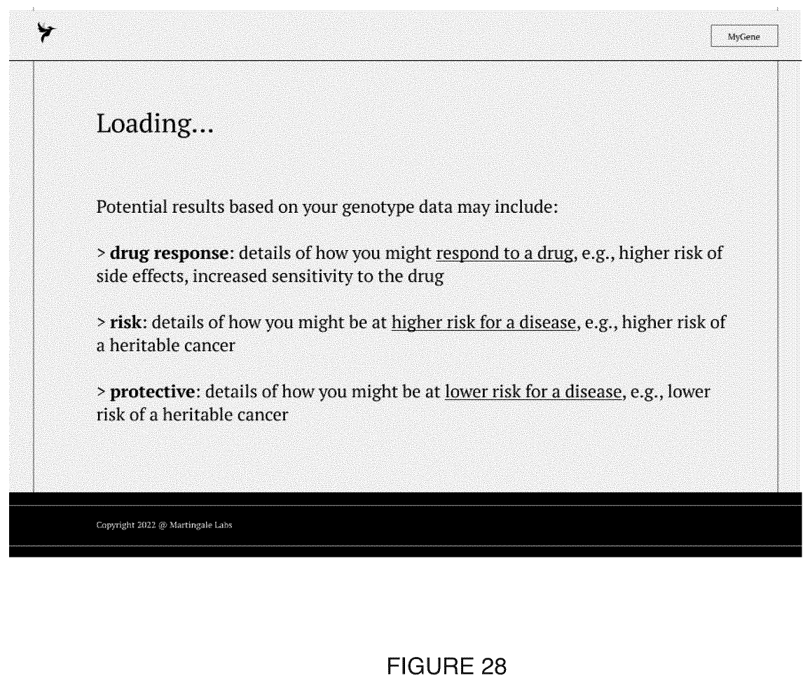

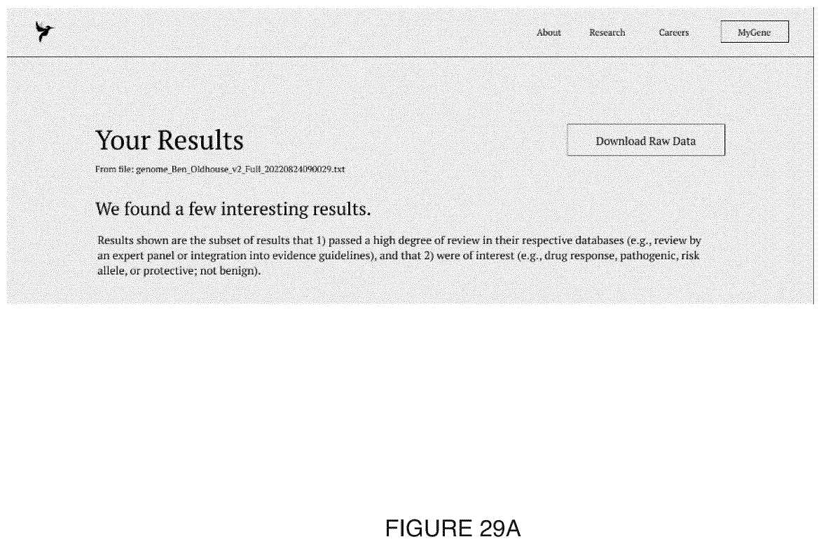

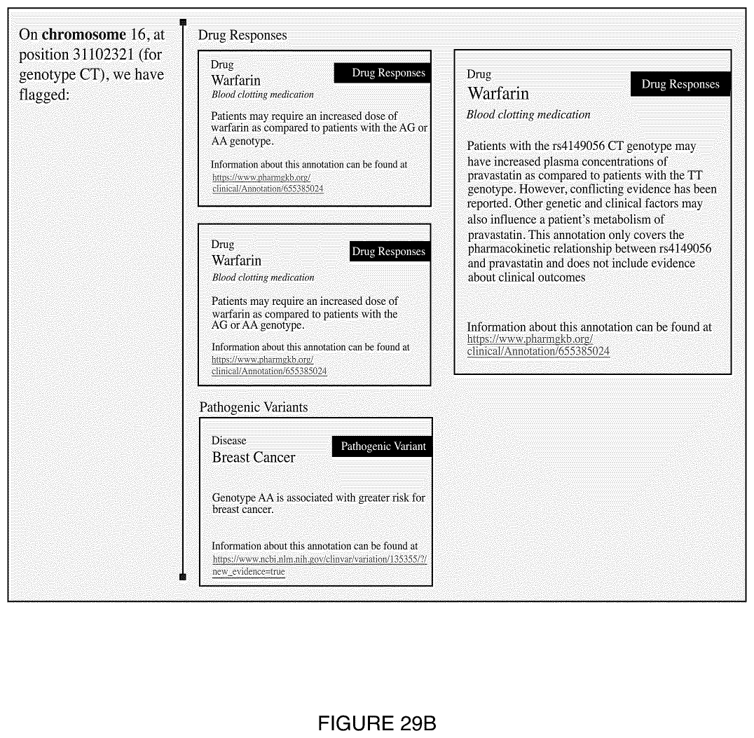

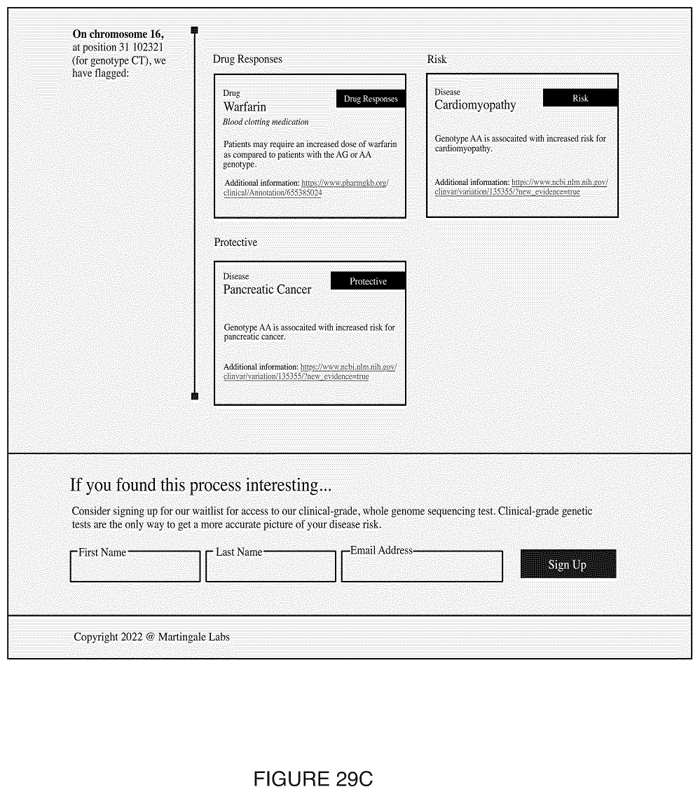

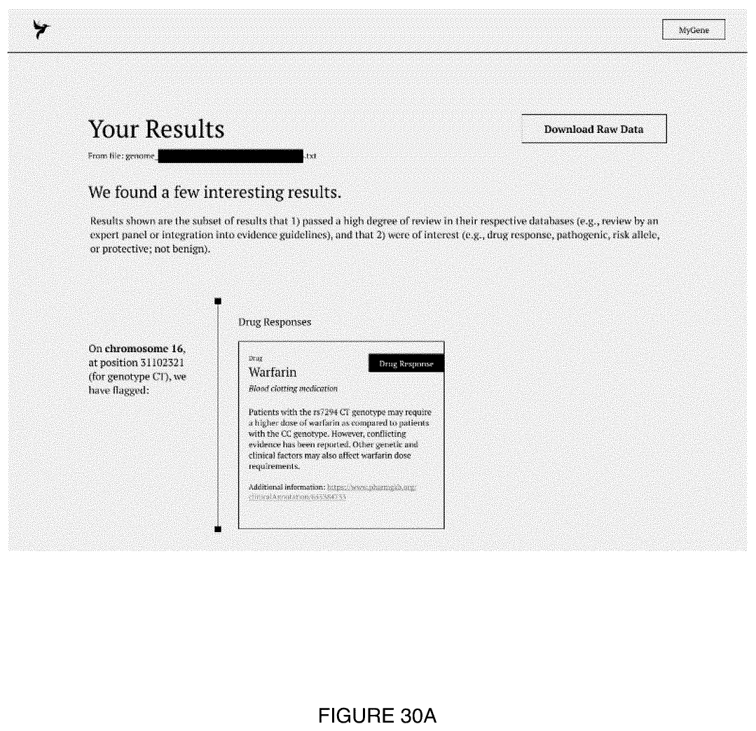

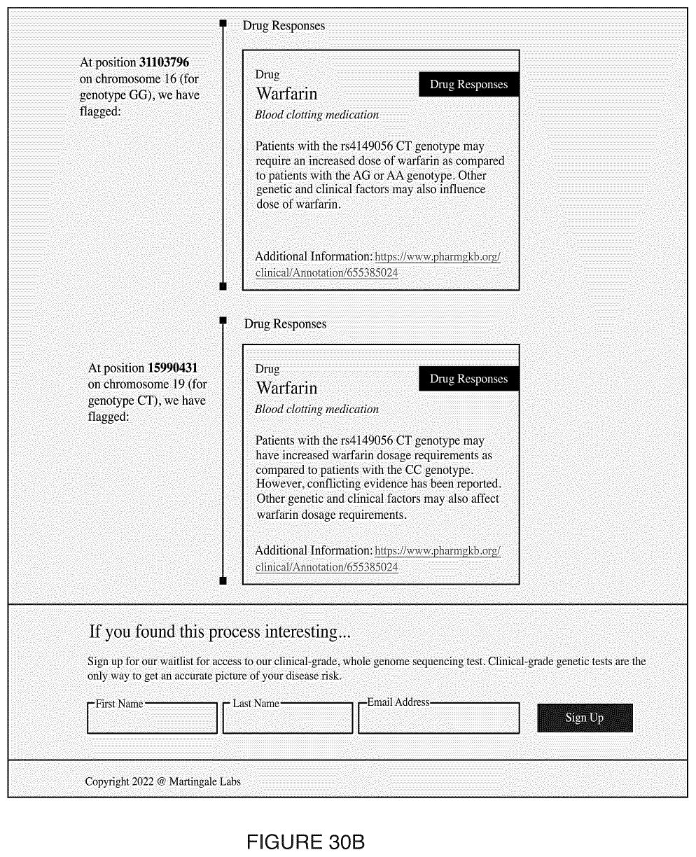

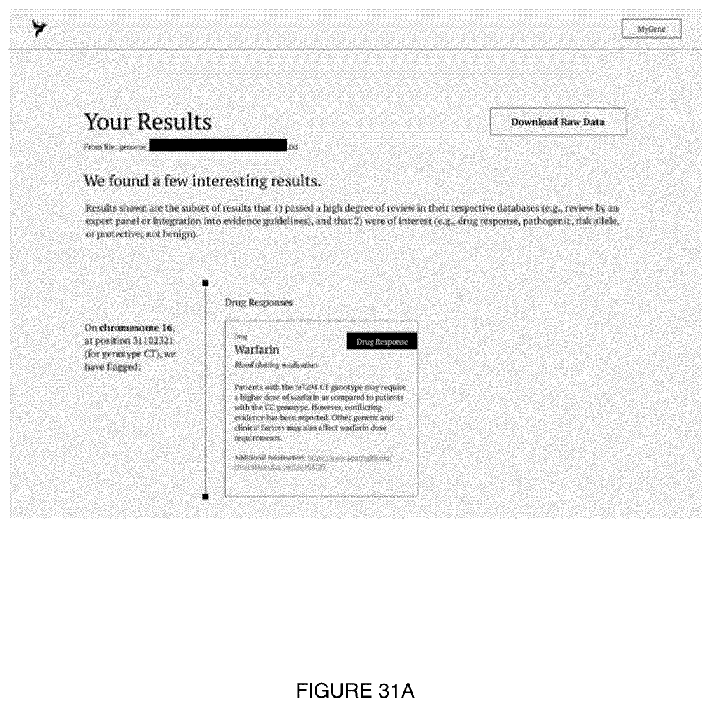

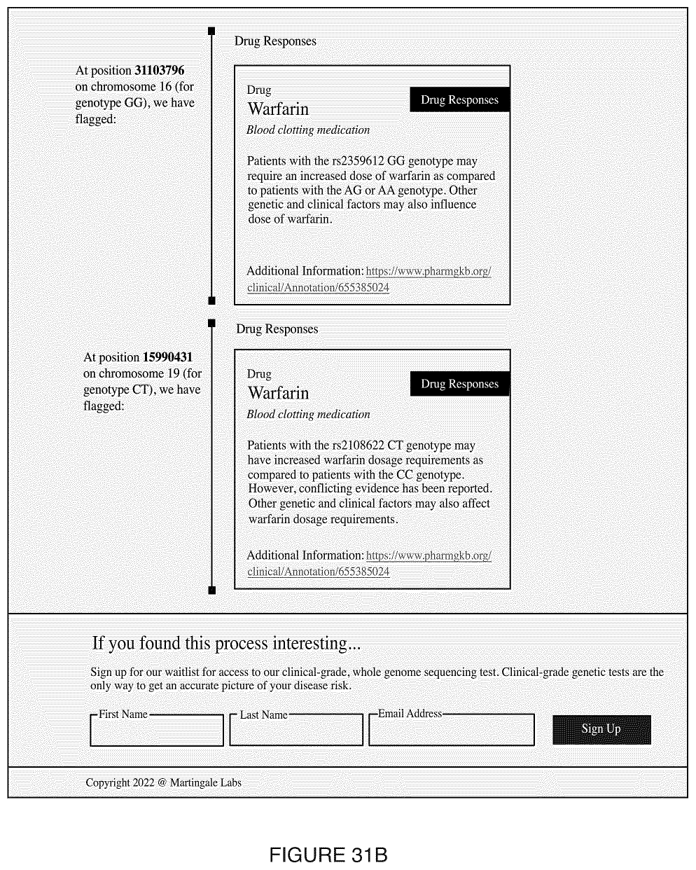

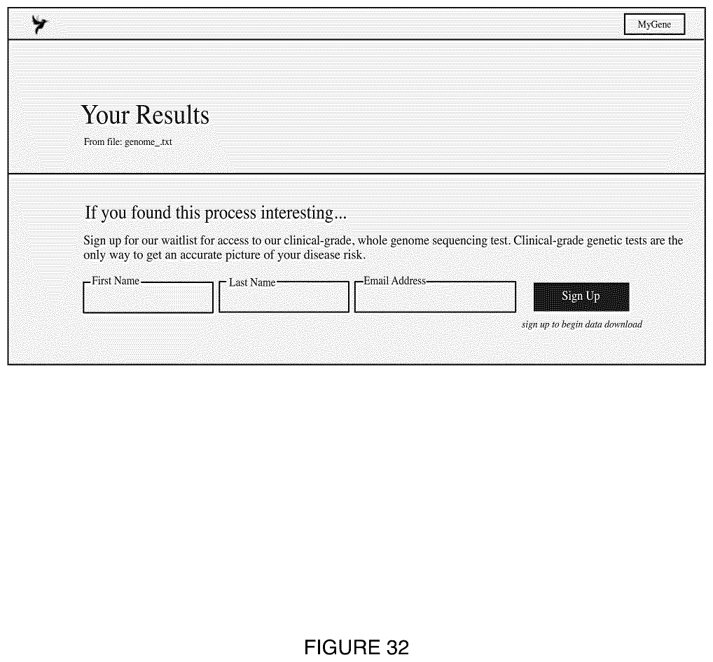

Examples of displays at the user interface 114 are shown in FIG. 16, FIG. 18, FIG. 19, FIG. 22, FIG. 23, FIG. 25A, FIG. 25B, FIG. 25C, FIG. 26, FIG. 27, FIG. 28, FIG. 29A, FIG. 29B, FIG. 29C, FIG. 30A, FIG. 30B, FIG. 31A, FIG. 31B, and FIG. 32.

The user 101 can use the communication application 112 on the user computing device 110, which may be, for example, a web browser application or a stand-alone application, to view, download, upload, or otherwise access documents or web pages through the user interface 114 via the network 105. The user computing device 110 can interact with the web servers or other computing devices connected to the network, including the data acquisition server 125 of the data acquisition system 120 and the genome server 135 of the genome system 130. In another example embodiment, the user computing device 110 communicates with devices in the data acquisition system 120 and/or the genome system 130 via any other suitable technology, including the example computing system described below.

The user computing device 110 also includes a data storage unit 113 accessible by the user interface 114, the communication application 112, or other applications. The example data storage unit 113 can include one or more tangible computer-readable storage devices. The data storage unit 113 can be stored on the user computing device 110 or can be logically coupled to the user computing device 110. For example, the data storage unit 113 can include on-board flash memory and/or one or more removable memory accounts or removable flash memory. In another example embodiments, the data storage unit 113 may reside in a cloud-based computing system.

An example data acquisition system 120 includes a data storage unit 123 and an acquisition server 125. The data storage unit 123 can include any local or remote data storage structure accessible to the data acquisition system 120 suitable for storing information. The data storage unit 123 can include one or more tangible computer-readable storage devices, or the data storage unit 123 may be a separate system, such as a different physical or virtual machine or a cloud-based storage service.

In one aspect, the data acquisition server 125 communicates with the user computing device 110 and/or the genome system 130 to transmit requested data. The data may include genomic data.

An example genome system 130 includes a machine learning system 133, a genome server 135, and a data storage unit 137. The genome server 135 communicates with the user computing device 110 and/or the data acquisition system 120 to request and receive data. The data may include the data types previously described in reference to the data acquisition server 125.

The genome network 133 receives an input of data from the genome server 135. The genome network 133 can include one or more functions to implement any of the mentioned methods (e.g., genome annotations methods to annotate genomic data of a subject, risk score methods to determine risk scores and/or risk score analyses, etc.). In a preferred embodiment, the genome network may include match-optimized variables. In an example embodiment, the genome network may include pre-segmenting unannotated genomic data into subsets. In an example embodiment, the genome network may include parsing the annotation data from the plurality of data sources into a matching structure to optimize a search speed of the annotation data. Any suitable architecture may be applied to annotate genomic data and/or determine risk scores.

The data storage unit 137 can include any local or remote data storage structure accessible to the genome system 130 suitable for storing information. The data storage unit 137 can include one or more tangible computer-readable storage devices, or the data storage unit 137 may be a separate system, such as a different physical or virtual machine or a cloud-based storage service.

In an alternate embodiment, the functions of either or both of the data acquisition system 120 and the genome system 130 may be performed by the user computing device 110.

It will be appreciated that the network connections shown are examples, and other means of establishing a communications link between the computers and devices can be used. Moreover, those having ordinary skill in the art having the benefit of the present disclosure will appreciate that the user computing device 110, data acquisition system 120, and the genome system 130 illustrated in FIG. 2 can have any of several other suitable computer system configurations. For example, a user computing device 110 embodied as a mobile phone or handheld computer may not include all the components described above.

In example embodiments, the network computing devices and any other computing machines associated with the technology presented herein may be any type of computing machine such as, but not limited to, those discussed in more detail with respect to FIG. 3. Furthermore, any modules associated with any of these computing machines, such as modules described herein or any other modules (scripts, web content, software, firmware, or hardware) associated with the technology presented herein may by any of the modules discussed in more detail with respect to FIG. 3. The computing machines discussed herein may communicate with one another as well as other computer machines or communication systems over one or more networks, such as network 105. The network 105 may include any type of data or communications network, including any of the network technology discussed with respect to FIG. 3.

### Example Processes

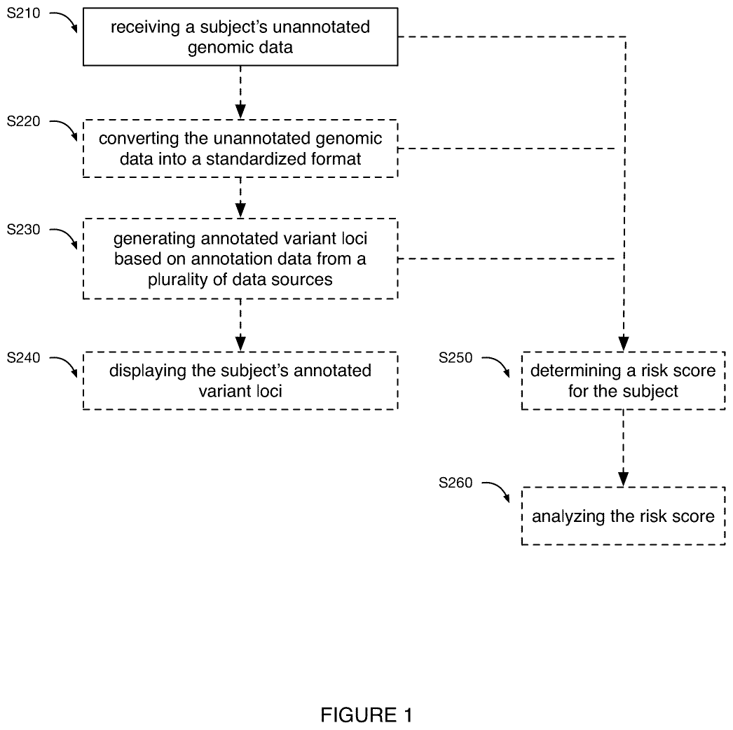

The example methods illustrated in FIG. 1 is described hereinafter with respect to the components of the example architecture 100. The example methods also can be performed with other systems and in other architectures including similar elements. All or portions of the method can be performed by one or more components of the system, using a computing system, using a database (e.g., a system database, a third-party database, etc.), by a user, and/or by any other suitable system.

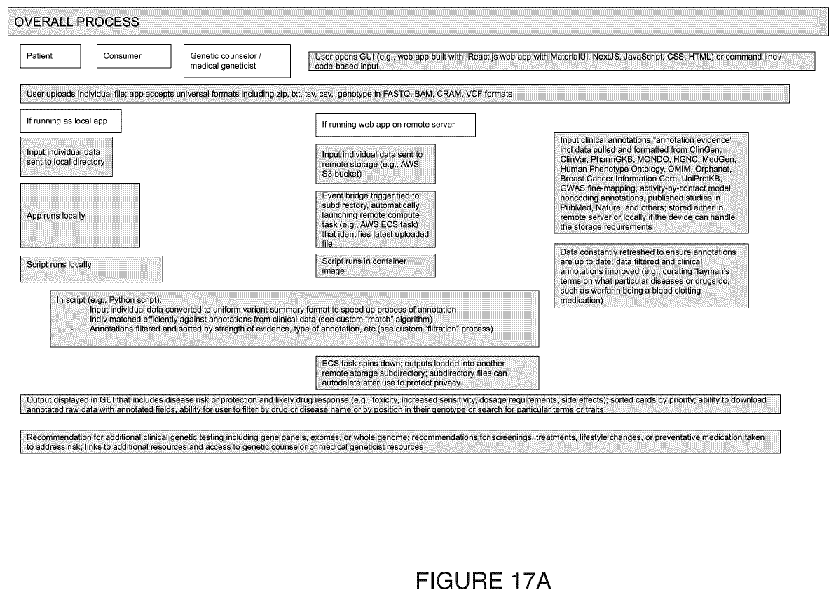

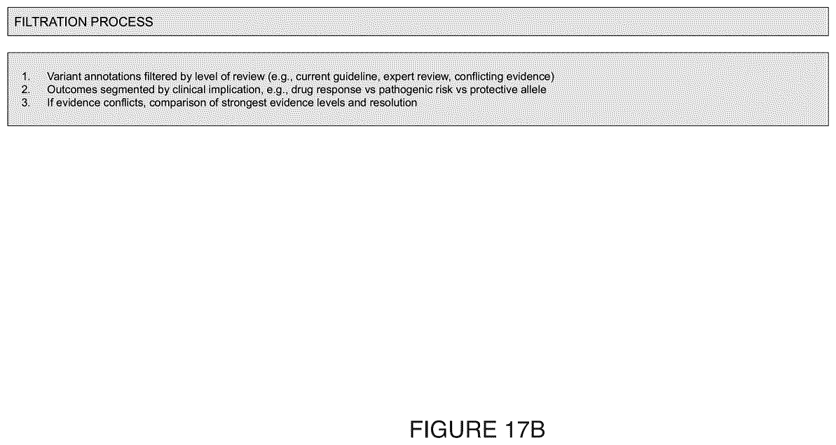

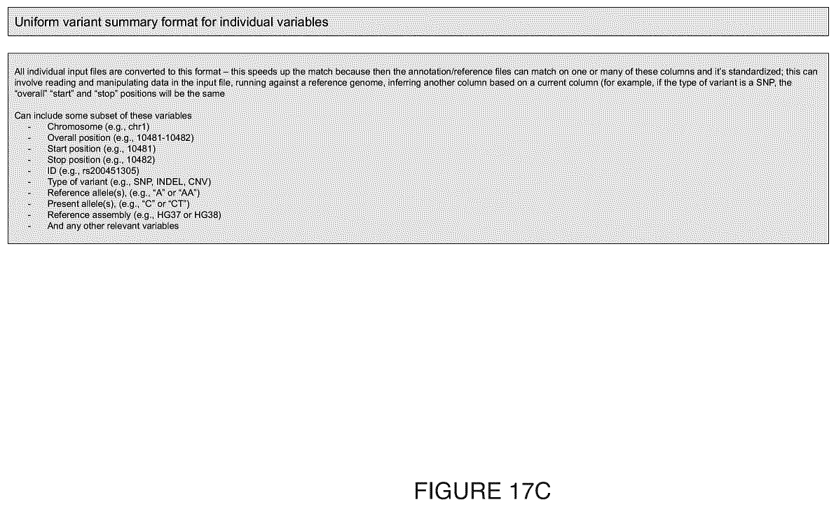

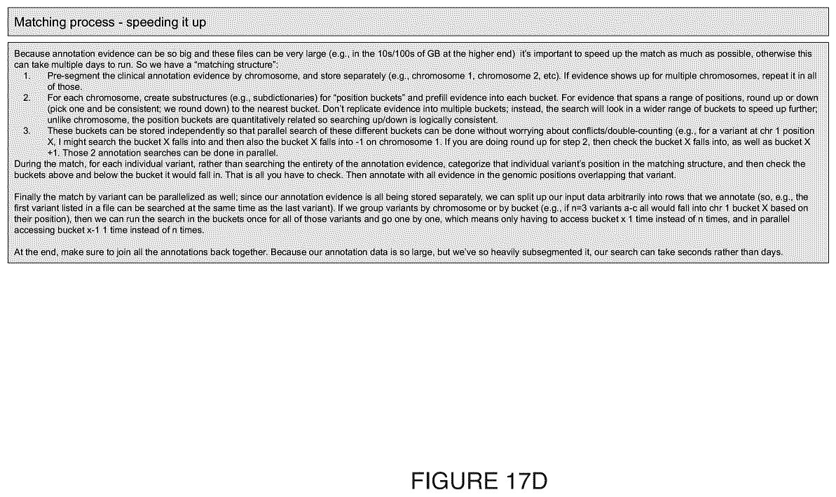

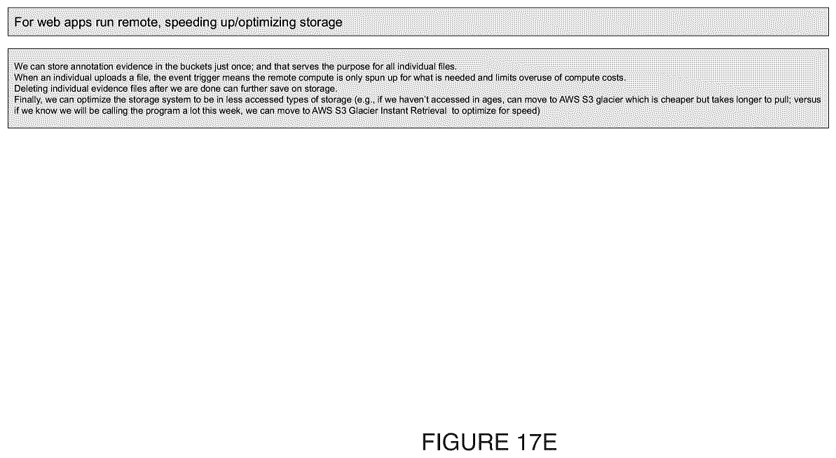

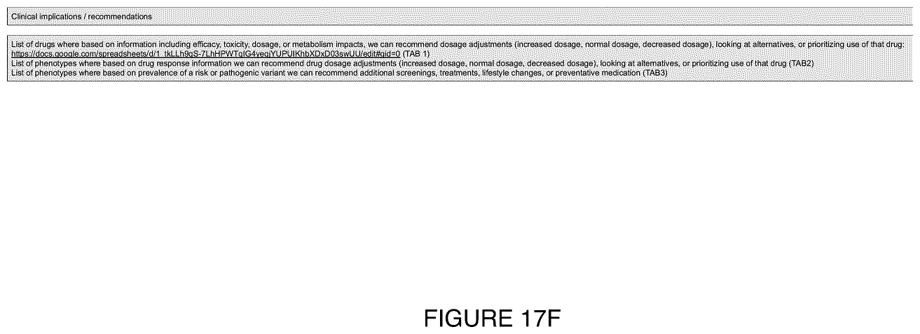

Referring to FIG. 1, and continuing to refer to FIG. 2 for context, a block flow diagram illustrates methods 200, in accordance with certain examples of the technology disclosed herein. Examples are shown in FIG. 17A, FIG. 17B, FIG. 17C, FIG. 17D, and FIG. 17E.

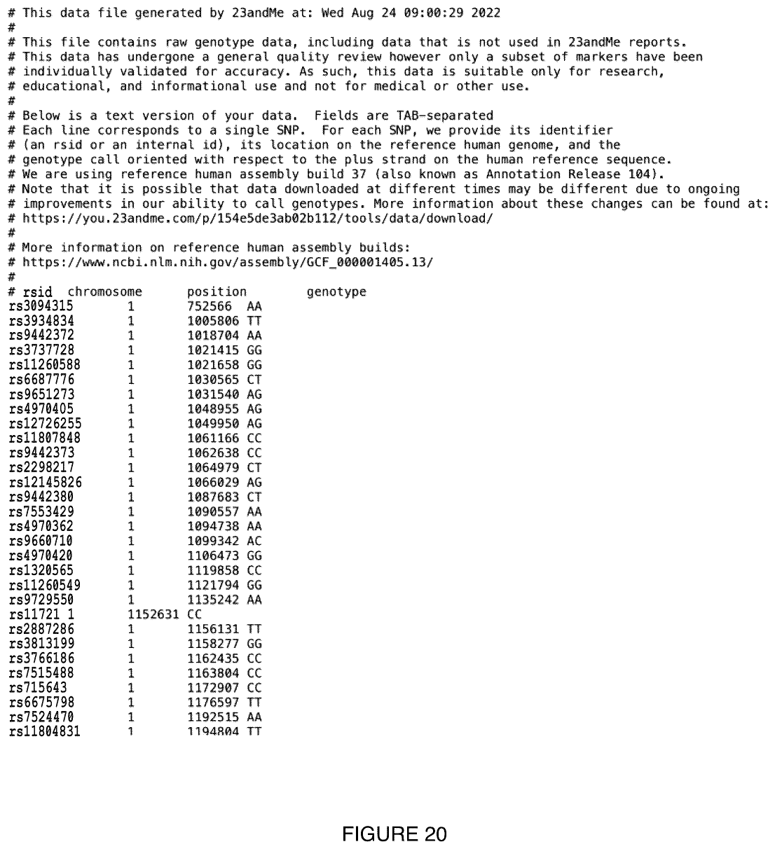

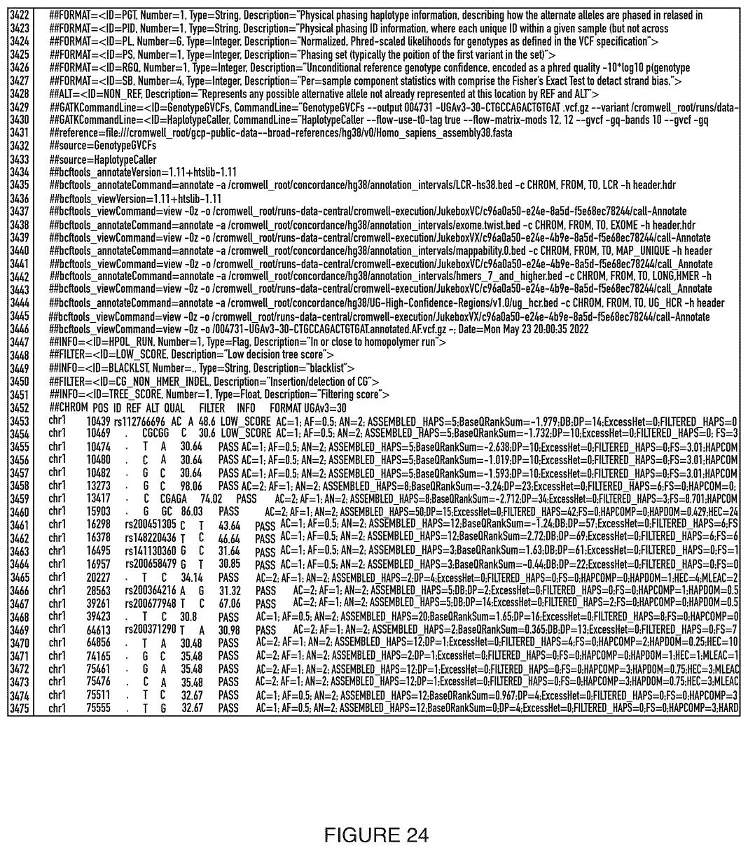

For example, in S210, the genome system 130 receives an input of unannotated genomic data. Examples are shown in FIG. 20 and FIG. 24. The genome system 130 may receive the unannotated genomic data from the user computing device 110, the data acquisition system 120, or any other suitable source of unannotated genomic data via the network 105 to the genome system 130, discussed in more detail in other sections herein. In example embodiments, the unannotated genomic data is received via an acquisition engine, the acquisition engine includes any software or hardware individually or in combination described herein that is capable of communicating with a user device, such as fetching, receiving, or sending information, thereby allowing access to the unannotated genomic data or annotated variant loci by the genome system 130 or the data acquisition system 120.

### Genomic Data

The unannotated genomic data received and/or used by these methods and systems includes the sequence of a subject's genome. The sequence may include the whole genome or a segment thereof. In a specific example, the sequence can be the whole genome imputed based on other information (e.g., genotypes, one or more segments of the whole genome, etc.).

In example embodiments, the unannotated genomic data does not include annotated information about the variant loci, wherein each variant locus can be specific locus corresponding to a genetic variant. In an example embodiment, the unannotated genomic data is not in a standardized format. Unannotated genomic data not in a standardized format cannot be readily matched to annotated data. In example embodiments, the unannotated genomic data includes genetic variants. The genetic variants can be in the nuclear genome. The genetic variants may also be present in the mitochondrial genome. The sequence may include any nucleotide sequence format. These formats may include, but are not limited to, plain sequence, FASTQ, EMBL, FASTA, GCG, GCG-RSF, GenBank, IG, Genomatix, annotation syntax, and/or 2 bit.

The unannotated genomic data may further include descriptive features that have not been standardized. For example, the descriptive features may include coordinates such as chromosome name, chromosome position, and/or chromosome strand. The descriptive features may include, for example, properties such as gene name and/or gene function. The unannotated genomic data comprising descriptive features may be any format such as BED, GTF2, GFF3, PSL, and/or BigBed for example.

The unannotated genomic data may further include quantitative data that has not been standardized. For example, the quantitative data may include features associated with a chromosomal position. An example of these features may be the degree of phylogenetic conservation. The unannotated genomic data comprising quantitative data may be any format such as bedGraph, wiggle, and/or BigWig for example.

The unannotated genomic data may further include read alignments that have not been standardized. For example, the read alignments may include short reads matched to genomic coordinates. An example of a read alignment may matching a short sequence of DNA to a region in a genome wherein the match is exact or share some amount of similarity. The unannotated genomic data comprising read alignments may be any format such as bowtie, SAM, PSL, and/or BAM for example.

In example embodiments, the unannotated genomic data is generated from sequencing, which includes high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi:10.1002/0471142727.mb0422s107 (2014). PMCID:4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In example embodiments, the unannotated genomic data is generated from whole genome sequencing. Whole genome sequencing (also known as WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast. “Whole genome amplification” (“WGA”) refers to any amplification method that aims to produce an amplification product that is representative of the genome from which it was amplified. Non-limiting WGA methods include Primer extension PCR (PEP) and improved PEP (I-PEP), Degenerated oligonucleotide primed PCR (DOP-PCR), Ligation-mediated PCR (LMP), T7-based linear amplification of DNA (TLAD), and Multiple displacement amplification (MDA).

In example embodiments, the unannotated genomic data is generated from whole exome sequencing. Exome sequencing, also known as whole exome sequencing (WES), is a genomic technique for sequencing all of the protein-coding genes in a genome (known as the exome) (see, e.g., Ng et al., 2009, Nature volume 461, pages 272-276). It consists of two steps: the first step is to select only the subset of DNA that encodes proteins. These regions are known as exons-humans have about 180,000 exons, constituting about 1% of the human genome, or approximately 30 million base pairs. The second step is to sequence the exonic DNA using any high-throughput DNA sequencing technology. In certain embodiments, whole exome sequencing is used to determine somatic mutations in genes associated with disease (e.g., cancer mutations).

In example embodiments, the unannotated genomic data is generated from targeted sequencing (see, e.g., Mantere et al., PLoS Genet 12 e1005816 2016; and Carneiro et al. BMC Genomics, 2012 13:375). Targeted gene sequencing panels are useful tools for analyzing specific mutations in a given sample. Focused panels contain a select set of genes or gene regions that have known or suspected associations with the disease or phenotype under study. In certain embodiments, targeted sequencing is used to detect mutations associated with a disease in a subject in need thereof. Targeted sequencing can increase the cost-effectiveness of variant discovery and detection.

In example embodiments, the unannotated genomic data is generated from the mitochondrial genome, which is specifically sequenced in a bulk sample using MitoRCA-seq (see e.g., Ni et al., MitoRCA-seq reveals unbalanced cytocine to thymine transition in Polg mutant mice. Sci Rep. 2015 Jul. 27; 5:12049. doi: 10.1038/srep12049). The method employs rolling circle amplification, which enriches the full-length circular mtDNA by either custom mtDNA-specific primers or a commercial kit, and minimizes the contamination of nuclear encoded mitochondrial DNA (Numts). In certain embodiments, RCA-seq is used to detect low-frequency mtDNA point mutations starting with as little as 1 ng of total DNA. In certain embodiments, mitochondrial DNA is sequenced using amplification by the amplicon approach (FIG. 25A, FIG. 25B, and FIG. 25C). In certain embodiments, mitochondrial DNA is sequenced using amplification by the rolling circle (RCA) approach (FIG. 26).

In example embodiments, single cell Mito-seq (scMito-seq) is used to sequence the mitochondrial genome in single cells. The method is based on performing rolling circle amplification of mitochondrial genomes in single cells.

In example embodiments, multiple displacement amplification (MDA) is used to generate the unannotated genomic data (e.g., single cell genome sequencing). Multiple displacement amplification (MDA, is a non-PCR-based isothermal method based on the annealing of random hexamers to denatured DNA, followed by strand-displacement synthesis at constant temperature (Blanco et al. J. Biol. Chem. 1989, 264, 8935-8940). It has been applied to samples with small quantities of genomic DNA, leading to the synthesis of high molecular weight DNA with limited sequence representation bias (Lizardi et al. Nature Genetics 1998, 19, 225-232; Dean et al., Proc. Natl. Acad. Sci. U.S.A. 2002, 99, 5261-5266). As DNA is synthesized by strand displacement, a gradually increasing number of priming events occur, forming a network of hyper-branched DNA structures. The reaction can be catalyzed by enzymes such as the Phi29 DNA polymerase or the large fragment of the Bst DNA polymerase. The Phi29 DNA polymerase possesses a proofreading activity resulting in error rates 100 times lower than Taq polymerase (Lasken et al. Trends Biotech. 2003, 21, 531-535).

In example embodiments, the unannotated genomic data is generated from Assay for Transposase Accessible Chromatin sequencing (ATAC-seq) or single cell ATAC-seq as described (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1). The term “tagmentation” refers to a step in the Assay for Transposase Accessible Chromatin using sequencing (ATAC-seq) as described. Specifically, a hyperactive Tn5 transposase loaded in vitro with adapters for high-throughput DNA sequencing, can simultaneously fragment and tag a genome with sequencing adapters. In certain embodiments, ATAC-seq is used on a bulk DNA sample to determine mitochondrial mutations.

In example embodiments, a transcriptome is sequenced to generate unannotated genomic data. The transcriptome may be used to genotype nuclear and mitochondrial genomes in addition to determining gene expression. As used herein the term “transcriptome” refers to the set of transcripts molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In example embodiments, the unannotated genomic data is generated from single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In example embodiments, the unannotated genomic data is generated from single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In example embodiments, the unannotated genomic data is generated from plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In example embodiments, the unannotated genomic data is generated from high-throughput single-cell RNA-seq where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “DropletBarcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In example embodiments, the unannotated genomic data is generated from single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 Oct; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; and Drokhlyansky E, Smillie C S, Van Wittenberghe N, et al. The Human and Mouse Enteric Nervous System at Single-Cell Resolution. Cell. 2020; 182(6):1606-1622.e23, which are herein incorporated by reference in their entirety.

In example embodiments, the unannotated genomic data is generated from Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

In example embodiments, the unannotated genomic data is generated from a single cell atlas, which includes single cell epigenetic data. A single cell atlas for a tissue may be constructed by measuring epigenetic marks on chromatin in single cells. The epigenetic marks can indicate genomic loci that are in active or silent chromatin states (see, e.g., Epigenetics, Second Edition, 2015, Edited by C. David Allis; Marie-Laure Caparros; Thomas Jenuwein; Danny Reinberg; Associate Editor Monika Lachlan). In certain embodiments, single cell ChIP-seq can be used to determine chromatin states in single cells (see, e.g., Rotem, et al., Single-cell ChIP-seq reveals cell subpopulations defined by chromatin state. Nat Biotechnol. 2015 November; 33(11): 1165-1172). In certain embodiments, single cell ChIP-seq is used to determine genomic loci that are occupied by histone modifications, histone variants, transcription factors and/or chromatin modifying enzymes. In certain embodiments, epigenetic features can be chromatin contact domains, chromatin loops, superloops, or chromatin architecture data, such as obtained by single cell HiC (see, e.g., Rao et al., Cell. 2014 Dec. 18; 159(7):1665-80; and Ramani, et al., Sci-Hi-C: A single-cell Hi-C method for mapping 3D genome organization in large number of single cells Methods. 2020 Jan. 1; 170: 61-68).

In example embodiments, the unannotated genomic data is generated from a single cell atlas, which includes spatially resolved single cell data (see, e.g., Li X, Wang C Y. From bulk, single-cell to spatial RNA sequencing. Int J Oral Sci. 2021; 13(1):36. Published 2021 Nov. 15. doi:10.1038/s41368-021-00146-0). The spatial data used in the present invention can be any spatial data. Methods of generating spatial data of varying resolution are known in the art, for example, ISS (Ke, R. et al. In situ sequencing for RNA analysis in preserved tissue and cells. Nat. Methods 10, 857-860 (2013)), MERFISH (Chen, K. H., Boettiger, A. N., Moffitt, J. R., Wang, S. & Zhuang, X. Spatially resolved, highly multiplexed RNA profiling in single cells. Science 348, (2015)), smFISH (Codeluppi, S. et al. Spatial organization of the somatosensory cortex revealed by cyclic smFISH. biorxiv.org/lookup/doi/10.1101/276097 (2018) doi:10.1101/276097), osmFISH (Codeluppi, S. et al. Spatial organization of the somatosensory cortex revealed by osmFISH. Nat. Methods 15, 932-935 (2018)), STARMap (Wang, X. et al. Three-dimensional intact-tissue sequencing of single-cell transcriptional states. Science 361, eaat5691 (2018)), Targeted ExSeq (Alon, S. et al. Expansion Sequencing: Spatially Precise In Situ Transcriptomics in Intact Biological Systems. biorxiv.org/lookup/doi/10.1101/2020.05.13.094268 (2020) doi:10.1101/2020.05.13.094268), seqFISH+(Eng, C.-H. L. et al. Transcriptome-scale super-resolved imaging in tissues by RNA seqFISH+. Nature (2019) doi:10.1038/s41586-019-1049-y.), Spatial Transcriptomics methods (e.g., Spatial Transcriptomics (ST))(see, e.g., Stihl, P. L. et al. Visualization and analysis of gene expression in tissue sections by spatial transcriptomics. Science 353, 78-82 (2016)) (now available commercially as Visium); Visium Spatial Capture Technology, 10× Genomics, Pleasanton, CA; WO2020047007A2; WO2020123317A2; WO2020047005A1; WO2020176788A1; and WO2020190509A9), Slide-seq (Rodriques, S. G. et al. Slide-seq: A scalable technology for measuring genome-wide expression at high spatial resolution. Science 363, 1463-1467 (2019)), or High Definition Spatial Transcriptomics (Vickovic, S. et al. High-definition spatial transcriptomics for in situ tissue profiling. Nat. Methods 16, 987-990 (2019)). In certain embodiments, proteomics and spatial patterning using antenna networks is used to spatially map a tissue specimen and this data can be further used to align single cell data to a larger tissue specimen (see, e.g., US20190285644A1). In certain embodiments, the spatial data can be immunohistochemistry data or immunofluorescence data.

The digital spatial profiler (DSP), GeoMx DSP, is built on Nanostring's digital molecular barcoding core technology and is further extended by linking the target complementary sequence probe to a unique DSP barcode through a UV cleavable linker (see, e.g., Li, et al., 2021). A pool of such barcode-labeled probes is hybridized to mRNA targets that are released from fresh or FFPE tissue sections mounted on a glass slide. The slide is also stained using fluorescent markers (i.e., fluorescently conjugated antibodies) and imaged to establish tissue “geography” using the GeoMx DSP instrument. After the regions-of-interest (ROIs) are selected, the DSP barcodes are released via UV exposure and collected from the ROIs on the tissue. These barcodes are sequenced through standard NGS procedures. The identity and number of sequenced barcodes can be translated into specific mRNA molecules and their abundance, respectively, and then mapped to the tissue section based on their geographic location. The DSP barcode can also be linked to antibodies to detect proteins.

In example embodiments, the unannotated genomic data is generated from a single cell atlas, which includes single cell proteomics data (see, e.g., Yang L, George J, Wang J. Deep Profiling of Cellular Heterogeneity by Emerging Single-Cell Proteomic Technologies. Proteomics. 2020; 20(13):e1900226. doi:10.1002/pmic.201900226). In certain embodiments, single cell proteomics can be used to generate the single cell data. In certain embodiments, the single cell proteomics data is combined with single cell transcriptome data. Non-limiting examples include multiplex analysis of single cell constituents (US20180340939A), single-cell proteomic assay using aptamers (US20180320224A1), and methods of identifying multiple epitopes in cells (US20170321251A1).

In example embodiments, the unannotated genomic data is generated from a single cell atlas, which includes single cell multimodal data. Multiomic review (see, e.g., Lee J, Hyeon D Y, Hwang D. Single-cell multiomics: technologies and data analysis methods. Exp Mol Med. 2020; 52(9):1428-1442. doi:10.1038/s12276-020-0420-2). In certain embodiments, SHARE-Seq (Ma, S. et al. Chromatin potential identified by shared single cell profiling of RNA and chromatin. bioRxiv 2020.06.17.156943 (2020) doi:10.1101/2020.06.17.156943) is used to generate single cell RNA-seq and chromatin accessibility data. In certain embodiments, CITE-seq (Stoeckius, M. et al. Simultaneous epitope and transcriptome measurement in single cells. Nat. Methods 14, 865-868 (2017)) (cellular proteins) is used to generate single cell RNA-seq and proteomics data. In certain embodiments, Patch-seq (Cadwell, C. R. et al. Electrophysiological, transcriptomic and morphologic profiling of single neurons using Patch-seq. Nat. Biotechnol. 34, 199-203 (2016)) is used to generate single cell RNA-seq and patch-clamping electrophysiological recording and morphological analysis of single neurons data (e.g., for the brain or enteric nervous system (ENS)) (see, e.g., van den Hurk, et al., Patch-Seq Protocol to Analyze the Electrophysiology, Morphology and Transcriptome of Whole Single Neurons Derived From Human Pluripotent Stem Cells, Front Mol Neurosci. 2018; 11: 261).

In example embodiments, the unannotated genomic data is generated from measuring mitochondrial mutations, nuclear genome mutations, and gene expression, which are all performed using a high-throughput single cell RNA sequencing library (e.g., scRNA-seq, Seq-well). The methods described herein are specifically designed for compatibility with high-throughput single-cell RNA-sequencing protocols (droplet or microwells, i.e. Seq-Well, Drop-Seq, 10×). In some embodiments, the library includes transcripts from a plurality of cells. In some embodiments, a plurality of cells includes about 100, 500, 1,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000 or 1,000,000 or more cells. In some embodiments, the library is prepared using any method described herein, e.g., the Seq-Well, InDrop, Drop-Seq, or 10× Genomics methods and a plurality of cells includes between 10,000 and 1,000,000 cells, e.g., 20,000-100,000 cells.

In example embodiments, the unannotated genomic data is generated from RNA sequencing. In example embodiments, the RNA sequencing is single cell RNA-sequencing. In example embodiments, a cDNA library is generated. The cDNA library may be used to generate sequencing libraries for determining mutations in the mitochondrial genome (genotyping), the nuclear genome (genotyping), or for determining gene expression (RNA-seq) (see, e.g., WO 2019/084055 FIG. 19A). For example, the RNA-seq library is generated using tagmentation and the sequencing reads are 3′ biased for identification of the gene only. For genotyping, the target sequence containing a site of interest is enriched and the sequencing reads include the target region. In the case of genotyping the mitochondrial genome, enrichment of all sites in the mitochondrial genome can be enriched by performing PCR enrichment using the primers disclosed herein (see, Table 1).

In example embodiments, the unannotated genomic data is generated from whole transcriptome amplification (WTA), which is used to generate the cDNA library. The cDNA library may also be referred to as the whole transcriptome amplification (WTA) library. The library may include “WTA products”. “Whole transcriptome amplification” (“WTA”) refers to any amplification method that aims to produce an amplification product that is representative of a population of RNA from the cell from which it was prepared. An illustrative WTA method entails production of cDNA bearing linkers on either end that facilitate unbiased amplification. In many implementations, WTA is carried out to analyze messenger (poly-A) RNA (this is also referred to as “RNAseq”). WTA may include reverse transcription (RT) to generate first strand cDNA. First strand synthesis may be followed by second strand synthesis. First strand synthesis may include priming of the RT on a 3′ adaptor linked to the RNA molecules. In example embodiments, each RNA in a library may be amplified to create a whole transcriptome amplified (WTA) RNA by reverse transcription with a primer comprising a sequence adapter. The reverse transcribed product may be amplified by PCR amplification with primers that bind both 5′ and 3′ sequence adapters. In example embodiments, the amplified RNA includes the orientation: 5′-sequencing adapter-cell barcode-UMI-UUUUUUU-mRNA-3′. In some embodiments, PCR amplification is conducted on the reverse transcribed products with primers that bind both sequence adapters and adding a library barcode and optionally additional sequence adapters.

In example embodiments, the unannotated genomic data is generated from single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 Oct; 14(10):955-958; and International patent application number PCT/US2016/059239, published as WO2017164936 on Sep. 28, 2017, which are herein incorporated by reference in their entirety.

In example embodiments, the unannotated genomic data is generated from any suitable RNA or DNA amplification technique may be used. In example embodiments, the RNA or DNA amplification is an isothermal amplification. In example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In example embodiments, non-isothermal amplification methods may be used which include, but are not limited to, PCR, multiple displacement amplification (MDA), rolling circle amplification (RCA), ligase chain reaction (LCR), or ramification amplification method (RAM).

In example embodiments, cells to be sequenced according to any of the methods herein are lysed under conditions specific to sequencing mitochondrial genomes. In example embodiments, lysis using mild conditions does not result in sequencing of all of the mitochondrial genomes. In example embodiments, use of harsher lysing conditions allows for increase sequencing of mitochondrial genomes due to improved lysis of mitochondria. In example embodiments, lysis buffers include one or more of NP-40, Triton X-100, SDS, guanidine isothiocyante, guanidine hydrochloride or guanidine thiocyanate. The use of more stringent lysis may not affect the nuclear genome transcripts.

In example embodiments, the sequencing cost is lower in sequencing mitochondrial genomes because of the size of the mitochondrial genome. The terms “depth” or “coverage” as used herein refers to the number of times a nucleotide is read during the sequencing process. In regards to single cell RNA sequencing, “depth” or “coverage” as used herein refers to the number of mapped reads per cell. Depth in regards to genome sequencing may be calculated from the length of the original genome (G), the number of reads (N), and the average read length (L) as N×L/G. For example, a hypothetical genome with 2,000 base pairs reconstructed from 8 reads with an average length of 500 nucleotides will have 2× redundancy.

The terms “low-pass sequencing” or “shallow sequencing” as used herein refers to a wide range of depths greater than or equal to 0.1× up to 1×. Shallow sequencing may also refer to about 5000 reads per cell (e.g., 1,000 to 10,000 reads per cell).

The term “deep sequencing” as used herein indicates that the total number of reads is many times larger than the length of the sequence under study. The term “deep” as used herein refers to a wide range of depths greater than 1× up to 100×. Deep sequencing may also refer to 100× coverage as compared to shallow sequencing (e.g., 100,000 to 1,000,000 reads per cell).

The term “ultra-deep” as used herein refers to higher coverage (>100-fold), which allows for detection of sequence variants in mixed populations.

In S220, the genome system 130 receives input of the unannotated genomic data and passes the unannotated genomic data to the genome server 135 wherein the genome annotation network 135 converts the unannotated genomic data into a standardized file format based, at least in part, on identified variant loci in the unannotated genomic data.

### Standardized File Formatting

In example embodiments, the unannotated genomic data is converted into a standardized file format based, at least in part, on identified variant loci in the unannotated genomic data. The identified variant loci can be identified using a reference genome (e.g., by comparing the genomic data for the subject to the reference genome). For example, the identified variant loci for a subject can be the loci in the subject's genomic data with genetic variants relative to the reference genome. The reference genome can be predetermined, determined based on a subset of population data (e.g., random subset, population representative subset, ancestry-specific subset, etc.), and/or otherwise determined. In a specific example, the reference genome can be determined based on an ancestry associated with the subject (e.g., a reference genome for the relevant ancestry) and/or other clinical feature.

Unannotated genomic data, further described herein, is generally not formatted for matching identified variant loci to annotated data from a plurality of data sources. Standardization, in general, is the process of creating a standard format and transforming data from different sources into a consistent format (i.e., converting unannotated genomic data to a standardized file format). Formatting may include spelling, such as capitalization; punctuation; and/or acronyms, alphanumeric characters, and numerical values. Standardization creates consistent structure across all data. Furthermore, standardization may include eliminating extraneous or erroneous data. Thereby increasing accuracy and speed of the method.

Standardizing data may include the initial steps of auditing and evaluating data sources, declutter data sources, assess data collection methods, and/or define the standards, e.g., formatting. Auditing and evaluating data sources may include, in general, identifying the necessary data and unnecessary data. Decluttering may include, in general, removing the unnecessary data, the unnecessary data may include duplicate data, irrelevant data, redundant data, inaccurate data, and/or low-quality data. Assessing data collection methods may include, in general, preventing low-quality data from entering a data set. Defining the standards may include, in general, defining rules for including and formatting each data element and formatting.

After the initial standardization steps have been performed, the data (e.g., unannotated genomic data) can then be standardize. In general, standardization may include source-to-target mapping, which includes identifying the data elements used in the method, or reconciliation, which includes comparing different data sets to each other and verifying they are aligned. Standardizing a file format improves data portability, such as data transfer without data corruption, and interoperability, such as integrating a plurality of data sets and matching identified variant loci to that plurality. It should be mentioned the unannotated genomic data standardization process may be performed on the annotation data from a plurality of sources.

### Match-Optimized Variables

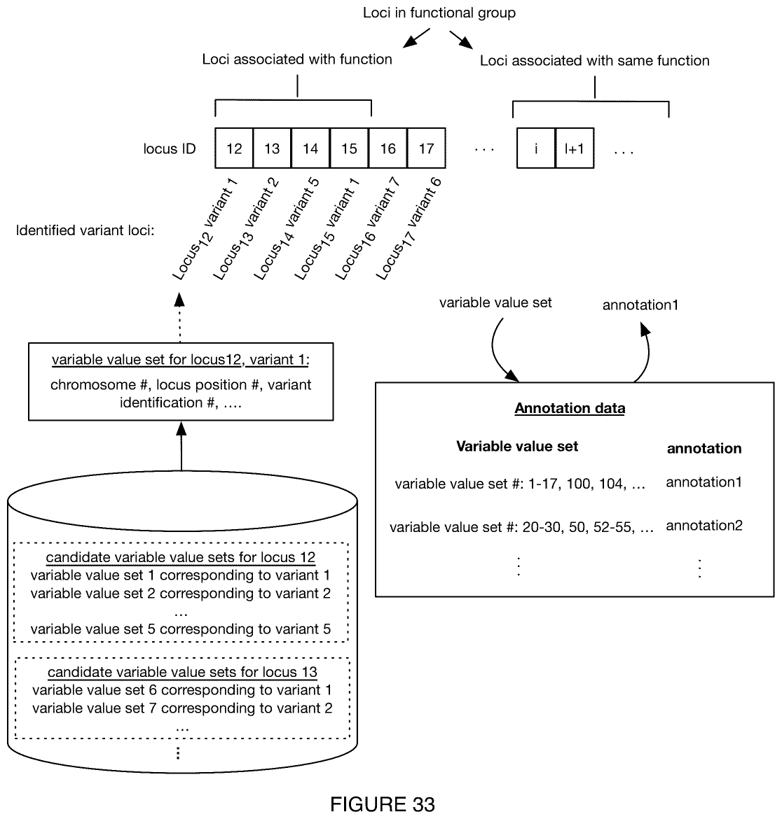

In an example embodiment, the standardized file format includes values for a set of match-optimized variables (e.g., a variable value set) for each identified variant locus and configured to optimize search of the annotations from the plurality of data sources. The match-optimized variables include data elements that describe genomic loci. In an illustrative example, these variables are match-optimized because they have been standardized across the annotation data and input unannotated genomic data. An example is shown in FIG. 33. An identified variant locus with values for match-optimized variable(s) can be rapidly and accurately (i.e. optimized) aligned with the annotation data because the data elements (e.g., variables) are the same (i.e. standardized).

In an example embodiment, the match-optimized variables include any characteristic feature of a genomic loci. In an example embodiment, the match-optimized variables include one or more variables selected from chromosome number, overall chromosome location (e.g., locus), variant start position, variant stop position, variant identification number, variant type, reference allele(s) (e.g., reference genotype), present allele(s) (e.g., present genotype), reference assembly number. In a specific example, a specific variant at a variant locus can be represented by values for the match-optimized variables (e.g., each variant locus can correspond to a variable value set). In an illustrative example, at locus 20 on chromosome 1, the reference genotype AA corresponds to a first variable value set, genotype AB (e.g., a single copy of variant B) corresponds to a second variable value set, genotype BB corresponds to a third variable value set, genotype AC (e.g., a single copy of variant C) corresponds to a fourth variable value set, genotype CC corresponds to a fifth variable value set, and genotype BC corresponds to a sixth variable value set.

All or portions of the method can increase the computational efficiency of training, searching, and/or comparison. In specific examples, the method can use parallelization (e.g., parallelizing by chromosome, parallelizing by search region within a chromosome, etc.), subsetting (e.g., into search regions), preprocessing data, filtering, pretraining models, and/or any other suitable methods. In a first example, lifetime risk models and/or percentile risk models (e.g., PRS distributions) can be pretrained such that a new subject's input can be compared efficiently (e.g., preprocessing ancestry-specific genomes to determine PRS distributions for each ancestry group; the PRS distributions can be more readily available while the raw genomic data can be kept in cold storage). In a second example, a database (e.g., ClinVar or other third-party database) can be parsed to filter for pathogenic and/or likely pathogenic variants of a certain gene. In a specific example, a pipeline for disease can map to a gene, which can map to pathogenic and/or likely pathogenic variants from the database, which can map to affected patients (with those variants). In a third example, variants (e.g., variable value sets) can be mapped to annotations (e.g., genes, RSID, pathways, diseases, etc.) prior to receiving genomic data for a subject.

### Pre-Segmenting Data

In example embodiment, during the standardized file formatting, the unannotated genomic data is pre-segmented into subsets. The pre-segmentation refers to separating, partitioning, or otherwise dividing the genomic data before matching identified/annotated variant loci to annotation data. In an example embodiment, the pre-segmentation occurs before the variant loci are identified. In example embodiments, the pre-segmentation occurs after the variant loci have been identified.

In an example embodiment, the annotation data from the plurality of data sources is parsed into a matching structure to optimize a search speed of the annotation data, wherein the matching structure includes pre-segmenting annotation data into subsets. In this context, pre-segmentation refers to separating, partitioning, or otherwise dividing the annotation data before matching identified/annotated variant loci to annotation data. In an example embodiment, this step occurs before any subject's unannotated genomic data is received. In an example embodiment, this step occurs in between receiving subjects' unannotated genomic data.

The genomic and annotation data may be segmented by similar data and grouped into subsets based on parameters. For example, the genomic data may be segmented and grouped into subsets by any of the match-optimized variables (e.g. parameters) described herein. For example, the genomic data may be segmented by chromosome number. The genomic data may then be segmented by location on the chromosome. Pre-segmenting data may include any one or more segmentation steps. In example embodiments, additional subsets include the match-optimized variables described herein.

### Subset Data Structure

In an example embodiment, the genomic and/or annotation data in each subset is stored in a multi-dimensional array data structure. A data structure is used to store and organize data. There many types of data structures. In general, data structures are cat categorized into two types: linear and non-linear. Linear data structures arrange data elements sequentially (i.e., linearly) wherein each element is linked to the previous and subsequent element. Example linear data structures include linear arrays, stacks, queues, and linked lists. Non-linear data structures arrange data elements non-linearly such that all the elements in the data structure cannot be traversed in a single pass. Example non-linear data structures include trees and graphs

A multidimensional array (e.g. a matrix) is an array includes linear multiple rows and columns. Multidimensional arrays are well known in the art and would be readily understood by one skilled in the art. The multidimensional array can be symmetric (e.g., 2×2, 3×3, 4×4) or asymmetric (e.g. 1×2, 3×5, 2×7). The dimensions will include a size proportionate to the division of the data.

In example embodiments, the subset is stored in a tree data structure. In example embodiments, the tree data structure is a dictionary data structure. In example embodiments, the dictionary data structure is a hash data structure. Dictionary and hash data structures are well known in the art and would be readily understood by one skilled in the art. In general, dictionary and hash data structures use keys to locate value(s) in the data structure. In an example embodiment, the name (i.e., title or field identifier) of the group the data is segmented into is the key and segmented genomic data is the value. In example embodiments, the match-optimized variable values (e.g., names) are the keys and the corresponding genomic data are the values.

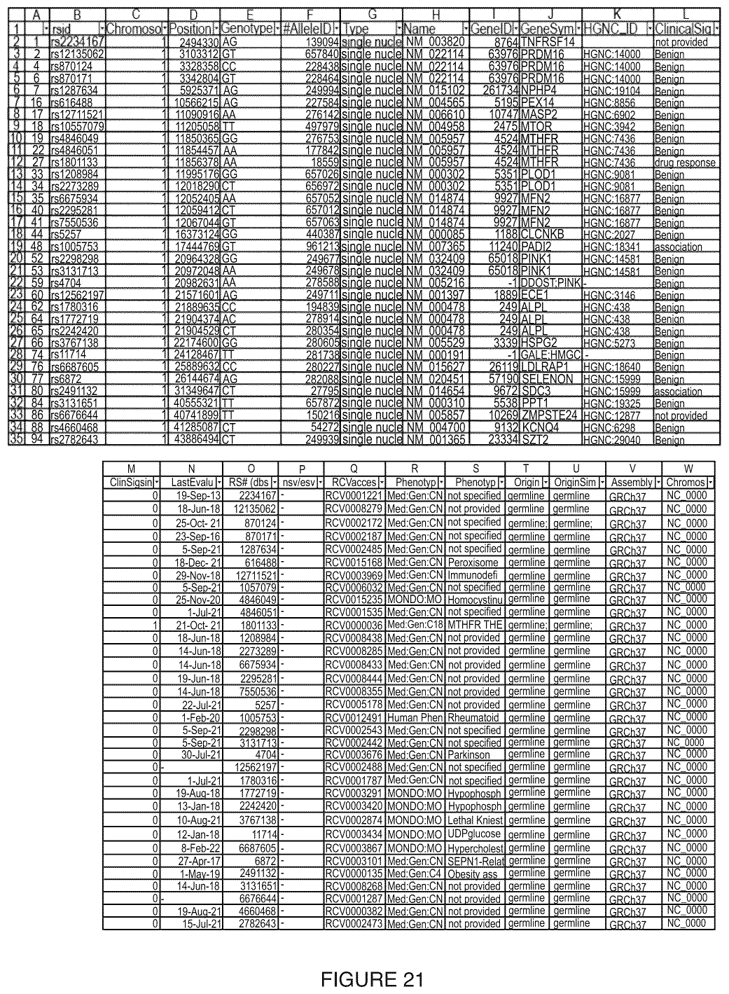

In S230, the genome network 133 can generate annotated variant loci based on annotation data from a plurality of data sources, which functions to label a user's variant loci with relevant functional information (e.g., increased and/or decreased risk for a disease, drug response information, etc.). For example, the annotated variant loci can be generated by matching annotation data from a plurality of data sources comprising different data types with the corresponding identified variant loci. An example of annotated variant loci for a subject is shown in FIG. 21.

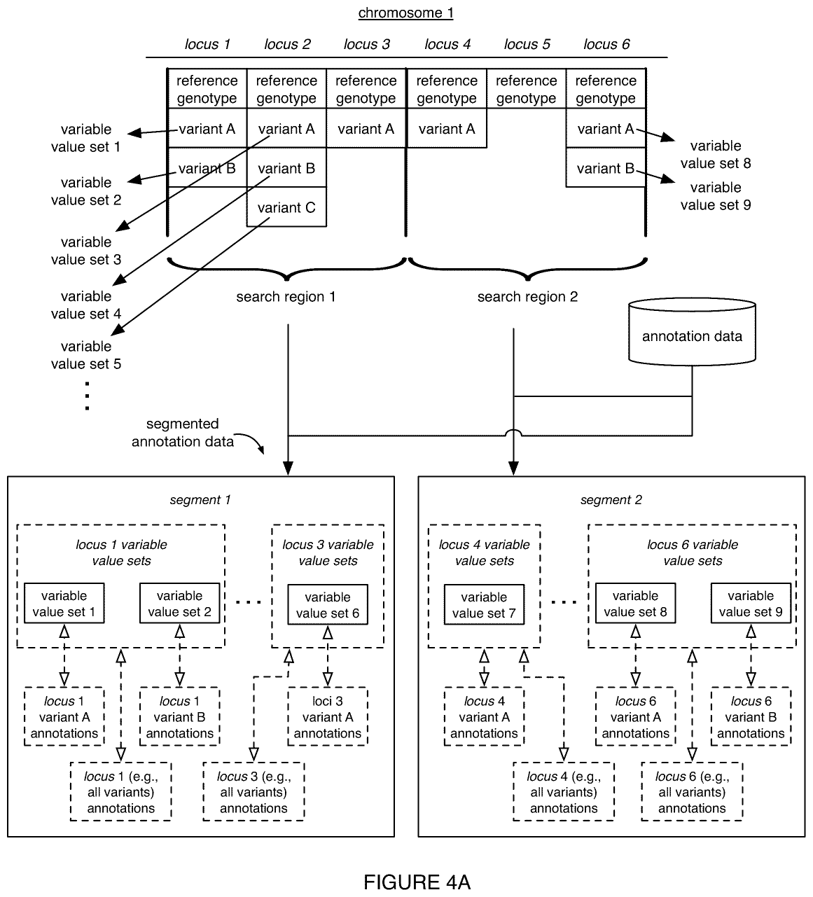

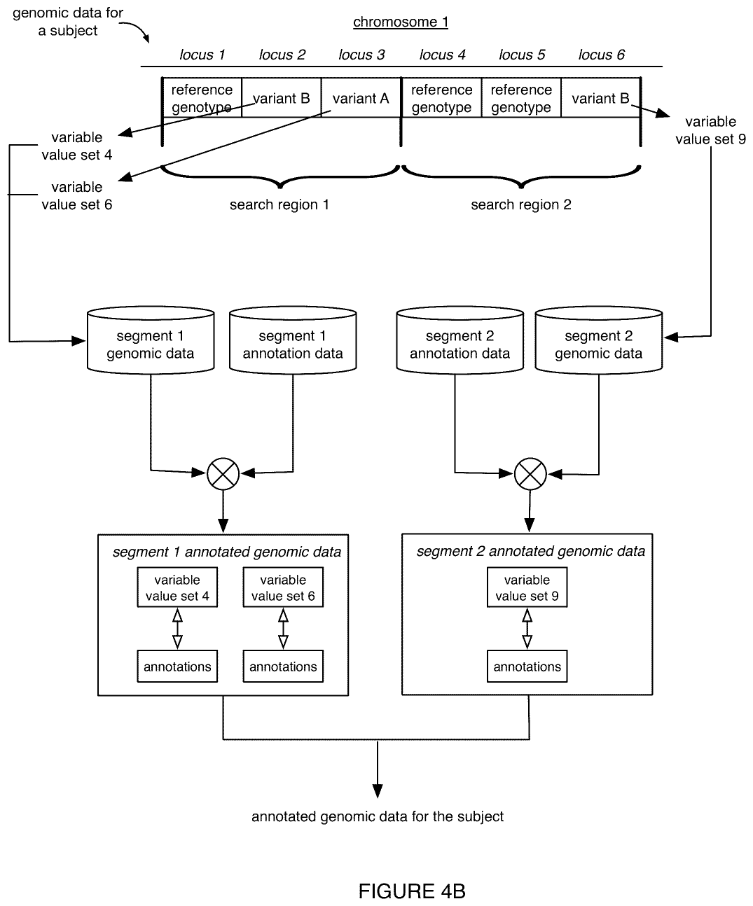

In an example, the method can include: generating annotation data (e.g., mapping annotations from a plurality of data sources to variable value sets); segmenting the annotation data into subsets of annotation data, each subset of annotation data corresponding to a search region in a set of search regions; receiving unannotated genomic data for a subject; determining a variable value set for each identified variant locus in the unannotated genomic data; and generating annotated variant loci for the subject based on the segmented annotation data and the variable value sets for each identified variant locus. For example, an annotation can be mapped to a specific variant (e.g., a variable value set) and/or a locus (e.g., mapping the annotation to all variable value sets associated with the locus). An example is shown in FIG. 4A and FIG. 4B. In a specific example, annotations can be mapped to a locus by default and can be mapped to a specific variant (e.g., a variant with base G at position X, relative to the reference genome with base A at position X) when the annotation data is sufficient (e.g., annotation data that includes data for a sufficient number of variants at a locus, a sufficient quantity of annotation data, etc.).

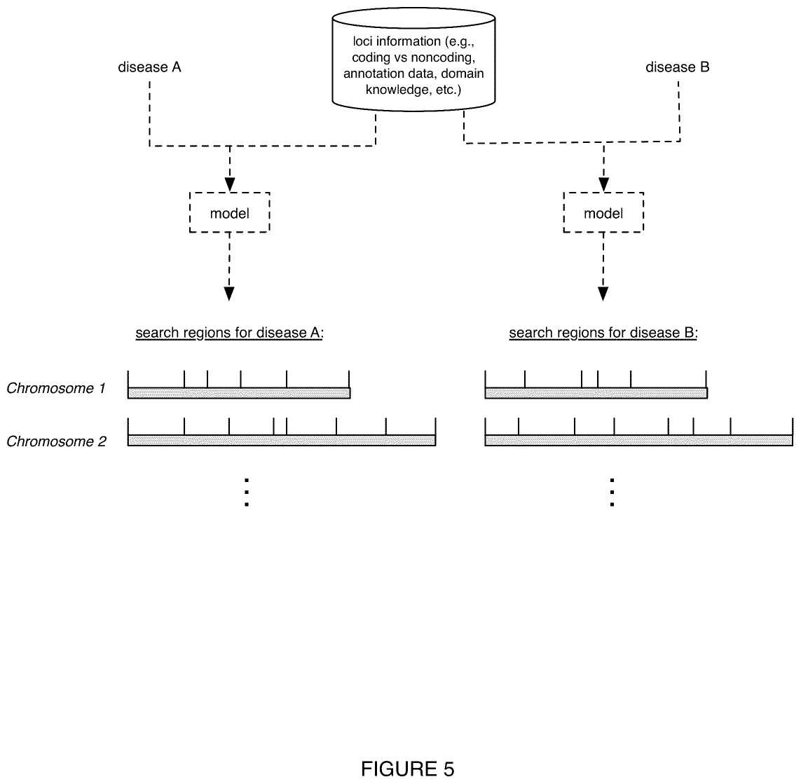

Each search region preferably includes a set of loci on a single chromosome (e.g., the search region includes a loci range within a chromosome), but can alternatively include a set of loci across multiple chromosomes. Search regions can be overlapping or nonoverlapping, contiguous or non-contiguous, same or different sizes (e.g., the same loci range length across search regions or varying lengths of loci ranges, etc.) and/or otherwise configured. The size of each search region can be between 10 bp-1,000 kbp or any range or value therebetween (e.g., less than 5000 bp, less than 1000 bp, 50 bp-500 bp, 100 bp, a chromosome length, etc.), but can alternatively be less than 10 bp or greater than 1,000 kbp. The size of each search region can be between 5 loci-50,000 loci or any range or value therebetween, but can alternatively be less than 5 loci or greater than 50,000 loci. For each search region, the size (e.g., length of the loci range) can optionally be determined based on: whether the search region includes a coding sequence or a noncoding sequence (e.g., increasing the search region size for coding sequences, increasing the search region size for noncoding sequences, etc.), the location of the search region in the chromosome, annotation data (e.g., the function associated with loci in the search region), a trait of interest (e.g., disease of interest) and/or any other loci information. The set of search regions (e.g., the size and/or location of each search region in the set) can optionally be determined based on the trait of interest. An example is shown in FIG. 5.

Segmenting annotation data can include sorting each annotation into one or more subsets of annotation data. For example, an annotation corresponding to multiple loci can be sorted into a single subset of annotation data (the subset corresponding to all or a portion of the multiple loci) or sorted into multiple subsets of annotation data (each subset corresponding to a portion of the multiple loci). In an illustrative example, Annotation A can correspond to loci 1-10 on chromosome 1 (and/or one or more specific variants at loci 1-10 on chromosome 1) and loci 2-3 on chromosome 2 (and/or one or more specific variants at loci 2-3 on chromosome 2). In specific examples, Annotation A can be sorted into: all annotation data subsets corresponding to chromosome 1 loci 1-10 and chromosome 2 loci 2-3; a maximum of one annotation data subset for chromosome 1 loci 1-10 and a maximum of one annotation data subset for chromosome 2 loci 2-3; one annotation data subset across both chromosome 1 loci 1-10 and chromosome 2 loci 2-3; and/or any other number of annotation data subsets.

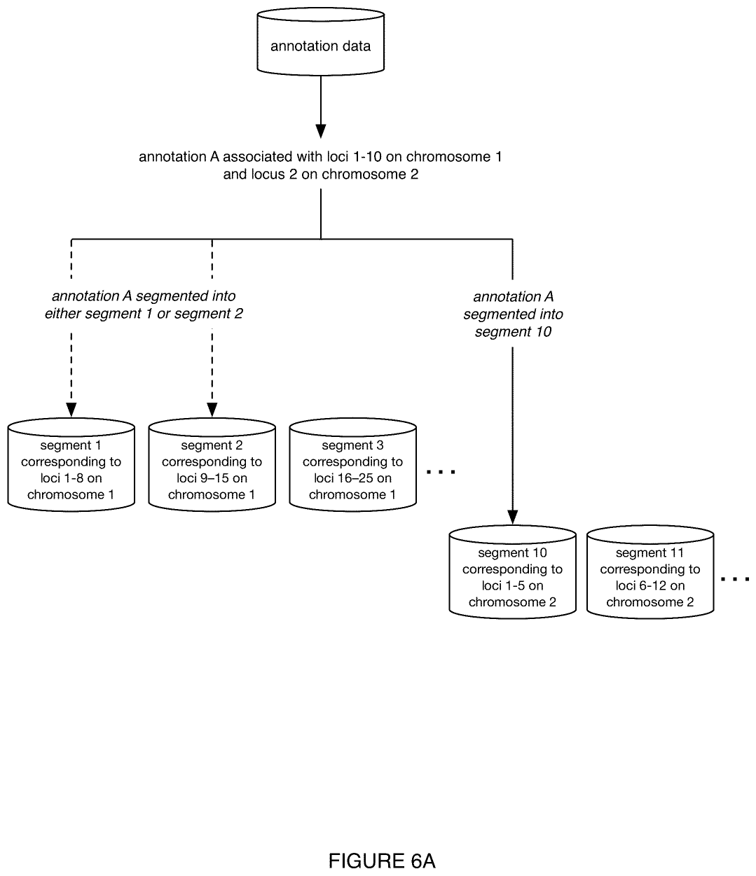

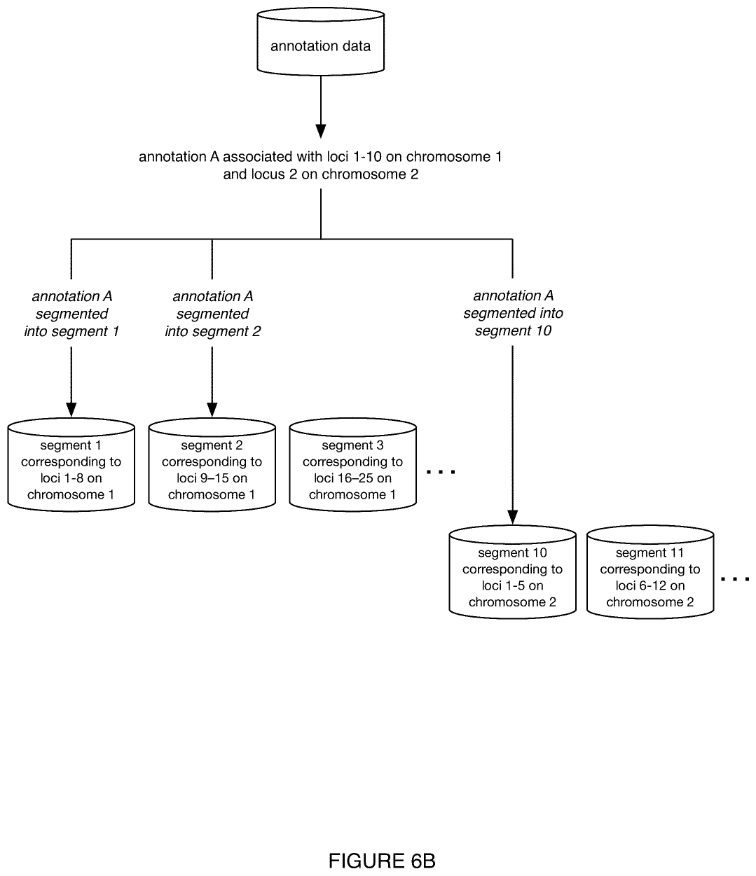

In an example, annotations can be duplicated across multiple annotation data subsets corresponding to different search regions within the same chromosome (e.g., an example is shown in FIG. 6B) or not duplicated across multiple annotation data subsets corresponding to different search regions within the same chromosome (e.g., an example is shown in FIG. 6A). In a specific example, when an annotation is mapped to variable value sets associated with multiple search regions within a chromosome, segmenting the annotation data can include sorting the annotation into a subset of annotation data for a single search region (e.g., not duplicating the annotation for multiple search regions associated with the same chromosome). In a first example, the method can include ‘rounding down’ within a chromosome, wherein an annotation is sorted into the annotation data subset corresponding to the search region with the lowest loci number within a chromosome. In an illustrative example, Annotation A corresponding to chromosome 1 loci 1-10 can be sorted into an annotation data subset corresponding to chromosome 1 loci 1-8 (e.g., and not sorted into an annidation data subset corresponding to chromosome 1 loci 9-15). In an illustrative example, an annotation data subset can correspond to a search region for loci 1-8, but include an annotation mapped to variable value sets corresponding to loci outside of the search region (e.g., variable value sets for loci 1-10). In a second example, the method can include ‘rounding up’ within a chromosome, wherein an annotation is sorted into the annotation data subset corresponding to the search region with the highest loci number within a chromosome. In an illustrative example, Annotation A corresponding to chromosome 1 loci 1-10 can be sorted into an annotation data subset corresponding to chromosome 1 loci 9-15 (e.g., and not sorted into an annidation data subset corresponding to chromosome 1 loci 1-8).

In an example, annotations can be duplicated across multiple annotation data subsets corresponding to search regions on different chromosomes or not duplicated across multiple annotation data subsets corresponding search regions on different chromosomes. In a specific example, when an annotation is mapped to variable value sets associated with multiple search regions across different chromosomes, segmenting the annotation data can include repeating the annotation across multiple subsets of annotation data (e.g., duplicating the annotation for multiple search regions across different chromosomes). An example is shown in FIG. 6A.

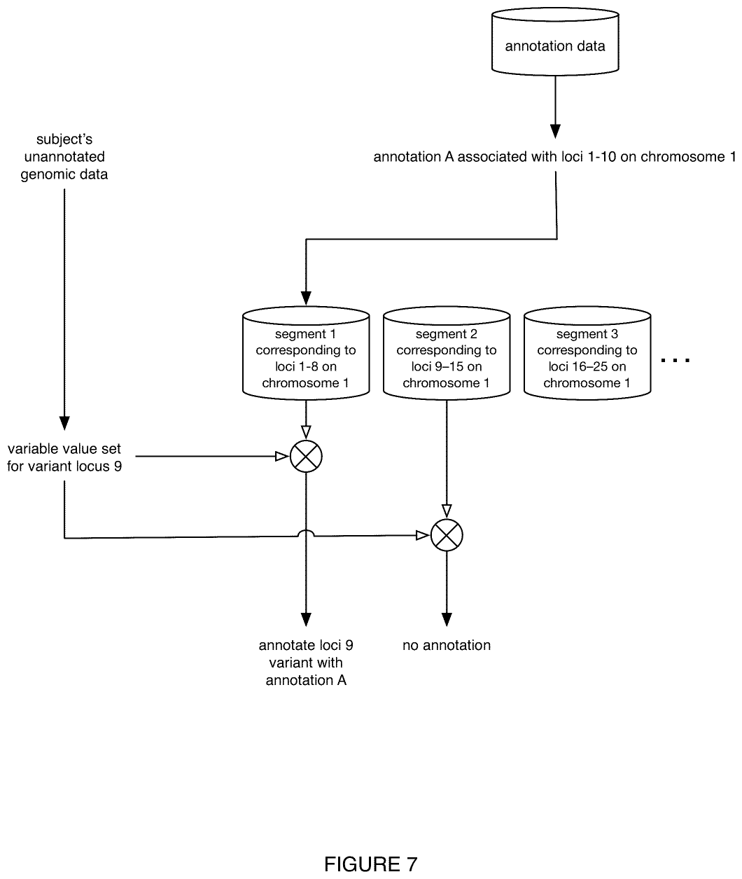

Generating annotated variant loci can include, for each identified variant locus, searching one or more subsets of annotation data (e.g., at least two subsets of annotation data) for annotations corresponding to the identified variant locus (e.g., corresponding to the variable value set representing a specific variant at the identified variant locus). In an example, generating annotated variant loci can include, for each variable value set associated with an identified variant locus for the subject: selecting a search region (e.g., a first search region) from the set of search regions based on the variable value set; searching within a subset of annotation data (e.g., a first subset of annotation data) corresponding to the selected search region to identify annotations associated with a matching variable value set; and annotating the identified variant locus with the identified annotations. Identified annotations can optionally include all annotations with a matching variable value set or a subset of annotations with a matching variable value set (e.g., annotations relevant to a trait of interest). In a specific example, generating the annotated variant loci can include: selecting a second search region from the set of search regions based on the variable value set (e.g., wherein the associated identified variant locus corresponds to a locus within the first search region or within the second search region), wherein the second search region corresponds to a second range of loci adjacent to the first range of loci; searching within a second subset of annotation data corresponding to the selected second search region to identify annotations in the second subset of annotation data associated with a matching variable value set; and annotating the identified variant locus with the identified annotations. In an illustrative example, when annotation data mapping to multiple search regions within a chromosome is segmented into the search region corresponding to lower loci values (e.g., ‘rounding down’), annotating a variant locus can include checking annotation data for the search region corresponding to the variant locus and the adjacent search region corresponding to lower loci values. An example is shown in FIG. 7. In an illustrative example, Annotation A associated with variable value sets for loci 1-10 (e.g., all or a subset of variants for loci 1-10) can be sorted into a first annotation data subset corresponding to loci 1-8 and not be sorted into a second annotation data subset corresponding to loci 9-15. A variable value set for a subject representing a variant at locus 9 can be annotated with Annotation A by searching both the first annotation data subset and the second annotation data subset, wherein the match between the variable value set and annotation A can be found within the first annotation data subset.

In a specific example, the search region can be selected based on all or a subset of variable values in the variable value set. In an illustrative example, when the variable value set includes a locus value, the search region is selected based on the locus value (e.g., the search region includes the locus value, the search region is adjacent to the search region that includes the locus value, etc.).

### Data Sources and Types

In example embodiments, the unannotated genetic data can be linked to annotation data. In example embodiments, the annotation data can be characterized by genotype information, phenotype information, evidence levels, drug efficacy data, drug toxicity data metabolic data, or any combination thereof. The annotation data may come from more than one source such as two or more databases, two or more experiments, or a combination thereof. The experiments or databases my include results from one or more of the sequencing methods described herein.

To link unannotated genetic data to annotation data a dataset that includes both annotation data and variant loci data for individual samples can be used. The dataset can be an existing dataset or can be generated de novo. In example embodiments, the dataset includes data from bulk tissue samples. The tissue samples are preferably derived from tissues associated with the annotation data such as genotype information, phenotype information, evidence levels, drug efficacy data, drug toxicity data metabolic data, or any combination thereof. In example embodiments, the dataset includes annotation data and genome data. The genome data is preferably from genomes associated with the annotation data.

In an example, annotation data can include annotations (e.g., received from a plurality of data sources) mapped to variable value sets for coding and/or non-coding DNA variants. In a specific example, at least a portion of the annotations mapped to variable value sets for non-coding DNA variants can be determined using at least one of: genome-wide association studies (GWAS), CRISPR-based functional screens, or by activity-by-contact models.

In example embodiments, the dataset includes genotype data and includes genetic variants. The genetic variants can be in the nuclear genome. The genetic variants may also be present in the mitochondrial genome. In an example embodiment, the annotation data is determined for a population of subjects having a disease (e.g., using a database described herein: UK Biobank, MGB Biobank, TOPMed, and All of Us). The specific variants that make up the annotation data can then be evaluated in a dataset comprising genotype data and molecular profiles (e.g., Genotype-Tissue Expression (GTEx) project). The specific variants that make up the annotation data can then be evaluated in samples without sequencing the whole genome of each sample. The samples can then be evaluated for a molecular profile either simultaneously or after determining annotated variant loci. The samples can be tissue samples obtained from a plurality of subjects. The samples can be cells that have the annotation data and are modified to have different annotation data. The cells having different annotation data can then be evaluated for a molecular profile.

In example embodiments, the dataset can be a cell atlas or single cell atlas. As used herein “atlas” refers to a collection of data from any tissue sample of interest having a phenotype of interest (see, e.g., Rozenblatt-Rosen O, Stubbington M J T, Regev A, Teichmann S A., The Human Cell Atlas: from vision to reality., Nature. 2017 Oct. 18; 550(7677):451-453; and Regev, A. et al. The Human Cell Atlas Preprint available at bioRxiv at dx.doi.org/10.1101/121202 (2017)). The atlas can include biological information, including medical records, histology, single cell profiles, and genetic information.

### Annotation Data

In example embodiments, annotation data includes any data that defines a distinct functional or pathobiological mechanism, such as markers that contribute to a disease, genotype information, phenotype information, evidence levels, drug efficacy data, drug toxicity data metabolic data, or any combination thereof. In example embodiments, samples having different levels for the genomic data can be distributed into categorical variables (e.g., samples having different numbers of markers).

In example embodiments, annotation data is preferably genetic (i.e., genotype data). The annotation data can include genome variants that are associated with the distinct functional or pathobiological mechanism. In example embodiments, the genome variants can be used to generate annotation data. In example embodiments, the annotation data is a partitioned and is enriched for variants that share a similar pattern of genome-wide associations, for example, across disease related traits for the disease (see, Udler M S, Kim J, von Grotthuss M, et al. Type 2 diabetes genetic loci informed by multi-trait associations point to disease mechanisms and subtypes: A soft clustering analysis. PLoS medicine 2018; 15(9): e1002654; and expanded pPS's described in Examples 1 and 2).

In example embodiments, the annotation data is enriched for variants linked to DNA regulatory elements active (e.g., enhancers) in the tissue associated with the genotype information, phenotype information, evidence levels, drug efficacy data, drug toxicity data metabolic data, or any combination thereof. Any methods of linking enhancers to genes expressed in tissues can be used. In example embodiments, an Activity-by-Contact (ABC) model is used to link variants to genes. This model is based on the simple biochemical notion that an element's quantitative effect on a gene should depend on its strength as an enhancer (“Activity”) weighted by how often it comes into 3D contact with the promoter of the gene (“Contact”), and that the relative contribution of an element on a gene's expression should depend on the element's effect divided by the total effect of all elements (see, e.g., Fulco, et al. Activity-by-contact model of enhancer-promoter regulation from thousands of CRISPR perturbations. Nat Genet. 2019; 51(12):1664-1669. doi:10.1038/s41588-019-0538-0; and Moonen, et al., 2020, KLF4 Recruits SWI/SNF to Increase Chromatin Accessibility and Reprogram the Endothelial Enhancer Landscape under Laminar Shear Stress. bioRxiv 2020.07.10.195768, doi.org/10.1101/2020.07.10.195768). In example embodiments, an epigenome model, such as Roadmap, is used to link variants to gene modules (see, e.g., Ernst, J., Kheradpour, P., Mikkelsen, T. et al. Mapping and analysis of chromatin state dynamics in nine human cell types. Nature 473, 43-49 (2011); Kundaje, A., Meuleman, W., Ernst, J. et al. Integrative analysis of 111 reference human epigenomes. Nature 518, 317-330 (2015); and egg2.wustl.edu/roadmap/web_portal/index.html). In example embodiments, an Enhancer-to-gene (E2G) strategy is a combined union of Activity-By-Contact and Roadmap Enhancer-to-gene (E2G) strategy (Roadmap-U-ABC E2G strategy) (see, e.g., US patent application publication US20210071255A1).

In example embodiments, the annotation data includes the most common variants associated with the genotype information, phenotype information, evidence levels, drug efficacy data, drug toxicity data, metabolic data, disease related traits, optionally, including additional variants that are progressively less common for the disease. In example embodiments, the annotation data includes less than 100 variants. In example embodiments, the annotation data includes 100 or more variants. In example embodiments, the annotation data includes between 100 to 400 variants. In example embodiments, the annotation data includes 1000 or more variants.

Identifying the presence of a risk loci can be done by any DNA detection method known in the art, including sequencing at least part of a genome of one or more cells from the subject. In example embodiments, detection of variants can be done by sequencing. Sequencing can be any of those described herein. Sequencing can be, for example, whole genome sequencing. In one example embodiment, the invention involves high-throughput and/or targeted nucleic acid profiling (for example, sequencing, quantitative reverse transcription polymerase chain reaction, and the like).

In example embodiments, sequencing includes high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi:10.1002/0471142727.mb0422s107 (2014). PMCID:4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In example embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In example embodiments, the present invention includes whole genome sequencing. Whole genome sequencing (also known as WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast. “Whole genome amplification” (“WGA”) refers to any amplification method that aims to produce an amplification product that is representative of the genome from which it was amplified. Non-limiting WGA methods include Primer extension PCR (PEP) and improved PEP (I-PEP), Degenerated oligonucleotide primed PCR (DOP-PCR), Ligation-mediated PCR (LMP), T7-based linear amplification of DNA (TLAD), and Multiple displacement amplification (MDA).

In example embodiments, targeted sequencing is used in the present invention (see, e.g., Mantere et al., PLoS Genet 12 e1005816 2016; and Carneiro et al. BMC Genomics, 2012 13:375). Targeted gene sequencing panels are useful tools for analyzing specific mutations in a given sample. Focused panels contain a select set of genes or gene regions that have known or suspected associations with the disease or phenotype under study. In example embodiments, targeted sequencing is used to detect mutations associated with a disease, genotype information, phenotype information, evidence levels, drug efficacy data, drug toxicity data metabolic data, or any combination thereof in a subject in need thereof. Targeted sequencing can increase the cost-effectiveness of variant discovery and detection.

Variants may also be detected through hybridization-based methods, including dynamic allele-specific hybridization (DASH), molecular beacons, and SNP microarrays, enzyme-based methods including RFLP, PCR-based, e.g., allelic-specific polymerase chain reaction (AS-PCR), polymerase chain reaction-restriction fragment length polymorphism (PCR-RFLP), multiplex PCR real-time invader assay (mPCR-RETINA), (amplification refractory mutation system (ARMS), Flap endonuclease, primer extension, 5′ nuclease, e.g., Taqman or 5′nuclease allelic discrimination assay, and oligonucleotide ligation assay, and methods such as single strand conformation polymorphism, temperature gradient gel electrophoresis, denaturing high performance liquid chromatography, high-resolution melting of the entire amplicon, use of DNA mismatch-binding proteins, SNPlex, and Surveyor nuclease assay.

### Molecular Profile Data

In example embodiments, the annotation data includes molecular profiles in the data set include a transcriptomic profile, a proteomic profile, a metabolomic profile, a cell-imaging based profile, a spatial transcriptomic profile, a spatial proteomics profile, a spatial metabolomics profile, an epigenomic profile, a clinical imaging profile, a lipodomic profile, or a combination thereof.

In example embodiments, the molecular profiles are obtained from single cell data. The single cell data is preferably from single cells associated with the disease of interest (e.g., originating from a tissue associated with the disease or specific cell types). In example embodiments, an endotype is linked to a molecular profile in single cell types associated with the disease. In example embodiments, the molecular profile that is linked to an endotype is a molecular profile from a single cell type that has the highest correlation with the endotype. For example, a molecular profile from a plurality of single cells are compared to an endotype score and a molecular profile in a single cell type that most closely correlates with the endotype score is selected.

### Transcriptomic Profile

In example embodiments, the molecular profile includes transcriptome data (e.g., gene expression). As used herein the term “transcriptome” refers to the set of transcript molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In example embodiments, transcriptome data includes bulk RNA sequencing (e.g., RNA-seq). In example embodiments, transcriptome data includes single cell RNA sequencing (e.g., scRNA-seq). In example embodiments, an endotype is linked to a signature in single cell types associated with the disease. In example embodiments, the signature that is linked to an endotype is a signature from a single cell type that has the highest correlation with the endotype. For example, a transcriptome from a plurality of single cells are compared to an endotype score and a gene signature in a single cell type that most closely correlates with the endotype score is selected.

In example embodiments, the invention involves single cell RNA sequencing (see, e.g., Qi Z, Barrett T, Parikh A S, Tirosh I, Puram S V. Single-cell sequencing and its applications in head and neck cancer. Oral Oncol. 2019; 99:104441; Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In example embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In example embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In example embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; and Drokhlyansky E, Smillie C S, Van Wittenberghe N, et al. The Human and Mouse Enteric Nervous System at Single-Cell Resolution. Cell. 2020; 182(6):1606-1622.e23, which are herein incorporated by reference in their entirety.

### Proteomic Profile

In example embodiments, the molecular profile includes proteome data. Proteome data may include mass spectrometry. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

Single cells can be analyzed by mass cytometry (CyTOF) and tissue samples can be analyzed by Multiplexed Ion Beam Imaging (MIBI) (see, e.g., Hartmann F J, Bendall S C. Immune monitoring using mass cytometry and related high-dimensional imaging approaches. Nat Rev Rheumatol. 2020; 16(2):87-99). Non-limiting examples include multiplex analysis of single cell constituents (US20180340939A), single-cell proteomic assay using aptamers (US20180320224A1), and methods of identifying multiple epitopes in cells (US20170321251A1). In example embodiments, CITE-seq (cellular proteins) is used to generate single cell RNA-seq and proteomics data (see, e.g., Stoeckius, M. et al. Simultaneous epitope and transcriptome measurement in single cells. Nat. Methods 14, 865-868 (2017)).

### Epigenomic Profiles

In example embodiments, the molecular profile includes epigenomic profiles. Epigenomic profiles have been described and are obtainable in databases (see, e.g., NIH Roadmap Epigenomics Mapping Consortium, ENCODE, Cistrome, and ChTP Atlas; ENCODE Project Consortium, Moore J E, Purcaro M J, et al. Expanded encyclopaedias of DNA elements in the human and mouse genomes. Nature. 2020; 583(7818):699-710; Li S, Wan C, Zheng R, et al. Cistrome-GO: a web server for functional enrichment analysis of transcription factor ChIP-seq peaks. Nucleic Acids Res. 2019; 47(W1):W206-W21 1; and Shinya Oki, Tazro Ohta, et al. ChIP-Atlas: a data-mining suite powered by full integration of public ChIP-seq data. EMBO Rep. (2018) e46255). The epigenomic profile can be a chromatin accessibility profile (e.g. ATAC-seq), a chromatin modification profile (e.g., ChIP-seq), a chromatin binding profile (e.g., ChIP-seq), a DNA methylation profile (e.g., Bisulfite-Seq), a DNase hypersensitivity profile (e.g., DNase-seq), or a DNA-DNA contact profile (e.g., Hi-C).

In example embodiments, epigenomic profiles are single cell profiles. In example embodiments, the invention involves the Assay for Transposase Accessible Chromatin using sequencing (ATAC-seq) (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1). In example embodiments, genome wide chromatin immunoprecipitation is used (ChIP) (see, e.g., Rotem, et al., Single-cell ChIP-seq reveals cell subpopulations defined by chromatin state, Nat Biotechnol 33, 1165-1172 (2015)). In example embodiments, epigenetic features can be chromatin contact domains, chromatin loops, superloops, or chromatin architecture data, such as obtained by single cell Hi-C (see, e.g., Rao et al., Cell. 2014 Dec. 18; 159(7):1665-80; and Ramani, et al., Sci-Hi-C: A single-cell Hi-C method for mapping 3D genome organization in large number of single cells Methods. 2020 Jan. 1; 170: 61-68). In example embodiments, SHARE-Seq is used to generate single cell RNA-seq and chromatin accessibility data (see, e.g., Ma, S. et al. Chromatin potential identified by shared single cell profiling of RNA and chromatin. bioRxiv 2020.06.17.156943 (2020) doi:10.1101/2020.06.17.156943).

### Spatial Detection Profiles

In example embodiments, the molecular profile includes spatial detection data. In example embodiments, spatially resolved molecular profiles are anchored to an endotype. For example, a pPS can be linked to gene or protein expression in specific cells located at the sites of disease or the location where the disease manifests. An example spatial detection platform includes the digital spatial profiler (DSP), GeoMx DSP, which is built on Nanostring's digital molecular barcoding core technology and is further extended by linking the target complementary sequence probe to a unique DSP barcode through a UV cleavable linker (see, e.g., Li X, Wang C Y. From bulk, single-cell to spatial RNA sequencing. Int J Oral Sci. 2021; 13(1):36). A pool of such barcode-labeled probes is hybridized to mRNA targets that are released from fresh or FFPE tissue sections mounted on a glass slide. The slide is also stained using fluorescent markers (i.e., fluorescently conjugated antibodies) and imaged to establish tissue “geography” using the GeoMx DSP instrument. After the regions-of-interest (ROIs) are selected, the DSP barcodes are released via UV exposure and collected from the ROIs on the tissue. These barcodes are sequenced through standard NGS procedures. The identity and number of sequenced barcodes can be translated into specific mRNA molecules and their abundance, respectively, and then mapped to the tissue section based on their geographic location. The DSP barcode can also be linked to antibodies to detect proteins. An example spatial detection platform includes the CosMx Spatial Molecular Imager (Nanostring) platform, which enables high-plex (˜1,000 genes) spatial transcriptomics and proteomics at single cell and subcellular resolution (see, e.g., He, et al., High-plex Multiomic Analysis in FFPE at Subcellular Level by Spatial Molecular Imaging, bioRxiv 2021.11.03.467020). Other spatial detection methods or platform applicable to the present invention have been described (see, e.g., Li X, Wang C Y. From bulk, single-cell to spatial RNA sequencing. Int J Oral Sci. 2021; 13(1):36. Published 2021 Nov. 15. doi:10.1038/s41368-021-00146-0). Additional non-limiting methods of generating spatial data of varying resolution are known in the art, for example, multiplexed ion beam imaging (MIBI) (see, e.g., Angelo et al., Nat Med. 2014 April; 20(4): 436-442), NanoString (DSP, digital spatial profiling) (see e.g., Li X, Wang C Y. From bulk, single-cell to spatial RNA sequencing. Int J Oral Sci. 2021; 13(1):36; and Geiss G K, et al., Direct multiplexed measurement of gene expression with color-coded probe pairs. Nat Biotechnol. 2008 March; 26(3):317-25), ISS (Ke, R. et al. In situ sequencing for RNA analysis in preserved tissue and cells. Nat. Methods 10, 857-860 (2013)), MERFISH (Chen, K. H., Boettiger, A. N., Moffitt, J. R., Wang, S. & Zhuang, X. Spatially resolved, highly multiplexed RNA profiling in single cells. Science 348, (2015)), smFISH (Codeluppi, S. et al. Spatial organization of the somatosensory cortex revealed by cyclic smFISH. biorxiv.org/lookup/doi/10.1101/276097 (2018) doi:10.1101/276097), osmFISH (Codeluppi, S. et al. Spatial organization of the somatosensory cortex revealed by osmFISH. Nat. Methods 15, 932-935 (2018)), STARMap (Wang, X. et al. Three-dimensional intact-tissue sequencing of single-cell transcriptional states. Science 361, eaat5691 (2018)), Targeted ExSeq (Alon, S. et al. Expansion Sequencing: Spatially Precise In Situ Transcriptomics in Intact Biological Systems. biorxiv.org/lookup/doi/10.1101/2020.05.13.094268 (2020) doi:10.1101/2020.05.13.094268), seqFISH+ (Eng, C.-H. L. et al. Transcriptome-scale super-resolved imaging in tissues by RNA seqFISH+. Nature (2019) doi:10.1038/s41586-019-1049-y.), Spatial Transcriptomics methods (e.g., Spatial Transcriptomics (ST))(see, e.g., St∪hl, P. L. et al. Visualization and analysis of gene expression in tissue sections by spatial transcriptomics. Science 353, 78-82 (2016)) (commercially as Visium); Visium Spatial Capture Technology, 10× Genomics, Pleasanton, CA; WO2020047007A2; WO2020123317A2; WO2020047005A1; WO2020176788A1; and WO2020190509A9), Slide-seq (Rodriques, S. G. et al. Slide-seq: A scalable technology for measuring genome-wide expression at high spatial resolution. Science 363, 1463-1467 (2019)), or High Definition Spatial Transcriptomics (Vickovic, S. et al. High-definition spatial transcriptomics for in situ tissue profiling. Nat. Methods 16, 987-990 (2019)). In example embodiments, proteomics and spatial patterning using antenna networks is used to spatially map a tissue specimen and this data can be further used to align single cell data to a larger tissue specimen (see, e.g., US20190285644A1). In example embodiments, the spatial data can be immunohistochemistry data or immunofluorescence data.

### Metabolic Profiles

In example embodiments, the dataset includes cellular metabolic states obtained from analyzing tissue samples or single cells. In example embodiments, metabolites are detected (see, e.g., Rappez L, Stadler M, Triana S, et al. SpaceM reveals metabolic states of single cells. Nat Methods. 2021; 18(7):799-805. doi:10.1038/s41592-021-01198-0). In example embodiments, the dataset includes cellular metabolic states based on RNA-seq or single-cell RNA sequencing (see, e.g., Wagner A, Wang C, Fessler J, et al. Metabolic modeling of single Th17 cells reveals regulators of autoimmunity. Cell. 2021; 184(16):4168-4185.e21).

### Cell-Imaging Based Profiles

In example embodiments, the data set includes morphological data obtained from differentiating stem cells for a plurality of subjects. The morphological data can be used to generate an endotype score for the subjects (e.g., by quantitating the number and intensity of features) or can be the molecular profile for the subjects. Morphological features can be identified by cell painting (see, e.g., Bray M A, Singh S, Han H, et al. Cell Painting, a high-content image-based assay for morphological profiling using multiplexed fluorescent dyes. Nat Protoc. 2016; 11(9):1757-1774); and Laber, et al., Discovering cellular programs of intrinsic and extrinsic drivers of metabolic traits using LipocyteProfiler, bioRxiv 2021.07.17.452050).

In example embodiments, the molecular profile includes histology data. Histology, also known as microscopic anatomy or microanatomy, is the branch of biology which studies the microscopic anatomy of biological tissues. Histology is the microscopic counterpart to gross anatomy, which looks at larger structures visible without a microscope. Although one may divide microscopic anatomy into organology, the study of organs, histology, the study of tissues, and cytology, the study of cells, modem usage places these topics under the field of histology. In medicine, histopathology is the branch of histology that includes the microscopic identification and study of diseased tissue. Biological tissue has little inherent contrast in either the light or electron microscope. Staining is employed to give both contrast to the tissue as well as highlighting particular features of interest. When the stain is used to target a specific chemical component of the tissue (and not the general structure), the term histochemistry is used. Antibodies can be used to specifically visualize proteins, carbohydrates, and lipids. This process is called immunohistochemistry, or when the stain is a fluorescent molecule, immunofluorescence. This technique has greatly increased the ability to identify categories of cells under a microscope. Other advanced techniques, such as nonradioactive in situ hybridization (ISH), can be combined with immunochemistry to identify specific DNA or RNA molecules with fluorescent probes or tags that can be used for immunofluorescence and enzyme-linked fluorescence amplification.

### Genotype and Phenotype Information

In example embodiments, the plurality of annotation sources include genotype information and/or phenotype information. Genotype information includes information regarding genetic material, such as the type of variant present at a locus (i.e., allele). In example embodiments, the genotype information includes symbols representing variant loci. In example embodiments, genotype information may include one or more variant loci. In example embodiments, genotype information may include the variant loci for the whole genome. Genotype information may include the presence or absence of a particular variant (e.g., determining whether a allele causing disease is present). In example embodiments, genotype information includes the ratio of a subject's genotype information to a population of genotype information. The population of genotype information may be derived from any of the sources described therein, such as databases or experimental procedures. Genotyping (i.e., method of determining genotype information) may be performed by any of the sequencing methods described herein. Genotypes are well known to one skilled in the art and will not be discussed in detail herein.

Phenotype information includes any observable trait determined or contributed by a genotype. Phenotype information may include the physical appearance (e.g., sex, ethnicity, eye color), biological development (e.g., levels of hormones or blood type) and behavior (e.g., cognitive patterns). In example embodiments, phenotype information may include the presence or absence of a particular observable trait. In example embodiments, phenotype information includes the ratio of a subject's phenotype information to a population of phenotype information. The population of phenotype information may be derived from any of the sources described therein, such as databases or experimental procedures. Phenotypes are well known to one skilled in the art and will not be discussed in detail herein.

### Pharmacogenomics

In an example embodiment, the annotation data includes pharmacogenomic information. Pharmacogenomics is the study of an individual's (i.e., a subject) or populations response (i.e., one or more database(s)) to a drug as a result of their genetics. Pharmacogenomic data may include drug efficacy data, drug toxicity data, and/or metabolic data. In an example embodiment, the plurality of annotation sources include drug efficacy data, drug toxicity data, and/or metabolic data. Examples of drugs or therapeutics affected by an individual's genes are clopidogrel resistance, warfarin sensitivity, warfarin resistance, malignant hyperthermia, Stevens-Johnson syndrome/toxic epidermal necrolysis, and thiopurine S-methyltransferase deficiency.

An individual's genetics may affect, for example, drug receptors, uptake, and/or breakdown. The amount and/or frequency of a drug required by an individual (i.e., dosage) may need to be increased or decreased beyond the recommended does because the individual produces less or more drug receptors than the average individual (i.e., an individual the normal, recommended dosage is assigned). The determination of an individual's drug receptors can be determined by various genomic loci. For example, breast cancer produces too many HER2 receptors and the dosage of T-DM1 must be increased.

In some instances, an individual's dosage may need to increase or decrease the amount or frequency required for a drug. An individual's tissues and cells may uptake a drug more readily or slowly than the average individual. Conversely, an individual's tissues and cells may remove drugs more slowly or readily than the average individual. In both instances, the determination of an individual's drug uptake and removal can be determined by various genomic loci. For example, some individuals have variations in their SLCO1B1 gene that reduced the uptake of statin (i.e., simvastatin) into the liver and must be reduced to prevent muscle issues from the excess build up of statin. Another reason the dosage required by an individual may need to be increased or decreased beyond the recommended dose is an individual's ability to breakdown the drug. More of a drug will be required if the individual's body decomposes the drug more readily, while less of the drug will be required if the individual's body breaks down the drug more slowly. For example, CYP2D6 and CYP2C19 influence an individual's ability to break down amitriptyline, an antidepressant. Therefore, the dosage may need to be varied based on the individual's expression of CYP2D6 and CYP2C19.

### Filtering and Weight Metrics

In example embodiments, the annotation data associated with each variant is filtered based on a weight metric. Filtering includes choosing a subset of annotation data and using the subset to annotate variant loci. Filtering is typically performed for increase the accuracy of the data and reduce lower quality annotations. Filtering data is commonly used by one skilled in the art and further details will not be described herein.

In example embodiments, the weight metric is computed based on number of published annotations, annotation data is clinical grade, whether these annotations are based on expert panel review presence and number of conflicting annotation. A weight metric is a numerical value given to a piece of information in the annotation data. In general, the numerical value is non-negative but, in some embodiments, a piece of annotation data may be an indicator of low quality information and may be assigned a negative numerical value. In example embodiments, a weight of zero is used to exclude annotation data. In the case of positive correlation, annotation data with large numerical values may refer to more accurate information while smaller numerical values may refer to less accurate information. The opposite is true for negatively correlated annotation data.

The weight metrics may be computed by any statistical analysis known in the art. For example, weight metrics may be computed as frequency weights, survey weights, or analytical weights. Frequency weights, in general, assign a variable proportional to the number of observables for a given piece of annotation data. Survey weights (e.g., sampling weights or probability weights), in general, assign a normalized variable to an observable for a given piece of annotation data as compared to other pieces of annotation data from the plurality of annotation data. Analytical weights (e.g., inverse variance weights or regression weights), in general, assign a value according to the piece of annotation data as it is organized in the plurality of annotation data with some variance.

Weighted metrics based on the number of published annotations (e.g., published articles), for example, may include frequency weights or analytical weights. The frequency weight may increase proportional to the number of published annotations. Separately or in combination with frequency weights, analytical weights may also be used to distinguish the quality of the published annotation data. Analytical weights may also be used for annotation data that is clinical grade, wherein clinical grade data is given priority weight over non-clinical grade data or varying levels of clinical grade data are given varying weights. In example embodiment, annotation based on expert panel review presence may include survey weights, wherein the level of expertise or the results of the panel determine the weight given to the annotation data.

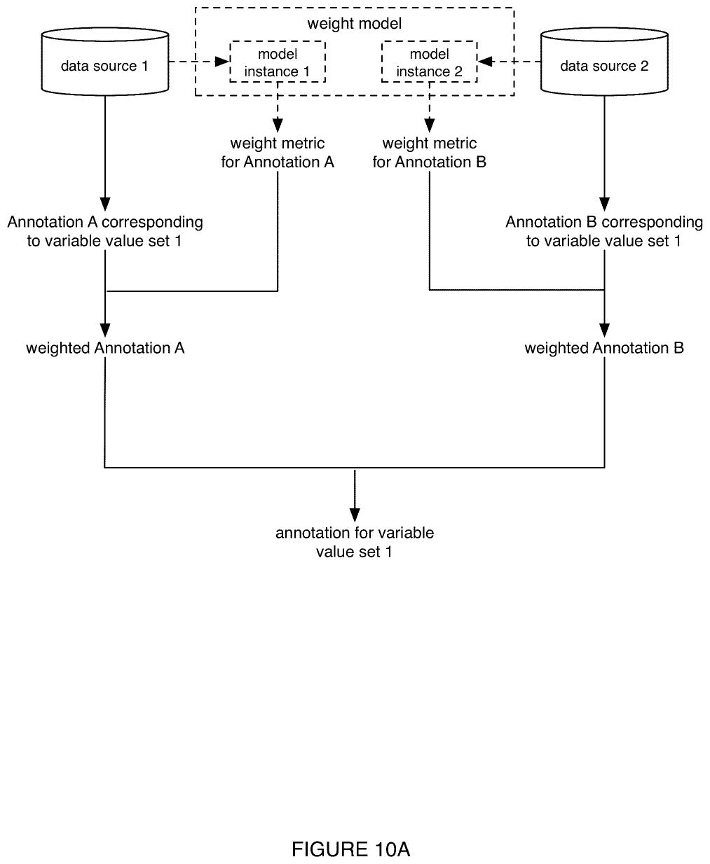

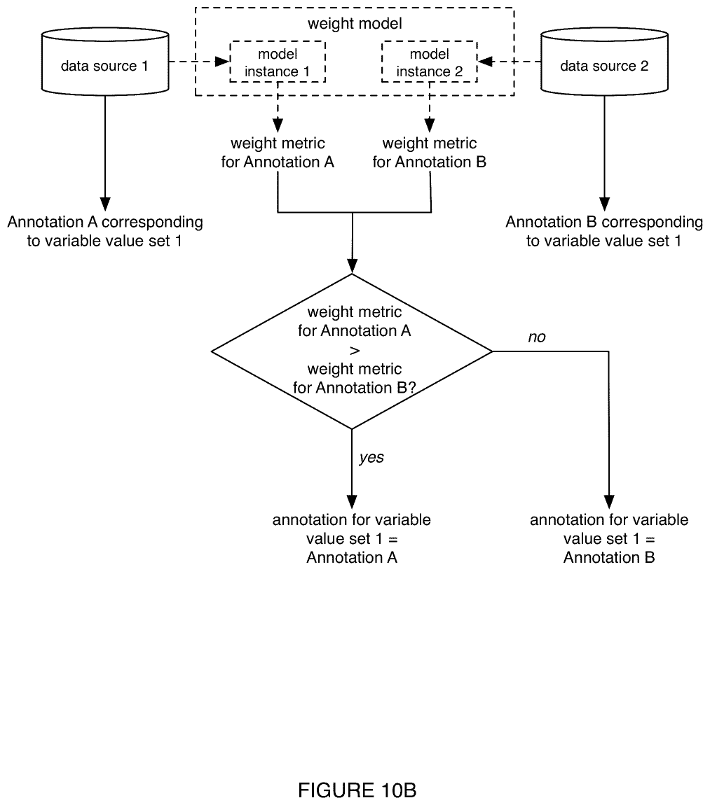

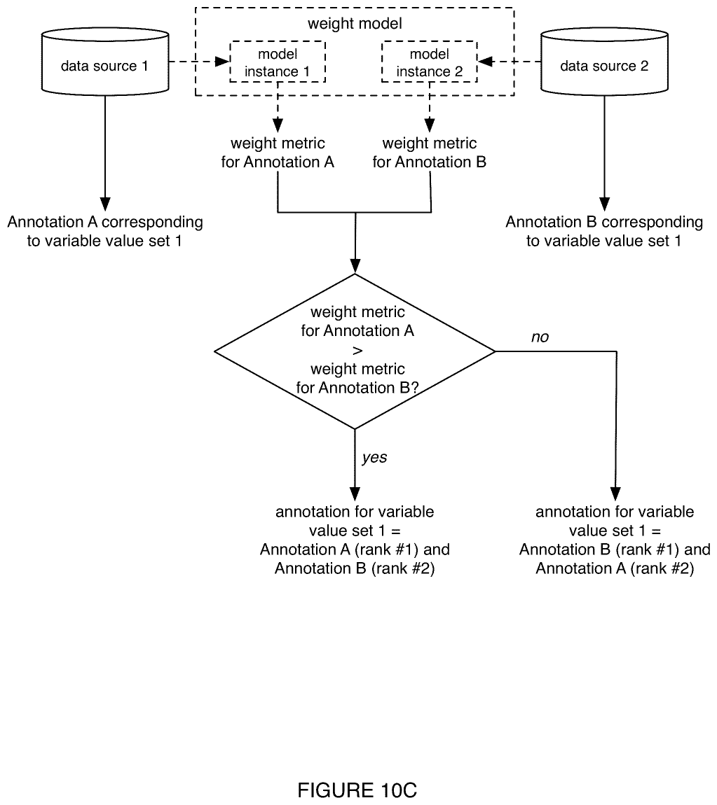

In variants, generating annotation data can include mapping annotations (received from a plurality of data sources) to variable value sets (e.g., match-optimized variable values), wherein each variable value set corresponds to a genomic variant, wherein the annotation for at least one variable value set comprises a weighted aggregation of multiple annotations, from different data sources, associated with the respective variable value set. Examples are shown in FIG. 10A, FIG. 10B, and FIG. 10C.

In an example, the weighted aggregation is performed based on a weight metric for each of the multiple data sources and/or for each annotation. In a specific example, the weight metric for a data source can be determined based on at least one of: a number of published annotations associated with the data source, whether annotations from the data source are clinical grade, whether annotations from the data source are based on expert panel review presence, whether the data source is FDA-recognized, whether the data source and/or annotations therefrom conform to practice guidelines, and/or any other information associated with the data source and/or annotation(s) from the data source. The weight metric can optionally include a predictive weight determined using a supervised learning model.

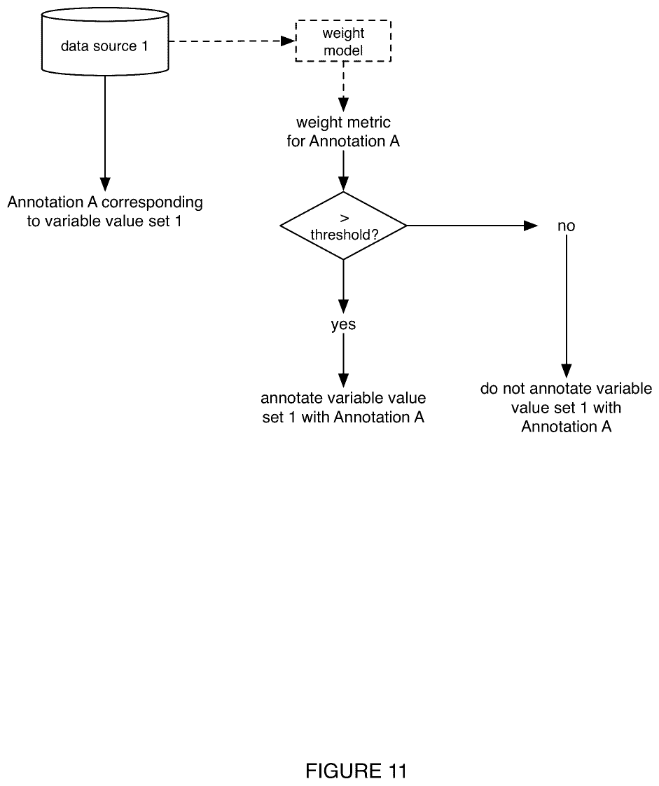

In another example, the weighted aggregation is performed based on a weight metric for each annotation of the multiple annotations. An example is shown in FIG. 11. In a first specific example, performing the weighted aggregation of the multiple annotations can include identifying conflicting annotations in the multiple annotations and selecting an annotation from the conflicting annotations with a higher weight metric. In a second specific example, performing the weighted aggregation of the multiple annotations can optionally include filtering out annotations with a weight metric below a threshold. In a third specific example, performing the weighted aggregation of the multiple annotations can include filtering out annotations conflicting with another annotation (e.g., filtering out all or a portion of conflicting annotations; filtering out conflicts from sources other than literature sources; filtering out conflicts from sources that are recent such as less than a threshold number of years old; etc.).

### Annotation Type

In an example embodiment, each annotation is categorized by annotation type. The annotation data may be separated, designated, enumerated or otherwise categorized. The annotation type may be any characteristic distinguishable from another. In example embodiments, the annotation type includes risk variant type, protective variant type, drug responsiveness, metabolic effects (i.e., how a subject or individuals body processes food, drugs/chemicals, or its own tissue), or any combination thereof. These annotation types are further described herein and one skilled in the art would recognize these annotation types.

### Additional Information

In example embodiments, the visual element further includes one or more links to additional information about the annotation. The additional information may include links to resources corresponding the annotated variant loci. These resources may be internal to the methods and system described herein, external to these methods and systems, or a combination thereof. For example, these resources may include genetic counseling, the sources from which the annotated variant loci were derived, additional sources further detailing the annotated variant loci (e.g., more information regarding a particular disease or drug), or any combination thereof.

### Non-Coding DNA

In example embodiments, the genotype information includes non-coding DNA variant information. Non-coding DNA includes regulators of cellular function as well as markers for diseases. Non-coding DNA variant information includes information regarding the cellular function regulated by the non-coding DNA variant as well as the markers for disease. For example, non-coding DNA variant information regarding cellular function may include regulatory elements, instructions for the formation of RNA molecules, structural elements of chromosomes, and introns.

Non-coding DNA sequences comprising regulatory elements, in general, determine the activation of one or more gene (i.e., which genes are turned off and on). These regulatory elements include, for example, promotors, enhancers, silencers, and insulators. Non-coding sequences that include promotors include binding sites for proteins that carry out transcription and may be located before coding sequences on the DNA (i.e., transcriptional start site). Non-coding sequences that include enhancers include binding sites for proteins that participate in activating transcription and may be located before or after the coding sequences they regulate. However, some enhancers may be found far away from coding sequence they regulate (e.g., SHH enhancer is located ˜1 Mb away from the gene it regulates).

Non-coding sequences that include silencers include binding sites for proteins that repress transcription and are located before or after the coding sequences they regulate. However, some silencers may be found far away from coding sequence they regulate. Non-coding sequences that include insulators may include binding sites for proteins that control transcription. For example, insulators may prevent enhancers from participating in transcription and are known as enhancer-blocker insulators. In another example, insulators may prevent structural changes in DNA thereby repressing gene activity and are known as barrier insulators. In some instances, insulators carry out the function of both enhancer-blocker and barrier insulators.

Non-coding sequences that are structural elements of chromosomes may form telomeres or satellite DNA. Non-coding sequences comprising satellite DNA may include of centromeres or heterochromatin. Centromeres are the constriction point of the X-shaped chromosome pair. Heterochromatin, densely packs DNA and maintains the structure of chromatin thereby regulating gene activity. Non-coding sequences comprising introns are located within protein-coding genes but are removed before translation. Additional non-coding sequences found between genes may include intergenic regions.

Non-coding DNA variant information comprising markers of diseases. It has been demonstrated, in multiple disease, disease-associated SNPs occur in the non-coding region (e.g. 90%). See e.g., Perenthaler, E.; Yousefi, S.; Niggl, E.; Barakat, T. S. Beyond the Exome: The Non-Coding Genome and Enhancers in Neurodevelopmental Disorders and Malformations of Cortical Development. Frontiers in Cellular Neuroscience, 2019, 13 hereby incorporated by reference.

### Genome Wide Association Studies

In an example embodiment, a connection to non-coding variants to coding genes or disease states is determined from genome-wide association studies (GWAS), CRISPR-based functional screens, or by activity-by-contact models. GWAS assess genetic variants across multiple genomes to identify phenotypes, genotypes, or diseases associated with the genetic variants. For non-coding regions, GWAS can identify the regulatory function or markers of disease located in these regions. In general, GWAS includes collecting DNA and phenotypic information from multiple individuals. Phenotypic information may include any biological information about a subject. The DNA of each subject is then genotyped. Genotyping may include using GWAS arrays or any sequencing method described herein. The resulting data is then processed, which includes one or more steps of: performing quality controls; assigning untyped variants using haplotype phasing and reference populations; performing statistical tests; conducting a meta-analysis; independent replication; interpreting the results; or any combination thereof.

See e.g., Uffelmann, E., Huang, Q. Q., Munung, N. S. et al. Genome-wide association studies. Nat Rev Methods Primers 1, 59 (2021) and Tak, Y. G., Farnham, P. J. Making sense of GWAS: using epigenomics and genome engineering to understand the functional relevance of SNPs in non-coding regions of the human genome. Epigenetics & Chromatin 8, 57 (2015)

CRISPR-based functional screens and activity-by-contact models are further described herein.

### Clinical Testing/Screening

In example embodiments, the methods and systems described herein provide a recommendation for further clinical testing. Clinical testing may include any health screen recommended by the methods and systems herein. These health screens are used to detect potential disorders or diseases corresponding the annotated variant loci. The health screenings may be multiphasic screening (i.e., two or more screening tests). Example health screens may include alcohol screening, blood pressure screening, cancer screening (e.g., breast, cervical, colorectal), cholesterol screening, dental exam, depression screening, osteoporosis screening.

### Method of Determining Disease Risk or Prognosis

The method can optionally include determining a risk score for the subject S250, which functions to determine a predisposition for a trait of interest. S250 can optionally be performed using the genome system 130. S250 can be performed once, multiple times (e.g., for each trait of interest in a set), and/or any other number of times. The trait of interest can be a disease of interest (e.g., breast cancer), a collection of diseases (e.g., all cancers), observable traits (e.g., height, eye color, etc.), and/or any other phenotype. The trait of interest can be predetermined, determined based on the genomic data and/or clinical features for the subject, input by the subject and/or other user, randomly determined, manually determined, and/or otherwise determined. The risk score (e.g., disease risk score) can be a genomic risk score (e.g., polygenic risk score), a composite risk score, a lifetime risk, a percentile risk, and/or any other risk score. In a specific example, the composite risk score accounts for clinical features in addition to genomic data. Examples of clinical features (e.g., clinical factors) include other genetic features (e.g., monogenic mutations and/or presence of genetic variants such as SNPs, CNVs, Insertions, Duplications, Deletions, etc.), demographic data (e.g., ancestry, sex, age, ethnicity, location, income, wealth, education, etc.), family history (e.g., presence or absence in first-degree family, number of first degree relatives, number of second degree relatives, number of third degree relatives, specific relatives, etc.), clinical results (e.g., lab results such as cholesterol levels, scans such as MRI, etc.), personal characteristics and/or risk factors (e.g., height, weight, BMI, alcohol use, smoking, physical activity, diet, age at menopause, age at pregnancy, pregnancies, parity (number of full term pregnancies), miscarriages, surgery history, hormone history including use of HRT/estrogen/progesterone, etc.), non-physical factors (e.g., mental health, political beliefs, relationship status, economic status, etc.), personal health history (e.g., disease history, drugs taken, surgery, hormone history, etc.), and/or any other factors. Clinical features can be extracted from medical records, input by a user (e.g., self-reported), determined based on genomic data, determined based on other clinical features, predetermined, manually determined, randomly determined, and/or otherwise determined. In a first specific example, demographic data (e.g., ancestry information) can be determined (e.g., inferred) based on the genomic data for the subject. In a second specific example, demographic data (e.g., ancestry information) can be received (e.g., self-reported from the subject). The risk score is preferably quantitative, but can additionally or alternatively be qualitative, relative, discrete, continuous, a classification, numeric, binary, and/or be otherwise characterized.

A risk score can be determined using a risk model (e.g., genomic risk model, composite risk model, lifetime risk model, percentile risk model, etc.). Inputs to the risk model (e.g., received by the genome system 130, output from other models in the genome system 130, etc.) can include: unannotated and/or annotated identified variant loci for the subject, variable values (e.g., a variable value set corresponding to an identified variant locus), genomic data, clinical features, other risk scores, population data, annotation data (e.g., clinical data), and/or any other suitable inputs. Outputs from the risk model can include the risk score and/or any other suitable outputs. The risk model can include classical or traditional approaches, machine learning approaches, and/or be otherwise configured. The risk model can be specific to a trait of interest, general across traits, specific to one or more clinical features, general across clinical features, and/or otherwise configured. The risk model can include regression (e.g., linear regression, non-linear regression, logistic regression, etc.), decision tree, random forest, LSA, clustering, association rules, dimensionality reduction (e.g., PCA, t-SNE, LDA, etc.), neural networks (e.g., CNN, DNN, CAN, LSTM, RNN, FNN, encoders, decoders, deep learning models, transformers, etc.), ensemble methods, optimization methods (e.g., Bayesian optimization), classification, rules, heuristics, equations (e.g., weighted equations, etc.), selection (e.g., from a library), lookups, regularization methods (e.g., ridge regression), Bayesian methods (e.g., Naiive Bayes, Markov), instance-based methods (e.g., k-nearest neighbor), kernel methods, support vectors (e.g., SVM, SVC, etc.), statistical methods (e.g., probability), boosting methods, bagging methods, comparison methods (e.g., matching, distance metrics, thresholds, etc.), deterministics, genetic programs, and/or any other suitable model. The risk model can include (e.g., be constructed using) a set of input layers, output layers, and hidden layers (e.g., connected in series, such as in a feed forward network; connected with a feedback loop between the output and the input, such as in a recurrent neural network; etc.; wherein the layer weights and/or connections can be learned through training); a set of connected convolution layers (e.g., in a CNN); a set of self-attention layers; and/or have any other suitable architecture.

Multiple risk models can optionally be arranged in series and/or parallel. For example, a first risk model can output a first risk score, wherein a downstream second risk model can output a second risk score based on the first risk score. Optionally, a downstream third risk model can output a third risk score based on the second risk score and/or the first risk score. The first, second, and/or third risk models can be: a genomic risk model (outputting a genomic risk score), a composite risk model (outputting a composite risk score), a lifetime risk model (outputting a lifetime risk), a percentile risk model (outputting a percentile risk), and/or any other risk model. In a first specific example, the first risk model can be a genomic risk model, and the second risk model can be a composite risk model, a lifetime risk model, and/or a percentile risk model. In an illustrative example, inputs to the second risk model can be a set of features, wherein the set of features can include a genomic risk score (output by a genomic risk model), clinical features, and/or any other model inputs. In a second specific example, the first risk model can be a composite risk model, and the second risk model can be a lifetime risk model and/or a percentile risk model. In a third specific example, the first risk model can be a genomic risk model, the second risk model can be a composite risk model, and the third risk model can be a lifetime risk model and/or a percentile risk model. However, one or more risk models can be otherwise arranged.

The risk model can be trained, learned, fit, predetermined, and/or can be otherwise determined. The risk model can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated. The risk model can be learned or trained on: labeled data (e.g., population data labeled with a trait label), unlabeled data, positive training sets (e.g., a set of data with true positive labels), negative training sets (e.g., a set of data with true negative labels), and/or any other suitable set of training data. In a specific example a risk model can optionally be trained by correlating against response variables (e.g., drugs, scans, interventions, recovery, etc.) and holding covariates constant (e.g., to achieve a more causal relationship).

The training data preferably includes population data (e.g., population genomic data, population clinical features, etc.) labeled with a trait label (e.g., disease label). For example, the risk model can be trained by: for each training subject, determining a target risk score based on the disease label for the training subject, and training the risk model to predict the target risk score based on data (e.g., genomic data, clinical features, etc.) corresponding to the training subject. The training data can optionally include supplemental labels (e.g., demographic information such as ancestry). Population genomic data can optionally be annotated using all or parts of the method. The training data can optionally include synthetic data to augment training. Synthetic data can be determined using upsampling, SMOTE, and/or any other augmentation method. In an illustrative example, specific demographics (e.g., black women) can be upsampled. The risk model can optionally correspond to one or more clinical features (e.g., ancestry), wherein the training data is determined (e.g., selected) to correspond to the clinical feature (e.g., upsampling population data corresponding to an ancestry of interest, augmenting the training data with synthetic data corresponding to an ancestry of interest, etc.). In a specific example, a different risk model (e.g., different genomic risk models, different composite risk models, etc.) can be used for each of a set of ancestries.

The risk model can optionally be validated (e.g., using cross-validation), verified, reinforced, calibrated, retrained, regularized, or otherwise updated based on newly received, up-to-date data and/or any other suitable data. The risk model can optionally be retrained and/or updated: once; at a predetermined frequency; every time the method is performed; every time an unanticipated input is received; or at any other suitable frequency. The risk model is preferably trained and/or validated before genomic data and/or other inputs for the subject is received, but can alternatively be trained and/or validated after inputs for the subject are received. In specific examples, methods of validating the risk model can include: regularizing by setting variables to 0 (e.g., Lasso), low (e.g., Ridge), and/or mix (e.g., Elastic net); comparing labels (e.g., using self-reporting, test results, and/or clinical notes; requiring concordance and/or loose concordance; etc.); analyzing the replication of null signals to determine if there are any patterns of bias; choosing a best model based on performance in a validation set (e.g., using accuracy, weighted accuracy, sensitivity, specificity, precision, recall, AUC, R\hat{ }2, etc.); and/or any other validation methods.

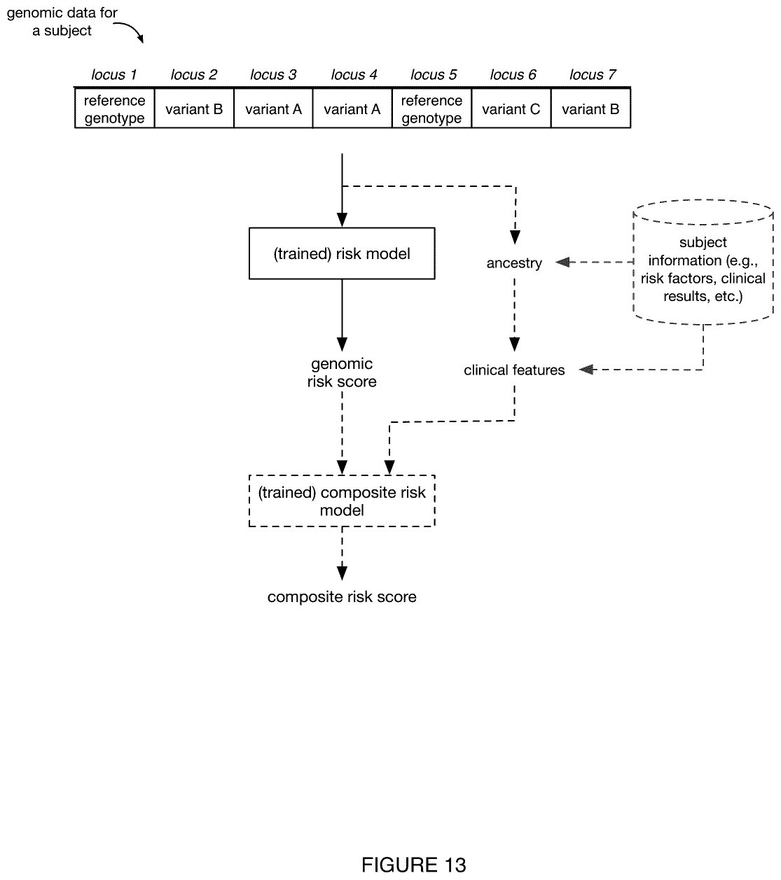

In a first embodiment, the risk model is a genomic risk model that outputs a genomic risk score for the trait of interest (e.g., disease(s) of interest) based on all or a subset of unannotated and/or annotated identified variant loci in the genomic data for the subject (e.g., unannotated and/or annotated variable value sets). An example is shown in FIG. 13.

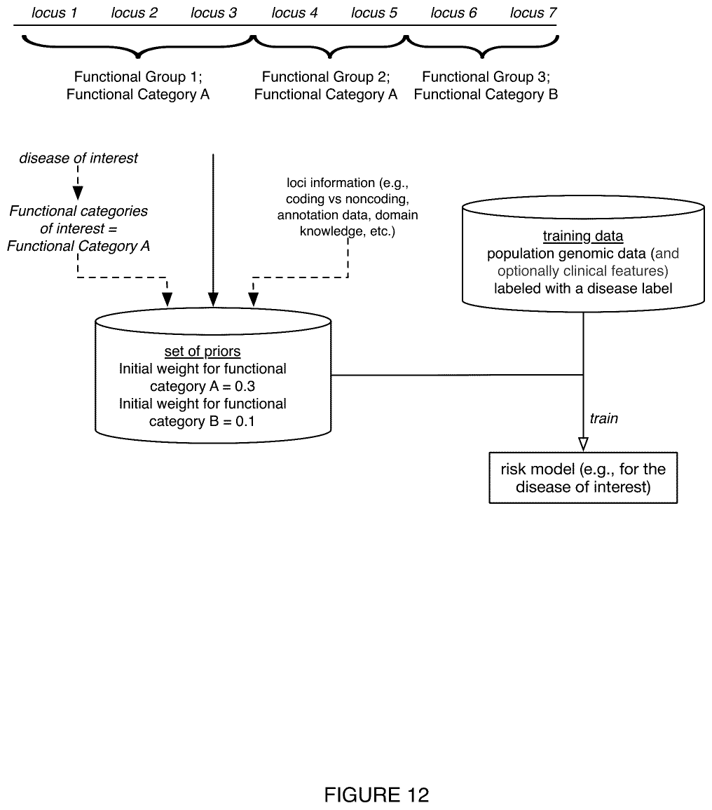

Training the genomic risk model can include determining an initial risk model using the set of priors, and updating the initial risk model using training data, wherein the training data includes population genomic data labeled with a trait label (e.g., disease label). The set of priors (e.g., functional priors) can be determined based on functional data (e.g., annotation data). For example, the set of priors can be determined based on functional annotations mapped to loci (e.g., coding and/or non-coding loci). In an example, the set of priors can include an initial set of weights, wherein each weight corresponds to a single locus and/or a group of loci. An example is shown in FIG. 12.

The set of priors can be determined by segmenting a set of loci (across all or a subset of chromosomes) into a set of functional groups, wherein each functional group corresponds to one or more functional categories. The loci can be segmented based on functional data. For example, the functional category can be determined for a locus based on an ABC score, results of CRISPR screen, results from a functional assay, and/or any other functional data. Functional categories can be disease pathways, categories of genetic function, and/or any other physically relevant and/or clinically relevant category. Illustrative examples of functional categories can include: coding versus noncoding, regulatory categories (e.g., promoter, enhancer, silencer, insulator, etc.), strength categories (e.g., enhancer versus strong enhancer), disease pathway categories (e.g., LDL cholesterol, inflammation, cellular proliferation, vascular remodeling for heart disease, hormone for breast cancer versus no hormone for breast cancer, DNA repair, etc.), and/or any other functional category. In an illustrative example, segmenting the set of loci into the set of functional groups includes segmenting the set of loci based on whether each locus in the set of loci is a coding locus or a noncoding locus. The functional groups can be: overlapping or nonoverlapping, contiguous or non-contiguous, and/or otherwise configured. In a first example, the set of loci can be segmented into overlapping functional groups. In a specific example, loci can be tagged with one or more functional categories, wherein a functional group corresponds to all loci tagged with the corresponding functional category. In an illustrative example, a first functional group corresponding to inflammation can include multiple (noncontiguous) sets of loci across one or more chromosomes; a second functional group corresponding to cellular proliferation can include multiple (noncontiguous) sets of loci across one or more chromosomes, wherein the sets of loci for the first functional group can overlap with the sets of loci for the second functional group. In a second example, the set of loci can be segmented into nonoverlapping functional groups. The functional categories can optionally be selected based on the trait of interest (e.g., wherein the loci are segmented into functional groups corresponding to the selected functional categories). In an illustrative example, for heart disease, the functional categories can include LDL cholesterol and vascular remodeling for heart disease.

In an example, the set of priors can include an initial weight corresponding to each functional group, wherein the initial weight for each functional group is determined based on the respective functional category. For example, the weight can be determined based on the relevance of the functional category to the trait of interest (e.g., determined using domain knowledge and/or the functional data), based on the type of functional category (e.g., whether the functional category is a genetic function category, a disease pathway category, etc.), determined using a model, and/or otherwise determined. In an illustrative example, functional categories of interest can be selected based on the trait of interest, wherein the initial weight for each functional group is determined based on whether the functional group corresponds to a functional category of interest. In a first specific example, the weight corresponds to the functional group as a whole. In a second specific example, the weight corresponds to each locus within the functional group. A weight for a given locus corresponding to multiple functional groups can optionally be a weight aggregated (e.g., averaged, summed, weighted average, weighted sum, any other statistical measure, etc.) across weights for the multiple functional groups.

Training the genomic risk model can include updating the initial weights. Updating the initial weights can include individually updating a weight for each locus in a set of loci, updating a weight corresponding to all loci within a functional group, and/or otherwise updating weights. For example, the genomic risk model can be a regression with the risk score as the dependent variable, wherein the trained genomic risk model is the fitted regression (e.g., updating the weights to minimize loss) to the population genomic data labeled with trait labels for the trait of interest (e.g., whether genomic data for an individual in the population had a disease of interest). In a first example, the independent variables for the regression include a variable value set (e.g., match-optimized variable values) at each locus. In a second example, the independent variables for the regression include a binary value corresponding to the variable value set at each locus (e.g., a presence or absence of an identified variant at the locus; 0 representing the reference genotype and 1 representing any variant). In a third example, the independent variables for the regression include a nonbinary value corresponding to the variable value set at each locus (e.g., 0 representing the reference genotype, 1 representing one copy of variant A, 2 representing two copies of variant A, etc.). In a fourth example, different variants for a given locus are linked to different initial weights (e.g., based on the functional data), wherein the independent variables for the regression include a binary value for each variant possibility for a given locus (e.g., if the subject has variant B, the subject would have a 0 value for variant A and the reference genotype, and a 1 for variant B).

In examples, the genomic risk model can be a regression (e.g., a Bayesian form of multivariate regression), a model using outputs (e.g., posterior probabilities) from a first layer to feed into another layer, any supervised learning method, and/or any other model. In a first specific example, the genomic risk model can be or include a regression trained using a LDpred-funct methods, including a Bayesian supervised learning method that leverages trait-specific functional prior annotations. In a second specific example, the genomic risk model can be or include a transformer (e.g., with self-attention across the full genome). In a third specific example, the genomic risk model can use an enhancer-gene connection framework. Thresholding for the genomic risk model can optionally be performed via pruning, LDpred, LDpred-funct, and/or any other thresholding methods. In an example, training the risk model can include: creating a list of trait-specific functional priors for variant importance, analytically estimating posterior mean causal effect sizes, and regularizing estimates (e.g., using cross validation).

In a second embodiment, the risk model is a composite risk model that outputs a composite risk score for the trait of interest. In a first example, the composite risk model inputs include the genomic risk score (e.g., disease risk score) and a set of clinical features for the subject. In a specific example, the genomic risk score and each clinical feature in the set of clinical features are treated as features in the composite risk model. In a second example, the composite risk model inputs include all or a subset of unannotated and/or annotated identified variant loci in the genomic data for the subject (e.g., annotated and/or unannotated variable value sets) and a set of clinical features for the subject. For example, the composite risk model can be a genomic risk model (e.g., as described in the first embodiment) that takes additional inputs, including clinical features.

However, the risk score can be otherwise determined.

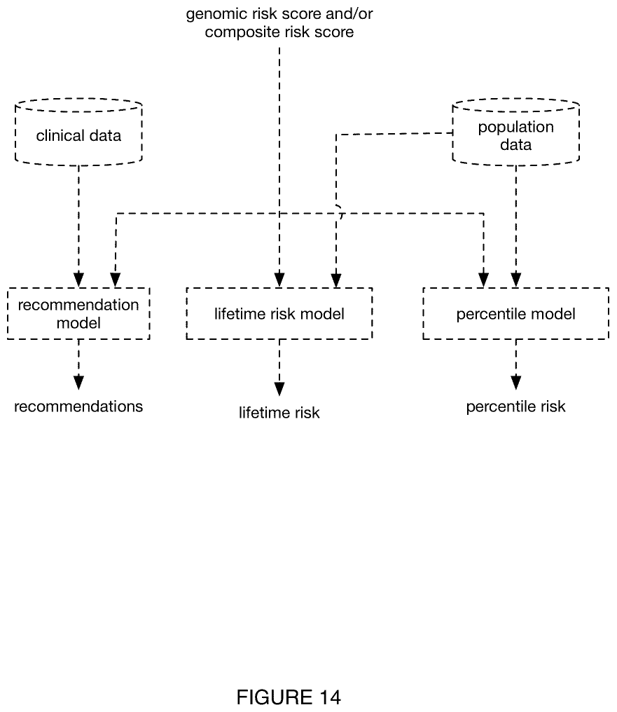

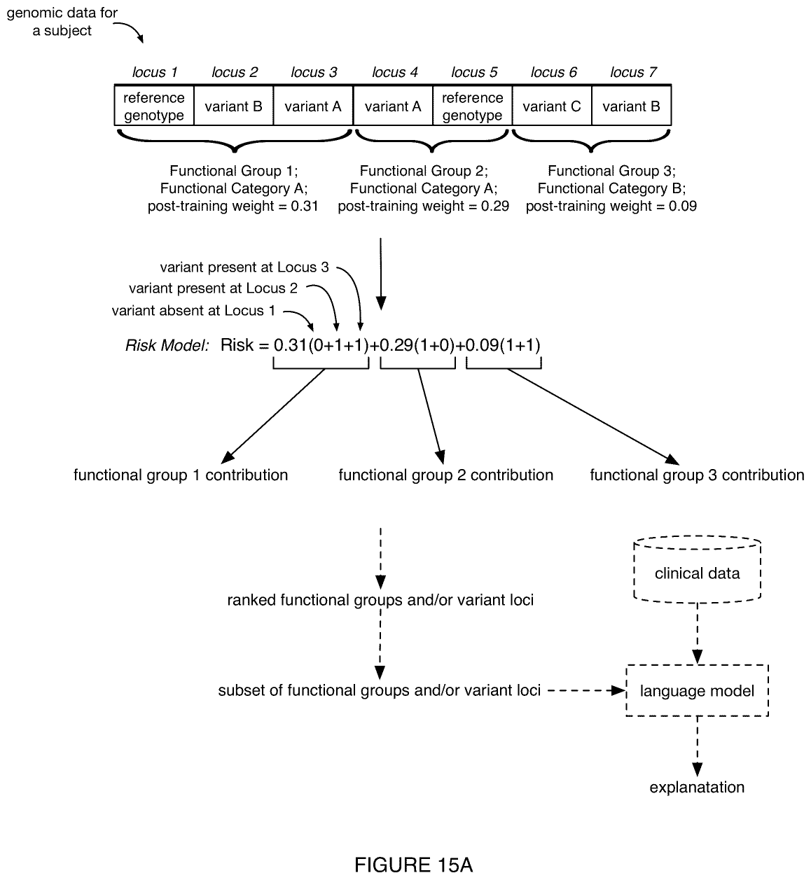

The risk score (e.g., the genomic risk score and/or the composite risk score) can optionally be used to determine: treatment recommendations, a lifetime risk, a percentile risk for the subject relative to a reference population, and/or any other trait information for the subject. An example is shown in FIG. 14. The treatment recommendations, lifetime risk, percentile risk, and/or other information can optionally be provided to the subject.

In a first example, a percentile risk for the subject can be determined based on the composite risk score and a set of population data (e.g., general population and/or population data selected based on an ancestry and/or other clinical features for the subject), using a percentile risk model.

In a second example, a lifetime risk for the subject can be determined based on a risk score (e.g., composite risk score and/or genomic risk score) and a set of population data, using a lifetime risk model. In a specific example, the lifetime risk model can be a model predicting the risk of the subject developing the trait over time. In examples, the lifetime risk model can be used to determine a 1-year risk, 5-year risk, 10-year risk, 20-year risk, lifetime risk, and/or a risk over any other period of time. In a specific example, determining the lifetime risk includes segmenting the training data by age groups, determining a risk for the subject for each age group using the respective training data segment, and projecting a lifetime risk based on the risk for each age group. Training data used to train (e.g., fit) the lifetime risk model can optionally include adjusted population data (e.g., augmented using upsampling, subsetting, etc.), such that the training data reflects a target population (e.g., target demographics, target incidence rate by age, etc.). Specific examples of lifetime risk models can be or include: Cox-proportional hazards model, iCare model, statistical models, and/or any other model. In a specific example, the lifetime risk model can calculate a time to event (e.g., disease incidence event, death event, etc.) based on one or more of: the genomic risk score (e.g., a classification of the genomic risk score), the composite risk score (e.g., a classification of the composite risk score), the percentile risk, clinical features (e.g., age, risk factors, etc.), covariates (e.g., healthy bias covariates), competing risk (e.g., age-specific incidence rates), and/or any other inputs.

In a third example, a set of recommendations can be determined based on a risk score (e.g., the lifetime risk) and a set of clinical data (e.g., annotation data), using a recommendation model. In a specific example, the set of recommendations can be determined based on whether the lifetime risk is above a threshold. Examples of recommendations (e.g., intervention recommendations) can include one or more of: a recommendation for further (e.g., follow up) clinical testing (e.g., scans such as MRIs, mammograms, etc.; GRAIL/guardant/blood biopsies; Prostate-Specific Antigen (PSA) Test; biopsy; etc.), a disease diagnosis, a recommended therapeutic regimen (e.g., a need and/or dosage for drugs such as statins, warfarin, beta blockers, tamoxifen, raxofilene, etc.), a recommended modification to an existing therapeutic regimen, a lifestyle change recommendation (e.g., egg freezing, IVF, exercise, changing diet such as avoiding dairy), a surgery recommendation (e.g., mastectomy, tumor removal surgery, etc.), a recommended preventative action (e.g., scans), and/or any other recommendations. In a specific example, the recommendation model can be a preventative surgery recommendation model.

However, the risk score can be otherwise used.

The method can optionally include analyzing the risk score S260, which can function to determine which functional groups and/or variants are contributing to the risk score and/or interpret other information associated with the risk score. In an illustrative example, S260 can determine which functional categories (e.g., disease pathways) are enriched, leading to increased risk. S260 can be performed after S250 and/or at any other time.

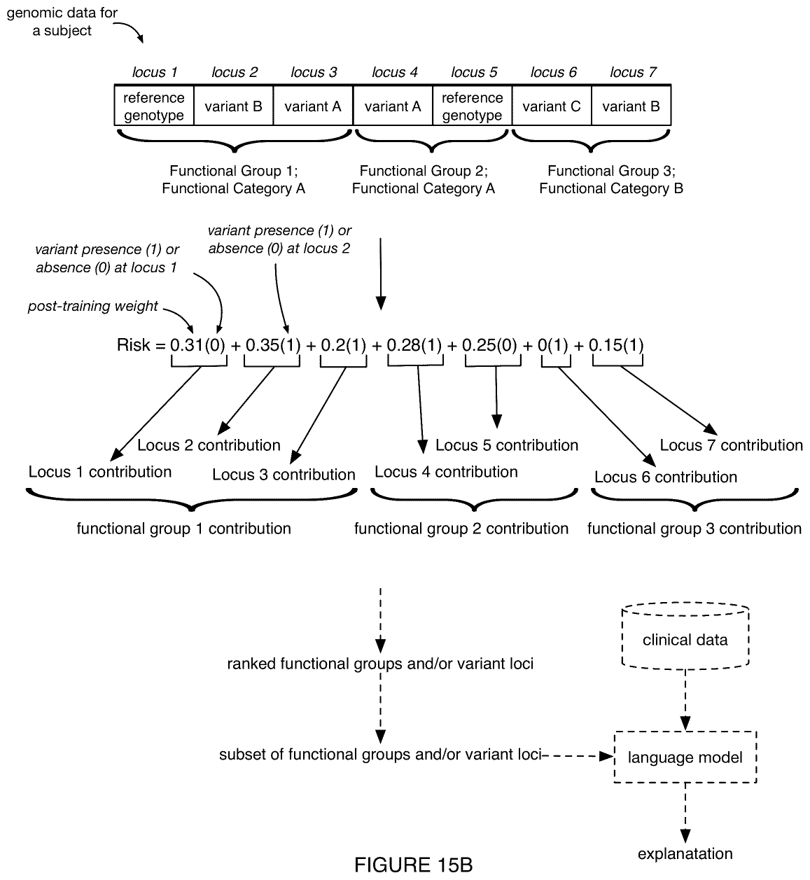

In a first variant, analyzing the risk score can include: determining a contribution to the risk score due to a functional group corresponding to a functional category and/or due to one or more variants in the genomic data (e.g., within a functional group). Examples are shown in FIG. 15A and FIG. 15B. The contribution can be qualitative, quantitative, relative, discrete, continuous, a classification, numeric, binary, and/or be otherwise characterized. The contribution can be positive (increasing the risk), negative (decreasing the risk), neutral, and/or otherwise characterized. For example, for each functional group, the contribution to the risk score for the functional group can be determined based on the risk model and identified variant loci in the genomic data corresponding to the functional group. In a first example, for each functional group, the contribution to the risk score is determined based on the number of identified variant loci in the genomic data corresponding to the functional group. In an illustrative example, a subject can have a given number of variant loci in a functional group, wherein the contribution to the risk score can be determined based on the (updated, post-training) weight for the functional group and the given number of variant loci (e.g., the contribution can be the weight multiplied by the number of variant loci). In a second example, for each functional group, the contribution to the risk score can be determined based on, for each locus in the functional group, the (updated, post-training) weight for the locus and a presence or absence of an identified variant at the locus. In a specific example, the contribution to the risk score for the functional group can be an aggregate (e.g., sum, weighted sum, average, weighted average, etc.) of the contribution to the risk score for each identified variant loci in the functional group, wherein the contribution to the risk score for each identified variant loci is based on the (updated, post-training) weight for the respective locus. In a third example, for each functional group, the contribution to the risk score can be determined based on a relative number of variant loci in the functional group (e.g., relative to a predetermined baseline, relative to a total number of variant loci for the subject, relative to a total number of loci in the functional group, etc.). In an illustrative example, the contribution can be a percentage of variant loci in the functional group (e.g., 10% of the subject's variant loci are in this functional group, the subject has 70% of all possible variants in this functional group, etc.). The contribution for a functional group can optionally be relative to other functional groups. In an illustrative example, the relative contribution for the functional group can be an absolute contribution for the functional group (e.g., the weight for the functional group multiplied by the number of identified variant loci in the functional group), divided by an aggregate of absolute contributions across all functional groups. The contribution for a variant locus can optionally be relative to other variant loci. In an illustrative example, the relative contribution for the variant locus can be an absolute contribution for the variant locus (e.g., the weight for the variant locus), divided by an aggregate of absolute contributions across all variant loci.

The contributions can be provided to the subject, used to identify a subset of functional groups, a subset of functional categories, and/or a subset of variant loci, used to rank functional groups and/or variant loci, and/or otherwise used. In a first example, analyzing the risk score can include ranking each functional category (and corresponding functional group) based on the contribution to the risk score for the respective functional group. In a specific example, analyzing the risk score includes determining a subset of functional categories based on the ranking, wherein the subset includes the one or more highest ranked functional categories. In a second example, analyzing the risk score can include ranking each variant locus based on the respective contribution to the risk score. In a specific example, analyzing the risk score includes determining a subset of variant loci based on the ranking, wherein the subset includes the one or more highest ranked variant loci. In a third example, variant loci within all or a subset of functional categories can be ranked, wherein a subset of variant loci within a subset of functional categories are determined (e.g., the highest contribution variant loci within the highest contribution functional categories).

In a second variant, analyzing the risk score can include using one or more interpretability and/or explainability methods to analyze the trained risk model. Interpretability and/or explainability methods can include: local interpretable model-agnostic explanations (LIME), Shapley Additive explanations (SHAP), Ancors, DeepLift, Layer-Wise Relevance Propagation, contrastive explanations method (CEM), counterfactual explanation, Protodash, Permutation importance (PIMP), L2X, partial dependence plots (PDPs), individual conditional expectation (ICE) plots, accumulated local effect (ALE) plots, Local Interpretable Visual Explanations (LIVE), breakDown, ProfWeight, Supersparse Linear Integer Models (SLIM), generalized additive models with pairwise interactions (GA2Ms), Boolean Rule Column Generation, Generalized Linear Rule Models, Teaching Explanations for Decisions (TED), and/or any other suitable method and/or approach.

In a third variant, analyzing the risk score can include classifying (e.g., categorizing) the risk score into one or more classes. In examples, the classes can be determined based on clinical guidelines, determined based on a threshold (e.g., lifetime risk threshold, a percentile risk threshold, etc.), predetermined, manually determined, randomly determined, and/or otherwise determined. In a specific example, the classes can include: pathogenic, likely pathogenic again, and/or not pathogenic.

In a fourth variant, analyzing the risk score can include determining an explanation based on one or more of: the risk score, risk score analyses (e.g., the subset of functional categories), annotation data, the trait of interest (e.g., disease of interest), and/or any other information. In examples, the explanation can include descriptions of recommendations, descriptions of the trait of interest, descriptions of the risk scores, resources (e.g., clinical papers), and/or any other information. The explanation can be determined using a language model (e.g., natural language processing (NLP), Generative Pre-Trained Transformer (GPT), etc.), and/or any other model.

In a fifth variant, analyzing the risk score includes a combination of the previous variants.

However, the risk score can be otherwise analyzed.

One or more risk scores and/or risk score analyses (e.g., analyses grouped by functional category) can optionally be provided to the subject. For example, the risk score(s) and/or risk score analyses can be displayed at a user interface. In a first illustrative example, the method can include providing: the highest-contribution functional categories contributing to the risk score, optionally the associated variant loci in the highest-contribution functional categories. In a second illustrative example, the method can include providing: the highest-contribution variant loci contributing to the risk score, and what the function (e.g., relevance) is for the variant loci. In example embodiments, the risk scores and/or risk score analyses can be transmitted back to the user via the network 105. In example embodiments, the risk scores and/or risk score analyses are stored on the data storage unit 137. In example embodiments, the risk scores and/or risk score analyses are transmitted (e.g., immediately transmitted) to the user's device. In example embodiments, the risk scores and/or risk score analyses are transmitted across the network 105 to the data acquisition system for subsequent access by the user associated device 100 or genome system 130.

The method can optionally include performing genetic tests using the genomic data for the subject, and providing the results of the genetic tests (e.g., at the user interface; in conjunction with annotated variant loci, risk scores, risk score analyses, and/or other outputs).

The method can optionally include one or more methods of cleaning data. The data can be input data, output data, training data, and/or any other data. In a first example, cleaning can include removing SNPs with minor allele frequency less than a threshold (e.g., less than 1%). In a specific example, risk model(s) can be trained on (only) common variants. In a second example, cleaning can include removing SNPs with imputation accuracy less than a threshold (e.g., less than 0.9). In a third example, cleaning can include removing A>T and/or C>G SNPs (e.g., to eliminate potential strand ambiguity). In a fourth example, A>T and/or C>G SNPs are retained, and a likelihood of each strand can be determined (e.g., probabilistically modeling the likelihood). In a fifth example, cleaning can include determining relationships between multiple subjects (e.g., familial relatedness) and correcting for associated dependence in the training data (e.g., removing subjects, adjusting the data, etc.). In a sixth example, all or portions of the method can be applied to both training and testing. In a sixth example, cleaning can include filtering based on a weight metric.

In one aspect, methods and system of determining disease risk or prognosis in a subject include: receiving genomic data from a subject; identifying disease-specific variant loci in the genomic data; matching annotation data from a plurality of data sources comprising different data types with the corresponding identified disease-specific variant loci; converting the annotation data into a polygenic risk score using a weighting algorithm; and providing a disease diagnosis or prognosis if the polygenic risk score is above a threshold value. In example embodiments, the genomic data may include genomic data described elsewhere herein. In example embodiments, the annotation data may include annotation data described elsewhere herein.

In an example embodiment, the annotation data includes signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature really was the proxy for a phenotype of interest, it could be used to find small molecules that effect that phenotype without knowledge of a validated drug target. The polygenic risk score of the present may be used to screen for drugs that reduce the signature in cells having a specific endotype as described herein. In example embodiments, the invention includes identifying one or more key regulatory features of the polygenic risk score by matching the polygenic risk score with one or more perturbation molecular signatures from a perturbation dataset using similarity scoring. In example embodiments, the signatures with the highest similarity are selected. In example embodiments, the signatures that match have a similarity or connectivity score greater than 0.3, 0.4, 0.5, 0.6, 0.7, 0.8, 0.9, or 0.95. In an example embodiment, the similarity or connectivity score is greater than 0.9 or less than −0.9, or greater than 0.95 or less than −0.95. In an example embodiment, the similarity or connectivity score has a false discovery rate (FDR) of less than 0.5, 0.1, or 0.01.

### Perturbation Datasets

In example embodiments, the perturbation datasets can be generated by perturbation of cells (e.g., cell lines, or primary cells) or complex cell populations (e.g., multicellular systems, such as, organoid, tissue explant, or organ on a chip). The perturbation datasets can include annotation data for therapeutic agents, such as drugs, small molecules, or antibodies. More generally, any compound screen with a molecular read-out as described herein (e.g., a read-out, such as differential gene expression, proteomic, metabolic, spatial, epigenetic, image-based profiling of morphology and cellular markers, or lipodomics, used to construct the polygenic risk score) can be used to nominate compounds by similarity or connectivity with the polygenic risk score. The perturbation datasets can include annotation data for gene knockdown, gene knockout, gene overexpression, gene repression or gene activation. In example embodiments, regulatory proteins, such as transcription factors are perturbed (e.g., by overexpression or knockdown). In one embodiment, perturbation is by deletion of regulatory elements.

In example embodiments, the perturbation datasets include pooled perturbation assays. Methods and tools for genome-scale screening of perturbations in single cells using CRISPR-Cas9 have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; Feldman et al., Lentiviral co-packaging mitigates the effects of intermolecular recombination and multiple integrations in pooled genetic screens, bioRxiv 262121, doi: doi.org/10.1101/262121; Datlinger, et al., 2017, Pooled CRISPR screening with single-cell transcriptome readout. Nature Methods. Vol. 14 No. 3 DOI: 10.1038/nmeth.4177; Hill et al., On the design of CRISPR-based single cell molecular screens, Nat Methods. 2018 April; 15(4): 271-274; Replogle, et al., “Combinatorial single-cell CRISPR screens by direct guide RNA capture and targeted sequencing” Nat Biotechnol (2020). doi.org/10.1038/s41587-020-0470-y; Schraivogel D, Gschwind A R, Milbank J H, et al. “Targeted Perturb-seq enables genome-scale genetic screens in single cells”. Nat Methods. 2020; 17(6):629-635; Frangieh C J, Melms J C, Thakore P I, et al. Multimodal pooled Perturb-CITE-seq screens in patient models define mechanisms of cancer immune evasion. Nat Genet. 2021; 53(3):332-341; US patent application publication number US20200283843A1; and U.S. Pat. No. 11,214,797B2).

In example embodiments, the polygenic risk score is compared to annotation data obtained in prior perturbation assays. For example, the Connectivity Map (cmap) is a comprehensive catalog of cellular signatures representing systematic perturbation with genetic (thus reflecting protein function) and pharmacologic (thus reflecting small-molecule function) perturbagens. Simple pattern-matching algorithms allow the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). As of 2022, CMap has generated a library containing over 1.5M gene expression profiles from ˜5,000 small-molecule compounds, and ˜3,000 genetic reagents, tested in multiple cell types. Cmap can be used to screen for a matching signature in silico. In another example, the JUMP-Cell Painting Consortium is a data-driven approach to drug discovery based on cellular imaging, image analysis, and high dimensional data analytics (see, e.g., jump-cellpainting.broadinstitute.org). The consortium will create a massive cell-imaging dataset, displaying more than 1 billion cells responding to over 140,000 small molecules and genetic perturbations. JUMP-Target provides lists and 384-well plate maps of 306 compounds and corresponding genetic perturbations, designed to assess connectivity in profiling assays. JUMP-MOA provides a list and a 384-well plate map of 90 compounds in quadruplicate (corresponding to 47 mechanism-of-action classes), designed to assess connectivity in profiling assays.

In an example embodiment, CRISPR systems may be used to perturb protein-coding genes or non-protein-coding DNA. CRISPR systems may be used to knockout protein-coding genes by frameshifts, point mutations, inserts, or deletions. In example embodiments, a CRISPR system is used to create an INDEL. CRISPRa/i/x technology may be used in perturbation assays (see, e.g., Konermann et al. “Genome-scale transcriptional activation by an engineered CRISPR-Cas9 complex” Nature. 2014 Dec 10. doi: 10.1038/nature14136; Qi, L. S., et al. (2013). “Repurposing CRISPR as an RNA-guided platform for sequence-specific control of gene expression”. Cell. 152 (5): 1173-83; Gilbert, L. A., et al., (2013). “CRISPR-mediated modular RNA-guided regulation of transcription in eukaryotes”. Cell. 154 (2): 442-51; Komor et al., 2016, Programmable editing of a target base in genomic DNA without double-stranded DNA cleavage, Nature 533, 420-424; Nishida et al., 2016, Targeted nucleotide editing using hybrid prokaryotic and vertebrate adaptive immune systems, Science 353(6305); Yang et al., 2016, Engineering and optimising deaminase fusions for genome editing, Nat Commun. 7:13330; Hess et al., 2016, Directed evolution using dCas9-targeted somatic hypermutation in mammalian cells, Nature Methods 13, 1036-1042; and Ma et al., 2016, Targeted AID-mediated mutagenesis (TAM) enables efficient genomic diversification in mammalian cells, Nature Methods 13, 1029-1035).

In an example embodiment, perturbation of genes is by RNAi. The RNAi may be shRNA's targeting genes. The shRNA's may be delivered by any methods known in the art. In one embodiment, the shRNA's may be delivered by a viral vector. The viral vector may be a lentivirus, adenovirus, or adeno associated virus (AAV).

In an example embodiment, perturbation is performed using small molecules. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

In example embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Epigenetic proteins can regulate many cellular pathways. In example embodiments, a perturbation signature identified using epigenetic protein targeting drugs are matched to a polygenic risk score. Small molecules targeting epigenetic proteins are currently being developed and/or used in the clinic to treat disease (see, e.g., Qi et al., HEDD: the human epigenetic drug database. Database, 2016, 1-10; and Ackloo et al., Chemical probes targeting epigenetic proteins: Applications beyond oncology. Epigenetics 2017, VOL. 12, NO. 5, 378-400). In certain embodiments, the one or more agents include a histone acetylation inhibitor, histone deacetylase (HDAC) inhibitor, histone lysine methylation inhibitor, histone lysine demethylation inhibitor, DNA methyltransferase (DNMT) inhibitor, inhibitor of acetylated histone binding proteins, inhibitor of methylated histone binding proteins, sirtuin inhibitor, protein arginine methyltransferase inhibitor or kinase inhibitor. In certain embodiments, any small molecule exhibiting the functional activity described above may be used in the present invention. In certain embodiments, the DNA methyltransferase (DNMT) inhibitor is selected from the group consisting of azacitidine (5-azacytidine), decitabine (5-aza-2′-deoxycytidine), EGCG (epigallocatechin-3-gallate), zebularine, hydralazine, and procainamide. In certain embodiments, the histone acetylation inhibitor is C646. In certain embodiments, the histone deacetylase (HDAC) inhibitor is selected from the group consisting of vorinostat, givinostat, panobinostat, belinostat, entinostat, CG-1521, romidepsin, ITF-A, ITF-B, valproic acid, OSU-HDAC-44, HC-toxin, magnesium valproate, plitidepsin, tasquinimod, sodium butyrate, mocetinostat, carbamazepine, SB939, CHR-2845, CHR-3996, JNJ-26481585, sodium phenylbutyrate, pivanex, abexinostat, resminostat, dacinostat, droxinostat, and trichostatin A (TSA). In certain embodiments, the histone lysine demethylation inhibitor is selected from the group consisting of pargyline, clorgyline, bizine, GSK2879552, GSK-J4, KDM5-C70, JIB-04, and tranylcypromine. In certain embodiments, the histone lysine methylation inhibitor is selected from the group consisting of EPZ-6438, GSK126, CPI-360, CPI-1205, CPI-0209, DZNep, GSK343, EI1, BIX-01294, UNC0638, EPZ004777, GSK343, UNC1999 and UNC0224. In certain embodiments, the inhibitor of acetylated histone binding proteins is selected from the group consisting of AZD5153 (see e.g., Rhyasen et al., AZD5153: A Novel Bivalent BET Bromodomain Inhibitor Highly Active against Hematologic Malignancies, Mol Cancer Ther. 2016 November; 15(11):2563-2574. Epub 2016 Aug. 29), PFI-1, CPI-203, CPI-0610, RVX-208, OTX015, I-BET151, I-BET762, I-BET-726, dBET1, ARV-771, ARV-825, BETd-260/ZBC260 and MZ1. In certain embodiments, the inhibitor of methylated histone binding proteins is selected from the group consisting of UNC669 and UNC1215. In certain embodiments, the sirtuin inhibitor includes nicotinamide.

### Converting Annotation Data to a Polygenic Risk Score

In example embodiments, a polygenic risk score is converted from annotation data by correlation analysis. In example embodiments, for each dataset weighing algorithm models are run with each variant loci value and every molecular profile variable as an outcome and producing an effect estimate (beta), Pvalue, and Qvalue. The regression beta represents the change in molecular profile variable level per change in the variant loci. In example embodiments, the analysis produces a vector of molecular profile variable changes per standard deviation change in the variant loci value. This vector represents a polygenic risk score for each sample in the dataset. The polygenic risk score can then be meta-analyzed in other datasets of tissues (e.g. SC adipose in MOBB and GTEx), shared cell types (e.g., single cell data sets), or shared spatial location of disease (e.g., spatial data sets). In example embodiments, the polygenic risk score with the largest magnitude of regression beta values, indicating largest mean expression changes are selected. In example embodiments, more than one polygenic risk score is generated. In example embodiments, the top 1, 2, 3, 4, 5, 6, 7, 8, 9, or 10 polygenic risk scores with the largest magnitude of regression beta values are used for identifying regulators, or therapeutic targets or agents. In example embodiments, the polygenic risk scores with the lowest p values and/or q values are selected. In example embodiments, a multi-gene expression polygenic risk score is selected rather than a single polygenic risk score.

In example embodiments, a polygenic risk score is further used to identify functional programs related to each disease endotype. In example embodiments, in silico functional characterization of the a polygenic risk score can be performed. For example, enrichment of established gene set libraries including Gene Ontologies (GO), Reactome, BioPlanet, KEGG can be tested using established gene-set enrichment tools including GSEA, the Ingenuity Pathway Analysis Tool, and Gene-List Network Enrichment Analysis (GeLiNEA). In example embodiments, the polygenic risk score are analyzed for enrichment of transcriptional regulators using an analysis such as, the “epigenetic Landscape In Silico deletion Analysis” (Lisa), which incorporates chromatin profile data and transcription factor/chromatin regulator ChIP-seq datasets from human and mouse studies to assess for enrichment of transcription factor and chromatin regulator binding sites across the top genes in an expression signature or the Ensembl Variant Effect Predictor (VEP) to assess for pathway interactions between genes. In example embodiments, enrichment analyses can be focused on results reaching significance after accounting for multiple testing using a Qvalue <0.001, 0.01, 0.1, or 0.5 threshold.

The polygenic risk score may encompass any gene or genes, protein or proteins, epigenetic element(s), clinical features, or morphological features whose expression profile or whose occurrence is correlated with a specific variant loci (e.g., a high or low disease risk polygenic score). For example, a specific variant loci may be correlated with genes, proteins, epigenetic element(s), clinical features or morphological features. Further, therapeutic agents can have similar signatures of genes, proteins, epigenetic element(s), clinical features, or morphological features and can be identified (e.g., using perturbation studies). The polygenic risk score of the present invention may be microenvironment specific, such as their expression in a particular spatio-temporal context. The polygenic risk score according to example embodiments of the present invention may include or consist of one or more genes, proteins, epigenetic elements, and/or features, such as for instance 1, 2, 3, 4, 5, 6, 7, 8, 9, 10, 25, 50, 100, 500, 1000 or more. In example embodiments, the polygenic risk score may include or consist of two or more genes, proteins and/or epigenetic elements, such as for instance 2, 3, 4, 5, 6, 7, 8, 9, 10, 25, 50, 100, 500, 1000 or more. It is to be understood that a polygenic risk score according to the invention may for instance also include genes or proteins as well as epigenetic elements combined. In this context, a polygenic risk score consists of one or more differentially expressed genes/proteins or differential epigenetic elements or features when comparing different cells or cell (sub)populations. It is to be understood that “differentially expressed” genes/proteins include genes/proteins which are up- or down-regulated as well as genes/proteins which are turned on or off. When referring to up- or down-regulation, in example embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells).

A polygenic risk score may be functionally validated as being uniquely associated with a particular disease diagnosis or prognosis. Induction or suppression of a particular signature may consequentially be associated with or causally drive a particular disease diagnosis or prognosis. Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere. Particular advantageous uses include methods for identifying agents capable of inducing or suppressing particular pathways based on the gene signatures, protein signature, and/or other genetic or epigenetic signature as defined herein.

### Weighing Algorithms

Different weighing algorithms have been contemplated to carry out the embodiments discussed herein. For example, linear regression (LiR) or logistic regression (LoR), a suitable statistical algorithm, and/or a heuristic system for weighing genomic variant loci.

### Linear Regression (LiR)

In one example embodiment, linear regression weighing algorithms are implemented. LiR is typically used to predict a result through the mathematical relationship between an independent and dependent variable, such as genomic data from a subject and a disease diagnosis or prognosis, respectively. A simple linear regression model would have one independent variable (x) and one dependent variable (y). A representation of an example mathematical relationship of a simple linear regression model would be y=mx+b. In this example, the weighing algorithm tries variations of the tuning variables m and b to optimize a line that includes all the given training data.

The tuning variables can be optimized, for example, with a cost function. A cost function takes advantage of the minimization problem to identify the optimal tuning variables. The minimization problem preposes the optimal tuning variable will minimize the error between the predicted outcome and the actual outcome. An example cost function may include summing all the square differences between the predicted and actual output values and dividing them by the total number of input values and results in the average square error.

To select new tuning variables to reduce the cost function, the machine learning module may use, for example, gradient descent methods. An example gradient descent method includes evaluating the partial derivative of the cost function with respect to the tuning variables. The sign and magnitude of the partial derivatives indicate whether the choice of a new tuning variable value will reduce the cost function, thereby optimizing the linear regression weighing algorithm. A new tuning variable value is selected depending on a set threshold. Depending on the weighing algorithm module, a steep or gradual negative slope is selected. Both the cost function and gradient descent can be used with other algorithms and modules mentioned throughout. For the sake of brevity, both the cost function and gradient descent are well known in the art and are applicable to other weighing algorithm and may not be mentioned with the same detail.

LiR models may have many levels of complexity comprising one or more independent variables. Furthermore, in an LiR function with more than one independent variable, each independent variable may have the same one or more tuning variables or each, separately, may have their own one or more tuning variables. The number of independent variables and tuning variables will be understood to one skilled in the art for the problem being solved. In example embodiments, genomic data are used as the independent variables to train a LiR machine learning module, which, after training, is used to estimate, for example, disease diagnosis or prognosis.

### Logistic Regression (LoR)

In one example embodiment, logestic regression weighing algorithms are implemented. Logistic Regression, often considered a LiR type model, is typically used in weighing algorithm to classify information, such as genomic data into categories such as disease diagnosis or prognosis. LoR takes advantage of probability to predict an outcome from input data. However, what makes LoR different from a LiR is that LoR uses a more complex logistic function, for example a sigmoid function. In addition, the cost function can be a sigmoid function limited to a result between 0 and 1. For example, the sigmoid function can be of the form ƒ(x)=1/(1+e^{-x}), where x represents some linear representation of input features and tuning variables. Similar to LiR, the tuning variable(s) of the cost function are optimized (typically by taking the log of some variation of the cost function) such that the result of the cost function, given variable representations of the input features, is a number between 0 and 1, preferably falling on either side of 0.5. As described in LiR, gradient descent may also be used in LoR cost function optimization and is an example of the process. In example embodiments, genomic data are used as the independent variables to train a LoR machine learning module, which, after training, is used to estimate, for example, disease diagnosis or prognosis.

To perform one or more of its functionalities, the weighing algorithm module may communicate with one or more other systems. For example, an integration system may integrate the weighing algorithm module with one or more email servers, web servers, one or more databases, or other servers, systems, or repositories. In addition, one or more functionalities may require communication between a user and the weighing algorithm module.

Any one or more of the module described herein may be implemented using hardware (e.g., one or more processors of a computer/machine) or a combination of hardware and software. For example, any module described herein may configure a hardware processor (e.g., among one or more hardware processors of a machine) to perform the operations described herein for that module. In some example embodiments, any one or more of the modules described herein may include one or more hardware processors and may be configured to perform the operations described herein. In example embodiments, one or more hardware processors are configured to include any one or more of the modules described herein.

Moreover, any two or more of these modules may be combined into a single module, and the functions described herein for a single module may be subdivided among multiple modules. Furthermore, according to various example embodiments, modules described herein as being implemented within a single machine, database, or device may be distributed across multiple machines, databases, or devices. The multiple machines, databases, or devices are communicatively coupled to enable communications between the multiple machines, databases, or devices. The modules themselves are communicatively coupled (e.g., via appropriate interfaces) to each other and to various data sources, to allow information to be passed between the applications to allow the applications to share and access common data.

In S240, the annotated variant loci is displayed to the user. In example embodiments, the annotated variant loci is transmitted back to the user via the network 105. In example embodiments, the annotated variant loci is stored on the data storage unit 137. In example embodiments, the annotated variant loci is transmitted (e.g., immediately transmitted) to the user's device. In example embodiments, the annotated variant loci is transmitted across the network 105 to the data acquisition system for subsequent access by the user associated device 100 or genome annotating system 130.

The ladder diagrams, scenarios, flowcharts and block diagrams in the figures and discussed herein illustrate architecture, functionality, and operation of example embodiments and various aspects of systems, methods, and computer program products of the present invention. Each block in the flowchart or block diagrams can represent the processing of information and/or transmission of information corresponding to circuitry that can be configured to execute the logical functions of the present techniques. Each block in the flowchart or block diagrams can represent a module, segment, or portion of one or more executable instructions for implementing the specified operation or step. In example embodiments, the functions/acts in a block can occur out of the order shown in the figures and nothing requires that the operations be performed in the order illustrated. For example, two blocks shown in succession can executed concurrently or essentially concurrently. In another example, blocks can be executed in the reverse order. Furthermore, variations, modifications, substitutions, additions, or reduction in blocks and/or functions may be used with any of the ladder diagrams, scenarios, flow charts and block diagrams discussed herein, all of which are explicitly contemplated herein.

The ladder diagrams, scenarios, flow charts and block diagrams may be combined with one another, in part or in whole. Coordination will depend upon the required functionality. Each block of the block diagrams and/or flowchart illustration as well as combinations of blocks in the block diagrams and/or flowchart illustrations can be implemented by special purpose hardware-based systems that perform the aforementioned functions/acts or carry out combinations of special purpose hardware and computer instructions. Moreover, a block may represent one or more information transmissions and may correspond to information transmissions among software and/or hardware modules in the same physical device and/or hardware modules in different physical devices.

The present techniques can be implemented as a system, a method, a computer program product, digital electronic circuitry, and/or in computer hardware, firmware, software, or in combinations of them. The system may include distinct software modules embodied on a computer readable storage medium; the modules can include, for example, any or all of the appropriate elements depicted in the block diagrams and/or described herein; by way of example and not limitation, any one, some or all of the modules/blocks and or sub-modules/sub-blocks described. The method steps can then be carried out using the distinct software modules and/or sub-modules of the system, as described above, executing on one or more hardware processors.

The computer program product can include a program tangibly embodied in an information carrier (e.g., computer readable storage medium or media) having computer readable program instructions thereon for execution by, or to control the operation of, data processing apparatus (e.g., a processor) to carry out aspects of one or more embodiments of the present invention. It will be understood that each block of the flowchart illustrations and/or block diagrams, and combinations of blocks in the flowchart illustrations and/or block diagrams, can be implemented by computer readable program instructions.

The computer readable program instructions can be performed on general purpose computing device, special purpose computing device, or other programmable data processing apparatus to produce a machine, such that the instructions, which execute via, at least partially, by one or more processors that are temporarily configured (e.g., by software) or permanently configured to perform the functions/acts specified in the flowchart and/or block diagram block or blocks. The processors, either: temporarily or permanently; or partially configured, may include processor-implemented modules. The present techniques referred to herein may, in example embodiments, include processor-implemented modules. Functions/acts of the processor-implemented modules may be distributed among the one or more processors. Moreover, the functions/acts of the processor-implements modules may be deployed across a number of machines, where the machines may be located in a single geographical location or distributed across a number of geographical locations.

The computer readable program instructions can also be stored in a computer readable storage medium that can direct one or more computer devices, programmable data processing apparatuses, and/or other devices to carry out the function/acts of the processor-implemented modules. The computer readable storage medium containing all or partial processor-implemented modules stored therein, includes an article of manufacture including instructions which implement aspects, operations, or steps to be performed of the function/act specified in the flowchart and/or block diagram block or blocks.

Computer readable program instructions described herein can be downloaded to a computer readable storage medium within a respective computing/processing devices from a computer readable storage medium. Optionally, the computer readable program instructions can be downloaded to an external computer device or external storage device via a network. A network adapter card or network interface in each computing/processing device can receive computer readable program instructions from the network and forward the computer readable program instructions for permanent or temporary storage in a computer readable storage medium within the respective computing/processing device.

Computer readable program instructions described herein can be assembler instructions, instruction-set-architecture (ISA) instructions, machine instructions, machine dependent instructions, microcode, firmware instructions, state-setting data, or either source code or object code. The computer readable program instructions can be written in any programming language such as compiled or interpreted languages. In addition, the programming language can be object-oriented programming language (e.g. “C++”, “Python”) or conventional procedural programming languages (e.g. “C”) or any combination thereof may be used to as computer readable program instructions. The computer readable program instructions can be distributed in any form, for example as a stand-alone program, module, subroutine, or other unit suitable for use in a computing environment. The computer readable program instructions can execute entirely on one computer or on multiple computers at one site or across multiple sites connected by a communication network, for example on user's computer, partly on the user's computer, as a stand-alone software package, partly on the user's computer and partly on a remote computer or entirely on a remote computer or server. If the computer readable program instructions are executed entirely remote, then the remote computer can be connected to the user's computer through any type of network or the connection can be made to an external computer. In examples embodiments, electronic circuitry including, but not limited to, programmable logic circuitry, field-programmable gate arrays (FPGA), or programmable logic arrays (PLA) can execute the computer readable program instructions. Electronic circuitry can utilize state information of the computer readable program instructions to personalize the electronic circuitry, to execute functions/acts of one or more embodiments of the present invention.

Example embodiments described herein include logic or a number of components, modules, or mechanisms. Modules may include either software modules or hardware-implemented modules. A software module may be code embodied on a non-transitory machine-readable medium or in a transmission signal. A hardware-implemented module is a tangible unit capable of performing certain operations and may be configured or arranged in a certain manner. In example embodiments, one or more computer systems (e.g., a standalone, client or server computer system) or one or more processors may be configured by software (e.g., an application or application portion) as a hardware-implemented module that operates to perform certain operations as described herein.

In example embodiments, a hardware-implemented module may be implemented mechanically or electronically. In example embodiments, hardware-implemented modules may include permanently configured dedicated circuitry or logic to execute certain functions/acts such as a special-purpose processor or logic circuitry (e.g., a field programmable gate array (FPGA) or an application-specific integrated circuit (ASIC)). In example embodiments, hardware-implemented modules may include temporary programmable logic or circuitry to perform certain functions/acts. For example, a general-purpose processor or other programmable processor.

The term “hardware-implemented module” encompasses a tangible entity. A tangible entity may be physically constructed, permanently configured, or temporarily or transitorily configured to operate in a certain manner and/or to perform certain functions/acts described herein. Hardware-implemented modules that are temporarily configured need not be configured or instantiated at any one time. For example, if the hardware-implemented modules include a general-purpose processor configured using software, then the general-purpose processor may be configured as different hardware-implemented modules at different times.

Hardware-implemented modules can provide, receive, and/or exchange information from/with other hardware-implemented modules. The hardware-implemented modules herein may be communicatively coupled. Multiple hardware-implemented modules operating concurrently, may communicate through signal transmission, for instance appropriate circuits and buses that connect the hardware-implemented modules. Multiple hardware-implemented modules configured or instantiated at different times may communicate through temporarily or permanently archived information, for instance the storage and retrieval of information in memory structures to which the multiple hardware-implemented modules have access. For example, one hardware-implemented module may perform an operation, and store the output of that operation in a memory device to which it is communicatively coupled. Consequently, another hardware-implemented module may, at some time later, access the memory device to retrieve and process the stored information. Hardware-implemented modules may also initiate communications with input or output devices, and can operate on information from the input or output devices.

In example embodiments, the present techniques can be at least partially implemented in a cloud or virtual machine environment.

### Example Computing Device

FIG. 3 depicts a block diagram of a computing machine 2000 and a module 2050 in accordance with certain examples. The computing machine 2000 may include, but are not limited to, remote devices, work stations, servers, computers, general purpose computers, Internet/web appliances, hand-held devices, wireless devices, portable devices, wearable computers, cellular or mobile phones, personal digital assistants (PDAs), smart phones, smart watches, tablets, ultrabooks, netbooks, laptops, desktops, multi-processor systems, microprocessor-based or programmable consumer electronics, game consoles, set-top boxes, network PCs, mini-computers, and any machine capable of executing the instructions. The module 2050 may include one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a router or other network node, a vehicular information system, one or more processors associated with a television, a customized machine, any other hardware platform, or any combination or multiplicity thereof. The computing machine 2000 may be a distributed system configured to function using multiple computing machines interconnected via a data network or bus system.

The one or more processor 2010 may be configured to execute code or instructions to perform the operations and functionality described herein, manage request flow and address mappings, and to perform calculations and generate commands. Such code or instructions could include, but is not limited to, firmware, resident software, microcode, and the like. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), tensor processing units (TPUs), a graphics processing unit (“GPU”), a field programmable gate array (“FPGA”), a programmable logic device (“PLD”), a radio-frequency integrated circuit (RFIC), a controller, a state machine, gated logic, discrete hardware components, any other processing unit, or any combination or multiplicity thereof. In example embodiments, each processor 2010 can include a reduced instruction set computer (RISC) microprocessor. The processor 2010 may be a single processing unit, multiple processing units, a single processing core, multiple processing cores, special purpose processing cores, co-processors, or any combination thereof. According to certain examples, the processor 2010 along with other components of the computing machine 2000 may be a virtualized computing machine executing within one or more other computing machines. Processors 2010 are coupled to system memory and various other components via a system bus 2020.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random-access memory (“RAM”), static random-access memory (“SRAM”), dynamic random-access memory (“DRAM”), and synchronous dynamic random-access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 is coupled to system bus 2020 and can include a basic input/output system (BIOS), which controls certain basic functions of the processor 2010 and/or operate in conjunction with, a non-volatile storage device such as the storage media 2040.

In example embodiments, the computing device 2000 includes a graphics processing unit (GPU) 2090. Graphics processing unit 2090 is a specialized electronic circuit designed to manipulate and alter memory to accelerate the creation of images in a frame buffer intended for output to a display. In general, a graphics processing unit 2090 is efficient at manipulating computer graphics and image processing and has a highly parallel structure that makes it more effective than general-purpose CPUs for algorithms where processing of large blocks of data is done in parallel.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any electromagnetic storage device, any semiconductor storage device, any physical-based storage device, any removable and non-removable media, any other data storage device, or any combination or multiplicity thereof. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The module 2050 may include one or more hardware or software elements, as well as an operating system, configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may include a computer software product. Each of the operating system, one or more application programs, other program modules, and program data or some combination thereof, may include an implementation of a networking environment. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also include hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for coupling in operation the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including cursor control devices, touch-screens, scanners, electronic digitizers, sensors, receivers, touchpads, trackballs, cameras, microphones, alphanumeric input devices, any other pointing devices, or any combinations thereof. The I/O interface 2060 may couple the computing machine 2000 to various output devices including video displays (The computing device 2000 may further include a graphics display, for example, a plasma display panel (PDP), a light emitting diode (LED) display, a liquid crystal display (LCD), a projector, a cathode ray tube (CRT), or any other display capable of displaying graphics or video), audio generation device, printers, projectors, tactile feedback devices, automation control, robotic components, actuators, motors, fans, solenoids, valves, pumps, transmitters, signal emitters, lights, and so forth. The I/O interface 2060 may couple the computing device 2000 to various devices capable of input and out, such as a storage unit. The devices can be interconnected to the system bus 2020 via a user interface adapter, which can include, for example, a Super I/O chip integrating multiple device adapters into a single integrated circuit.

The computing machine 2000 may operate in a networked environment using logical connections through the network interface 2070 to one or more other systems or computing machines across the network 2080. The network 2080 may include a local area network (“LAN”), a wide area network (“WAN”), an intranet, an Internet, a mobile telephone network, storage area network (“SAN”), personal area network (“PAN”), a metropolitan area network (“MAN”), a wireless network (“WiFi;”), wireless access networks, a wireless local area network (“WLAN”), a virtual private network (“VPN”), a cellular or other mobile communication network, Bluetooth, near field communication (“NFC”), ultra-wideband, wired networks, telephone networks, optical networks, copper transmission cables, or combinations thereof or any other appropriate architecture or system that facilitates the communication of signals and data. The network 2080 may be packet switched, circuit switched, of any topology, and may use any communication protocol. The network 2080 may include routers, firewalls, switches, gateway computers and/or edge servers. Communication links within the network 2080 may involve various digital or analog communication media such as fiber optic cables, free-space optics, waveguides, electrical conductors, wireless links, antennas, radio-frequency communications, and so forth.

Information for facilitating reliable communications can be provided, for example, as packet/message sequencing information, encapsulation headers and/or footers, size/time information, and transmission verification information such as cyclic redundancy check (CRC) and/or parity check values. Communications can be made encoded/encrypted, or otherwise made secure, and/or decrypted/decoded using one or more cryptographic protocols and/or algorithms, such as, but not limited to, Data Encryption Standard (DES), Advanced Encryption Standard (AES), a Rivest-Shamir-Adelman (RSA) algorithm, a Diffie-Hellman algorithm, a secure sockets protocol such as Secure Sockets Layer (SSL) or Transport Layer Security (TLS), and/or Digital Signature Algorithm (DSA). Other cryptographic protocols and/or algorithms can be used as well or in addition to those listed herein to secure and then decrypt/decode communications.

The processor 2010 may be connected to the other elements of the computing machine 2000 or the various peripherals discussed herein through the system bus 2020. The system bus 2020 represents one or more of any of several types of bus structures, including a memory bus or memory controller, a peripheral bus, an accelerated graphics port, and a processor or local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus. It should be appreciated that the system bus 2020 may be within the processor 2010, outside the processor 2010, or both. According to certain examples, any of the processor 2010, the other elements of the computing machine 2000, or the various peripherals discussed herein may be integrated into a single device such as a system on chip (“SOC”), system on package (“SOP”), or ASIC device.

Examples may include a computer program that embodies the functions described and illustrated herein, wherein the computer program is implemented in a computer system that includes instructions stored in a machine-readable medium and a processor that executes the instructions. However, it should be apparent that there could be many different ways of implementing examples in computer programming, and the examples should not be construed as limited to any one set of computer program instructions. Further, a skilled programmer would be able to write such a computer program to implement an example of the disclosed examples based on the appended flow charts and associated description in the application text. Therefore, disclosure of a particular set of program code instructions is not considered necessary for an adequate understanding of how to make and use examples. Further, those ordinarily skilled in the art will appreciate that one or more aspects of examples described herein may be performed by hardware, software, or a combination thereof, as may be embodied in one or more computing systems. Moreover, any reference to an act being performed by a computer should not be construed as being performed by a single computer as more than one computer may perform the act.

The examples described herein can be used with computer hardware and software that perform the methods and processing functions described herein. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

A “server” may include a physical data processing system (for example, the computing device 2000 as shown in FIG. 3) running a server program. A physical server may or may not include a display and keyboard. A physical server may be connected, for example by a network, to other computing devices. Servers connected via a network may operate in the capacity of a server machine or a client machine in a server-client network environment, or as a peer machine in a distributed (e.g., peer-to-peer) network environment. The computing device 2000 can include clients' servers. For example, a client and server can be remote from each other and interact through a network. The relationship of client and server arises by virtue of computer programs in communication with each other, running on the respective computers.

The example systems, methods, and acts described in the examples and described in the figures presented previously are illustrative, not intended to be exhaustive, and not meant to be limiting. In alternative examples, certain acts can be performed in a different order, in parallel with one another, omitted entirely, and/or combined between different examples, and/or certain additional acts can be performed, without departing from the scope and spirit of various examples. Plural instances may implement components, operations, or structures described as a single instance. Structures and functionality that may appear as separate in example embodiments may be implemented as a combined structure or component. Similarly, structures and functionality that may appear as a single component may be implemented as separate components. Accordingly, such alternative examples are included in the scope of the following claims, which are to be accorded the broadest interpretation to encompass such alternate examples. The terminology used herein was chosen to best explain the principles of the embodiments, the practical application or technical improvement over technologies found in the marketplace, or to enable others of ordinary skill in the art to understand the embodiments disclosed herein.

### Parallel Computing

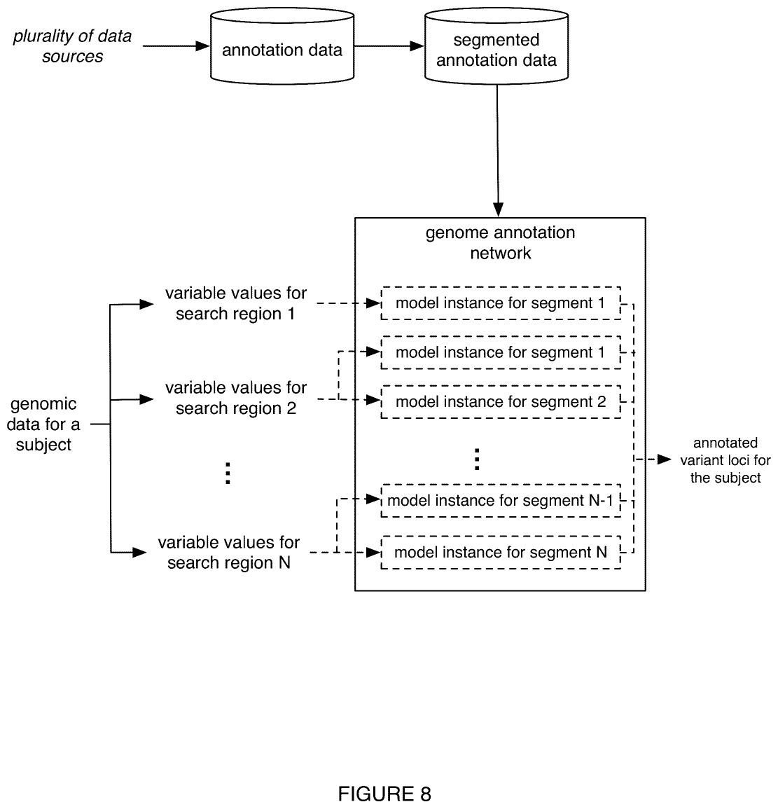

In example embodiments, the genomic data is searched against the annotated data in parallel. An example is shown in FIG. 8. In example embodiments, pre-segmented genomic data is searched in parallel against the annotated data. In example embodiments, pre-segmented genomic data is searched against pre-segmented annotated data in parallel.

Parallel computing, in general, includes a type of computation wherein two or more calculations or processes are carried out at the same time i.e., simultaneously. Parallel computing allows large problems, for example annotating genomic data, to be divided into many small problems, which can be solved at the same time. Parallel computing can occur across multiple cores, multiple processors, or any combination thereof.

There a multiple implementations of parallel computing. For example, there is bit-level, instruction-level, task, and superword level. Bit-level parallel computing includes using multiple cores, processors, or a combination thereof to compute larger bit information with smaller bit architecture, e.g., using two 8 bit processors to compute two 16 bit integers.

Instruction-level parallel computing includes organizing computation instructions into groups which are carried out in parallel. Common implementations of instruction level computing includes the scoreboarding algorithm and Tomasulo algorithm.

Task parallel computing includes performing the same calculation on the same or different sets of data. Task parallel computing may further include separating a task into sub-tasks. The sub-tasks are then distributed to two or more cores, processors, or combination thereof for concurrent processing.

Superword level parallel computing includes vectorization, e.g., converting computing instructions into a scalar format. Vectorizing allows for processing an operation on multiple operands simultaneously. Superword level vectorization includes loop unrolling and basic block vectorization. These methods are known in the art and will not be discussed in detail herein

Further embodiments are illustrated in the following Examples which are given for illustrative purposes only and are not intended to limit the scope of the invention.

### EXAMPLES

### Example 1—Genetic Analysis

The overall product can be a piece of software that takes in an individual's genomic data, cleans/parses through it, annotates that data (quickly+with efficient use of compute) based on best possible evidence about what those genomic sequences do, prioritizes what matters most and then displays output to user in nice GUI or as raw downloadable data, and then can recommend getting clinical testing, or if the input data was already clinical grade, can 1) based on drug response information including efficacy, toxicity, dosage, or metabolism impacts, can recommend personalized dosage adjustments (increased dosage, normal dosage, decreased dosage), looking at alternatives, or prioritizing use of that drug; or 2) based on phenotype risk or protective alleles recommend kicking off next steps in screening and treatment for that phenotype/condition.

Making this possible also involves some innovation around storage/compute/algorithm speed for an application like this.

### Parts

1. Method to automatically convert input unannotated genomic data into similar file format that lists out variants:
a. can have user upload file, connect to another service, or be part of a sequencing pipeline that takes in the results from clinical sequence
b. convert file formats as necessary: unzip files, convert FASTQ to VCF, align formats, ensure consistency in assembly (e.g., human reference genome 37 or 28)
c. handling compressed inputs/outputs means it takes up less storage and can upload/download faster
d. can handle data from subset of genome up to whole genome
e. will parse through, can be simple checking for hash marks or more complex language processing on headers
f. preprocess and clean up inputs
2. Aggregate all possible phenotypic/drug/outcome/clinical annotations and filter/prioritize them:
a. data pulls include from ClinGen, ClinVar, PharmGKB, MONDO, HGNC, MedGen, Human Phenotype Ontology, OMIM, Orphanet, Breast Cancer Information Core, UniProtKB, GWAS fine-mapping, activity-by-contact model noncoding annotations, published studies in PubMed, Nature, and others
b. automatically refresh sources to keep them up to date and make sure results are best possible
c. improve clinical annotations by curating “layman's terms” on what particular diseases or drugs do (e.g., warfarin is “blood clotting medication”)
d. pre-filtering and pre-sorting these source files means merge against individual input files is faster
3. In web version: automatically upload new individual input files to S3 storage buckets, split into subdirectories, have an event bridge trigger tied to a subdirectory, when a file is uploaded to subdirectory it launches an ECS task where container image is script and identifies latest uploaded file, pulls that file along with other static files stored in S3 bucket, runs script and then uploads output files to another directory
a. optimizes for scaling up and down and storage
b. can autodelete files after it has detected to persist longer than X days
4. Annotating raw files with database data:
a. matching variants in input data against clinical database, which is optimized for speed; one version that is working well is start by labeling the input data by SNP id or rsid and then match on that so it's a 1 variable match; another version was to prioritize starting by chromosome and then for each position that has a variant, loop through “subdictionaries” of ranges in the clinical data; the slower version would be to go chromosome by chromosome, start site by start site, but this subdictionary approach can speed up from days to seconds of running this match

e.g., here is one iteration of code for searching against clinical data with ranges.

Function to generate variant buckets:
For each chromosome, create a bucket; and
Within each of these chromosome buckets, create sub chromosome buckets of 100 bp in size.

To search for variants in the buckets:
identify the range of buckets that may include relevant evidence based on chromosome and position; and
only search in those buckets.

For each variant prioritizing which results have strongest evidence (e.g., already in clinical guidelines, expert panel review, conflicting study submissions, just 1 submission) and number of studies slash work done on them.

Categorizing and organizing results by type of interpretation (e.g., drug response versus risk versus protective)—for drug evidence, combine with individual study data and count number of studies in each weighed by strength of evidence.

When results conflict, weighting evidence strength to come up with overall result.

Linking results to resources to learn more.

privacy-preserving benefit in that no human has to see their raw genomic data and it's deleted afterwards.
5. Creating a graphical user interface for the results to be easier to interpret and visualize, including creating cards for output data by bucket:
a. runs quickly
b. can also directly download raw annotated csvs/tsvs data, a subset of “attention” prioritized data, or json/dictionaries
c. mock output for cards (See FIG. 16):
d. will also link to genetic counseling/further resources/next steps
e. for raw data dump, outputs a .csv/.xslx/.tsv/.txt with a row for each variant, and hundreds of columns including variant IDs for each of the queried databases (ClinGen, ClinVar, PharmGKB, MONDO, HGNC, MedGen, Human Phenotype Ontology, OMIM, Orphanet, Breast Cancer Information Core, UniProtKB), genotype details, phenotypes, evidence levels
6. Output used to recommend in some cases eg a consumer test that individual should get a clinical test, refer to a genetic counselor
7. Output used in other case eg if input data was already clinical grade that they should either get follow up clinical confirmation, or implement a different screening/testing schedule, or take a particular drug; or if they are found to have a drug sensitivity, to see pharmacist about that result

### Filtration Process

1. Variant annotations filtered by level of review (e.g., current guideline, expert review, conflicting evidence)
2. Outcomes segmented by clinical implication, e.g., drug response vs pathogenic risk vs protective allele
3. If evidence conflicts, comparison of strongest evidence levels and resolution

### Uniform Variant Summary Format for Individual Variables

All individual input files are converted to this format—this speeds up the match because then the annotation/reference files can match on one or many of these columns and it's standardized; this can involve reading and manipulating data in the input file, running against a reference genome, inferring another column based on a current column (for example, if the type of variant is a SNP, the “overall” “start” and “stop” positions will be the same)

Can include some subset of these variables:
Chromosome (e.g., chr1)
Overall position (e.g., 10481-10482)
Start position (e.g., 10481)
Stop position (e.g., 10482)
ID (e.g., rs200451305)
Type of variant (e.g., SNP, INDEL, CNV)
Reference allele(s), (e.g., “A” or “AA”)
Present allele(s), (e.g., “C” or “CT”)
Reference assembly (e.g., HG37 or HG38)
And any other relevant variables

### Matching Process—Speeding it Up

Because annotation evidence can be so big and these files can be very large (e.g., in the 10s/100s of GB at the higher end) it's important to speed up the match as much as possible, otherwise this can take multiple days to run. So Applicants have a “matching structure”:
1. Pre-segment the clinical annotation evidence by chromosome, and store separately (e.g., chromosome 1, chromosome 2, etc.). If evidence shows up for multiple chromosomes, repeat it in all of those.
2. For each chromosome, create substructures (e.g., subdictionaries) for “position buckets” and prefill evidence into each bucket. For evidence that spans a range of positions, round up or down (pick one and be consistent) to the nearest bucket. Don't replicate evidence into multiple buckets; instead, the search will look in a wider range of buckets to speed up further; unlike chromosome, the position buckets are quantitatively related so searching up/down is logically consistent.
3. These buckets can be stored independently so that parallel search of these different buckets can be done without worrying about conflicts/double-counting (e.g., for a variant at chr 1 position X, the bucket that X falls into can be searched as well as the bucket that X falls into −1 on chromosome 1. In an illustrative example, if round up is being used for step 2, the bucket that X falls into can be checked, as well as bucket X+1. Those 2 annotation searches can optionally be performed in parallel.

During the match, for each individual variant, rather than searching the entirety of the annotation evidence, categorize that individual variant's position in the matching structure, and then check the bucket it would fall in as well as buckets above and/or below the bucket it would fall in (e.g., optionally only checking those buckets). Then annotate with all evidence in the genomic positions overlapping that variant.

Finally the match by variant can be parallelized as well; since the annotation evidence is all being stored separately, Applicants can split up the input data arbitrarily into rows that Applicants annotate (so, e.g., the first variant listed in a file can be searched at the same time as the last variant). If Applicants group variants by chromosome or by bucket (e.g., if n=3 variants a-c all would fall into chr 1 bucket X based on their position), then Applicants can run the search in the buckets once for all of those variants and go one by one, which means only having to access bucket x 1 time instead of n times, and in parallel accessing bucket x-1 1 time instead of n times.

Finally if all of this is all done with arrays instead of dataframes, it further speeds up the process.

At the end, make sure to join all the annotations back together. Because the annotation data is so large, but Applicants have so heavily subsegmented it, the search can take seconds rather than days.

### For Web Apps Run Remote, Speeding Up/Optimizing Storage

Applicants can store annotation evidence in the buckets just once; and that serves the purpose for all individual files.

When an individual uploads a file, the event trigger means the remote compute is only spun up for what is needed and limits overuse of compute costs.

Deleting individual evidence files after Applicants are done can further save on storage.

Finally, Applicants can optimize the storage system to be in less accessed types of storage (e.g., if Applicants haven't accessed in ages, can move to AWS S3 glacier which is cheaper but takes longer to pull; versus if Applicants know Applicants will be calling the program a lot this week, Applicants can move to AWS S3 Glacier Instant Retrieval to optimize for speed)

### Clinical Outcomes

Table 1—List of drugs where based on information including efficacy, toxicity, dosage, or metabolism impacts, Applicants can recommend dosage adjustments (increased dosage, normal dosage, decreased dosage), looking at alternatives, or prioritizing use of that drug:

TABLE 1

| | | | | |
|---|---|---|---|---|
| (R)-methadone | clavulanate | folic acid | minocycline | salmeterol |
| 3,4- | clindamycin | follitropin beta | mirtazapine | salvianolic acid b |
| methylenedioxymethamphetamine | | | | |
| abacavir | clobazam | furosemide | mitotane | selective beta-2- |
| | | | | adrenoreceptor |
| | | | | agonists |
| abiraterone | clodronate | gabapentin | mitoxantrone | Selective |
| | | | | serotonin |
| | | | | reuptake |
| | | | | inhibitors |
| ABT-751 | clomipramine | galantamine | mizolastine | selegiline |
| acamprosate | clonidine | gefitinib | modafinil | sertraline |
| Ace Inhibitors, Plain | clopidogrel | geldanamycin | montelukast | sevoflurane |
| acenocoumarol | clozapine | gemcitabine | morphine | sibutramine |
| acetaldehyde | cocaine | gemtuzumab | muraglitazar | sildenafil |
| | | ozogamicin | | |
| acetaminophen | codeine | gentamicin | mycophenolate | silibinin |
| | | | mofetil | |
| acetazolamide | conjugated | glatiramer acetate | mycophenolic acid | simeprevir |
| | estrogens | | | |
| acetylcholine | coptisine | glibenclamide | n- | simvastatin |
| | | | desmethyltamoxifen | |
| acetylcysteine | corticosteroids | gliclazide | naloxone | simvastatin acid |
| adalimumab | cotinine | glimepiride | naltrexone | sipoglitazar |
| adefovir dipivoxil | cotinine | glipizide | naproxen | siponimod |
| | glucuronide | | | |
| adrenergics, inhalants | coumarin | Glucarpidase | nedocromil | sirolimus |
| agomelatine | creatine | glucocorticoids | nefazodone | sitagliptin |
| alemtuzumab | curcumin | granisetron | nelfinavir | SN-38 |
| alendronate | cyanocobalamin | haloperidol | nemonapride | sofosbuvir |
| alfentanil | cyclophosphamide | halothane | neomycin | somatropin |
| | | | | recombinant |
| Alkylating Agents | cyclosporine | hdl cholesterol | nevirapine | sorafenib |
| allopurinol | cysteamine | heparin | nicotine | sotalol |
| alprazolam | cytarabine | Hepatitis vaccines | nifedipine | sparteine |
| amantadine | Dabigatran | heroin | nilotinib | spironolactone |
| amikacin | dacarbazine | highly active | nimodipine | stavudine |
| | | antiretroviral | | |
| | | therapy (haart) | | |
| amiloride | daclatasvir | hmg coa | nitrendipine | streptomycin |
| | | reductase | | |
| | | inhibitors | | |
| aminoglycoside | dalcetrapib | HMG CoA | nitrofurantoin | succinylcholine |
| antibacterials | | reductase | | |
| | | inhibitors, other | | |
| | | combinations | | |
| amiodarone | dapoxetine | hormonal | nitroprusside | sufentanil |
| | | contraceptives for | | |
| | | systemic use | | |
| amisulpride | dapsone | hydralazine | nitrous oxide | sulfadoxine |
| amitriptyline | daptomycin | hydralazine/ | nortriptyline | sulfamethoxazole |
| | | isosorbide | | |
| | | dinitrate | | |
| amlodipine | dasatinib | hydrochlorothiazide | Nucleoside and | sulfamethoxazole/ |
| | | | nucleotide reverse | trimethoprim |
| | | | transcriptase | |
| | | | inhibitors | |
| amodiaquine | daunorubicin | hydrocodone | O- | sulfametopyrazine |
| | | | desmethyltramadol | |
| amoxicillin | debrisoquine | hydroxychloroquine | olanzapine | sulfasalazine |
| amphetamine | deferasirox | hydroxyurea | olmesartan | sulfinpyrazone |
| amprenavir | deferiprone | ibuprofen | omeprazole | sulfonamides, |
| | | | | urea derivatives |
| Analgesics | deleobuvir | icotinib | ondansetron | sulindac |
| anastrozole | desflurane | idarubicin | Opioid anesthetics | sumatriptan |
| angiotensin II | desipramine | iloperidone | opioids | sunitinib |
| Angiotensin II Antagonists | desloratadine | imatinib | opipramol | Sympathomimetics |
| anthracyclines and related | desmethylnaproxen | imidapril | Opium alkaloids | tacrolimus |
| substances | | | and derivatives | |
| Antibiotics | dexamethasone | imipramine | oseltamivir | tafenoquine |
| Anticholinergics | dexlansoprazole | imiquimod | Other general | talinolol |
| | | | anesthetics | |
| antidepressants | dexmedetomidine | indinavir | oxaliplatin | tamoxifen |
| antiepileptics | dexrazoxane | indomethacin | oxazepam | tapentadol |
| Antihypertensives | dextroamphetamine | infliximab | oxcarbazepine | taxanes |
| Antihypertensives And | dextromethorphan | Influenza | oxycodone | tegafur |
| Diuretics In Combination | | vaccines | | |
| Antiinflammatory agents, | dextropropoxyphene | insulin | paclitaxel | tegafur/ |
| non-steroids | | recombinant | | gimeracil/ |
| | | | | oteracil |
| antineoplastic agents | diazepam | interferon alfa-2a, | paliperidone | telaprevir |
| | | recombinant | | |
| antipsychotics | diclofenac | interferon alfa-2b, | palonosetron | telmisartan |
| | | recombinant | | |
| antithymocyte globulin | dicloxacillin | interferon beta-1a | pamidronate | temozolomide |
| Antithyroid Preparations | didanosine | interferon beta-1b | panitumumab | temsirolimus |
| Antivirals for treatment of | difluorodeoxyuridine | interferons | pantoprazole | tenofovir |
| HIV infections, | | | | |
| combinations | | | | |
| apixaban | digoxin | irbesartan | paroxetine | tenofovir |
| | | | | disoproxil |
| | | | | fumarate |
| aripiprazole | dihydrocodeine | irinotecan | pazopanib | tenoxicam |
| artemether | Dihydropyridine | isepamicin | pegaptanib | terbutaline |
| | derivatives | | | |
| artesunate | dihydrostreptomycin | isoflurane | pegaspargase | testosterone |
| asparaginase | diltiazem | isoniazid | peginterferon alfa- | tezacaftor |
| | | | 2a | |
| aspirin | dimercaprol | isoproterenol | peginterferon alfa- | thalidomide |
| | | | 2b | |
| ataluren | dimethyl | itopride | pegloticase | Thiazides, plain |
| | fumarate | | | |
| atazanavir | Dipeptidy1 | ivacaftor | pemetrexed | thiazolidinediones |
| | peptidase 4 | | | |
| | (DPP-4) | | | |
| | inhibitors | | | |
| atazanavir/ritonavir | dipyrone | ivacaftor/ | penicillin g | thioguanine |
| | | lumacaftor | | |
| atenolol | direct acting | ivacaftor/ | penicillin v | thioridazine |
| | antivirals | tezacaftor | | |
| atomoxetine | disopyramide | kanamycin | perindopril | thiotepa |
| atorvastatin | disulfiram | ketamine | perphenazine | thyrotropin alfa |
| atrasentan | diuretics | ketoprofen | Pertussis vaccines | tianeptine |
| axitinib | dobutamine | ketorolac | phenazepam | ticagrelor |
| azathioprine | docetaxel | 1-methylfolate | phenazopyridine | ticlopidine |
| benazepril | dolasetron | 1-phenylalanine | phenobarbital | tiludronate |
| benzodiazepine derivatives | dolutegravir | 1-tryptophan | phenprocoumon | timolol |
| berberine | donepezil | lamivudine | phenylephrine | tiotropium |
| Beta Blocking Agents | Dopamine | lamotrigine | phenytoin | tipifarnib |
| | agonists | | | |
| Beta blocking agents, | doxepin | lansoprazole | photodynamic | tipiracil |
| selective | | | therapy | hydrochloride |
| bevacizumab | doxorubicin | lapatinib | pioglitazone | tobramycin |
| bilirubin | doxorubicinol | latanoprost | piroxicam | tocilizumab |
| bisoprolol | Drugs For | ledipasvir | pitavastatin | tolbutamide |
| | Treatment Of | | | |
| | Tuberculosis | | | |
| Bisphosphonates | Drugs used in | leflunomide | pitrakinra | tolperisone |
| | alcohol | | | |
| | dependence | | | |
| bleomycin | Drugs Used In | lenalidomide | platinum | tolterodine |
| | Diabetes | | | |
| boceprevir | Drugs used in | letermovir | Platinum | topiramate |
| | nicotine | | compounds | |
| | dependence | | | |
| bortezomib | duloxetine | letrozole | pramipexole | topoisomerase I |
| | | | | inhibitors |
| botulinum toxin type a | eculizumab | leucovorin | prasugrel | topotecan |
| brivaracetam | efavirenz | levetiracetam | pravastatin | torasemide |
| bromperidol | egfr inhibitors | levodopa | prednisolone | tramadol |
| bucindolol | enalapril | liothyronine | prednisone | trandolapril |
| budesonide | endoxifen | liraglutide | pregabalin | tranilast |
| bufuralol | enflurane | lisinopril | primaquine | trastuzumab |
| bumetanide | entacapone | lithium | prochlorperazine | triamcinolone |
| buprenorphine | Enzymes | lonafarnib | progesterone | trichloroethylene |
| buprenorphine/naloxone | ephedrine | lopinavir | propafenone | trifluoperazine |
| bupropion | epirubicin | lorazepam | propionic acid | trifluridine |
| | | | derivatives | |
| busulfan | Ergot alkaloids | lornoxicam | propofol | triglycerides |
| butorphanol | erlotinib | losartan | propranolol | trimipramine |
| caffeine | erythromycin | lovastatin | propylthiouracil | troglitazone |
| calcein | escitalopram | lovastatin acid | protease inhibitors | tropisetron |
| calcium | esomeprazole | loxoprofen | purine analogues | Tumor necrosis |
| | | | | factor alpha |
| | | | | (TNF-alpha) |
| | | | | inhibitors |
| calcium channel blockers | estradiol | lumacaftor | pyrazinamide | urofollitropin |
| candesartan | etanercept | lumefantrine | Pyrazolones | ustekinumab |
| cangrelor | ethambutol | lurasidone | pyrimethamine | valganciclovir |
| cannabinoids | ethanol | maprotiline | quetiapine | valproic acid |
| capecitabine | ethosuximide | maraviroc | quinapril | vancomycin |
| captopril | ethylmorphine | Measles vaccines | quinidine | vardenafil |
| carbamazepine | etidronic acid | medroxyprogesterone | rabeprazole | varenicline |
| carbimazole | etoposide | mefloquine | radiotherapy | velpatasvir |
| carbocisteine | etravirine | meloxicam | raloxifene | venlafaxine |
| carboplatin | everolimus | melphalan | raltitrexed | verapamil |
| carisoprodol | exemestane | memantine | ramipril | vildagliptin |
| carvedilol | faldaprevir | meperidine | ranibizumab | vinblastine |
| catecholamines | Farglitazar | mephenytoin | rasagiline | vincristine |
| cavosonstat | febuxostat | mercaptopurine | rasburicase | vindesine |
| cefotaxime | FEC100 | metformin | regadenoson | vinorelbine |
| ceftriaxone | fenofibrate | methacholine | remifentanil | Vitamin B- |
| | | | | complex, Incl. |
| | | | | Combinations |
| celecoxib | fentanyl | methadone | repaglinide | vitamin b- |
| | | | | complex, plain |
| cephalexin | fesoterodine | methamphetamine | rhodamine 123 | vitamin e |
| cerivastatin | fexofenadine | methazolamide | ribavirin | Vitamin K |
| certolizumab pegol | flecainide | methimazole | rifampin | Vitamin K1 |
| cetuximab | flucloxacillin | methotrexate | risedronate | volatile |
| | | | | anesthetics |
| chlorambucil | fludarabine | methoxsalen | risperidone | voriconazole |
| chloramphenicol | fluindione | methoxyflurane | ritodrine | vortioxetine |
| chlorocresol | flunisolide | methylene blue | ritonavir | voxilaprevir |
| chloroquine | fluorouracil | methylphenidate | rituximab | warfarin |
| chlorothiazide | fluoxetine | methylphenobarbital | rivaroxaban | XK469 |
| chlorproguanil | fluphenazine | methylprednisolone | rivastigmine | zafirlukast |
| chlorpromazine | flupirtine | metoprolol | rocuronium | zidovudine |
| chlorthalidone | flurbiprofen | metronidazole | rofecoxib | zileuton |
| cidofovir | fluticasone | mexiletine | rosiglitazone | zinc acetate |
| | propionate | | | |
| cilostazol | fluvastatin | mianserin | rosuvastatin | ziprasidone |
| ciprofloxacin | fluvoxamine | micronomicin | S-EDDP | zoledronate |
| cisplatin | FOLFIRI | midazolam | sacubitril | zonisamide |
| citalopram | FOLFIRINOX | migalastat | salbutamol | zuclopenthixol |
| cladribine | FOLFOX | milnacipran | salicylamide | |

Table 2—List of phenotypes where based on drug response information Applicants can recommend drug dosage adjustments (increased dosage, normal dosage, decreased dosage), looking at alternatives, or prioritizing use of that drug

TABLE 2

| | | | | |
|---|---|---|---|---|
| ability to | Breast | Drug Hypersensitivity | HIV | Neoplasms; |
| concentrate; Depressive | Neoplasms; | | Infections; Toxic | Neutropenia |
| Disorder, | Lymphopenia | | liver disease | |
| Major; Diarrhea; | | | | |
| Dizziness; Tremor | | | | |
| Abortion, Spontaneous | Breast | Drug Hypersensitivity; drug | HIV | Neoplasms; |
| | Neoplasms; | reaction with eosinophilia and | Infections; | Osteonecrosis |
| | Menopause | systemic | Tuberculosis | |
| | | symptoms; Epidermal | | |
| | | Necrolysis, | | |
| | | Toxic; Maculopapular | | |
| | | Exanthema; severe cutaneous | | |
| | | adverse reactions; Stevens- | | |
| | | Johnson Syndrome | | |
| Acquired | Breast | Drug Hypersensitivity; drug | Hyperalgesia | Neoplasms; |
| Immunodeficiency | Neoplasms; | reaction with eosinophilia and | | Osteosarcoma; |
| Syndrome | mucositis | systemic | | Ototoxicity; |
| | | symptoms; Epidermal | | Testicular |
| | | Necrolysis, | | Neoplasms |
| | | Toxic; Maculopapular | | |
| | | Exanthema; Stevens-Johnson | | |
| | | Syndrome | | |
| Acquired | Breast | Drug Hypersensitivity; drug | Hyperbilirubinemia | Neoplasms; |
| Immunodeficiency | Neoplasms; | reaction with eosinophilia and | | Ototoxicity; |
| Syndrome; HIV | Neoplasms | systemic | | Testicular |
| Infections; | | symptoms; Epidermal | | Neoplasms |
| nephrolithiasis | | Necrolysis, Toxic; severe | | |
| | | cutaneous adverse | | |
| | | reactions; Stevens-Johnson | | |
| | | Syndrome | | |
| Acquired Long QT | Breast | Drug Hypersensitivity; drug | Hyperbilirubinemia; | Neoplasms; |
| Syndrome (aLQTS) | Neoplasms; | reaction with eosinophilia and | Neoplasms | Ovarian |
| | Neutropenia | systemic | | Neoplasms |
| | | symptoms; Leprosy; Maculopapular | | |
| | | Exanthema; severe | | |
| | | cutaneous adverse | | |
| | | reactions; Stevens-Johnson | | |
| | | Syndrome | | |
| Acute coronary syndrome | Breast | Drug | Hypercholesterolemia | Neoplasms; |
| | Neoplasms; | Hypersensitivity; Epidermal | | Ovarian |
| | Ovarian | Necrolysis, Toxic; erythema | | Neoplasms; |
| | Neoplasms | exudativum | | Stomach |
| | | multiforme; Maculopapular | | Neoplasms |
| | | Exanthema; Stevens-Johnson | | |
| | | Syndrome | | |
| Acute coronary | Breast | Drug Hypersensitivity; HIV | Hypercholesterolemia; | Neoplasms; |
| syndrome; Coronary | Neoplasms; | Infections | Myocardial | Pain |
| Artery Disease | Ovarian | | Infarction | |
| | Neoplasms; | | | |
| | Peripheral | | | |
| | Nervous | | | |
| | System | | | |
| | Diseases | | | |
| Acute coronary | Breast | Drug | Hyperglycemia | Neoplasms; |
| syndrome; major adverse | Neoplasms; | Hypersensitivity; Stevens- | | Pain; Pain, |
| cardiac events (mace) | Peripheral | Johnson Syndrome | | Postoperative |
| | Nervous | | | |
| | System | | | |
| | Diseases | | | |
| Adenocarcinoma; Carcinoma, | Burkitt | Drug interaction with | Hyperglycemia; | Neoplasms; |
| Non-Small-Cell | Lymphoma; | drug; Drug Toxicity | Hypertension | Precursor Cell |
| Lung; Drug | Drug | | | Lymphoblastic |
| Resistance; Lung | Toxicity; | | | Leukemia- |
| Neoplasms | Lymphoma, | | | Lymphoma |
| | T-Cell; | | | |
| | Osteosarcoma; | | | |
| | Precursor Cell | | | |
| | Lymphoblastic | | | |
| | Leukemia- | | | |
| | Lymphoma | | | |
| Adenocarcinoma; Carcinoma, | Burkitt | drug reaction with | Hyperlipidemias | Neoplasms; |
| Non-Small-Cell | Lymphoma; Drug | eosinophilia and systemic | | Stomach |
| Lung; Drug | Toxicity; Lymphoma, | symptoms | | Neoplasms |
| Toxicity; Exanthema; Toxic | T-Cell; | | | |
| liver disease | Precursor Cell | | | |
| | Lymphoblastic | | | |
| | Leukemia- | | | |
| | Lymphoma | | | |
| Adrenocortical | Burkitt | drug reaction with | Hyperlipoproteinemia | Nephrosclerosis |
| Carcinoma | Lymphoma; Leukemia; | eosinophilia and systemic | Type II | |
| | Lymphoma, | symptoms; Epidermal | | |
| | T-Cell; | Necrolysis, | | |
| | Neoplasms; | Toxic; Exanthema; | | |
| | Osteosarcoma; | Hypersensitivity; Stevens- | | |
| | Precursor Cell | Johnson Syndrome | | |
| | Lymphoblastic | | | |
| | Leukemia- | | | |
| | Lymphoma | | | |
| adverse events | Burkitt | drug reaction with | Hypernatremia; | Nephrotic |
| | Lymphoma; | eosinophilia and systemic | Hypertension | Syndrome |
| | Leukemia; | symptoms; Epidermal | | |
| | Lymphoma; | Necrolysis, | | |
| | Lymphoma, T- | Toxic; Maculopapular | | |
| | Cell; Precursor | Exanthema; severe cutaneous | | |
| | Cell | adverse reactions; Stevens- | | |
| | Lymphoblastic | Johnson Syndrome | | |
| | Leukemia- | | | |
| | Lymphoma | | | |
| adverse | Burkitt | drug reaction with | Hyperprolactinemia | nephrotoxicity |
| events; Alcoholism; | Lymphoma; | eosinophilia and systemic | | |
| Anxiety Disorders | Lymphoma, | symptoms; Epidermal | | |
| | Non-Hodgkin; | Necrolysis, Toxic; severe | | |
| | Lymphoma, | cutaneous adverse | | |
| | T-Cell; | reactions; Stevens-Johnson | | |
| | Precursor Cell | Syndrome | | |
| | Lymphoblastic | | | |
| | Leukemia- | | | |
| | Lymphoma | | | |
| adverse events; Arthritis, | Burkitt | drug reaction with | Hyperprolactinemia; | nephrotoxicity; |
| Rheumatoid | Lymphoma; | eosinophilia and systemic | Schizophrenia | Osteosarcoma |
| | Lymphoma, T- | symptoms; Epidermal | | |
| | Cell; Osteosarcoma; | Necrolysis, Toxic; Stevens- | | |
| | Precursor | Johnson Syndrome | | |
| | Cell | | | |
| | Lymphoblastic | | | |
| | Leukemia- | | | |
| | Lymphoma | | | |
| adverse events; Bipolar | Burkitt | drug reaction with | Hypersensitivity | neuropathic |
| Disorder; Depression; | Lymphoma; | eosinophilia and systemic | | pain |
| Depressive Disorder, | Lymphoma, T- | symptoms; HIV | | |
| Major | Cell; Precursor | Infections; Stevens-Johnson | | |
| | Cell | Syndrome; Toxic liver disease | | |
| | Lymphoblastic | | | |
| | Leukemia- | | | |
| | Lymphoma | | | |
| adverse | Carcinoma, | drug reaction with | Hypertension | Neurotoxicity |
| events; Carcinoma, Non- | Basal Cell | eosinophilia and systemic | | Syndromes |
| Small-Cell Lung | | symptoms; severe cutaneous | | |
| | | adverse reactions; Stevens- | | |
| | | Johnson Syndrome | | |
| adverse | Carcinoma, | drug reaction with | Hypertension; | Neutropenia |
| events; Constipation; | Hepatocellular | eosinophilia and systemic | Hypertrophy, Left | |
| Delirium; Dizziness; | | symptoms; Stevens-Johnson | Ventricular | |
| Nausea; Pain, | | Syndrome | | |
| Postoperative; | | | | |
| Postoperative Nausea | | | | |
| and Vomiting; Pruritus; | | | | |
| Respiratory | | | | |
| Insufficiency; somnolence; | | | | |
| Urinary | | | | |
| Retention; Vomiting | | | | |
| adverse | Carcinoma, | Drug Toxicity | Hypertension; | Neutropenia; |
| events; Constipation; | Hepatocellular; | | Kidney | Pancreatic |
| Delirium; Nausea; Pruritus; | Carcinoma, | | Diseases; | Neoplasms |
| somnolence; Urinary | Renal Cell | | Nephrosclerosis | |
| Retention | | | | |
| adverse events; Epilepsy | Carcinoma, | Drug | Hypertension; | Neutropenia; |
| | Hepatocellular; | Toxicity; Gastrointestinal | Myocardial | Precursor |
| | hand-foot | Stromal Tumors | Infarction | Cell |
| | syndrome | | | Lymphoblastic |
| | | | | Leukemia- |
| | | | | Lymphoma; |
| | | | | Thrombocytopenia |
| adverse | Carcinoma, | Drug | hypertensive | Neutropenia; |
| events; gastrointestinal | Hepatocellular; | Toxicity; hematotoxicity; | nephrosclerosis | Urinary |
| toxicity; Myelosuppression; | Hyperbilirubinemia | Leukopenia; Lymphoma; mucositis; | | Bladder |
| Urinary Bladder | | Neoplasms; Neutropenia; | | Neoplasms |
| Neoplasms | | Osteosarcoma; Precursor Cell | | |
| | | Lymphoblastic Leukemia- | | |
| | | Lymphoma; primary central | | |
| | | nervous system | | |
| | | lymphoma; Thrombocytopenia; | | |
| | | Toxic liver disease | | |
| adverse | Carcinoma, | Drug Toxicity; Inflammatory | Hypertriglyceridemia | Obesity |
| events; Hypersensitivity | Hepatocellular; | Bowel Diseases; Pancreatitis | | |
| | Liver | | | |
| | Neoplasms | | | |
| adverse | Carcinoma, | Drug | Hypertriglyceridemia; | Obesity; |
| events; Hypersensitivity; | Non-Small- | Toxicity; Lymphoma; Osteosarcoma; | schizoaffective | Polycystic Ovary |
| severe cutaneous adverse | Cell Lung | Precursor Cell | disorder; | Syndrome |
| reactions | | Lymphoblastic Leukemia- | Schizophrenia; | |
| | | Lymphoma | Weight gain | |
| adverse | Carcinoma, | Drug Toxicity; Neoplasms | Hypertriglyceridemia; | Obsessive- |
| events; Nausea; Vomiting | Non-Small- | | Weight gain | Compulsive |
| | Cell Lung; | | | Disorder |
| | Colorectal | | | |
| | Neoplasms; | | | |
| | Gastrointestinal | | | |
| | Neoplasms; | | | |
| | Ovarian | | | |
| | Neoplasms | | | |
| adverse | Carcinoma, | Drug | Hypertrophy, | Opioid- |
| events; neuropathic pain | Non-Small- | Toxicity; Neoplasms; Neutropenia; | Left Ventricular | Related |
| | Cell Lung; | Peripheral Nervous | | Disorders |
| | Colorectal | System Diseases | | |
| | Neoplasms; | | | |
| | Neoplasms; | | | |
| | Pancreatic | | | |
| | Neoplasms | | | |
| adverse events; Opioid- | Carcinoma, | Drug | Hypotension | Opioid- |
| Related Disorders | Non-Small- | Toxicity; Neoplasms; Ototoxicity | | Related |
| | Cell Lung; | | | Disorders; |
| | Diarrhea | | | Pruritus |
| adverse events; Pain, | Carcinoma, | Drug Toxicity; Neurotoxicity | Infection | Opioid- |
| Postoperative | Non-Small- | Syndromes | | Related |
| | Cell | | | Disorders; |
| | Lung; Drug | | | Sexual |
| | Resistance | | | Dysfunctions, |
| | | | | Psychological |
| adverse events; Premature | Carcinoma, | Drug | Infection; Nausea; | Opioid- |
| Birth | Non-Small- | Toxicity; Neutropenia; | Testicular | Related |
| | Cell Lung; | Peripheral Nervous System | Neoplasms | Disorders; |
| | Drug | Diseases; Toxic liver disease | | Sleep |
| | Toxicity; | | | Disorders |
| | Leukemia, B- | | | |
| | Cell, Acute | | | |
| agitation; Alcohol-Related | Carcinoma, | Drug Toxicity; overall | Infertility, | Organ |
| Disorders; cardiotoxicity; | Non-Small- | survival | Female; Ovarian | Transplantation |
| Depression; Depressive | Cell Lung; | | hyperstimulation | |
| Disorder; Depressive | Exanthema | | syndrome | |
| Disorder, Major; Drug | | | | |
| Toxicity; dysphoria; Edema; | | | | |
| Nausea; Obsessive- | | | | |
| Compulsive | | | | |
| Disorder; Tachycardia; | | | | |
| Vomiting | | | | |
| Agranulocytosis | Carcinoma, | Drug Toxicity; Precursor Cell | Inflammatory | Organ |
| | Non-Small- | Lymphoblastic Leukemia- | Bowel Diseases | Transplantation; |
| | Cell Lung; | Lymphoma | | Transplantation |
| | gastrointestinal | | | |
| | toxicity; | | | |
| | Hematologic | | | |
| | Diseases; | | | |
| | Leukopenia | | | |
| Agranulocytosis; Graves | Carcinoma, | Drug Toxicity; Psoriasis | Inflammatory | Osteitis |
| Disease | Non-Small- | | Bowel | Deformans |
| | Cell Lung; | | Diseases; | |
| | Mesothelioma | | Myelosuppression | |
| Alcohol-Related | Carcinoma, | Drug Toxicity; Thalassemia | Inflammatory | Osteonecrosis |
| Disorders | Non-Small- | | Bowel | |
| | Cell Lung; | | Diseases; Psoriasis | |
| | Mesothelioma; | | | |
| | Pancreatic | | | |
| | Neoplasms | | | |
| Alcoholism | Carcinoma, | Drug Toxicity; Tuberculosis | insomnia | osteonecrosis |
| | Non-Small- | | | of jaw |
| | Cell Lung; | | | caused by |
| | Neoplasms | | | drug |
| Alcoholism; Anxiety | Carcinoma, | Drug Toxicity; Urinary | Irritable Bowel | Osteonecrosis; |
| Disorders | Non-Small- | Bladder Neoplasms | Syndrome | Precursor Cell |
| | Cell Lung; | | | Lymphoblastic |
| | Ovarian | | | Leukemia- |
| | Neoplasms | | | Lymphoma |
| Alcoholism; Attention | Carcinoma, | drug-induced liver injury | Irritable Bowel | Osteoporosis |
| Deficit Disorder with | Non-Small- | | Syndrome; | |
| Hyperactivity | Cell Lung; | | Leukopenia | |
| | pneumonitis | | | |
| Alcoholism; Bipolar | Carcinoma, | drug-induced liver | Kidney Failure | Osteoporosis; |
| Disorder | Non-Small- | injury; Hepatitis, Toxic; Toxic | | Osteoporosis, |
| | Cell Lung; | liver disease; Tuberculosis | | Postmenopausal |
| | Thrombocytopenia | | | |
| Alcoholism; Death | Carcinoma, | drug-induced liver injury; HIV | Kidney | Osteosarcoma |
| | Non-Small- | Infections; Tuberculosis | Neoplasms | |
| | Cell | | | |
| | Lung; Toxic | | | |
| | liver disease | | | |
| Alcoholism; hypersexuality | Carcinoma, | drug-induced liver | Kidney | Osteosarcoma; |
| state; Tobacco Use | Renal Cell | injury; Leukopenia; Precursor | Transplantation | Precursor |
| Disorder | | Cell Lymphoblastic | | Cell |
| | | Leukemia- | | Lymphoblastic |
| | | Lymphoma; Thrombocytopenia | | Leukemia- |
| | | | | Lymphoma |
| Alcoholism; Substance- | Carcinoma, | drug-induced liver | Kidney | Ototoxicity |
| Related Disorders | Renal Cell; | injury; Multiple Sclerosis | Transplantation; | |
| | hand-foot | | liver | |
| | syndrome | | transplantation | |
| Alopecia; Mesothelioma | Carcinoma, | drug-induced liver | Kidney | Ovarian |
| | Renal Cell; | injury; Toxic liver disease | Transplantation; | Neoplasms |
| | Hypertension | | liver | |
| | | | transplantation; | |
| | | | Proteinuria | |
| Alopecia; Pain; Testicular | Carcinoma, | drug-induced liver | Kidney | overall |
| Neoplasms | Renal | injury; Tuberculosis | Transplantation; | survival |
| | Cell; Neutropenia | | lung | |
| | | | transplantation | |
| Alopecia; Testicular | Carcinoma, | Dyspepsia; Pain, | Kidney | overall |
| Neoplasms | Small Cell | Postoperative | Transplantation; | survival; |
| | | | Myasthenia | progression-free |
| | | | Gravis | survival |
| Alzheimer Disease | Carcinoma, | Dystonia | Kidney | overall |
| | Squamous | | Transplantation; | survival; |
| | Cell; overall | | Organ | Thrombocytopenia |
| | survival | | Transplantation | |
| Alzheimer | Carcinoma, | Endometrial Neoplasms | Kidney | Overdose |
| Disease; cognitive | Squamous | | Transplantation; | |
| dysfunction | Cell; | | transplant | |
| | progression-free | | rejection | |
| | survival | | | |
| Alzheimer Disease; Lewy | Cardiomyopathies | eosinophilic esophagitis | Leukemia, B- | Paim |
| Body Disease | | | Cell, Acute | |
| Amenorrhea | Cardiomyopathies; | Epidermal Necrolysis, | Leukemia, B- | Pain |
| | Neoplasms | Toxic; Epilepsy; severe | Cell, Acute; | |
| | | cutaneous adverse | Osteosarcoma; | |
| | | reactions; Stevens-Johnson | Precursor Cell | |
| | | Syndrome | Lymphoblastic | |
| | | | Leukemia- | |
| | | | Lymphoma | |
| Amyotrophic Lateral | Cardiomyopathy, | Epidermal Necrolysis, | Leukemia, | Pain and |
| Sclerosis | Dilated; Death | Toxic; HIV | Lymphocytic, | Arthritis |
| | | Infections; Stevens-Johnson | Chronic, B-Cell | |
| | | Syndrome | | |
| anaphylactoid reaction | Cardiomyopathy, | Epidermal Necrolysis, | Leukemia, | Pain and |
| | Dilated; Heart | Toxic; Maculopapular | Lymphoid; | Cough |
| | Failure | Exanthema; severe cutaneous | Precursor Cell | |
| | | adverse reactions; Stevens- | Lymphoblastic | |
| | | Johnson Syndrome | Leukemia- | |
| | | | Lymphoma | |
| Anemia, Hemolytic | cardiotoxicity; | Epidermal Necrolysis, | Leukemia, | Pain, |
| | Neoplasms | Toxic; Maculopapular | Myelogenous, | Postoperative |
| | | Exanthema; Stevens-Johnson | Chronic, BCR- | |
| | | Syndrome | ABL Positive | |
| Anemia, | cardiotoxicity; | Epidermal Necrolysis, | Leukemia, | Pain; Pain, |
| Hemolytic; Hemolysis | Osteosarcoma | Toxic; severe cutaneous | Myeloid | Postoperative |
| | | adverse reactions; Stevens- | | |
| | | Johnson Syndrome | | |
| Anemia, | Cardiovascular | Epidermal Necrolysis, | Leukemia, | Pain; Tobacco |
| Hemolytic; Hemolysis; | Diseases | Toxic; Stevens-Johnson | Myeloid, Acute | Use Disorder |
| Protein Deficiency | | Syndrome | | |
| Anemia, Sickle Cell | Cardiovascular | Epilepsies, | Leukemia, | Pancreatic |
| | Diseases; Coronary | Partial; Epilepsy; Epilepsy, | Myeloid, | Neoplasms |
| | Disease; Stroke | Generalized | Acute; Neoplasms | |
| Anemia; Dermatitis; | Cardiovascular | Epilepsy | Leukemia; | Pancreatic |
| Leukopenia; mucositis; | Diseases; | | Lymphoma | Neoplasms; |
| Myelosuppression; Neutropenia; | Rhabdomyolysis | | | Thrombocytopenia |
| Thrombocytopenia | | | | |
| Anemia; Dermatitis; | Central | Epilepsy; Psychotic Disorders | Leukemia; | Pancreatitis; |
| mucositis; Myelosuppression; | Nervous | | Lymphoma; | Precursor |
| Nasopharyngeal | System | | Osteosarcoma; | Cell |
| Neoplasms; Neutropenia; | Infections | | Precursor Cell | Lymphoblastic |
| Thrombocytopenia | | | Lymphoblastic | Leukemia- |
| | | | Leukemia- | Lymphoma |
| | | | Lymphoma | |
| Anemia; Hepatitis C, | cessation; | Epilepsy; Seizures | Leukopenia | Pancytopenia; |
| Chronic | Tobacco Use | | | Thrombocytopenia |
| | Disorder | | | |
| Anemia; Leukopenia; | Chemotherapy | Erectile Dysfunction | Leukopenia; | Parkinson |
| Lymphoma, Non- | | | Mesothelioma | Disease |
| Hodgkin; mucositis; | | | | |
| Osteosarcoma; Precursor | | | | |
| Cell Lymphoblastic | | | | |
| Leukemia- | | | | |
| Lymphoma; Thrombocytopenia; | | | | |
| Toxic liver disease | | | | |
| Anemia; Leukopenia; | Chemotherapy | Erythema | Leukopenia; | paroxysmal |
| Nausea; Neutropenia; | and | | Myelosuppression | nocturnal |
| Thrombocytopenia; Vomiting | Immunosuppressive | | | hemoglobinuria |
| Anemia; Mesothelioma | Choroidal | Esophageal Neoplasms | Leukopenia; | Peptic Ulcer |
| | Neovascularization | | Myelosuppression; | |
| | | | Neutropenia; | |
| | | | Thrombocytopenia | |
| Anemia; mucositis; | chronic lung | Esophageal | Leukopenia; | Peripheral |
| Osteosarcoma | allograft | Neoplasms; Ovarian | Neoplasms | Nervous |
| | dysfunction; | Neoplasms; Stomach | | System |
| | lung | Neoplasms | | Diseases |
| | transplantation | | | |
| Anemia; Nasopharyngeal | Cluster | Esophagitis; Gastroesophageal | Leukopenia; | Phenotype(s) |
| Neoplasms | Headache | Reflux; Helicobacter | Neutropenia; | |
| | | Infections; Peptic Ulcer | Ovarian Neoplasms | |
| Anemia; Nasopharyngeal | cns | Essential hypertension | Leukopenia; | pneumonitis |
| Neoplasms; Neutropenia | depression; | | Neutropenia; | |
| | Drug Toxicity | | Precursor Cell | |
| | | | Lymphoblastic | |
| | | | Leukemia- | |
| | | | Lymphoma | |
| Anemia; Ovarian | Cocaine- | Essential | Leukopenia; | pneumonitis; |
| Neoplasms | Related | hypertension; Hypertension | Osteosarcoma | progression- |
| | Disorders | | | free survival |
| Anemia; Testicular | Cocaine- | Exanthema | Leukopenia; | postanesthesia |
| Neoplasms | Related | | Precursor Cell | apnea |
| | Disorders; | | Lymphoblastic | |
| | Heroin | | Leukemia- | |
| | Dependence | | Lymphoma | |
| Angina Pectoris; Heart | Colitis | Exanthema; Opioid-Related | Leukopenia; | Postoperative |
| Failure | Ulcerative | Disorders | Testicular | Nausea and |
| | | | Neoplasms | Vomiting |
| Angioedema | Colitis, | Fabry Disease | Liver Cirrhosis | Postoperative |
| | Ulcerative; | | | Nausea and |
| | Crohn | | | Vomiting; |
| | Disease; | | | Vomiting |
| | Irritable Bowel | | | |
| | Syndrome; | | | |
| | Leukopenia; | | | |
| | Neutropenia | | | |
| Anticoagulant | Colitis, | familial hypercholesterolemia | Liver Failure, | Precursor |
| | Ulcerative; | | Acute | Cell |
| | Inflammatory | | | Lymphoblastic |
| | Bowel | | | Leukemia- |
| | Diseases | | | Lymphoma |
| Antidepressant and Nerve | Colitis, | Fanconi Syndrome; HIV | Liver Neoplasms | Pregnancy |
| Pain | Ulcerative; | Infections | | |
| | Kidney | | | |
| | Transplantation | | | |
| Antifungal | Colonic | Fatigue | liver | Prostatic |
| | Neoplasms | | transplantation | Neoplasms |
| Anxiety Disorders | Colorectal | febrile neutropenia; Testicular | Long QT | Psoriasis |
| | Neoplasms | Neoplasms | Syndrome | |
| Anxiety | Colorectal | Fever and Pain | Low Back Pain | Psychomotor |
| Disorders; Depressive | Neoplasms; Drug | | | Agitation |
| Disorder | Toxicity | | | |
| Anxiety | Colorectal | Gastroesophageal Reflux | Lung Neoplasms | Psychotic |
| Disorders; Depressive | Neoplasms; | | | Disorders |
| Disorder, Major | Esophageal | | | |
| | Neoplasms; | | | |
| | Osteosarcoma; | | | |
| | Ovarian | | | |
| | Neoplasms; | | | |
| | Pancreatic | | | |
| | Neoplasms | | | |
| Apnea | Colorectal | Gastroesophageal | lung | Psychotic |
| | Neoplasms; | Reflux; Helicobacter | transplantation; | Disorders; |
| | hand-foot | Infections | overall survival | schizoaffective |
| | syndrome | | | disorder; |
| | | | | Schizophrenia |
| Arrhythmias, Cardiac | Colorectal | Gastroesophageal | lung | Psychotic |
| | Neoplasms; | Reflux; Helicobacter | transplantation; | Disorders; |
| | Head and Neck | Infections; Peptic Ulcer | transplant | Schizophrenia |
| | Neoplasms | | rejection | |
| Arrhythmias, | Colorectal | Gastroesophageal | Lupus | Psychotic |
| Cardiac; Drug | Neoplasms; | Reflux; Transplantation | erythematosus | Disorders; |
| Toxicity; Lymphoma, | Rectal | | | Substance- |
| Non-Hodgkin | Neoplasms | | | Related |
| | | | | Disorders |
| Arrhythmias, | Confusion; Drug | Gastrointestinal Stromal | Lupus | Pulmonary |
| Cardiac; Tachycardia | Toxicity; Head | Tumors | Erythematosus, | Fibrosis |
| | ache; Muscle | | Systemic | |
| | Rigidity; | | | |
| | Schizophrenia; | | | |
| | sedation; | | | |
| | Seizures; | | | |
| | Tachycardia | | | |
| Arteriosclerosis; Coronary | Congenital | Gastrointestinal Stromal | Lupus Nephritis | Rectal |
| Disease; Essential | Abnormalities; | Tumors; Leukemia, | | Neoplasms |
| hypertension; glomerular | Craniofacial | Myelogenous, Chronic, BCR- | | |
| disease; Glomerulonephritis, | Abnormalities | ABL Positive | | |
| IGA; Kidney Diseases | | | | |
| Arthralgia; Breast | Constipation; | gastrointestinal | Lymphoma, B- | Respiratory |
| Neoplasms | Delirium; Lung | toxicity; Hallucinations; Parkin | Cell | Insufficiency |
| | Neoplasms; | son Disease | | |
| | Nausea; Pain; | | | |
| | Post operative | | | |
| | Nausea and | | | |
| | Vomiting; | | | |
| | Pruritus; | | | |
| | Respiratory | | | |
| | Insufficiency; | | | |
| | somnolence; | | | |
| | Urinary Retention | | | |
| Arthritis | Coronary | gastrointestinal | Lymphoma, | Retinal |
| | Artery Disease | toxicity; mucositis; | Large B-Cell, | Diseases |
| | | Neutropenia; Precursor Cell | Diffuse | |
| | | Lymphoblastic Leukemia- | | |
| | | Lymphoma | | |
| Arthritis and Pain | Coronary | gastrointestinal | Lymphoma, | Rhabdomyolysis |
| | Artery | toxicity; Myelosuppression; | Non-Hodgkin | |
| | Disease; | Urinary Bladder Neoplasms | | |
| | Coronary | | | |
| | Disease; | | | |
| | Myocardial | | | |
| | Infarction | | | |
| Arthritis, Juvenile | Coronary | GERD, Damaged Esophagus, | Lymphoma, | Sarcoma |
| Rheumatoid | Artery | and Stomach Acid | Non- | |
| | Disease; | | Hodgkin; Precurs | |
| | Diabetes | | or Cell | |
| | Mellitus; | | Lymphoblastic | |
| | Hypercholesterolemia | | Leukemia- | |
| | | | Lymphoma | |
| Arthritis, Juvenile | Coronary | Glaucoma, Open-Angle | Lymphoma; | schizoaffective |
| Rheumatoid; Arthritis, | Artery | | mucositis; | disorder; |
| Psoriatic; Arthritis, | Disease; | | Osteosarcoma; | Schizophrenia |
| Rheumatoid; Drug | Hypercholesterolemia | | Precursor Cell | |
| Toxicity | | | Lymphoblastic | |
| | | | Leukemia- | |
| | | | Lymphoma | |
| Arthritis, Juvenile | Coronary | Glioma | Lymphoma; | Schizophrenia |
| Rheumatoid; Arthritis, | Artery | | Osteosarcoma; | |
| Rheumatoid | Disease; | | Precursor Cell | |
| | Hypertension | | Lymphoblastic | |
| | | | Leukemia- | |
| | | | Lymphoma | |
| Arthritis, Psoriatic | Coronary | Gout | Macular | Schizophrenia, |
| | Artery | | Degeneration | Bipolar |
| | Disease; Myalgia | | | disorder, |
| | unspecified | | | Depression |
| Arthritis, | Coronary | Graft vs Host | Maculopapular | Schizophrenia; |
| Psoriatic; Arthritis, | Artery | Disease; Leukemia; Leukemia, | Exanthema | tardive |
| Rheumatoid | Disease; | Myelogenous, Chronic, BCR- | | dyskinesia |
| | Myocardial | ABL Positive | | |
| | Infarction | | | |
| Arthritis, | Coronary | Graves Disease | Maculopapular | Schizophrenia; |
| Psoriatic; Arthritis, | Disease | | Exanthema; severe | Weight gain |
| Rheumatoid; Crohn | | | cutaneous | |
| Disease; Inflammation; | | | adverse | |
| Psoriasis; Spondylitis, | | | reactions; Stevens- | |
| Ankylosing | | | Johnson | |
| | | | Syndrome | |
| Arthritis, | Coronary | hand-foot | Maculopapular | sedation; |
| Psoriatic; Psoriasis | Disease; | syndrome; Hypertension | Exanthema; | Urticaria |
| | Hypercholesterolemia; | | Tuberculosis | |
| | Myocardial | | | |
| | Infarction | | | |
| Arthritis, Rheumatoid | Coronary | hand-foot | major adverse | severe |
| | Disease; | syndrome; Neoplasms | cardiac events | cutaneous |
| | Hyperlipidemias | | (mace) | adverse |
| | | | | reactions |
| Arthritis, | Coronary | Head and Neck Neoplasms | Malaria | severe |
| Rheumatoid; Crohn | Disease; | | | cutaneous |
| Disease; Psoriasis; | Osteoporosis | | | adverse |
| Spondylitis, Ankylosing | | | | reactions; |
| | | | | Stevens- |
| | | | | Johnson |
| | | | | Syndrome |
| Arthritis, | Cough | Headache Disorders; Migraine | Malaria; Malaria, | Severe Pain |
| Rheumatoid; Drug | | with Aura; Migraine without | Falciparum | |
| Toxicity | | Aura | | |
| Arthritis, | Cough; | Heart | Malignant | Sexual |
| Rheumatoid; Drug | Essential | Arrest; Overdose; Respiratory | Hyperthermia | Dysfunctions, |
| Toxicity; hematopoietic | hypertension | Insufficiency | | Psychological |
| stem cell | | | | |
| transplantation; Psoriasis; | | | | |
| Toxic liver disease | | | | |
| Arthritis, | Cough; | Heart Diseases | Marijuana Abuse | short qt |
| Rheumatoid; Neuromyelitis | Hypertension | | | syndrome 1 |
| Optica | | | | |
| Arthritis, | Crohn Disease | Heart Failure | Medulloblastoma; | Shortened |
| Rheumatoid; Precursor | | | Neoplasms; | QT interval |
| Cell Lymphoblastic | | | Ototoxicity; | |
| Leukemia-Lymphoma | | | Testicular Neoplasms | |
| Arthritis, | Crohn | Heart Failure; Neoplasms | Menopause; | Sleep Apnea |
| Rheumatoid; Psoriasis | Disease; | | Schizophrenia | Syndromes |
| | Inflammatory | | | |
| | Bowel | | | |
| | Diseases | | | |
| aspirin-induced asthma | Cystic | heart | Mental Disorders | Sleep |
| | Fibrosis | transplantation; hematopoietic | | Disorders |
| | | stem cell | | |
| | | transplantation; Kidney | | |
| | | Transplantation; lung | | |
| | | transplantation | | |
| aspirin-induced | Cystitis; | heart | Mental | somnolence |
| asthma; Asthma | Transplantation | transplantation; hemopoietic | Disorders; | |
| | | stem cell transplant; Kidney | Schizophrenia | |
| | | Transplantation; liver | | |
| | | transplantation; lung | | |
| | | transplantation | | |
| Asthenia | Deafness; | heart transplantation; Kidney | Mesothelioma | Spondylitis, |
| | Neoplasms; | Transplantation; laparoscopic | | Ankylosing |
| | Ototoxicity | sleeve gastrectomy; liver | | |
| | | transplantation; lung | | |
| | | transplantation | | |
| Asthenia; Nausea; | Deafness; | Heartburn, GERD, | Mesothelioma; | SSRI |
| Neoplasms; Vomiting | Ototoxicity; | Esophageal Damage | Precursor Cell | |
| | Testicular | | Lymphoblastic | |
| | Neoplasms | | Leukemia- | |
| | | | Lymphoma | |
| Asthenia; Neoplasms | Death; Opioid- | Helicobacter Infections | Mesothelioma; | statin-related |
| | Related | | Thrombocytopenia | myopathy |
| | Disorders | | | |
| Asthma | Dementia | Hematologic Neoplasms | Metabolic | Stevens- |
| | | | Syndrome | Johnson |
| | | | | Syndrome |
| Atrial Fibrillation | Depression | hematopoietic stem cell | Metabolic | Stomach |
| | | transplantation; Kidney | Syndrome; | Neoplasms |
| | | Transplantation; transplant | Schizophrenia | |
| | | rejection | | |
| Attention Deficit | Depression; | hematopoietic stem cell | methamphetamine | Stroke |
| Disorder with | Depressive | transplantation; Kidney | dependence | |
| Hyperactivity | Disorder | Transplantation; Urinary | | |
| | | Bladder Neoplasms | | |
| Autism Spectrum | Depression; De | hematopoietic stem cell | Migraine NOS | Stroke and |
| Disorder | pressive | transplantation; Neurotoxicity | | Heart Attack |
| | Disorder, | Syndromes | | Prevention |
| | Major | | | |
| Autism Spectrum | Depression; | Hemolysis | Migraine with | Stroke; |
| Disorder; Mood Disorders | Depressive | | Aura | Venous |
| | Disorder; | | | Thrombosis |
| | Depressive | | | |
| | Disorder, | | | |
| | Major | | | |
| Autism Spectrum | Depression; | Hemolysis; Lead Poisoning, | Mood Disorders | Substance |
| Disorder; Psychotic | Depressive | Nervous System, Childhood | | Withdrawal |
| Disorders; Schizophrenia | Disorder; | | | Syndrome |
| | Depressive | | | |
| | Disorder, | | | |
| | Major; | | | |
| | Hypotension | | | |
| Autism Spectrum | Depression; | Hemolysis; Methemoglobinemia | mucositis; | Substance- |
| Disorder; Schizophrenia | suicide | | Osteosarcoma | Related |
| | | | | Disorders |
| Autistic Disorder | Depressive | hemopoietic stem cell | Multiple | sustained |
| | Disorder | transplant | Myeloma | virological |
| | | | | response (svr) |
| beta-Thalassemia | Depressive | Hemorrhage; venous | Multiple | Tachycardia |
| | Disorder, | thromboembolism | Myeloma; | |
| | Major | | Osteonecrosis | |
| Bipolar Disorder | Depressive | heparin-induced | Multiple | tardive |
| | Disorder, | thrombocytopenia | Myeloma; | dyskinesia |
| | Major; Mental | | progression-free | |
| | Disorders | | survival | |
| Bipolar | Depressive | Hepatic Veno-Occlusive | Multiple | Temporomandibular |
| Disorder; Depression | Disorder, | Disease; Transplantation | Sclerosis | joint-pain- |
| | Major; Nausea; | | | dysfunction |
| | Vomiting | | | syndrome |
| Bipolar | Depressive | Hepatitis B, Chronic | Muscular | Testicular |
| Disorder; Depression; | Disorder, | | Diseases | Neoplasms; |
| Depressive Disorder, Major | Major; | | | Vomiting |
| | Obsessive- | | | |
| | Compulsive | | | |
| | Disorder | | | |
| Bipolar | Depressive | Hepatitis C | Myalgia | Thalassemia |
| Disorder; Depression; | Disorder, | | unspecified | |
| Psychotic | Major; Sexual | | | |
| Disorders; Schizophrenia; | Dysfunctions, | | | |
| Substance-Related | Psychological | | | |
| Disorders | | | | |
| Bipolar | Depressive | Hepatitis C, Chronic | Myelodysplastic | Thrombocytopenia |
| Disorder; Depression; Sub | Disorder, | | Syndrome (MDS) | |
| stance-Related Disorders | Major; suicidal | | | |
| | ideation | | | |
| Bipolar | Depressive | Hepatitis C, Chronic; HIV | Myeloproliferative | Thromboembolism |
| Disorder; Depressive | Disorder; | Infections | Disorders | |
| Disorder; Psychotic | Depressive | | | |
| Disorders; schizoaffective | Disorder, | | | |
| disorder | Major | | | |
| Bipolar | Depressive | Hepatitis C, | Myocardial | Thrombosis |
| Disorder; Psychotic | Disorder; | Chronic; Recurrence | Infarction | |
| Disorders | Mental Disorders | | | |
| Bipolar | Depressive | Hepatitis C; HIV Infections | Myotonia | Tobacco Use |
| Disorder; Schizophrenia | Disorder; | | | Disorder |
| | Mood Disorders | | | |
| Blood Clotting | Depressive | Hepatitis C; Liver Neoplasms | Myotonic | tonsillectomy |
| | Disorder; | | Disorders | |
| | Narcolepsy | | | |
| Blood Thinner | Depressive | Heroin Dependence | Narcolepsy | Torsades de |
| | Disorder; | | | Pointes |
| | Obsessive- | | | |
| | Compulsive | | | |
| | Disorder | | | |
| bone density | Diabetes | Heroin Dependence; Memory | Nasopharyngeal | Toxic liver |
| | Mellitus | Disorders | Neoplasms | disease |
| Bone Diseases | Diabetes | Heroin Dependence; Opioid- | Nasopharyngeal | Toxic liver |
| | Mellitus, Type | Related Disorders | Neoplasms; | disease; |
| | 1; Heart | | Neutropenia | Tuberculosis |
| | Failure | | | |
| Bradycardia | Diabetes | High Blood Pressure | Nausea | transplant |
| | Mellitus, | | | rejection |
| | Type 2 | | | |
| Brain Diseases; Drug | Diabetes | High Cholesterol | Nausea and | Transplantation |
| Toxicity; Osteosarcoma; | Mellitus, Type | | Vomiting | |
| Precursor Cell | 2; Heart | | | |
| Lymphoblastic | Failure; | | | |
| Leukemia-Lymphoma | Pulmonary Disease, | | | |
| | Chronic | | | |
| | Obstructive | | | |
| Brain Neoplasms | Diabetes | HIV Infections | Nausea; Neoplasms; | Tuberculosis |
| | Mellitus, Type | | Vomiting | |
| | 2; Hypoglycemia | | | |
| Brain | Diabetes | HIV | Nausea; Pancreatic | Turner |
| Neoplasms; Deafness; | Mellitus, Type | Infections; Hyperbilirubinemia | Neoplasms | Syndrome |
| Ototoxicity | 2; Polycystic | | | |
| | Ovary | | | |
| | Syndrome | | | |
| Breast Neoplasms | Diabetes | HIV | Nausea; Vomiting | Urinary |
| | Mellitus, Type | Infections; Hyperlipidemias | | Bladder |
| | 2; Weight gain | | | Neoplasms |
| Breast | Diabetes | HIV | Neonatal | Urticaria |
| Neoplasms; Colorectal | Mellitus; Edema; | Infections; Hyperlipidemias; | Abstinence | |
| Neoplasms | Hyperlipidemias | Hypertriglyceridemia | Syndrome | |
| Breast Neoplasms; Drug | Diabetes | HIV | Neoplasm | Uterine |
| Toxicity | Mellitus; | Infections; | Metastasis | Cervical |
| | Hypertension | Hypertriglyceridemia | | Neoplasms |
| Breast Neoplasms; heart | Diarrhea | HIV Infections; Kidney | Neoplasm | Vasculitis |
| transplantation; Kidney | | Diseases | Metastasis; | |
| Neoplasms; Kidney | | | Stomach Neoplasms | |
| Transplantation; lung | | | | |
| transplantation; | | | | |
| Neuroendocrine Tumors | | | | |
| Breast | Diarrhea; | HIV | Neoplasm, | Vomiting |
| Neoplasms; Hyperglycemia; | Neoplasms | Infections; nephrolithiasis | Residual; | |
| Leukopenia | | | Precursor Cell | |
| | | | Lymphoblastic | |
| | | | Leukemia- | |
| | | | Lymphoma | |
| Breast | discontinuation | HIV Infections; nephrotoxicity | Neoplasms | Weight gain |
| Neoplasms; Kidney | | | | |
| Neoplasms; Kidney | | | | |
| Transplantation; | | | | |
| Neuroendocrine Tumors | | | | |
| Breast | Dizziness | HIV Infections; Pancreatic | Neoplasms; | |
| Neoplasms; Kidney | | Neoplasms | nephrotoxicity | |
| Neoplasms; | | | | |
| Neuroendocrine Tumors | | | | |
| Breast | dose reduction | HIV Infections; Peripheral | Neoplasms; | |
| Neoplasms; Leukopenia; | | Nervous System Diseases | Neurotoxicity | |
| Neutropenia | | | Syndromes | |

Table 3—Example list of phenotypes where based on prevalence of a risk or pathogenic variant Applicants can recommend additional screenings, treatments, lifestyle changes, or preventative medication

TABLE 3

| | | | | |
|---|---|---|---|---|
| 10q11.22q11.23 | Coenzyme Q10 | Guanidinoacetate | Leukoencephalopathy, | Porphyria, acute |
| microdeletion | deficiency, primary, 1 | methyltransferase | motor delay, | hepatic, digenic |
| including CHAT | | (GAMT) deficiency | spasticity, and | |
| and SLC18A3 | | | dysarthria | |
| | | | syndrome | |
| 22q11.2 central | COGNITIVE | Harel-Yoon syndrome | LIM DOMAIN | PPARG-related |
| duplication | IMPAIRMENT | | ONLY-1 | familial partial |
| syndrome | WITHOUT | | POLYMORPHISM | lipodystrophy |
| | CEREBELLAR | | | |
| | ATAXIA | | | |
| 3M syndrome 3 | COL9A1-Related | Hearing loss, | Lipoatrophy with | Pregnancy loss, |
| | Disorders | autosomal dominant | Diabetes, Hepatic | recurrent, |
| | | 74 | Steatosis, | susceptibility to, 2 |
| | | | Hypertrophic | |
| | | | Cardiomyopathy, | |
| | | | and | |
| | | | Leukomelanodermic | |
| | | | Papules | |
| 46, XY sex reversal 9 | Color vision defect | Hearing loss, | LMBRD2-related | Premature ovarian |
| | | autosomal recessive | disorder | failure 2B |
| | | 99 | | |
| Abdominal | Colton-null | Hemimegalencephaly | Long QT syndrome | Primary ciliary |
| obesity-metabolic | phenotype | | 2, acquired, | dyskinesia 12 |
| syndrome 4 | | | susceptibility to | |
| Abnormal cerebral | Combined oxidative | HEMOGLOBIN A(2) | LOW DENSITY | Primary ciliary |
| cortex morphology | phosphorylation | GROVETOWN | LIPOPROTEIN | dyskinesia 7 |
| | defect type 17 | | CHOLESTEROL | |
| | | | LEVEL | |
| | | | QUANTITATIVE | |
| | | | TRAIT LOCUS 7 | |
| Abnormal lactate | Combined oxidative | HEMOGLOBIN | LTBP2-related | Primary |
| dehydrogenase | phosphorylation | ADANA | Disorder | microcephaly type 2 |
| level | deficiency 40 | | | |
| Abnormal | Complement | HEMOGLOBIN | Lymphatic | Programmed death |
| thrombosis | component 3 | ATLANTA- | malformation | ligand-1 (PD-L1) |
| | deficiency | COVENTRY | | blocking antibody |
| | | | | response |
| Abnormality of the | Compton-North | HEMOGLOBIN | Macrocephaly | Progressive |
| cardiovascular | congenital | BIRMINGHAM | | familial heart |
| system | myopathy | (USA) | | block |
| Abnormality of the | Cone-rod dystrophy 7 | HEMOGLOBIN | MACULAR | Progressive |
| upper limb | | CAEN | DEGENERATION, | scapulohumeroperoneal |
| | | | AGE-RELATED, 1, | distal |
| | | | SUSCEPTIBILITY | myopathy |
| | | | TO | |
| ACCES syndrome | Congenital bilateral | HEMOGLOBIN | Malaria, severe, | Prostate cancer, |
| | aplasia of vas | CHONGQING | susceptibility to | hereditary, 12 |
| | deferens from | | | |
| | CFTR mutation | | | |
| ACO2-related | Congenital diarrhea | HEMOGLOBIN D | Malignant | PROTHROMBIN |
| disorders | 7 with exudative | (IRAN) | neoplastic disease | TYPE 3 |
| | enteropathy | | | |
| Acromesomelic | Congenital | HEMOGLOBIN | MAP2-associated | Pseudo-TORCH |
| dysplasia 2B | dyserythropoietic | DURHAM-N.C. | Neurodevelopmental | syndrome 3 |
| | anemia, type I | | Disorder | |
| Acute episodes of | CONGENITAL | HEMOGLOBIN F | Marfan syndrome, | Psoriasis 13, |
| neuropathic | HEART DEFECTS, | (COSENZA) | severe classic | susceptibility to |
| symptoms | MULTIPLE | | | |
| | TYPES, 8, WITH | | | |
| | OR WITHOUT | | | |
| | HETEROTAXY | | | |
| Adams-Oliver | Congenital | HEMOGLOBIN F | Matthew-Wood | Pulmonary |
| syndrome 2 | microvillous | (MEINOHAMA) | syndrome | alveolar |
| | atrophy | | | proteinosis |
| Adolescent | Congenital | HEMOGLOBIN | Meckel syndrome | Pulmonary |
| alopeciam | myasthenic | FORT DE FRANCE | 14 | hypertension, |
| dentogingival | syndrome 19 | | | primary, 3 |
| abnormalitites and | | | | |
| intellectual | | | | |
| disability | | | | |
| Adult-onset | Congenital NAD | HEMOGLOBIN G | MEDNIK | Pyloric stenosis, |
| proximal spinal | deficiency disorder | (PHILADELPHIA) | syndrome | infantile |
| muscular atrophy, | | | | hypertrophic, 5 |
| autosomal | | | | |
| dominant | | | | |
| Age related | Congenital | HEMOGLOBIN | Megalencephaly- | RAB23-related |
| macular | sideroblastic | GUIZHOU | polymicrogyria- | Carpenter |
| degeneration 15 | anemia-B-cell | | polydactyly- | syndrome |
| | immunodeficiency- | | hydrocephalus | |
| | periodic fever- | | syndrome 2 | |
| | developmental | | | |
| | delay syndrome | | | |
| Aicardi-Goutieres | Conjunctival | HEMOGLOBIN | Melanoma, | Rare genetic |
| syndrome 4 | telangiectasia | HINSDALE | cutaneous | intellectual |
| | | | malignant, | disability |
| | | | susceptibility to, 3 | |
| ALBUMIN | Corneal dystrophy, | HEMOGLOBIN J | Menkes kinky-hair | Recurrent |
| COARI I | Fuchs endothelial, 6 | | syndrome | infections |
| ALBUMIN | Coronary heart | HEMOGLOBIN J | Metachromatic | Refractory anemia |
| TOCHIGI | disease, | (LENS) | leukodystrophy, | with ringed |
| | susceptibility to, 1 | | late-onset | sideroblasts |
| | | | | (clinical) |
| ALKALINE | Cowden syndrome 1 | HEMOGLOBIN | Methylcobalamin | Renal dysplasia, |
| PHOSPHATASE, | | JOHNSTOWN | deficiency type | cystic, |
| PLACENTAL, | | | cblE | susceptibility to |
| ALLELE-1 | | | | |
| POLYMORPHISM | | | | |
| Alpha-N- | Cranioosteoarthropathy | HEMOGLOBIN | MGAT2-CDG | Renier-Gabreels- |
| acetylgalactos- | | KUROSAKI | | Jasper syndrome |
| aminidase deficiency | | | | |
| Alzheimer disease 3 | Crohn disease- | HEMOGLOBIN M | Microcephaly 23, | Retinal cone |
| | associated growth | (HYDE PARK) | primary, autosomal | dystrophy 3A |
| | failure, | | recessive | |
| | susceptibility to | | | |
| Amelogenesis | Curly hair, | HEMOGLOBIN | Microcephaly, | Retinitis |
| imperfecta type 1F | ankyloblepharon, | MELUSINE | cortical | pigmentosa 17 |
| | nail dysplasia | | malformations, and | |
| | syndrome | | intellectual | |
| | | | disability | |
| Amyloidosis, | Cutis laxa, recessive | HEMOGLOBIN N | Microphthalmia | Retinitis |
| primary localized | | (COSENZA) | | pigmentosa 45 |
| cutaneous, 2 | | | | |
| Amyotrophic | Cystic parathyroid | HEMOGLOBIN O | Microvascular | Retinitis |
| lateral sclerosis- | adenoma | (OLIVIERE) | complications of | pigmentosa 71 |
| parkinsonism- | | | diabetes, | |
| dementia complex | | | susceptibility to, 5 | |
| Anemia, hereditary | DDX3X-Related | HEMOGLOBIN | Mild | Retinitis |
| sideroblastic 1, | Disorder | PERTH | macrothrombocytopenia | pigmentosa- |
| pyridoxine | | | | hearing loss- |
| refractory | | | | premature aging- |
| | | | | short stature-facial |
| | | | | dysmorphism |
| | | | | syndrome |
| Anhydramnios | Deafness, | HEMOGLOBIN | Mitochondrial | Rhabdomyolysis |
| | neurosensory | RAINIER | complex 1 | |
| | autosomal recessive | | deficiency, nuclear | |
| | 21 | | type 15 | |
| Anterior creases of | Deep venous | HEMOGLOBIN | Mitochondrial | Rieger anomaly |
| earlobe | thrombosis, | SAINT NAZAIRE | complex 1 | |
| | protection against | | deficiency, nuclear | |
| | | | type 7 | |
| Aortic aneurysm | Deficiency of | HEMOGLOBIN | Mitochondrial | Robinow-Sorauf |
| | steroid 17-alpha- | SHIMONOSEKI | complex III | syndrome |
| | monooxygenase | | deficiency nuclear | |
| | | | type 1 | |
| APC-related | delta Thalassemia | HEMOGLOBIN SUN | Mitochondrial | Rudimentary fibula |
| attenuated familial | | PRAIRIE | DNA depletion | |
| adenomatous | | | syndrome 15 | |
| polyposis | | | (hepatocerebral | |
| | | | type) | |
| APOLIPOPROTEIN | Dermatofibrosarcoma | HEMOGLOBIN | Mitochondrial | Sarcoma |
| A-I | protuberans | TOKONAME | myopathy, | |
| (BALTIMORE) | | | episodic, with optic | |
| | | | atrophy and | |
| | | | reversible | |
| | | | leukoencephalopathy | |
| APP | Developmental and | HEMOGLOBIN | MN1 C-terminal | Schizophrenia 18 |
| POLYMORPHISM | epileptic | VANCOUVER | truncation (MCTT) | |
| | encephalopathy 96 | | syndrome | |
| Arrhinia with | Developmental and | HEMOGLOBIN | Mosaic | SCNIB-Related |
| choanal atresia and | epileptic | YORK | supernumerary | Disorder |
| microphthalmia | encephalopathy, 32 | | isodicentric | |
| syndrome | | | chromosome 10 | |
| Arthritis | Developmental and | Hemolytic-uremic | MPV17-Related | Scrotal hypoplasia |
| | epileptic | syndrome | Disorders | |
| | encephalopathy, 57 | | | |
| ARX-associated | Developmental and | Hepatitis C virus | Mucopolysaccharidosis | Seizure |
| condition | epileptic | infection, response to | type 2, severe | |
| | encephalopathy, 81 | therapy of | form | |
| asthenozoospermia | developmental | Hereditary cerebral | Multicentric carpo- | Senior-Loken |
| | encephalopathy | hemorrhage with | tarsal osteolysis | syndrome 9 |
| | with epilepsy | amyloidosis | with or without | |
| | | | nephropathy | |
| Ataxia with | Diabetes mellitus, | Hereditary intrinsic | Multiple endocrine | SETBP1-Related |
| oculomotor apraxia | type 1, | factor deficiency | neoplasia, type 2 | Disorder |
| type 3 | susceptibility to | | | |
| atorvastatin | Diaphragmatic | Hereditary sclerosing | Multiple myeloma, | Severe combined |
| response - | hernia 3 | poikiloderma with | resistance to | immunodeficiency, |
| Metabolism/PK | | tendon and pulmonary | | B cell-negative |
| | | involvement | | |
| Atrial fibrillation, | Diffuse interstitial | Hereditary spastic | Muscular atrophy | Severe muscular |
| familial, 18 | pulmonary fibrosis | paraplegia 3A | | hypotonia |
| Atrioventricular | Dilated | Hereditary spastic | Muscular | Shashi-Pena |
| septal defect 4 | cardiomyopathy 1L | paraplegia 75 | dystrophy- | syndrome |
| | | | dystroglycanopathy | |
| | | | type B5 | |
| Atypical Leigh | Disease of | Hermansky-Pudlak | Mycotic Aneurysm, | Short stature due to |
| syndrome | glomerular | syndrome 9 | Intracranial | primary acid-labile |
| | basement | | | subunit deficiency |
| | membrane | | | |
| Autism, | Distal myopathy | Heyn-Sproul-Jackson | Myocardial | Short-rib thoracic |
| susceptibility to, 16 | | syndrome | infarction, | dysplasia 15 with |
| | | | susceptibility to | polydactyly |
| Autoimmune | DNM1L-related | Hirschsprung disease, | myoglobinopathy | Sialuria |
| lymphoproliferative | movement disorder | susceptibility to, 3 | | |
| syndrome, type 1a | | | | |
| Autosomal | Downslanted | Holoprosencephaly 5 | Myopathy, distal, | simvastatin acid |
| dominant Charcot- | palpebral fissures | | 6, adult-onset, | response - |
| Marie-Tooth | | | autosomal | Metabolism/PK |
| disease type 2M | | | dominant | |
| Autosomal | Duffy Blood group | HPRT | Myopia 26, X- | Skeletal muscle |
| dominant keratitis- | system | EVANSVILLE | linked, female- | hypertrophy |
| ichthyosis-hearing | | | limited | |
| loss syndrome | | | | |
| Autosomal | Dysfibrinogenemia | HSPG2-Related | NADH- | Skin/hair/eye |
| dominant | | Disorders | CYTOCHROME | pigmentation, |
| nonsyndromic | | | b5 REDUCTASE | variation in, 9 |
| hearing loss 21 | | | POLYMORPHISM | |
| Autosomal | dysmorphy | Hydatidiform mole, | Nasopharyngeal | Slurred speech |
| dominant | | recurrent, 3 | carcinoma, | |
| nonsyndromic | | | susceptibility to, 3 | |
| hearing loss 68 | | | | |
| Autosomal | Dystonia, adult- | Hyper-IgM syndrome | Nemaline | SMS-Related |
| dominant striatal | onset | type 1 | myopathy 8 | Disorder |
| neurodegeneration | | | | |
| type 1 | | | | |
| Autosomal | Early-onset epilepsy | Hypercholesterolemia, | Neoplasm of | SPAST-related |
| recessive | | familial, 4 | stomach | spastic paraplegia |
| congenital | | | | |
| ichthyosis 4A | | | | |
| autosomal | Ectodermal | Hyperinsulinism due | Nephronophthisis | Spastic paraplegia- |
| recessive isolated | dysplasia 4, | to INSR deficiency | 19 | Paget disease of |
| fingernail dysplasia | hair/nail type | | | bone syndrome |
| Autosomal | EEM syndrome | Hyperornithinemia | Nephrotic | Spermatogenic |
| recessive lower | | | syndrome, type 21 | failure 10 |
| motor neuron | | | | |
| disease with | | | | |
| childhood onset | | | | |
| Autosomal | Ehlers-Danlos | Hyperreflexia | Neurodegeneration | Spermatogenic |
| recessive | syndrome, | | | failure 35 |
| nonsyndromic | spondylodysplastic | | | |
| hearing loss 31 | type, 1 | | | |
| Autosomal | Embryonic calcium | Hypertrophic | Neurodevelopmental | Spermatogenic |
| recessive | dysregulation | cardiomyopathy 14 | disorder and | failure 62 |
| nonsyndromic | | | structural brain | |
| hearing loss 84B | | | anomalies with or | |
| | | | without seizures | |
| | | | and spasticity | |
| Autosomal | Encephalopathy | Hypoalphalipoproteinemia, | Neurodevelopmental | Spherocytosis, |
| recessive retinitis | | primary, 2 | disorder with | type 1, autosomal |
| pigmentosa | | | epilepsy | recessive |
| Autosomal | enflurane response - | Hypogonadotropic | Neurodevelopmental | SPINK5 |
| systemic lupus | Toxicity | hypogonadism 17 | disorder with | POLYMORPHISM |
| erythematosus type | | with or without | language delay and | |
| 16 | | anosmia | seizures | |
| B lymphoblastic | Epidermolysis | Hypohidrosis | Neurodevelopmental | Spinocerebellar |
| leukemia | bullosa pruriginosa, | | disorder with or | ataxia type 27 |
| lymphoma, no | autosomal dominant | | without variable | |
| ICD-O subtype | | | brain abnormalities | |
| Barber-Say | Epidermolysis | Hypomyelinating | Neurodevelopmental | Spinocerebellar |
| syndrome | bullosa, junctional | leukodystrophy 6 | disorder, | ataxia, autosomal |
| | 4, intermediate | | mitochondrial, with | recessive, with |
| | | | abnormal | axonal neuropathy 2 |
| | | | movements and | |
| | | | lactic acidosis, with | |
| | | | or without seizures | |
| Bardet-biedl | Epilepsy, familial | Hypoplastic aortic | Neuromotor delay | Spondylocostal |
| syndrome 6/10, | focal, with variable | arch | | dysostosis 6, |
| digenic | foci 2 | | | autosomal |
| | | | | recessive |
| Basal ganglia | Epilepsy, | Hypotonia, | Neuronopathy, | Spondyloepiphyseal |
| calcification | progressive | hypoventilation, | distal hereditary | dysplasia, |
| | myoclonic, 12 | impaired intellectual | motor, type 7B | Kimberley type |
| | | development, | | |
| | | dysautonomia, | | |
| | | epilepsy, and eye | | |
| | | abnormalities | | |
| Becker muscular | Episodic | Ichthyosis hystrix of | Neutropenia, severe | SPTBN1-related |
| dystrophy | kinesigenic | Curth-Macklin | congenital, 2, | neurodevelopmental |
| | dyskinesia 1 | | autosomal | disease |
| | | | dominant | |
| BEST1-Related | Erythrokeratodermia | IFN-gamma receptor | Night blindness, | STAT1-Related |
| Disorders | variabilis et | 1 deficiency | congenital | Disorder |
| | progressiva 2 | | stationary, type1i | |
| Bicuspid aortic | Even-plus | Immunodeficiency | Nonarteritic | Stormorken |
| valve | syndrome | 106, susceptibility to | anterior ischemic | syndrome |
| | | viral infections | optic neuropathy, | |
| | | | susceptibility to | |
| Bile duct cancer | Exudative | Immunodeficiency 49 | Noonan syndrome 1 | Subcortical band |
| | vitreoretinopathy 2, | | | heterotopia |
| | X-linked | | | |
| Blepharophimosis - | Facial hypotonia | Immunodeficiency 86 | Normophosphatemic | Sulfite oxidase |
| intellectual | | | familial tumoral | deficiency due to |
| disability | | | calcinosis | molybdenum |
| syndrome, MKB | | | | cofactor deficiency |
| type | | | | type C |
| Blue rubber bleb | FACTOR XII | Immunodeficiency, | NUCLEOSIDE | Susceptibility to |
| nevus | POLYMORPHISM | common variable, 12 | PHOSPHORYLASE | severe coronavirus |
| | | | POLYMORPHISM | disease (COVID- |
| | | | | 19) due to high |
| | | | | plasma levels of |
| | | | | TNF, TNFR, |
| | | | | and/or TNFR6 |
| Bone mineral | Familial | Inability to walk | Obsessive- | Syndromic |
| density quantitative | Candidiasis, | | compulsive | hydrocephalus due |
| trait locus 1 | Recessive | | disorder | to diffuse villous |
| | | | | hyperplasia of |
| | | | | choroid plexus |
| Brachydactyly type | Familial exudative | INDIAN BLOOD | Oculodentodigital | Synpolydactyly |
| A2 | vitreoretinopathy | GROUP SYSTEM | dysplasia, | |
| | | POLYMORPHISM | autosomal | |
| | | | recessive | |
| Brain | Familial | Infantile Nystagmus | Oligohydramnios | tacrolimus |
| pseudoatrophy, | hypobetalipoproteinemia | | | response - |
| reversible, | | | | Metabolism/PK |
| valproate-induced, | | | | |
| susceptibility to | | | | |
| Breast cancer, | Familial juvenile | Inflammatory skin | Oppositional | TBX2-related |
| susceptibility to | hyperuricemic | and bowel disease, | defiant disorder | condition |
| | nephropathy type 2 | neonatal, 1 | | |
| Brown-Vialetto- | Familial | intellectual deficiency | Ornithine | Terminal osseous |
| van Laere | spontaneous | | aminotransferase | dysplasia- |
| syndrome 1 | pneumothorax | | deficiency | pigmentary defects |
| | | | | syndrome |
| BSCL2-related | Fanconi anemia | Intellectual | Orthostatic | Thiel-Behnke |
| Developmental and | complementation | developmental | hypotension 2 | corneal dystrophy |
| epileptic | group J | disorder with or | | |
| encephalopathy | | without peripheral | | |
| | | neuropathy | | |
| Cachexia | Fatal infantile | Intellectual | Osteogenesis | Thrombocytopenia |
| | hypertonic | developmental | imperfecta type 8 | X-linked, |
| | myofibrillar | disorder, X-linked | | intermittent |
| | myopathy | 108 | | |
| Camptodactyly-tall | Feingold syndrome | Intellectual disability, | Osteoporosis | Thyroid cancer, |
| stature-scoliosis- | type 1 | anterior maxillary | | nonmedullary, 5 |
| hearing loss | | protrusion, and | | |
| syndrome | | strabismus | | |
| captopril response - | FGFR2 related | Intellectual disability, | Ovarian dysgenesis 6 | Tibial muscular |
| Efficacy | craniosynostosis | autosomal dominant | | dystrophy |
| | | 46 | | |
| Cardiac valvular | FIBRINOGEN | Intellectual disability, | PACS1-related | tobramycin |
| dysplasia, X-linked | LILLE 1 | autosomal recessive | syndrome | response - Toxicity |
| | | 44 | | |
| Cardiomyopathy, | Fibrosis, | Intellectual disability, | PAN2-related | TP63-Related |
| familial | neurodegeneration, | profound | multiple congenital | Spectrum |
| hypertrophic 27 | and cerebral | | anomalies | Disorders |
| | angiomatosis | | syndrome | |
| Carnitine palmitoyl | Fluorouracil | Intellectual disability, | Papillorenal | Transient Neonatal |
| transferase II | response | X-linked 72 | syndrome with | Diabetes, |
| deficiency, severe | | | macular | Recessive |
| infantile form | | | abnormalities | |
| Cataract 13 with | Focal T2 | Internal carotid artery | Parkes Weber | Trichohepatoenteric |
| adult I phenotype | hyperintense basal | dissection | syndrome | syndrome |
| | ganglia lesion | | | |
| Cataract 45 | Freeman-Sheldon | Intrahepatic | Parkinsonism- | Trigonocephaly 1 |
| | syndrome | cholestasis with | dystonia, infantile, 2 | |
| | | episodic jaundice | | |
| Cavernous | Fructose- | Irregular respiration | Patent ductus | TRRAP-Related |
| hemangioma | biphosphatase | | arteriosus 3 | Disorder |
| | deficiency | | | |
| Cenani-Lenz | G6PD CHATHAM | Isolated macular | Pedal edema | Tuberous sclerosis |
| syndactyly | | dystrophy | | syndrome |
| syndrome | | | | |
| Cerebellar ataxia, | G6PD | JARID2-associated | Penile hypospadias | TYPE 1 |
| intellectual | METAPONTO | Neurodevelopmental | | DIABETES |
| disability, and | | disorder | | MELLITUS, |
| dysequilibrium | | | | INSULIN- |
| syndrome 1 | | | | DEPENDENT, 10 |
| Cerebral autosomal | G6PD TAIWAN- | Joubert syndrome 15 | Peripheral schisis | UCP3 |
| dominant | HAKKA 2 | | | POLYMORPHISM |
| arteriopathy with | | | | G/A |
| subcortical infarcts | | | | |
| and | | | | |
| leukoencephalopathy | | | | |
| Cerebrofaciothoracic | Galloway-Mowat | Joubert syndrome 40 | Peroxisome | Unverricht- |
| dysplasia | syndrome 3 | | biogenesis disorder | Lundborg |
| | | | 11A (Zellweger) | syndrome |
| Charcot-Marie- | Gastrointestinal | Juvenile onset | Perrault syndrome | Usher syndrome |
| Tooth disease | defects and | psychosis | | type 1J |
| axonal type 2F | immunodeficiency | | | |
| | syndrome 1 | | | |
| Charcot-Marie- | Geleophysic | Kartagener syndrome | Pfeiffer syndrome | VACTERL |
| Tooth disease type 2 | dysplasia | | | association, X- |
| | | | | linked, with or |
| | | | | without |
| | | | | hydrocephalus |
| Charcot-Marie- | Generalized | KCNT1-related | Phosphoenolpyruvate | Vein of Galen |
| Tooth disease X- | hypotonia | channelopathy | carboxykinase | aneurysmal |
| linked dominant 1 | | | deficiency, | malformation |
| | | | mitochondrial | |
| Charcot-Marie- | GFPT1-related | Keratosis pilaris | PI | Vertebral |
| Tooth, Intermediate | myasthenic | | NULL(NEWPORT) | hypersegmentation |
| | syndrome | | | and orofacial |
| | | | | anomalies |
| Childhood | Glaucoma 1, open | Klippel-Feil | PIDD1-associated | VISS |
| ganglioglioma | angle, a, digenic | syndrome 2, | neurodevelopmental | SYNDROME |
| | | autosomal recessive | disorder | |
| Cholestasis, | Glioma | Lactase persistence | Pilomatrixoma | Vitiligo-associated |
| progressive | susceptibility 1 | | | multiple |
| familial | | | | autoimmune |
| intrahepatic, 5 | | | | disease |
| | | | | susceptibility 1 |
| Choroid plexus | Glucocorticoid | Laron syndrome with | Plasma factor XI | Waardenburg |
| papilloma | deficiency with | undetectable serum | deficiency | syndrome type 2E, |
| | achalasia | GH-binding protein | | without neurologic |
| | | | | involvement |
| Chromosome | Glycemia variation | Leber congenital | PLOD3-Related | WDR19-Related |
| 1q21.1 duplication | | amaurosis 10 | Disorder | Disorders |
| syndrome | | | | |
| Chronic | Glycogen storage | Left ventricular | Poly (ADP-Ribose) | WHITE- |
| mucocutaneous | disease type 1 due | noncompaction 10 | polymerase | KERNOHAN |
| candidiasis | to SLC37A4 | | inhibitor response | SYNDROME |
| | mutation | | | |
| Ciliary dyskinesia, | Gm2- | Leprosy, protection | Polydactyly, | Wolfram syndrome |
| primary, 46 | gangliosidosis, | against | postaxial, type a7 | |
| | adult-onset | | | |
| Cleft lip | Gonadal tissue | Lethal | polysyndactyly | X-linked complex |
| | inappropriate for | Encephalopathy | | neurodevelopmental |
| | external genitalia or | | | disorder |
| | chromosomal sex | | | |
| CNNM2-related | Greig | Leukocyte adhesion | Pontocerebellar | X-linked |
| neurodevelopmental | cephalopolysyndactyly | deficiency 1 | hypoplasia, type 14 | intellectual |
| disorder and | syndrome, | | | disability-short |
| hypomagnesemia | severe | | | stature-overweight |
| | | | | syndrome |
| | | | X-linked spasticity- | ZAKI |
| | | | intellectual | SYNDROME |
| | | | disability-epilepsy | |
| | | | syndrome | |

Table 4—Example if layman's translation of annotation data:

TABLE 4
pgkb_clinanns.loc[pgkb_clinanns[‘Drug(s)’] == “telmisartan”, ‘Phenotype(s)’] = “High Blood Pressure”
pgkb_clinanns.loc[pgkb_clinanns[‘Drug(s)’] == “simvastatin”, ‘Phenotype(s)’] = “High Cholesterol”
pgkb_clinanns.loc[pgkb_clinanns[‘Drug(s)’] == “lovastatin acid”, ‘Phenotype(s)’] = “High Cholesterol”
pgkb_clinanns.loc[pgkb_clinanns[‘Drug(s)’] == “lovastatin; lovastatin acid”, ‘Phenotype(s)’] = “High Cholesterol”
pgkb_clinanns.loc[pgkb_clinanns[‘Drug(s)’] == “lovastatin”, ‘Phenotype(s)’] = “High Cholesterol”
pgkb_clinanns.loc[pgkb_clinanns[‘Drug(s)’] == “simvastatin acid”, ‘Phenotype(s)’] = “High Cholesterol”
pgkb_clinanns.loc[pgkb_clinanns[‘Drug(s)’] == “simvastatin”, ‘Phenotype(s)’] = “High Cholesterol”
pgkb_clinanns.loc[pgkb_clinanns[‘Drug(s)’] == “fentanyl”, ‘Phenotype(s)’] = “Severe Pain”
pgkb_clinanns.loc[pgkb_clinanns[‘Drug(s)’] == “remifentanil”, ‘Phenotype(s)’] = “Pain”
pgkb_clinanns.loc[pgkb_clinanns[‘Drug(s)’] == “clopidogrel”, ‘Phenotype(s)’] = “Stroke and Heart Attack Prevention”
pgkb_clinanns.loc[pgkb_clinanns[‘Drug(s)’] == “acenocoumarol”, ‘Phenotype(s)’] = “Anticoagulant”
pgkb_clinanns.loc[pgkb_clinanns[‘Drug(s)’] == “celecoxib”, ‘Phenotype(s)’] = “Paim”
pgkb_clinanns.loc[pgkb_clinanns[‘Drug(s)’] == “clopidogrel”, ‘Phenotype(s)’] = “Stroke and Heart Attack Prevention”
pgkb_clinanns.loc[pgkb_clinanns[‘Drug(s)’] == “codeine”, ‘Phenotype(s)’] = “Pain and Cough”
pgkb_clinanns.loc[pgkb_clinanns[‘Drug(s)’] == “dexlansoprazole”, ‘Phenotype(s)’] = “Heartburn, GERD, Esophageal Damage”
pgkb_clinanns.loc[pgkb_clinanns[‘Drug(s)’] == “doxepin”, ‘Phenotype(s)’] = “Antidepressant and Nerve Pain”
pgkb_clinanns.loc[pgkb_clinanns[‘Drug(s)’] == “fluorouracil”, ‘Phenotype(s)’] = “Chemotherapy”
pgkb_clinanns.loc[pgkb_clinanns[‘Drug(s)’] == “flurbiprofen”, ‘Phenotype(s)’] = “Pain and Arthritis”
pgkb_clinanns.loc[pgkb_clinanns[‘Drug(s)’] == “fluvastatin”, ‘Phenotype(s)’] = “High Cholesterol”
pgkb_clinanns.loc[pgkb_clinanns[‘Drug(s)’] == “hydrocodone”, ‘Phenotype(s)’] = “Pain and Cough”
pgkb_clinanns.loc[pgkb_clinanns[‘Drug(s)’] == “ibuprofen”, ‘Phenotype(s)’] = “Fever and Pain”
pgkb_clinanns.loc[pgkb_clinanns[‘Drug(s)’] == “irinotecan”, ‘Phenotype(s)’] = “Chemotherapy”
pgkb_clinanns.loc[pgkb_clinanns[‘Drug(s)’] == “lornoxicam”, ‘Phenotype(s)’] = “Pain and Arthritis”
pgkb_clinanns.loc[pgkb_clinanns[‘Drug(s)’] == “meloxicam”, ‘Phenotype(s)’] = “Arthritis”
pgkb_clinanns.loc[pgkb_clinanns[‘Drug(s)’] == “mercaptopurine”, ‘Phenotype(s)’] = “Chemotherapy and Immunosuppressive”
pgkb_clinanns.loc[pgkb_clinanns[‘Drug(s)’] == “methoxyflurane”, ‘Phenotype(s)’] = “Pain”
pgkb_clinanns.loc[pgkb_clinanns[‘Drug(s)’] == “pantoprazole”, ‘Phenotype(s)’] = “GERD, Damaged Esophagus, and Stomach Acid”
pgkb_clinanns.loc[pgkb_clinanns[‘Drug(s)’] == “paroxetine”, ‘Phenotype(s)’] = “SSRI”
pgkb_clinanns.loc[pgkb_clinanns[‘Drug(s)’] == “phenprocoumon”, ‘Phenotype(s)’] = “Blood Thinner”
pgkb_clinanns.loc[pgkb_clinanns[‘Drug(s)’] == “piroxicam”, ‘Phenotype(s)’] = “Pain”
pgkb_clinanns.loc[pgkb_clinanns[‘Drug(s)’] == “pitavastatin”, ‘Phenotype(s)’] = “High Cholesterol”
pgkb_clinanns.loc[pgkb_clinanns[‘Drug(s)’] == “pravastatin”, ‘Phenotype(s)’] = “High Cholesterol”
pgkb_clinanns.loc[pgkb_clinanns[‘Drug(s)’] == “quetiapine”, ‘Phenotype(s)’] = “Schizophrenia, Bipolar disorder, Depression”
pgkb_clinanns.loc[pgkb_clinanns[‘Drug(s)’] == “rosuvastatin”, ‘Phenotype(s)’] = “High Cholesterol”
pgkb_clinanns.loc[pgkb_clinanns[‘Drug(s)’] == “simvastatin”, ‘Phenotype(s)’] = “High Cholesterol”
pgkb_clinanns.loc[pgkb_clinanns[‘Drug(s)’] == “siponimod”, ‘Phenotype(s)’] = “Multiple Sclerosis”
pgkb_clinanns.loc[pgkb_clinanns[‘Drug(s)’] == “tenoxicam”, ‘Phenotype(s)’] = “Arthritis and Pain”
pgkb_clinanns.loc[pgkb_clinanns[‘Drug(s)’] == “tramadol”, ‘Phenotype(s)’] = “Pain”
pgkb_clinanns.loc[pgkb_clinanns[‘Drug(s)’] == “trimipramine”, ‘Phenotype(s)’] = “Myelodysplastic Syndrome (MDS)”
pgkb_clinanns.loc[pgkb_clinanns[‘Drug(s)’] == “tropisetron”, ‘Phenotype(s)’] = “Nausea and Vomiting”
pgkb_clinanns.loc[pgkb_clinanns[‘Drug(s)’] == “voriconazole”, ‘Phenotype(s)’] = “Antifungal”
pgkb_clinanns.loc[pgkb_clinanns[‘Drug(s)’] == “warfarin”, ‘Phenotype(s)’] = “Blood Clotting”

Table 5—Example of non-coding cell types to disease states:

TABLE 5

| | |
|---|---|
| Mononuclear Phagocytes (38/38) | Immune conditions: e.g., irritable bowel disease (IBD), |
| | Crohn's disease, inflammation |
| THP1_LPS_4hr-Engreitz | Immune conditions: e.g., irritable bowel disease (IBD), |
| | Crohn's disease, inflammation |
| THP_pmaLPS_ATAC_6h | Immune conditions: e.g., irritable bowel disease (IBD), |
| | Crohn's disease, inflammation |
| CD14-positive_mono- | Immune conditions: e.g., irritable bowel disease (IBD), |
| cyte_treated_with_LPS_4h-Novakovic2016 | Crohn's disease, inflammation |
| CD14-positive_mono- | Immune conditions: e.g., irritable bowel disease (IBD), |
| cyte_treated_with_BG_1h-Novakovic2016 | Crohn's disease, inflammation |
| CD14-positive_mono- | Immune conditions: e.g., irritable bowel disease (IBD), |
| cyte_treated_with_RPMI_d6-Novakovic2016 | Crohn's disease, inflammation |
| THP1-Engreitz | Immune conditions: e.g., irritable bowel disease (IBD), |
| | Crohn's disease, inflammation |
| THP_pmaLPS_ATAC_96h | Immune conditions: e.g., irritable bowel disease (IBD), |
| | Crohn's disease, inflammation |
| CD14-positive_mono- | Immune conditions: e.g., irritable bowel disease (IBD), |
| cyte_treated_with_BG_d1-Novakovic2016 | Crohn's disease, inflammation |
| THP_pmaLPS_ATAC_72h | Immune conditions: e.g., irritable bowel disease (IBD), |
| | Crohn's disease, inflammation |
| THP-1_monocyte-VanBortle2017 | Immune conditions: e.g., irritable bowel disease (IBD), |
| | Crohn's disease, inflammation |
| CD14-positive_mono- | Immune conditions: e.g., irritable bowel disease (IBD), |
| cyte_treated_with_LPS_d1-Novakovic2016 | Crohn's disease, inflammation |
| CD14-positive_monocyte-ENCODE | Immune conditions: e.g., irritable bowel disease (IBD), |
| | Crohn's disease, inflammation |
| THP_pmaLPS_ATAC_2h | Immune conditions: e.g., irritable bowel disease (IBD), |
| | Crohn's disease, inflammation |
| dendritic_cell_treated_with_Lipopoly- | Immune conditions: e.g., irritable bowel disease (IBD), |
| saccharide_0_ng-mL_for_0_hour-Garber2017 | Crohn's disease, inflammation |
| CD14-positive_mono- | Immune conditions: e.g., irritable bowel disease (IBD), |
| cyte_treated_with_RPMI_1h-Novakovic2016 | Crohn's disease, inflammation |
| U937_LPS_4hr-Engreitz | Immune conditions: e.g., irritable bowel disease (IBD), |
| | Crohn's disease, inflammation |
| CD14-positive_monocyte-Novakovic2016 | Immune conditions: e.g., irritable bowel disease (IBD), |
| | Crohn's disease, inflammation |
| dendritic_cell_treated_with_Lipopoly- | Immune conditions: e.g., irritable bowel disease (IBD), |
| saccharide_100_ng-mL_for_6_hour-Garber2017 | Crohn's disease, inflammation |
| CD14-positive_mono- | Immune conditions: e.g., irritable bowel disease (IBD), |
| cyte_treated_with_BG_4h-Novakovic2016 | Crohn's disease, inflammation |
| THP_pmaLPS_ATAC_0h | Immune conditions: e.g., irritable bowel disease (IBD), |
| | Crohn's disease, inflammation |
| HAP1 | Immune conditions: e.g., irritable bowel disease (IBD), |
| | Crohn's disease, inflammation |
| THP-1_macrophage-VanBortle2017 | Immune conditions: e.g., irritable bowel disease (IBD), |
| | Crohn's disease, inflammation |
| dendritic_cell_treated_with_Lipopoly- | Immune conditions: e.g., irritable bowel disease (IBD), |
| saccharide_100_ng-mL_for_1_hour-Garber2017 | Crohn's disease, inflammation |
| U937-Engreitz | Immune conditions: e.g., irritable bowel disease (IBD), |
| | Crohn's disease, inflammation |
| CD14-positive_mono- | Immune conditions: e.g., irritable bowel disease (IBD), |
| cyte_treated_with_RPMI_d1-Novakovic2016 | Crohn's disease, inflammation |
| CD14-positive_mono- | Immune conditions: e.g., irritable bowel disease (IBD), |
| cyte_treated_with_RPMI_4h-Novakovic2016 | Crohn's disease, inflammation |
| CD14-positive_mono- | Immune conditions: e.g., irritable bowel disease (IBD), |
| cyte_treated_with_LPS_d6-Novakovic2016 | Crohn's disease, inflammation |
| CD14-positive_mono- | Immune conditions: e.g., irritable bowel disease (IBD), |
| cyte_treated_with_LPS_1h-Novakovic2016 | Crohn's disease, inflammation |
| THP_pmaLPS_ATAC_24h | Immune conditions: e.g., irritable bowel disease (IBD), |
| | Crohn's disease, inflammation |
| THP_pmaLPS_ATAC_1h | Immune conditions: e.g., irritable bowel disease (IBD), |
| | Crohn's disease, inflammation |
| dendritic_cell_treated_with_Lipopoly- | Immune conditions: e.g., irritable bowel disease (IBD), |
| saccharide_100_ng-mL_for_2_hour-Garber2017 | Crohn's disease, inflammation |
| CD14-positive_mono- | Immune conditions: e.g., irritable bowel disease (IBD), |
| cyte_treated_with_BG_d6-Novakovic2016 | Crohn's disease, inflammation |
| dendritic_cell_treated_with_Lipopoly- | Immune conditions: e.g., irritable bowel disease (IBD), |
| saccharide_100_ng-mL_for_30_minute-Garber2017 | Crohn's disease, inflammation |
| THP_pmaLPS_ATAC_120h | Immune conditions: e.g., irritable bowel disease (IBD), |
| | Crohn's disease, inflammation |
| dendritic_cell_treated_with_Lipopoly | Immune conditions: e.g., irritable bowel disease (IBD), |
| saccharide_100_ng-mL_for_4_hour-Garber2017 | Crohn's disease, inflammation |
| THP_pmaLPS_ATAC_48h | Immune conditions: e.g., irritable bowel disease (IBD), |
| | Crohn's disease, inflammation |
| CD14-positive_monocytes-Roadmap | Immune conditions: e.g., irritable bowel disease (IBD), |
| | Crohn's disease, inflammation |
| THP_pmaLPS_ATAC_12h | Immune conditions: e.g., irritable bowel disease (IBD), |
| | Crohn's disease, inflammation |
| B Cells (9/9) | Immune conditions: e.g., irritable bowel disease (IBD), |
| | Crohn's disease, inflammation |
| Karpas-422-ENCODE | Immune conditions: e.g., irritable bowel disease (IBD), |
| | Crohn's disease, inflammation |
| BJAB-Engreitz | Immune conditions: e.g., irritable bowel disease (IBD), |
| | Crohn's disease, inflammation |
| BJAB_anti-IgM_anti-CD40_4hr-Engreitz | Immune conditions: e.g., irritable bowel disease (IBD), |
| | Crohn's disease, inflammation |
| B_cell-ENCODE | Immune conditions: e.g., irritable bowel disease (IBD), |
| | Crohn's disease, inflammation |
| OCI-LY7-ENCODE | Immune conditions: e.g., irritable bowel disease (IBD), |
| | Crohn's disease, inflammation |
| GM12878-Roadmap | Immune conditions: e.g., irritable bowel disease (IBD), |
| | Crohn's disease, inflammation |
| MM.1S-ENCODE | Immune conditions: e.g., irritable bowel disease (IBD), |
| | Crohn's disease, inflammation |
| CD19-positive_B_cell-Roadmap | Immune conditions: e.g., irritable bowel disease (IBD), |
| | Crohn's disease, inflammation |
| CD8-positive_alpha-beta_T_cell-ENCODE | Immune conditions: e.g., irritable bowel disease (IBD), |
| | Crohn's disease, inflammation |
| T Cells (8/8) | Immune conditions: e.g., irritable bowel disease (IBD), |
| | Crohn's disease, inflammation |
| Jurkat_anti-CD3_PMA_4hr-Engreitz | Immune conditions: e.g., irritable bowel disease (IBD), |
| | Crohn's disease, inflammation |
| Jurkat-Engreitz | Immune conditions: e.g., irritable bowel disease (IBD), |
| | Crohn's disease, inflammation |
| CD3-positive_T_cell-Roadmap | Immune conditions: e.g., irritable bowel disease (IBD), |
| | Crohn's disease, inflammation |
| CD4-positive_helper_T_cell-ENCODE | Immune conditions: e.g., irritable bowel disease (IBD), |
| | Crohn's disease, inflammation |
| CD8-positive_alpha-beta_T_cell-Corces2016 | Immune conditions: e.g., irritable bowel disease (IBD), |
| | Crohn's disease, inflammation |
| thymus_fetal-Roadmap | Immune conditions: e.g., irritable bowel disease (IBD), |
| | Crohn's disease, inflammation |
| CD4-positive_helper_T_cell-Corces2016 | Immune conditions: e.g., irritable bowel disease (IBD), |
| | Crohn's disease, inflammation |
| T-cell-ENCODE | Immune conditions: e.g., irritable bowel disease (IBD), |
| | Crohn's disease, inflammation |
| Other haematopoietic cells (8/8) | Hematological conditions; Immune conditions: e.g., |
| | irritable bowel disease (IBD), Crohn's disease, |
| | inflammation |
| spleen-ENCODE | Hematological conditions; Immune conditions: e.g., |
| | irritable bowel disease (IBD), Crohn's disease, |
| | inflammation |
| K562-Roadmap | Hematological conditions, e.g., Chronic myeloid leukemia; |
| | Immune conditions: e.g., irritable bowel disease (IBD), |
| | Crohn's disease, inflammation |
| CD56-positive_natural_killer_cells-Roadmap | Hematological conditions; Immune conditions: e.g., |
| | irritable bowel disease (IBD), Crohn's disease, |
| | inflammation |
| erythroblast-Corces2016 | Hematological conditions; Immune conditions: e.g., |
| | irritable bowel disease (IBD), Crohn's disease, |
| | inflammation |
| CD34-positive_mobilized-Roadmap | Hematological conditions; Immune conditions: e.g., |
| | irritable bowel disease (IBD), Crohn's disease, |
| | inflammation |
| megakaryocyte-erythroid_progenitor-Corces2016 | Hematological conditions; Immune conditions: e.g., |
| | irritable bowel disease (IBD), Crohn's disease, |
| | inflammation |
| natural_killer_cell-Corces2016 | Hematological conditions; Immune conditions: e.g., |
| | irritable bowel disease (IBD), Crohn's disease, |
| | inflammation |
| IMR90-Roadmap | Hematological conditions; Immune conditions: e.g., |
| | irritable bowel disease (IBD), Crohn's disease, |
| | inflammation |
| Fibroblasts (5/5) | Connective tissue disorders; Immune conditions: e.g., |
| | irritable bowel disease (IBD), Crohn's disease, |
| | inflammation |
| foreskin_fibroblast-Roadmap | Connective tissue disorders; Immune conditions: e.g., |
| | irritable bowel disease (IBD), Crohn's disease, |
| | inflammation |
| fibroblast_of_dermis-Roadmap | Connective tissue disorders; Immune conditions: e.g., |
| | irritable bowel disease (IBD), Crohn's disease, |
| | inflammation |
| fibroblast_of_arm-ENCODE | Connective tissue disorders; Immune conditions: e.g., |
| | irritable bowel disease (IBD), Crohn's disease, |
| | inflammation |
| astrocyte-ENCODE | Connective tissue disorders; Immune conditions: e.g., |
| | irritable bowel disease (IBD), Crohn's disease, |
| | inflammation |
| fibroblast_of_lung-Roadmap | Connective tissue disorders; Immune conditions: e.g., |
| | irritable bowel disease (IBD), Crohn's disease, |
| | inflammation |
| Epithelial (42/42) | Immune conditions: e.g., irritable bowel disease (IBD), |
| | Crohn's disease, inflammation |
| small_intestine_fetal-Roadmap | Immune conditions: e.g., irritable bowel disease (IBD), |
| | Crohn's disease, inflammation |
| epithelial_cell_of_prostate-ENCODE | Prostate cancer; Immune conditions: e.g., irritable bowel |
| | disease (IBD), Crohn's disease, inflammation |
| HCT116-ENCODE | Immune conditions: e.g., irritable bowel disease (IBD), |
| | Crohn's disease, inflammation |
| MCF-7-ENCODE | Immune conditions: e.g., irritable bowel disease (IBD), |
| | Crohn's disease, inflammation |
| adrenal_gland_fetal-ENCODE | Immune conditions: e.g., irritable bowel disease (IBD), |
| | Crohn's disease, inflammation |
| H7 | Immune conditions: e.g., irritable bowel disease (IBD), |
| | Crohn's disease, inflammation |
| keratinocyte-Roadmap | Psoriasis; Immune conditions: e.g., irritable bowel disease |
| | (IBD), Crohn's disease, inflammation |
| MCF10A-Ji2017 | Immune conditions: e.g., irritable bowel disease (IBD), |
| | Crohn's disease, inflammation |
| pancreas-Roadmap | Pancreatic cancer; Immune conditions: e.g., irritable bowel |
| | disease (IBD), Crohn's disease, inflammation |
| mammary_epithelial_cell-Roadmap | Breast cancer; Dysplasia; Immune conditions: e.g., irritable |
| | bowel disease (IBD), Crohn's disease, inflammation |
| stomach-Roadmap | Stomach cancer; Immune conditions: e.g., irritable bowel |
| | disease (IBD), Crohn's disease, inflammation |
| LoVo | Colorectal cancer; Immune conditions: e.g., irritable bowel |
| | disease (IBD), Crohn's disease, inflammation |
| A549_treated_with_ethanol_0.02_per- | Immune conditions: e.g., irritable bowel disease (IBD), |
| cent_for_1_hour-Roadmap | Crohn's disease, inflammation |
| PC-9-ENCODE | Immune conditions: e.g., irritable bowel disease (IBD), |
| | Crohn's disease, inflammation |
| H1-hESC-Roadmap | Immune conditions: e.g., irritable bowel disease (IBD), |
| | Crohn's disease, inflammation |
| thyroid_gland-ENCODE | Immune conditions: e.g., irritable bowel disease (IBD), |
| | Crohn's disease, inflammation |
| HT29 | Immune conditions: e.g., irritable bowel disease (IBD), |
| | Crohn's disease, inflammation |
| MDA-MB-231 | Immune conditions: e.g., irritable bowel disease (IBD), |
| | Crohn's disease, inflammation |
| iPS_DF_19.11_Cell_Line-Roadmap | Immune conditions: e.g., irritable bowel disease (IBD), |
| | Crohn's disease, inflammation |
| large_intestine_fetal-Roadmap | Immune conditions: e.g., irritable bowel disease (IBD), |
| | Crohn's disease, inflammation |
| Panc1-ENCODE | Immune conditions: e.g., irritable bowel disease (IBD), |
| | Crohn's disease, inflammation |
| adrenal_gland-ENCODE | Immune conditions: e.g., irritable bowel disease (IBD), |
| | Crohn's disease, inflammation |
| MCF10A_treated_with_TAM24hr-Ji2017 | Immune conditions: e.g., irritable bowel disease (IBD), |
| | Crohn's disease, inflammation |
| trophoblast_cell-ENCODE | Immune conditions: e.g., irritable bowel disease (IBD), |
| | Crohn's disease, inflammation |
| H1_BMP4_Derived_Tropho- | Immune conditions: e.g., irritable bowel disease (IBD), |
| blast_Cultured_Cells-Roadmap | Crohn's disease, inflammation |
| uterus-ENCODE | Immune conditions: e.g., irritable bowel disease (IBD), |
| | Crohn's disease, inflammation |
| body_of_pancreas-ENCODE | Immune conditions: e.g., irritable bowel disease (IBD), |
| | Crohn's disease, inflammation |
| stomach_fetal-Roadmap | Immune conditions: e.g., irritable bowel disease (IBD), |
| | Crohn's disease, inflammation |
| H9-Roadmap | Immune conditions: e.g., irritable bowel disease (IBD), |
| | Crohn's disease, inflammation |
| transverse_colon-ENCODE | Immune conditions: e.g., irritable bowel disease (IBD), |
| | Crohn's disease, inflammation |
| H1_BMP4_Derived_Mesendo- | Immune conditions: e.g., irritable bowel disease (IBD), |
| derm_Cultured_Cells-Roadmap | Crohn's disease, inflammation |
| breast_epithelium-ENCODE | Immune conditions: e.g., irritable bowel disease (IBD), |
| | Crohn's disease, inflammation |
| induced_pluripotent_stem_cell-ENCODE | Immune conditions: e.g., irritable bowel disease (IBD), |
| | Crohn's disease, inflammation |
| LNCAP | Immune conditions: e.g., irritable bowel disease (IBD), |
| | Crohn's disease, inflammation |
| H1_Derived_Mesenchymal_Stem_Cells-Roadmap | Immune conditions: e.g., irritable bowel disease (IBD), |
| | Crohn's disease, inflammation |
| ovary-Roadmap | Immune conditions: e.g., irritable bowel disease (IBD), |
| | Crohn's disease, inflammation |
| sigmoid_colon-ENCODE | Immune conditions: e.g., irritable bowel disease (IBD), |
| | Crohn's disease, inflammation |
| skeletal_muscle_myoblast-Roadmap | Immune conditions: e.g., irritable bowel disease (IBD), |
| | Crohn's disease, inflammation |
| bipolar_neuron_from_iPSC-ENCODE | Immune conditions: e.g., irritable bowel disease (IBD), |
| | Crohn's disease, inflammation |
| coronary_artery_smooth_muscle_cell-Miller2016 | Immune conditions: e.g., irritable bowel disease (IBD), |
| | Crohn's disease, inflammation |
| SK-N-SH-ENCODE | Immune conditions: e.g., irritable bowel disease (IBD), |
| | Crohn's disease, inflammation |
| muscle_of_trunk_fetal-Roadmap | Immune conditions: e.g., irritable bowel disease (IBD), |
| | Crohn's disease, inflammation |
| Other (23/23) | Immune conditions: e.g., irritable bowel disease (IBD), |
| | Crohn's disease, inflammation |
| adipose_tissue-ENCODE | Immune conditions: e.g., irritable bowel disease (IBD), |
| | Crohn's disease, inflammation |
| HeLa-S3-Roadmap | Immune conditions: e.g., irritable bowel disease (IBD), |
| | Crohn's disease, inflammation |
| hepatocyte-ENCODE | Immune conditions: e.g., irritable bowel disease (IBD), |
| | Crohn's disease, inflammation |
| heart_ventricle-ENCODE | Immune conditions: e.g., irritable bowel disease (IBD), |
| | Crohn's disease, inflammation |
| spinal_cord_fetal-ENCODE | Immune conditions: e.g., irritable bowel disease (IBD), |
| | Crohn's disease, inflammation |
| coronary_artery-ENCODE | Immune conditions: e.g., irritable bowel disease (IBD), |
| | Crohn's disease, inflammation |
| HepG2-Roadmap | Immune conditions: e.g., irritable bowel disease (IBD), |
| | Crohn's disease, inflammation |
| muscle_of_leg_fetal-Roadmap | Immune conditions: e.g., irritable bowel disease (IBD), |
| | Crohn's disease, inflammation |
| gastrocnemius_medialis-ENCODE | Immune conditions: e.g., irritable bowel disease (IBD), |
| | Crohn's disease, inflammation |
| brite_adipose-Loft2014 | Immune conditions: e.g., irritable bowel disease (IBD), |
| | Crohn's disease, inflammation |
| placenta-Roadmap | Immune conditions: e.g., irritable bowel disease (IBD), |
| | Crohn's disease, inflammation |
| liver-ENCODE | Immune conditions: e.g., irritable bowel disease (IBD), |
| | Crohn's disease, inflammation |
| A673-ENCODE | Immune conditions: e.g., irritable bowel disease (IBD), |
| | Crohn's disease, inflammation |
| white_adipose-Loft2014 | Immune conditions: e.g., irritable bowel disease (IBD), |
| | Crohn's disease, inflammation |
| cardiac_muscle_cell-ENCODE | Immune conditions: e.g., irritable bowel disease (IBD), |
| | Crohn's disease, inflammation |
| osteoblast-ENCODE | Immune conditions: e.g., irritable bowel disease (IBD), |
| | Crohn's disease, inflammation |
| H1_Derived_Neuronal_Progen- | Immune conditions: e.g., irritable bowel disease (IBD), |
| itor_Cultured_Cells-Roadmap | Crohn's disease, inflammation |
| endothelial_cell_of_umbilical_vein-Roadmap | Immune conditions: e.g., irritable bowel disease (IBD), |
| | Crohn's disease, inflammation |
| NCCIT | Immune conditions: e.g., irritable bowel disease (IBD), |
| | Crohn's disease, inflammation |
| myotube_originated_from_skeletal_muscle_myo- | Immune conditions: e.g., irritable bowel disease (IBD), |
| blast-Roadmap | Crohn's disease, inflammation |
| psoas_muscle-Roadmap | Immune conditions: e.g., irritable bowel disease (IBD), |
| | Crohn's disease, inflammation |
| endothelial_cell_of_umbil- | Immune conditions: e.g., irritable bowel disease (IBD), |
| ical_vein_vegf_stim_4_hours-zhang2013 | Crohn's disease, inflammation |
| endothelial_cell_of_umbil- | Immune conditions: e.g., irritable bowel disease (IBD), |
| ical_vein_vegf_stim_12_hours-zhang2013 | Crohn's disease, inflammation |

Table 6—Example variants and corresponding annotation data:

TABLE 6

| | | | | |
|---|---|---|---|---|
| #AlleleID | 15041 | 15042 | 15043 | 15044 |
| Type | Indel | Deletion | single | single |
| | | | nucleotide | nucleotide |
| | | | variant | variant |
| Name | NM_014855.3 | NM_014855.3 | NM_014630.3 | NM_017547.4 |
| | (AP5Z1): | (AP5Z1): | (ZNF592): | (FOXRED1): |
| | c.80_83delins | c.1413_ | c.3136G > A | c.694C > T |
| | TGCTGTAAACT | 1426del | (p.Gly | (p.Gln232Ter) |
| | GTAACTGTAAA | (p.Leu473fs) | 1046Arg) | |
| | (p.Arg27_ | | | |
| | Ile28delinsLeu | | | |
| | LeuTer) | | | |
| GeneID | 9907 | 9907 | 9640 | 55572 |
| GeneSymbol | AP5Z1 | AP5Z1 | ZNF592 | FOXRED1 |
| HGNC_ID | HGNC: | HGNC: | HGNC: | HGNC: |
| | 22197 | 22197 | 28986 | 26927 |
| Clinical | Pathogenic | Pathogenic | Uncertain | Pathogenic |
| Significance | | | significance | |
| ClinSigSimple | 1 | 1 | 0 | 1 |
| LastEvaluated | — | 29 Jun. 2010 | 29 Jun. 2015 | 30 Dec. 2019 |
| RS# (dbSNP) | 397704705 | 397704709 | 150829393 | 267606829 |
| nsv/esv | — | — | — | — |
| (db Var) | | | | |
| RCVaccession | RCV000000012 | RCV000000013 | RCV000000014 | RCV000000015\| |
| | | | | RCV000578659\| |
| | | | | RCV001194045 |
| PhenotypeIDS | MONDO: | MONDO: | MONDO: | MONDO: |
| | MONDO: | MONDO: | MONDO: | MONDO: |
| | 0013342, | 0013342, | 0033005, | 0032624, |
| | MedGen: | MedGen: | MedGen: | MedGen: |
| | C3150901, | C3150901, | C4551772, | C4748791, |
| | OMIM: | OMIM: | OMIM: | OMIM: |
| | 613647, | 613647, | 251300, | 618241\| |
| | Orphanet: | Orphanet: | Orphanet: | MedGen: |
| | 306511 | 306511 | 2065, | CN517202\| |
| | | | Orphanet: | MONDO: |
| | | | 83472 | MONDO: |
| | | | | 0009723, |
| | | | | MedGen: |
| | | | | C0023264, |
| | | | | OMIM: |
| | | | | 256000, |
| | | | | Orphanet: |
| | | | | 506 |
| Phenotype | Hereditary | Hereditary | Galloway- | Mitochondrial |
| List | spastic | spastic | Mowatsyndrome 1 | complex |
| | paraplegia | paraplegia | | 1deficiency, |
| | 48 | 48 | | nuclear |
| | | | | type19\| |
| | | | | not provided\| |
| | | | | Leigh |
| | | | | syndrome |
| Origin | germline; | germline | germline | germline |
| | unknown | | | |
| Origin | germline | germline | germline | germline |
| Simple | | | | |
| Assembly | GRCh37 | GRCh37 | GRCh37 | GRCh37 |
| Chromosome | NC_000007.13 | NC_000007.13 | NC_000015.9 | NC 000011.9 |
| Accession | | | | |
| Chromosome | 7 | 7 | 15 | 11 |
| Start | 4820844 | 4827361 | 85342440 | 126145284 |
| Stop | 4820847 | 4827374 | 85342440 | 126145284 |
| Reference | na | na | na | na |
| Allele | | | | |
| Alternate | na | na | na | na |
| Allele | | | | |
| Cytogenetic | 7p22.1 | 7p22.1 | 15q25.3 | 11q24.2 |
| ReviewStatus | criteria provided, | no assertion | no assertion | criteria |
| | singlesubmitter | criteria | criteria | provided, |
| | | provided | provided | multiple |
| | | | | submitters, |
| | | | | noconflicts |
| Number | 2 | 1 | 1 | 3 |
| Submitters | | | | |
| Guidelines | — | — | — | — |
| TestedInGTR | N | N | N | N |
| OtherIDs | ClinGen: | ClinGen: | OMIM: | ClinGen: |
| | CA215070, | CA215072, | 613624.0001, | CA113792, |
| | OMIM: | OMIM: | ClinGen: | OMIM: |
| | 613653.0001 | 613653.0002 | CA210674, | 613622.0001 |
| | | | UniProtKB: | |
| | | | Q92610#VAR_ | |
| | | | 064583 | |
| Submitter | 3 | 1 | 1 | 3 |
| Categories | | | | |
| VariationID | 2 | 3 | 4 | 5 |
| PositionVCF | 4820844 | 4827360 | 85342440 | 126145284 |
| Reference | GGAT | GCTGCTGG | G | C |
| Allele | | ACCTGCC | | |
| VCF | | | | |
| Alternate | TGCTGTA | G | A | T |
| Allele VCF | AACTGTAA | | | |
| | CTGTAAA | | | |
| rs | rs397704705 | rs397704709 | rs150829393 | rs267606829 |

Table 7—Example of Annotation data comprising a genotype and its corresponding information.

TABLE 7

| Clinical | | | |
|---|---|---|---|
| Annotation | Genotype/ | | Allele |
| ID | Allele | Annotation Text | Function |
| 981755803 | AA | Patients with the rs75527207 AA genotype (two copies of the CFTR G551D variant) and cystic | |
| | | fibrosis may respond to ivacaftor treatment. FDA-approved drug labeling information and CPIC | |
| | | guidelines indicate use of ivacaftor in cystic fibrosis patients with at least one copy of a list of 33 | |
| | | CFTR genetic variants, including G551D. Other genetic and clinical factors may also influence | |
| | | response to ivacaftor. | |
| 981755803 | AG | Patients with the rs75527207 AG genotype (one copy of the CFTR G551D variant) and cystic | |
| | | fibrosis may respond to ivacaftor treatment. FDA-approved drug labeling information and CPIC | |
| | | guidelines indicate use of ivacaftor in cystic fibrosis patients with at least one copy of a list of 33 | |
| | | CFTR genetic variants, including G551D. Other genetic and clinical factors may also influence | |
| | | response to ivacaftor. | |
| 981755803 | GG | Patients with the rs75527207 GG genotype (do not have a copy of the CFTR G551D variant) | |
| | | and cystic fibrosis have an unknown response to ivacaftor treatment, as response may depend on | |
| | | the presence of other CFTR variants. FDA-approved drug labeling information and CPIC | |
| | | guidelines indicate use of ivacaftor in cystic fibrosis patients with at least one copy of a list of 33 | |
| | | CFTR genetic variants, including G551D. Other genetic and clinical factors may also influence | |
| | | response to ivacaftor. | |
| 1449311190 | CC | Patients with the CC genotype and Precursor Cell Lymphoblastic Leukemia-Lymphoma may | |
| | | need a decreased dose of mercaptopurine, or methotrexate, as compared to children with the TT | |
| | | genotype. Other clinical and genetic factors may also influence dose of mercaptopurine or | |
| | | methotrexate in children with Precursor Cell Lymphoblastic Leukemia-Lymphoma. | |
| 1449311190 | CT | Patients with the CT genotype and Precursor Cell Lymphoblastic Leukemia-Lymphoma may | |
| | | need a decreased dose of mercaptopurine, or methotrexate, as compared to children with the TT | |
| | | genotype. Other clinical and genetic factors may also influence dose of mercaptopurine or | |
| | | methotrexate in children with Precursor Cell Lymphoblastic Leukemia-Lymphoma. | |
| 1449311190 | TT | Patients with the TT genotype and Precursor Cell Lymphoblastic Leukemia-Lymphoma may | |
| | | need an increased dose of mercaptopurine, or methotrexate, as compared to children with the CC | |
| | | or CT genotypes. Other clinical and genetic factors may also influence dose of mercaptopurine | |
| | | or methotrexate in children with Precursor Cell Lymphoblastic Leukemia-Lymphoma. | |
| 981204774 | AA | Patients with AA genotype may have an increased likelihood of smoking cessation when treated | |
| | | with nicotine replacement therapy (transdermal nicotine patch) as compared to patients with the | |
| | | AG and GG genotype. However, contradictory findings reporting the opposite association for | |
| | | this genotype with decreased likelihood of smoking cessation have been published. Other | |
| | | genetic and clinical factors may influence a patient's likelihood of smoking cessation. | |
| 981204774 | AG | Patients with AG genotype may have a decreased likelihood of smoking cessation when treated | |
| | | with nicotine replacement therapy (transdermal nicotine patch) as compared to patients with the | |
| | | AA genotype. However, contradictory findings reporting the opposite association for this | |
| | | genotype with increased likelihood of smoking cessation have been published. Other genetic and | |
| | | clinical factors may influence a patient's likelihood of smoking cessation. | |
| 981204774 | GG | Patients with GG genotype may have a decreased likelihood of smoking cessation when treated | |
| | | with nicotine replacement therapy (transdermal nicotine patch) as compared to patients with the | |
| | | AA genotype. However, contradictory findings reporting the opposite association for this | |
| | | genotype with increased likelihood of smoking cessation have been published. Other genetic and | |
| | | clinical factors may influence a patient's likelihood of smoking cessation. Other genetic and | |
| | | clinical factors may influence a patient's likelihood of smoking cessation. | |
| 1449191690 | CC | Patients with the CC genotype (do not have a copy of the CFTR S977F variant) and cystic | |
| | | fibrosis have an unknown response to ivacaftor treatment, as response may depend on the | |
| | | presence of other CFTR variants. FDA-approved drug labeling information and CPIC guidelines | |
| | | indicate use of ivacaftor in cystic fibrosis patients with at least one copy of a list of 33 CFTR | |
| | | genetic variants, including S977F. Other genetic and clinical factors may also influence response | |
| | | to ivacaftor. | |
| 1449191690 | CT | Patients with the CT genotype (one copy of the CFTR S977F variant) and cystic fibrosis may | |
| | | respond to ivacaftor treatment. FDA-approved drug labeling information and CPIC guidelines | |
| | | indicate use of ivacaftor in cystic fibrosis patients with at least one copy of a list of 33 CFTR | |
| | | genetic variants, including S977F. Other genetic and clinical factors may also influence response | |
| | | to ivacaftor. | |
| 1449191690 | TT | Patients with the TT genotype (two copies of the CFTR S977F variant) and cystic fibrosis may | |
| | | respond to ivacaftor treatment. FDA-approved drug labeling information and CPIC guidelines | |
| | | indicate use of ivacaftor in cystic fibrosis patients with at least one copy of a list of 33 CFTR | |
| | | genetic variants, including S977F. Other genetic and clinical factors may also influence response | |
| | | to ivacaftor. | |
| 1449191746 | AA | Patients with the AA genotype (two copies of the CFTR R1070Q variant) and cystic fibrosis | |
| | | may respond to ivacaftor treatment. FDA-approved drug labeling information and CPIC | |
| | | guidelines indicate use of ivacaftor in cystic fibrosis patients with at least one copy of a list of 33 | |
| | | CFTR genetic variants, including R1070Q. Other genetic and clinical factors may also influence | |
| | | response to ivacaftor. | |
| 1449191746 | AG | Patients with the AG genotype (one copy of the CFTR R1070Q variant) and cystic fibrosis may | |
| | | respond to ivacaftor treatment. FDA-approved drug labeling information and CPIC guidelines | |
| | | indicate use of ivacaftor in cystic fibrosis patients with at least one copy of a list of 33 CFTR | |
| | | genetic variants, including R1070Q. Other genetic and clinical factors may also influence | |
| | | response to ivacaftor. | |
| 1449191746 | GG | Patients with the GG genotype (do not have a copy of the CFTR R1070Q variant) and cystic | |
| | | fibrosis have an unknown response to ivacaftor treatment, as response may depend on the | |
| | | presence of other CFTR variants. FDA-approved drug labeling information and CPIC guidelines | |
| | | indicate use of ivacaftor in cystic fibrosis patients with at least one copy of a list of 33 CFTR | |
| | | genetic variants, including R1070Q. Other genetic and clinical factors may also influence | |
| | | response to ivacaftor. | |
| 981419532 | CC | Patients with the CC genotype may have decreased risk of statin-related muscle symptoms as | |
| | | compared to patients with genotype GG or CG. Other genetic and clinical factors may also | |
| | | influence a patient's risk of toxicity. | |
| 981419532 | CG | Patients with the CG genotype may have increased risk of statin-related muscle symptoms as | |
| | | compared to patients with genotype CC. Other genetic and clinical factors may also influence a | |
| | | patient's risk of toxicity. | |
| 981419532 | GG | Patients with the GG genotype may have increased risk of statin-related muscle symptoms as | |
| | | compared to patients with genotype CC. Other genetic and clinical factors may also influence a | |
| | | patient's risk of toxicity. | |
| 1449566379 | AA | Patients with the AA genotype and hypertension may have an increased response to | |
| | | hydrochlorothiazide treatment, as measured by decreases in systolic and diastolic blood pressure, | |
| | | as compared to patients with the AG or GG genotypes. Other genetic and clinical factors may | |
| | | also affect a patient's response to hydrochlorothiazide. | |
| 1449566379 | AG | Patients with the AG genotype and hypertension may have an increased response to | |
| | | hydrochlorothiazide treatment, as measured by decreases in systolic and diastolic blood pressure, | |
| | | as compared to patients with the GG genotype, but a decreased response as compared to patients | |
| | | with the AA genotype. Other genetic and clinical factors may also affect a patient's response to | |
| | | hydrochlorothiazide. | |
| 1449566379 | GG | Patients with the GG genotype and hypertension may have a decreased response to | |
| | | hydrochlorothiazide treatment, as measured by decreases in systolic and diastolic blood pressure, | |
| | | as compared to patients with the AA or AG genotypes. Other genetic and clinical factors may | |
| | | also affect a patient's response to hydrochlorothiazide. | |
| 981419266 | *15:02:01 | Patients with one or two copies of the HLA-B*15:02:01 allele may have an increased risk of | Presence |
| | | Severe Cutaneous Adverse Reactions, such as Stevens-Johnson Syndrome and Toxic Epidermal | |
| | | Necrolysis, when treated with phenytoin as compared to patients with no HLA-B*15:02:01 | |
| | | alleles or negative for the HLA-B*15:02:01 test. However, conflicting evidence has been | |
| | | reported. Other genetic and clinical factors may also influence risk of phenytoin-induced adverse | |
| | | reactions. | |
| 1451259580 | *1 | The CYP2D6*1 allele is assigned as a normal function allele by CPIC. Patients carrying the | Normal |
| | | CYP2D6*1 allele in combination with alleles that result in a normal metabolizer phenotype who | function |
| | | are treated with amitriptyline may have decreased likelihood of side effects as compared to | |
| | | patients with a combination of alleles that result in intermediate or poor metabolizer phenotype. | |
| | | Other genetic and clinical factors may also influence response to amitriptyline. | |
| 1451259580 | *1 × N | The CYP2D6*1 × N alleles (*1 × 2 and *1 × ≥3) have been assigned as | Increased |
| | | increased function alleles by CPIC. Patients carrying the CYP2D6*1 × N | function |
| | | allele in combination with alleles that result in a normal | |
| | | metabolizer phenotype who are treated with amitriptyline may have | |
| | | decreased likelihood of side effects as compared to patients with a | |
| | | combination of alleles that result in intermediate or poor | |
| | | metabolizer phenotype. Other genetic and clinical factors may also | |
| | | influence response to amitriptyline. | |
| 1451259580 | *2 | The CYP2D6*2 allele is assigned as a normal function allele by CPIC. Patients carrying the | Normal |
| | | CYP2D6*2 allele in combination with alleles that result in a normal metabolizer phenotype who | function |
| | | are treated with amitriptyline may have decreased likelihood of side effects as compared to | |
| | | patients with a combination of alleles that result in intermediate or poor metabolizer phenotype. | |
| | | Other genetic and clinical factors may also influence response to amitriptyline. | |
| 1451259580 | *3 | The CYP2D6*3 allele is assigned as a no function allele by CPIC. Patients carrying the | No |
| | | CYP2D6*3 allele in combination with with alleles that result in intermediate or poor metabolizer | function |
| | | phenotype who are treated with amitriptyline may have increased likelihood of side effects as | |
| | | compared to patients with alleles that result in a normal metabolizer phenotype. Other genetic | |
| | | and clinical factors may also influence response to amitriptyline. | |
| 1451259580 | *4 | The CYP2D6*4 allele is assigned as a no function allele by CPIC. Patients carrying the | No |
| | | CYP2D6*4 allele in combination with with alleles that result in intermediate or poor metabolizer | function |
| | | phenotype who are treated with amitriptyline may have increased likelihood of side effects as | |
| | | compared to patients with alleles that result in a normal metabolizer phenotype. Other genetic | |
| | | and clinical factors may also influence response to amitriptyline. | |
| 1451259580 | *5 | The CYP2D6*5 allele is assigned as a no function allele by CPIC. Patients carrying the | No |
| | | CYP2D6*5 allele in combination with with alleles that result in intermediate or poor metabolizer | function |
| | | phenotype who are treated with amitriptyline may have increased likelihood of side effects as | |
| | | compared to patients with alleles that result in a normal metabolizer phenotype. Other genetic | |
| | | and clinical factors may also influence response to amitriptyline. | |
| 1451259580 | *6 | The CYP2D6*6 allele is assigned as a no function allele by CPIC. Patients carrying the | No |
| | | CYP2D6*6 allele in combination with with alleles that result in intermediate or poor metabolizer | function |
| | | phenotype who are treated with amitriptyline may have increased likelihood of side effects as | |
| | | compared to patients with alleles that result in a normal metabolizer phenotype. Other genetic | |
| | | and clinical factors may also influence response to amitriptyline. | |
| 1451259580 | *10 | The CYP2D6*10 allele is assigned as a decreased function allele with an activity value of 0.25 | Decreased |
| | | by CPIC. Patients carrying the CYP2D6*10 allele in combination with with alleles that result in | function |
| | | intermediate or poor metabolizer phenotype who are treated with amitriptyline may have | (AV 0.25) |
| | | increased likelihood of side effects as compared to patients with alleles that result in a normal | |
| | | metabolizer phenotype. Other genetic and clinical factors may also influence response to | |
| | | amitriptyline. | |
| 1451259580 | *41 | The CYP2D6*41 allele is assigned as a decreased function allele with an activity value of 0.5 by | Decreased |
| | | CPIC. Patients carrying the CYP2D6*41 allele in combination with alleles that result in | function |
| | | intermediate or poor metabolizer phenotype who are treated with amitriptyline may have | |
| | | increased likelihood of side effects as compared to patients with alleles that result in a normal | |
| | | metabolizer phenotype. Other genetic and clinical factors may also influence response to | |
| | | amitriptyline. | |
| 1451265560 | *1 | The CYP2D6*1 allele is assigned as a normal function allele by CPIC. Patients carrying the *1 | Normal |
| | | allele in combination with alleles that result in a normal metabolizer phenotype may have | function |
| | | decreased imipramine dose requirements as compared to patients carrying two increased | |
| | | function alleles or an increased function allele in combination with a normal function allele or a | |
| | | decreased function allele with an activity value of 0.5. Patients carrying the *1 allele in | |
| | | combination with alleles that result in a normal metabolizer phenotype may also have decreased | |
| | | imipramine dose requirements as compared to patients carrying an increased function allele with | |
| | | an activity value of 3 or greater in combination with a no function allele or a decreased function | |
| | | allele with an activity value of 0.25 but increased imipramine dose requirements as compared to | |
| | | patients with a no function allele in combination with a decreased or normal function allele or | |
| | | two decreased or no function alleles. Other genetic and clinical factors may also influence | |
| | | imipramine dose requirements. | |
| 1451265560 | *1 × N | The CYP2D6*1 × N alleles (*1 × 2 and *1 × ≥3) have been assigned as | Increased |
| | | increased function alleles by CPIC. Patients carrying a *1 × N allele in | function |
| | | combination with a normal or increased function allele or a | |
| | | decreased function allele with an activity value of 0.5 may have | |
| | | increased imipramine dose requirements as compared to patients | |
| | | with alleles that result in a normal metabolizer phenotype. Patients | |
| | | carrying a *1 × N allele with an activity value of 3 or greater in | |
| | | combination with a decreased function allele with an activity value | |
| | | of 0.25 or a no function allele may also have increased imipramine | |
| | | dose requirements as compared to patients with alleles that result | |
| | | in a normal metabolizer phenotype, while patients carrying a *1 × N | |
| | | allele with an activity value of 2 in combination with a decreased | |
| | | function allele with an activity value of 0.25 or a no function allele | |
| | | may have similar imipramine dose requirements as compared to | |
| | | patients with other alleles that result in a normal metabolizer | |
| | | phenotype. Other genetic and clinical factors may also influence | |
| | | imipramine dose requirements. | |
| 1451265560 | *2 × N | The CYP2D6*2 × N alleles (*2 × 2 and *2 × ≥3) have been assigned as | Increased |
| | | increased function alleles by CPIC. Patients carrying a *2 × N allele in | function |
| | | combination with a normal or increased function allele or a | |
| | | decreased function allele with an activity value of 0.5 may have | |
| | | increased imipramine dose requirements as compared to patients | |
| | | with alleles that result in a normal metabolizer phenotype. Patients | |
| | | carrying a *2 × N allele with an activity value of 3 or greater in | |
| | | combination with a decreased function allele with an activity value | |
| | | of 0.25 or a no function allele may also have increased imipramine | |
| | | dose requirements as compared to patients with alleles that result | |
| | | in a normal metabolizer phenotype, while patients carrying a *2 × N | |
| | | allele with an activity value of 2 in combination with a decreased | |
| | | function allele with an activity value of 0.25 or a no function allele | |
| | | may have similar imipramine dose requirements as compared to | |
| | | patients with other alleles that result in a normal metabolizer | |
| | | phenotype. Other genetic and clinical factors may also influence | |
| | | imipramine dose requirements. | |
| 1451265560 | *4 | The CYP2D6*4 allele is assigned as a no function allele by CPIC. Patients carrying the *4 allele | No |
| | | in combination with a normal, decreased or no function allele may have decreased imipramine | function |
| | | dose requirements as compared to patients with alleles that result in a normal metabolizer | |
| | | phenotype, while patients carrying the *4 allele in combination with an increased function allele | |
| | | with an activity value of 3 or greater may have increased imipramine dose requirements as | |
| | | compared to patients with alleles that result in a normal metabolizer phenotype. Patients carrying | |
| | | the *4 allele in combination with an increased function allele with an activity score of 2 may | |
| | | have similar imipramine dose requirements as compared to patients with alleles that result in a | |
| | | normal metabolizer phenotype. Other genetic and clinical factors may also influence imipramine | |
| | | dose requirements. | |
| 1451265560 | *5 | The CYP2D6*5 allele is assigned as a no function allele by CPIC. Patients carrying the *5 allele | No |
| | | in combination with a normal, decreased or no function allele may have decreased imipramine | function |
| | | dose requirements as compared to patients with alleles that result in a normal metabolizer | |
| | | phenotype, while patients carrying the *5 allele in combination with an increased function allele | |
| | | with an activity value of 3 or greater may have increased imipramine dose requirements as | |
| | | compared to patients with alleles that result in a normal metabolizer phenotype. Patients carrying | |
| | | the *5 allele in combination with an increased function allele with an activity score of 2 may | |
| | | have similar imipramine dose requirements as compared to patients with alleles that result in a | |
| | | normal metabolizer phenotype. Other genetic and clinical factors may also influence imipramine | |
| | | dose requirements. | |
| 1451288200 | *1 | The CYP2D6*1 allele is assigned as a normal function allele by CPIC. Patients carrying the *1 | Normal |
| | | allele in combination with alleles that result in a normal metabolizer phenotype may have | function |
| | | decreased risk of toxicity when treated with codeine as compared to patients carrying two | |
| | | increased function alleles or an increased function allele in combination with a normal function | |
| | | allele or a decreased function allele with an activity value of 0.5. Patients carrying the *1 allele | |
| | | in combination with alleles that result in a normal metabolizer phenotype may also have | |
| | | decreased risk of toxicity when treated with codeine as compared to patients carrying an | |
| | | increased function allele with an activity value of 3 or greater in combination with a no function | |
| | | allele or a decreased function allele with an activity value of 0.25 but a similar risk of toxicity | |
| | | when treated with codeine as compared to patients with a no function allele in combination with | |
| | | a decreased or normal function allele or two decreased or no function alleles. Other genetic and | |
| | | clinical factors may also influence risk of codeine toxicity. | |
| 1451288200 | *1 × N | The CYP2D6*1 × N alleles (*1 × 2 and *1 × ≥3) are assigned as increased | Increased |
| | | function alleles by CPIC. Patients carrying a *1 × N allele with an | function |
| | | activity value of 3 or greater in combination with a normal, | |
| | | increased, decreased or no function allele may have an increased risk | |
| | | of toxicity when treated with codeine as compared to patients with | |
| | | alleles that result in a normal metabolizer phenotype. Patients | |
| | | carrying a *1 × N allele with an activity value of 2 in combination with | |
| | | an increased or normal function allele or a decreased function allele | |
| | | with an activity value of 0.5 may also have an increased risk of | |
| | | toxicity when treated with codeine as compared to patients with | |
| | | alleles that result in a normal metabolizer phenotype, while patients | |
| | | carrying a *1 × N allele with an activity value of 2 in combination with | |
| | | a no function allele or a decreased function allele with an activity | |
| | | value of 0.25 may have a similar risk of toxicity when treated with | |
| | | codeine as compared to patients with alleles that result in a normal | |
| | | metabolizer phenotype. Other genetic and clinical factors may also | |
| | | influence risk of codeine toxicity. | |
| 1451288200 | *2 | The CYP2D6*2 allele is assigned as a normal function allele by CPIC. Patients carrying the *2 | Normal |
| | | allele in combination with alleles that result in a normal metabolizer phenotype may have | function |
| | | decreased risk of toxicity when treated with codeine as compared to patients carrying two | |
| | | increased function alleles or an increased function allele in combination with a normal function | |
| | | allele or a decreased function allele with an activity value of 0.5. Patients carrying the *2 allele | |
| | | in combination with alleles that result in a normal metabolizer phenotype may also have | |
| | | decreased risk of toxicity when treated with codeine as compared to patients carrying an | |
| | | increased function allele with an activity value of 3 or greater in combination with a no function | |
| | | allele or a decreased function allele with an activity value of 0.25 but a similar risk of toxicity | |
| | | when treated with codeine as compared to patients with a no function allele in combination with | |
| | | a decreased or normal function allele or two decreased or no function alleles. Other genetic and | |
| | | clinical factors may also influence risk of codeine toxicity. | |
| 1451288200 | *2 × N | The CYP2D6*2 × N alleles (*2 × 2 and *2 × ≥3) are assigned as increased | |
| | | function alleles by CPIC. Patients carrying a *2 × N allele with an | |
| | | activity value of 3 or greater in combination with a normal, | |
| | | increased, decreased or no function allele may have an increased risk | Increased |
| | | of toxicity when treated with codeine as compared to patients with | function |
| | | alleles that result in a normal metabolizer phenotype. Patients | |
| | | carrying a *2 × N allele with an activity value of 2 in combination with | |
| | | an increased or normal function allele or a decreased function allele | |
| | | with an activity value of 0.5 may also have an increased risk of | |
| | | toxicity when treated with codeine as compared to patients with | |
| | | alleles that result in a normal metabolizer phenotype, while patients | |
| | | carrying a *2 × N allele with an activity value of 2 in combination with | |
| | | a no function allele or a decreased function allele with an activity | |
| | | value of 0.25 may have a similar risk of toxicity when treated with | |
| | | codeine as compared to patients with alleles that result in a normal | |
| | | metabolizer phenotype. Other genetic and clinical factors may also | |
| | | influence risk of codeine toxicity. | |
| 1451282240 | *1 | The CYP2D6*1 allele is assigned as a normal function allele by CPIC. Patients carrying the *1 | Normal |
| | | allele in combination with alleles that result in a normal metabolizer phenotype who are treated | function |
| | | with atomoxetine may have a decreased, but not absent, risk for treatment related side effects as | |
| | | compared to patients with two no function alleles. Other genetic and clinical factors may also | |
| | | influence response to atomoxetine. | |
| 1451282240 | *3 | The CYP2D6*3 allele is assigned as a no function allele by CPIC. Patients carrying the *3 allele | No |
| | | in combination with another no function allele who are treated with atomoxetine may have an | function |
| | | increased risk for treatment related side effects as compared to patients with alleles that result in | |
| | | a normal metabolizer phenotype. Other genetic and clinical factors may also influence response | |
| | | to atomoxetine. | |
| 1451282240 | *4 | The CYP2D6*4 allele is assigned as a no function allele by CPIC. Patients carrying the *4 allele | No |
| | | in combination with another no function allele who are treated with atomoxetine may have an | function |
| | | increased risk for treatment related side effects as compared to patients with alleles that result in | |
| | | a normal metabolizer phenotype. Other genetic and clinical factors may also influence response | |
| | | to atomoxetine. | |
| 1451282240 | *4 × N | The CYP2D6*4 × N allele (*4 × 2) is assigned as a no function allele by CPIC. Patients carrying | No |
| | | the *4 × N allele in combination with another no function allele who are treated with atomoxetine | function |
| | | may have an increased risk for treatment related side effects as compared to patients with alleles | |
| | | that result in a normal metabolizer phenotype. Other genetic and clinical factors may also | |
| | | influence response to atomoxetine. | |
| 1451282240 | *5 | The CYP2D\hat{ }*5 allele is assigned as a no function allele by CPIC. Patients carrying the *5 allele | No |
| | | in combination with another no function allele who are treated with atomoxetine may have an | function |
| | | increased risk for treatment related side effects as compared to patients with alleles that result in | |
| | | a normal metabolizer phenotype. Other genetic and clinical factors may also influence response | |
| | | to atomoxetine. | |
| 1451282240 | *6 | The CYP2D6*6 allele is assigned as a no function allele by CPIC. Patients carrying the *6 allele | No |
| | | in combination with another no function allele who are treated with atomoxetine may have an | function |
| | | increased risk for treatment related side effects as compared to patients with with alleles that | |
| | | result in a normal metabolizer phenotype. Other genetic and clinical factors may also influence | |
| | | response to atomoxetine. | |
| 1451285240 | *1 | The CYP2D6*1 allele is assigned as a normal function allele by CPIC. Patients with breast | Normal |
| | | cancer and carrying the CYP2D6*1 allele in combination with alleles that result in a normal | function |
| | | metabolizer phenotype may have decreased likelihood of recurrence and increased event-free | |
| | | and recurrence-free survival when treated with tamoxifen in an adjuvant setting as compared to | |
| | | patients with a no function allele in combination with a decreased or normal function allele or | |
| | | two no or decreased function alleles. However, conflicting evidence has been reported. Be aware | |
| | | that the DPWG guideline for tamoxifen and CYP2D6 has a ‘no recommendation’ for CYP2D6 | |
| | | ultrarapid metabolizers. Other genetic and clinical factors may also influence response to | |
| | | tamoxifen treatment. | |
| 1451285240 | *2 | The CYP2D6*2 allele is assigned as a normal function allele by CPIC. Patients with breast | Normal |
| | | cancer and carrying the CYP2D6*2 allele in combination with alleles that result in a normal | function |
| | | metabolizer phenotype may have decreased likelihood of recurrence and increased event-free | |
| | | and recurrence-free survival when treated with tamoxifen in an adjuvant setting as compared to | |
| | | patients with a no function allele in combination with a decreased or normal function allele or | |
| | | two no or decreased function alleles. However, conflicting evidence has been reported. Be aware | |
| | | that the DPWG guideline for tamoxifen and CYP2D6 has a ‘no recommendation’ for CYP2D6 | |
| | | ultrarapid metabolizers. Other genetic and clinical factors may also influence response to | |
| | | tamoxifen treatment. | |
| 1451285240 | *3 | The CYP2D6*3 allele is assigned as a no function allele by CPIC. Patients with breast cancer | No |
| | | and carrying the CYP2D6*3 allele in combination with a no, decreased or normal function allele | function |
| | | may have increased likelihood of recurrence and lower event-free and recurrence-free survival | |
| | | when treated with tamoxifen in an adjuvant setting as compared to patients with alleles that | |
| | | result in a normal metabolizer phenotype. However, conflicting evidence has been reported. Be | |
| | | aware that the DPWG guideline for tamoxifen and CYP2D6 has a ‘no recommendation’ for | |
| | | CYP2D6 ultrarapid metabolizers. Other genetic and clinical factors may also influence response | |
| | | to tamoxifen treatment. | |
| 1451285240 | *4 | The CYP2D6*4 allele is assigned as a no function allele by CPIC. Patients with breast cancer | No |
| | | and carrying the CYP2D6*4 allele in combination with a no, decreased or normal function allele | function |
| | | may have increased likelihood of recurrence and lower event-free and recurrence-free survival | |
| | | when treated with tamoxifen in an adjuvant setting as compared to patients with alleles that | |
| | | result in a normal metabolizer phenotype. However, conflicting evidence has been reported. Be | |
| | | aware that the DPWG guideline for tamoxifen and CYP2D6 has a 'no recommendation' for | |
| | | CYP2D6 ultrarapid metabolizers. Other genetic and clinical factors may also influence response | |
| | | to tamoxifen treatment. | |
| 1451285240 | *5 | The CYP2D6*5 allele is assigned as a no function allele by CPIC. Patients with breast cancer | No |
| | | and carrying the CYP2D6*5 allele in combination with a no, decreased or normal function allele | function |
| | | may have increased likelihood of recurrence and lower event-free and recurrence-free survival | |
| | | when treated with tamoxifen in an adjuvant setting as compared to patients with alleles that | |
| | | result in a normal metabolizer phenotype. However, conflicting evidence has been reported. Be | |
| | | aware that the DPWG guideline for tamoxifen and CYP2D6 has a ‘no recommendation’ for | |
| | | CYP2D6 ultrarapid metabolizers. Other genetic and clinical factors may also influence response | |
| | | to tamoxifen treatment. | |
| 1451285240 | *6 | The CYP2D6*6 allele is assigned as a no function allele by CPIC. Patients with breast cancer | No |
| | | and carrying the CYP2D6*6 allele in combination with a no, decreased or normal function allele | function |
| | | may have increased likelihood of recurrence and lower event-free and recurrence-free survival | |
| | | when treated with tamoxifen in an adjuvant setting as compared to patients with alleles that | |
| | | result in a normal metabolizer phenotype. However, conflicting evidence has been reported. Be | |
| | | aware that the DPWG guideline for tamoxifen and CYP2D6 has a ‘no recommendation’ for | |
| | | CYP2D6 ultrarapid metabolizers. Other genetic and clinical factors may also influence response | |
| | | to tamoxifen treatment. | |
| 1451285240 | *7 | The CYP2D6*7 allele is assigned as a no function allele by CPIC. Patients with breast cancer | No |
| | | and carrying the CYP2D6*7 allele in combination with a no, decreased or normal function allele | function |
| | | may have increased likelihood of recurrence and lower event-free and recurrence-free survival | |
| | | when treated with tamoxifen in an adjuvant setting as compared to patients with alleles that | |
| | | result in a normal metabolizer phenotype. However, conflicting evidence has been reported. Be | |
| | | aware that the DPWG guideline for tamoxifen and CYP2D6 has a ‘no recommendation’ for | |
| | | CYP2D6 ultrarapid metabolizers. Other genetic and clinical factors may also influence response | |
| | | to tamoxifen treatment. | |
| 1451285240 | *9 | The CYP2D6*9 allele is assigned as a decreased function allele with an activity value of 0.5 by | Decreased |
| | | CPIC. Patients with breast cancer and carrying the CYP2D6*9 allele in combination with a no or | function |
| | | decreased function allele may have increased likelihood of recurrence and lower event-free and | |
| | | recurrence-free survival when treated with tamoxifen in an adjuvant setting as compared to | |
| | | patients with alleles that result in a normal metabolizer phenotype. However, conflicting | |
| | | evidence has been reported. Be aware that the DPWG guideline for tamoxifen and CYP2D6 has | |
| | | a ‘no recommendation’ for CYP2D6 ultrarapid metabolizers. Other genetic and clinical factors | |
| | | may also influence response to tamoxifen treatment. | |
| 1451285240 | *10 | The CYP2D6*10 allele is assigned as a decreased function allele with an activity value of 0.25 | Decreased |
| | | by CPIC. Patients with breast cancer and carrying the CYP2D6*10 allele in combination with a | function |
| | | no or decreased function allele may have increased likelihood of recurrence and lower event-free | (AV 0.25) |
| | | and recurrence-free survival when treated with tamoxifen in an adjuvant setting as compared to | |
| | | patients with alleles that result in a normal metabolizer phenotype. However, conflicting | |
| | | evidence has been reported. Be aware that the DPWG guideline for tamoxifen and CYP2D6 has | |
| | | a ‘no recommendation’ for CYP2D6 ultrarapid metabolizers. Other genetic and clinical factors | |
| | | may also influence response to tamoxifen treatment. | |
| 1451285240 | *10 × 2 | The CYP2D6*10 × 2 allele is assigned as a decreased function allele with an activity value of 0.5 | Decreased |
| | | by CPIC. Patients with breast cancer and carrying the CYP2D6*10 × 2 allele in combination with | function |
| | | a no or decreased function allele may have increased likelihood of recurrence and lower event- | |
| | | free and recurrence-free survival when treated with tamoxifen in an adjuvant setting as | |
| | | compared to patients with alleles that result in a normal metabolizer phenotype. However, | |
| | | conflicting evidence has been reported. Be aware that the DPWG guideline for tamoxifen and | |
| | | CYP2D6 has a ‘no recommendation’ for CYP2D6 ultrarapid metabolizers. Other genetic and | |
| | | clinical factors may also influence response to tamoxifen treatment. | |
| 1451285240 | *11 | The CYP2D6*11 allele is assigned as a no function allele by CPIC. Patients with breast cancer | No |
| | | and carrying the CYP2D6*11 allele in combination with a no, decreased or normal function | function |
| | | allele may have increased likelihood of recurrence and lower event-free and recurrence-free | |
| | | survival when treated with tamoxifen in an adjuvant setting as compared to patients with alleles | |
| | | that result in a normal metabolizer phenotype. However, conflicting evidence has been reported. | |
| | | Be aware that the DPWG guideline for tamoxifen and CYP2D6 has a ‘no recommendation’ for | |
| | | CYP2D6 ultrarapid metabolizers. Other genetic and clinical factors may also influence response | |
| | | to tamoxifen treatment. | |
| 1451285240 | *17 | The CYP2D6*17 allele is assigned as a decreased function allele with an activity value of 0.5 by | Decreased |
| | | CPIC. Patients with breast cancer and carrying the CYP2D6*17 allele in combination with a no | function |
| | | or decreased function allele may have increased likelihood of recurrence and lower event-free | |
| | | and recurrence-free survival when treated with tamoxifen in an adjuvant setting as compared to | |
| | | patients with alleles that result in a normal metabolizer phenotype. However, conflicting | |
| | | evidence has been reported. Be aware that the DPWG guideline for tamoxifen and CYP2D6 has | |
| | | a ‘no recommendation’ for CYP2D6 ultrarapid metabolizers. Other genetic and clinical factors | |
| | | may also influence response to tamoxifen treatment. | |
| 1451285240 | *21 | The CYP2D6*21 allele is assigned as a no function allele by CPIC. Patients with breast cancer | No |
| | | and carrying the CYP2D6*21 allele in combination with a no, decreased or normal function | function |
| | | allele may have increased likelihood of recurrence and lower event-free and recurrence-free | |
| | | survival when treated with tamoxifen in an adjuvant setting as compared to patients with alleles | |
| | | that result in a normal metabolizer phenotype. However, conflicting evidence has been reported. | |
| | | Be aware that the DPWG guideline for tamoxifen and CYP2D6 has a ‘no recommendation’ for | |
| | | CYP2D6 ultrarapid metabolizers. Other genetic and clinical factors may also influence response | |
| | | to tamoxifen treatment. | |
| 1451285240 | *36 | The CYP2D6*36 allele is assigned as a no function allele by CPIC. Patients with breast cancer | No |
| | | and carrying the CYP2D6*36 allele in combination with a no, decreased or normal function | function |
| | | allele may have increased likelihood of recurrence and lower event-free and recurrence-free | |
| | | survival when treated with tamoxifen in an adjuvant setting as compared to patients with alleles | |
| | | that result in a normal metabolizer phenotype. However, conflicting evidence has been reported. | |
| | | Be aware that the DPWG guideline for tamoxifen and CYP2D6 has a ‘no recommendation’ for | |
| | | CYP2D6 ultrarapid metabolizers. Other genetic and clinical factors may also influence response | |
| | | to tamoxifen treatment. | |
| 1451285240 | *41 | The CYP2D6*41 allele is assigned as a decreased function allele with an activity value of 0.5 by | Decreased |
| | | CPIC. Patients with breast cancer and carrying the CYP2D6*41 allele in combination with a no | function |
| | | or decreased function allele may have increased likelihood of recurrence and lower event-free | |
| | | and recurrence-free survival when treated with tamoxifen in an adjuvant setting as compared to | |
| | | patients with alleles that result in a normal metabolizer phenotype. However, conflicting | |
| | | evidence has been reported. Be aware that the DPWG guideline for tamoxifen and CYP2D6 has | |
| | | a ‘no recommendation’ for CYP2D6 ultrarapid metabolizers. Other genetic and clinical factors | |
| | | may also influence response to tamoxifen treatment. | |
| 1451226160 | AA | Infants who have been exposed to methadone in utero and who have the rs4680 AA genotype | |
| | | may have an increased severity of neonatal abstinence syndrome as compared to infants with the | |
| | | AG or GG genotypes. However, conflicting evidence has been reported. Other genetic and | |
| | | clinical factors may also affect severity of neonatal abstinence syndrome. | |
| 1451226160 | AG | Infants who have been exposed to methadone in utero and who have the rs4680 AG genotype | |
| | | may have a decreased severity of neonatal abstinence syndrome as compared to infants with the | |
| | | AA genotype. However, conflicting evidence has been reported. Other genetic and clinical | |
| | | factors may also affect severity of neonatal abstinence syndrome. | |
| 1451226160 | GG | Infants who have been exposed to methadone in utero and who have the rs4680 GG genotype | |
| | | may have a decreased severity of neonatal abstinence syndrome as compared to infants with the | |
| | | AA genotype. However, conflicting evidence has been reported. Other genetic and clinical | |
| | | factors may also affect severity of neonatal abstinence syndrome. | |
| 1451226166 | AA | Infants who have been exposed to methadone in utero and who are born to women with the AA | |
| | | genotype may be more likely to require treatment with at least two medications for neonatal | |
| | | abstinence syndrome as compared to infants born to women with the AG or GG genotypes. Be | |
| | | aware that this annotation is on the mother's genotype, even though the phenotype is observed in | |
| | | the infant and that this was not a statistically significant association. Other genetic and clinical | |
| | | factors may also affect severity of neonatal abstinence syndrome. | |
| 1451226166 | AG | Infants who have been exposed to methadone in utero and who are born to women with the AG | |
| | | genotype may be less likely to require treatment with at least two medications for neonatal | |
| | | abstinence syndrome as compared to infants born to women with the AA genotype. Be aware | |
| | | that this annotation is on the mother's genotype, even though the phenotype is observed in the | |
| | | infant and that this was not a statistically significant association. Other genetic and clinical | |
| | | factors may also affect severity of neonatal abstinence syndrome. | |
| 1451226166 | GG | Infants who have been exposed to methadone in utero and who are born to women with the GG | |
| | | genotype may be less likely to require treatment with at least two medications for neonatal | |
| | | abstinence syndrome as compared to infants born to women with the AA genotype. Be aware | |
| | | that this annotation is on the mother's genotype, even though the phenotype is observed in the | |
| | | infant and that this was not a statistically significant association. Other genetic and clinical | |
| | | factors may also affect severity of neonatal abstinence syndrome. | |
| 1451226200 | AA | Infants who have been exposed to buprenorphine in utero and who have the AA genotype may | |
| | | be more likely to require medication to treat neonatal abstinence syndrome as compared to | |
| | | infants with the CC genotype. However, this was not a statistically significant association. Other | |
| | | genetic and clinical factors may also affect severity of neonatal abstinence syndrome. | |
| 1451226200 | AC | Infants who have been exposed to buprenorphine in utero and who have the AC genotype may | |
| | | be more likely to require medication to treat neonatal abstinence syndrome as compared to | |
| | | infants with the CC genotype. However, this was not a statistically significant association. Other | |
| | | genetic and clinical factors may also affect severity of neonatal abstinence syndrome. | |
| 1451226200 | CC | Infants who have been exposed to buprenorphine in utero and who have the CC genotype may | |
| | | be less likely to require medication to treat neonatal abstinence syndrome as compared to infants | |
| | | with the AA or AC genotypes. However, this was not a statistically significant association. Other | |
| | | genetic and clinical factors may also affect severity of neonatal abstinence syndrome. | |
| 1451226206 | AA | Infants who have been exposed to methadone in utero and who have the AA genotype may be | |
| | | more likely to require medication to treat neonatal abstinence syndrome as compared to infants | |
| | | with the CC genotype. However, this was not a statistically significant association. Other genetic | |
| | | and clinical factors may also affect severity of neonatal abstinence syndrome. | |
| 1451226206 | AC | Infants who have been exposed to methadone in utero and who have the AC genotype may be | |
| | | more likely to require medication to treat neonatal abstinence syndrome as compared to infants | |
| | | with the CC genotype. However, this was not a statistically significant association. Other genetic | |
| | | and clinical factors may also affect severity of neonatal abstinence syndrome. | |
| 1451226206 | CC | Infants who have been exposed to methadone in utero and who have the CC genotype may be | |
| | | less likely to require medication to treat neonatal abstinence syndrome as compared to infants | |
| | | with the AA or AC genotypes. However, this was not a statistically significant association. Other | |
| | | genetic and clinical factors may also affect severity of neonatal abstinence syndrome. | |
| 1451226212 | AA | Infants who have been exposed to buprenorphine in utero and who are born to women with the | |
| | | rs740603 AA genotype may be less likely to require medication to treat neonatal abstinence | |
| | | syndrome as compared to infants born to women with the GG genotype. Be aware that this | |
| | | annotation is on the mother's genotype, even though the phenotype is observed in the infant and | |
| | | that this was not a statistically significant association. Other genetic and clinical factors may also | |
| | | affect severity of neonatal abstinence syndrome. | |
| 1451226212 | AG | Infants who have been exposed to buprenorphine in utero and who are born to women with the | |
| | | rs740603 AG genotype may be less likely to require medication to treat neonatal abstinence | |
| | | syndrome as compared to infants born to women with the GG genotype. Be aware that this | |
| | | annotation is on the mother's genotype, even though the phenotype is observed in the infant and | |
| | | that this was not a statistically significant association. Other genetic and clinical factors may also | |
| | | affect severity of neonatal abstinence syndrome. | |
| 1451226212 | GG | Infants who have been exposed to buprenorphine in utero and who are born to women with the | |
| | | rs740603 GG genotype may be more likely to require medication to treat neonatal abstinence | |
| | | syndrome as compared to infants born to women with the AA or AG genotypes. Be aware that | |
| | | this annotation is on the mother's genotype, even though the phenotype is observed in the infant | |
| | | and that this was not a statistically significant association. Other genetic and clinical factors may | |
| | | also affect severity of neonatal abstinence syndrome. | |
| 1451226220 | AA | Infants who have been exposed to methadone in utero and who are born to women with the AA | |
| | | genotype may be less likely to require medication to treat neonatal abstinence syndrome as | |
| | | compared to infants born to women with the GG genotype. Be aware that this annotation is on | |
| | | the mother's genotype, even though the phenotype is observed in the infant and that this was not | |
| | | a statistically significant association. Other genetic and clinical factors may also affect severity | |
| | | of neonatal abstinence syndrome. | |
| 1451226220 | AG | Infants who have been exposed to methadone in utero and who are born to women with the AG | |
| | | genotype may be less likely to require medication to treat neonatal abstinence syndrome as | |
| | | compared to infants born to women with the GG genotype. Be aware that this annotation is on | |
| | | the mother's genotype, even though the phenotype is observed in the infant and that this was not | |
| | | a statistically significant association. Other genetic and clinical factors may also affect severity | |
| | | of neonatal abstinence syndrome. | |
| 1451226220 | GG | Infants who have been exposed to methadone in utero and who are born to women with the GG | |
| | | genotype may be more likely to require medication to treat neonatal abstinence syndrome as | |
| | | compared to infants born to women with the AA or AG genotypes. Be aware that this annotation | |
| | | is on the mother's genotype, even though the phenotype is observed in the infant and that this | |
| | | was not a statistically significant association. Other genetic and clinical factors may also affect | |
| | | severity of neonatal abstinence syndrome. | |
| 1451340300 | *1 | Patients carrying the UGT1A3*1 allele in combination with a normal function allele may have | |
| | | increased exposure of telmisartan as compared to patients with one or more copies of the | |
| | | UGT1A3*2 or *3 allele. Other genetic and clinical factors may also influence metabolism of | |
| | | telmisartan. This annotation only covers the pharmacokinetic relationship between UGT1A3 and | |
| | | telmisartan and does not include evidence about clinical outcomes. | |
| 1451340300 | *2 | Patients carrying the UGT1A3*2 allele in combination with a UGT1A3*1 or a UGT1A3*2 | |
| | | allele may have decreased exposure of telmisartan as compared to patients with with two normal | |
| | | function alleles. Other genetic and clinical factors may also influence metabolism of telmisartan. | |
| | | This annotation only covers the pharmacokinetic relationship between UGT1A3 and telmisartan | |
| | | and does not include evidence about clinical outcomes. | |
| 1451340300 | *3 | Patients carrying the UGT1A3*3 allele in combination with a UGT1A3*1 or a UGT1A3*3 | |
| | | allele may have decreased exposure of telmisartan as compared to patients with with two normal | |
| | | function alleles. Other genetic and clinical factors may also influence metabolism of telmisartan. | |
| | | This annotation only covers the pharmacokinetic relationship between UGT1A3 and telmisartan | |
| | | and does not include evidence about clinical outcomes. | |
| 1451340320 | CC | Patients with the CC genotype may have increased concentrations of telmisartan as compared to | |
| | | patients with the GG genotype. Other genetic and clinical factors may also influence metabolism | |
| | | of telmisartan. This annotation only covers the pharmacokinetic relationship between SLCO1B3 | |
| | | and telmisartan and does not include evidence about clinical outcomes. | |
| 1451340320 | CG | Patients with the CG genotype may have increased concentrations of telmisartan as compared to | |
| | | patients with the GG genotype. Other genetic and clinical factors may also influence metabolism | |
| | | of telmisartan. This annotation only covers the pharmacokinetic relationship between SLCO1B3 | |
| | | and telmisartan and does not include evidence about clinical outcomes. | |
| 1451340320 | GG | Patients with the GG genotype may have decreased concentrations of telmisartan as compared to | |
| | | patients with the CC or CG genotype. Other genetic and clinical factors may also influence | |
| | | metabolism of telmisartan. This annotation only covers the pharmacokinetic relationship | |
| | | between SLCO1B3 and telmisartan and does not include evidence about clinical outcomes. | |
| 1450930839 | AA | Patients with the AA genotype may have decreased severity of nicotine dependence, as | |
| | | measured by FTND score, as compared to patients with the GG genotype. Other genetic and | |
| | | clinical factors may also affect severity of nicotine dependence. | |
| 1450930839 | AG | Patients with the AG genotype may have decreased severity of nicotine dependence, as | |
| | | measured by FTND score, as compared to patients with the GG genotype. Other genetic and | |
| | | clinical factors may also affect severity of nicotine dependence. | |
| 1450930839 | GG | Patients with the GG genotype may have increased severity of nicotine dependence, as measured | |
| | | by FTND score, as compared to patients with the AA or AG genotypes. Other genetic and | |
| | | clinical factors may also affect severity of nicotine dependence. | |
| 1451356840 | CC | Patients with the rs696 CC genotype may require decreased doses of sufentanil as compared to | |
| | | patients with the TT genotype. Other genetic and clinical factors may also influence sufentanil | |
| | | dosage requirements. | |
| 1451356840 | CT | Patients with the rs696 CT genotype may require decreased doses of sufentanil as compared to | |
| | | patients with the TT genotype. Other genetic and clinical factors may also influence sufentanil | |
| | | dosage requirements. | |
| 1451356840 | TT | Patients with the rs696 TT genotype may require increased doses of sufentanil as compared to | |
| | | patients with the CC or CT genotypes. Other genetic and clinical factors may also influence | |
| | | sufentanil dosage requirements. | |
| 1451225360 | AA | Infants who have been exposed to buprenorphine in utero and who have the rs4680 AA genotype | |
| | | may have an increased severity of neonatal abstinence syndrome as compared to infants with the | |
| | | AG or GG genotypes. Other genetic and clinical factors may also affect severity of neonatal | |
| | | abstinence syndrome. | |
| 1451225360 | AG | Infants who have been exposed to buprenorphine in utero and who have the rs4680 AG genotype | |
| | | may have a decreased severity of neonatal abstinence syndrome as compared to infants with the | |
| | | AA genotype. Other genetic and clinical factors may also affect severity of neonatal abstinence | |
| | | syndrome. | |
| 1451225360 | GG | Infants who have been exposed to buprenorphine in utero and who have the rs4680 GG genotype | |
| | | may have a decreased severity of neonatal abstinence syndrome as compared to infants with the | |
| | | AA genotype. Other genetic and clinical factors may also affect severity of neonatal abstinence | |
| | | syndrome. | |
| 1450930464 | CC | Patients with the CC genotype may begin using heroin at a later age as compared to patients with | |
| | | the TT genotype. Other genetic and clinical factors may also affect a patient's age at first use of | |
| | | heroin. | |
| 1450930464 | CT | Patients with the CT genotype may begin using heroin at a later age as compared to patients with | |
| | | the TT genotype. Other genetic and clinical factors may also affect a patient's age at first use of | |
| | | heroin. | |
| 1450930464 | TT | Patients with the TT genotype may begin using heroin at an earlier age as compared to patients | |
| | | with the CC or CT genotypes. Other genetic and clinical factors may also affect a patient's age at | |
| | | first use of heroin. | |
| 1451290960 | CC | Patients with diabetes mellitus and the CC genotype who are taking gliclazide may have | |
| | | decreased response as compared to patients with the TT genotype. Other clinical and genetic | |
| | | factors may also influence response to gliclazide in patients with diabetes mellitus. | |
| 1451290960 | CT | Patients with diabetes mellitus and the CT genotype who are taking gliclazide may have | |
| | | improved response as compared to patients with the CC genotype and decreased response | |
| | | compared to the TT genotype. Other clinical and genetic factors may also influence response to | |
| | | gliclazide in patients with diabetes mellitus. | |

Table 8—Example Annotation Data

TABLE 8

| Clinical | | | |
|---|---|---|---|
| Annotation ID | 981755803 | 1449311190 | 981204774 |
| Variant/Haplotypes | rs75527207 | rs4149056 | rs1799971 |
| Gene | CFTR | SLCO1B1 | OPRM1 |
| Level of | 1A | 3 | 4 |
| Evidence | | | |
| Level Override | | | |
| Level Modifiers | Rare Variant; | Tier 1 VIP | |
| | Tier 1 VIP | | |
| Score | 234.875 | 2 | −2 |
| Phenotype | Efficacy | Dosage | Efficacy |
| Category | | | |
| PMID Count | 28 | 1 | 2 |
| Evidence Count | 30 | 1 | 3 |
| Drug(s) | ivacaftor | mercaptopurine; | Drugs used in |
| | | methotrexate | nicotine |
| | | | dependence; |
| | | | nicotine |
| Phenotype(s) | Cystic Fibrosis | Precursor Cell | Tobacco Use |
| | | Lymphoblastic | Disorder |
| | | Leukemia- | |
| | | Lymphoma | |
| Latest History Date | Mar. 24, 2021 | Mar. 24, 2021 | Mar. 24, 2021 |
| (YYYY-MM-DD) | | | |
| URL | https://www.pharmgkb.org/ | https://www.pharmgkb.org/ | https://www.pharmgkb.org/ |
| | clinicalAnnotation/981755803 | clinicalAnnotation/1449311190 | clinicalAnnotation/981204774 |
| Specialty | Pediatric | Pediatric | |
| Population | | | |

| Clinical | | | |
|---|---|---|---|
| Annotation ID | 981419532 | 981419266 | 1451340300 |
| Variant/Haplotypes | rs4693075 | HLA-B*15:02:01 | UGT1A3*1, |
| | | | UGT1A3*2, |
| | | | UGT1A3*3 |
| Gene | COQ2 | HLA-B | UGT1A3 |
| Level of | 3 | 1A | 3 |
| Evidence | | | |
| Level Override | | | |
| Level Modifiers | | Tier 1 VIP | |
| Score | 3.25 | 315.75 | 4 |
| Phenotype | Toxicity | Toxicity | Metabolism/PK |
| Category | | | |
| PMID Count | 3 | 18 | 1 |
| Evidence Count | 3 | 23 | 2 |
| Drug(s) | atorvastatin; hmg | phenytoin | telmisartan |
| | coa reductase | | |
| | inhibitors; | | |
| | rosuvastatin | | |
| Phenotype(s) | Muscular | drug reaction | |
| | Diseases | with eosinophilia | |
| | | and systemic | |
| | | symptoms; Epidermal | |
| | | Necrolysis, Toxic; | |
| | | severe cutaneous | |
| | | adverse reactions; | |
| | | Stevens-Johnson | |
| | | Syndrome | |
| Latest History Date | Mar. 24, 2021 | Jun. 22, 2022 | Mar. 24, 2021 |
| (YYYY-MM-DD) | | | |
| URL | https://www.pharmgkb.org/ | https://www.pharmgkb.org/ | https://www.pharmgkb.org/ |
| | clinicalAnnotation/981419532 | clinicalAnnotation/981419266 | clinicalAnnotation/1451340300 |
| Specialty | | Pediatric | |
| Population | | | |

### Example 2—Noncoding Genomics Writeup

Method to annotate whole genomic sequences based on variants across the genome, including noncoding variants

Noncoding variant annotations are tied to individual genotype and used to encourage clinical testing, or supervised next steps such as for the example of breast cancer screenings (e.g., mammographies/MRIs), treatment (e.g., birth control as a preventative medication, surgery), or lifestyle changes (e.g., limit dairy intake)

For noncoding variants, one implementation is to take data that connects noncoding variants to coding genes by cell type, and tie cell types they analyze to disease states (e.g., in breast epithelial cell, Applicants can optionally take those markers and link them to breast cancer) to make a disease risk prediction; another is to directly associate noncoding variants to a cell type, and to tie cell to disease states (e.g., in breast epithelial cell, Applicants can optionally take those markers and link them to breast cancer) to make a disease risk prediction; another method is to directly associate noncoding variants to a phenotype, trait, or disease state

Data to connect noncoding variants to coding genes or to connect noncoding variants to disease states can come from large GWAS in biobanks or population studies, or from CRISPR experiments in which pieces of the noncoding genome are inhibited or amplified to measure their effect on coding sequences, or from an activity-by-contact model approach based on the folding of DNA in 3D cellular space that takes some measure of activity (e.g. DNAse I Hypersensitive sites or H3K27ac chromatin immunoprecipitation sequencing (ChIP-seq) or some combination thereof such as a geometric mean or another measure of activity) and some measure of contact (e.g., Hi-C, including KR-normalized Hi-C contact frequency between a noncoding genomic sequence and a gene promoter or 3C) to determine the functional relationship between a noncoding sequence and a coding sequence

e.g., ABC data from source here: flekschas.github.io/enhancer-gene-vis/?daet=Uz1 tEABQf-uzktblvBKSQ %3Arg %3ADNAAccessibility&dals=indicator&darn=true&dasi=true&dasp=false &e=chr10.81230693&egce=max-score&egi=true&egp=false&erc=solid&erhu=false&eri=true&erso=0d05&ert=e31pYv5LSIiik7C FtuAMTw %3Arg %3A1%3A4%3A4%3A0%3A3%3A5%3AEnhancer %20regions&f=rs125056 6&g=&s=chr10.80993117&vs=pValue&vt=VF5-RDXWTxidGMJU7FeaxA %3Arg %3A7%3A8&w=0

If noncoding variant data has many noncoding elements linking to a particular gene for a given cell type, then a method to prioritize which noncoding element has the most predictive connection can be created using supervised learning models (e.g., neural networks, random forest, naive Bayes, linear regression, logistic regression, k nearest neighbors, support vector machines (SVMs))

These models can be trained or finetuned on a collection of biobank data (e.g., UK Biobank, Finland Biobank, UK10, Japan Biobank, MEC, 1000 genomes, BioME, BioVU, and others) or with synthetic data (e.g., upsampling data, SMOTE, resampling, performing unsupervised learning methods to cluster and create new data)

Annotations can also be converted into a function-based polygenic risk score by modifying a weighting algorithm (e.g., an existing weighting method like the LDpred-funct algorithm (www.nature.com/articles/s41467-021-25171-9) to include noncoding functional annotations as an input) or creating a new weighting algorithm.

If the input genetic sequence has a variant that falls in a noncoding area that links to a gene in a set of cell types, Applicants can optionally tie that to the disease(s) implicated by the cell types.

If the raw noncoding variant data is in the form of ranges (e.g., between X-Y BP on Z chromosome) then for an individual genotype, Applicants can optionally annotate any variants that fall in the ranges of importance.

LDpred-funct improvement writeup (www.nature.com/articles/s41467-021-25171-9):
1) use connections between noncoding and coding variants, such as from the activity-by-contact model for noncoding-coding genomic connections, to create a list of trait-specific functional priors for variant importance
2) analytically estimate posterior mean causal effect sizes
3) regularize these estimates using a method like cross-validation

practically, use the noncoding method to get new functional enrichment estimates, which are an input straight into LDpred-funct here: github.com/carlaml/LDpred-funct

### LDpred-inf

The LDpred-inf method estimates posterior mean causal effect sizes under an infinitesimal model, accounting for LD (Vilhjálmsson, B. J. et al. Modeling linkage disequilibrium increases accuracy of polygenic risk scores. Am J. Hum Genet. 97, 576-592 (2015)). The infinitesimal model assumes that normalized causal effect sizes have prior distribution βi˜N(0,σ²), where σ²=h_{g}²/M, h_{g}², is the SNP-heritability, and M is the number of SNPs. The posterior mean causal effect sizes are

E(β|^{˜}β,D)=((N/(1−hₗ²))*D+(1/σ²)I)⁻¹N*^{˜}β, (2)

where D is the LD matrix between markers, I is the identity matrix, N is the training sample size, ^{˜}β is the vector of marginal association statistics, and hₗ²≈kh²/M is the heritability of the k SNPs in the region of LD; following Vilhjilmsson, B. J. et al. Modeling linkage disequilibrium increases accuracy of polygenic risk scores. Am J. Hum Genet. 97, 576-592 (2015). The approximation 1−hₗ²≈1 can optionally be used, which is appropriate when M>>k. D is typically estimated using validation data, restricting to non-overlapping LD windows. A default LD window size (e.g., M/3000) can optionally be used. h_{g}² can be estimated from raw genotype/phenotype data (Loh, P.-R., Kichaev, G., Gazal, S., Schoech, A. P. & Price, A. L. Mixed-model association for biobank-scale datasets. Nat. Genet. 50, 906-908 (2018), Ge, T., Chen, C.-Y., Neale, B. M., Sabuncu, M. R. & Smoller, J. W. Phenome-wide heritability analysis of the UK Biobank. PLOS Genetics 13, e1006711 (2017).) (the approach that Applicants use here; see below), or can be estimated from summary statistics using the aggregate estimator as described in Vilhjilmsson, B. J. et al. Modeling linkage disequilibrium increases accuracy of polygenic risk scores. Am J. Hum Genet. 97, 576-592 (2015). To approximate the normalized marginal effect size Vilhjilmsson, B. J. et al. Modeling linkage disequilibrium increases accuracy of polygenic risk scores. Am J. Hum Genet. 97, 576-592 (2015) uses the p-values to obtain absolute Z scores and then multiplies absolute Z scores by the sign of the estimated effect size. When sample sizes are very large, p-values may be rounded to zero, in which case Applicants approximate normalized marginal effect sizes βᵢ\hat{ } by bᵢ\hat{ }√(2*pi*(1−pi))/√(σ_{Y}²) where bᵢ\hat{ } is the per-allele marginal effect size estimate, pi is the minor allele frequency of SNP i, and σ_{Y}² is the phenotypic variance in the training data. This applies to all the methods that use normalized effect sizes. Although the published version of LDpred requires a matrix inversion (Eq. (2)), Applicants have implemented a computational speedup that computes the posterior mean causal effect sizes by efficiently solving (Bycroft, C. et al. The UK biobank resource with deep phenotyping and genomic data. Nature 562, 203-209 (2018)) the system of linear equations ((1/σ2)I+N*D)E(β|β,D)=Nβ.

### LDpred

The LDpred method is an extension of LDpred-inf that uses a point-normal prior to estimating posterior mean effect sizes via Markov Chain Monte Carlo (MCMC) Vilhjilmsson, B. J. et al. Modeling linkage disequilibrium increases accuracy of polygenic risk scores. Am J. Hum Genet. 97, 576-592 (2015). It assumes a Gaussian mixture prior: βᵢ˜N(0,h_{g}²/M*p) with probability p, and βᵢ˜0 with probability 1−p, where p is the proportion of causal SNPs. The method is optimized by considering different values of p (1E-4, 3E-4, 1E-3, 3E-3, 0.01,0.03,0.1,0.3,1); in the special case where 100% of SNPs are assumed to be causal, LDpred is roughly equivalent to LDpred-inf. SNPs can optionally be excluded from long-range LD regions (reported in Bycroft, C. et al. The UK biobank resource with deep phenotyping and genomic data. Nature 562, 203-209 (2018)), as the secondary analyses showed that including these regions were suboptimal, consistent with ref Lloyd-Jones, L. R. et al. Improved polygenic prediction by bayesian multiple regression on summary statistics. Nat. Commun. 10, 5086 (2019).

### AnnoPred

AnnoPred (Hu, Y. et al. Leveraging functional annotations in genetic risk prediction for human complex diseases. PLOS Comput. Biol. 13, 1-16 (2017)) uses a Bayesian framework to incorporate functional priors while accounting for LD, optimizing prediction R2 over different assumed values of the proportion of causal SNPs. Hu et al. proposed two different priors for use with AnnoPred. The first prior assumes the same proportion of causal SNPs but different causal effect size variance across functional annotations, and uses a point-normal prior to estimating posterior mean effect sizes via Markov Chain Monte Carlo (MCMC). In the special case where 100% of SNPs are assumed to be causal, AnnoPred is roughly equivalent to LDpred-funct-inf (see below). The second prior assumes different proportions of causal SNPs but the same causal effect size variance across functional annotations. In a specific example, only the first prior is considered, since the second prior cannot be extended to incorporate continuous-valued annotations from the baseline-LD model. However, other priors may be considered. SNPs can optionally be excluded from long-range LD regions (as reported in Bycroft, C. et al. The UK biobank resource with deep phenotyping and genomic data. Nature 562, 203-209 (2018)) when running AnnoPred. In an illustrative example, a default LD window size (e.g., M/3000) can be used.

### LDpred-Funct-inf

LDpred-inf can optionally be modified to incorporate functionally informed priors on causal effect sizes using the baseline-LD mode125, which includes coding, conserved, regulatory, and LD-related annotations, whose enrichments are jointly estimated using stratified LD score regression (Finucane, H. K. et al. Partitioning heritability by functional annotation using genome-wide association summary statistics. Nat. Genet. 47, 1228-1235 (2015), Gazal, S. et al. Linkage disequilibrium-dependent architecture of human complex traits shows action of negative selection. Nature Genetics 49, 1421 EP-(2017)). Specifically, Applicants can optionally assume that normalized causal effect sizes have prior distribution βi˜N(0,c*σ2i), where σ2i is the expected per-SNP heritability under the baseline-LD model (fit using training data only) and c is a normalizing constant such that ΣMi=1 [symbol] {σ2i>0}cσ2i=h2g; SNPs with σ2i≤0 are removed, which is equivalent to setting σ2i=0. The posterior mean casual effect sizes are

E[β|^{˜}β,D,σ21, . . . ,σ2M+]=W−1N*^{˜}β(N*D+1c(1σ21 [symbol] 0 . . . [symbol] 0 [symbol] 1σ2M+)⁻¹N*^{˜}β (4)

where M+ is the number of SNPs with σ2i>0σi2>0. The posterior mean causal effect sizes are computed by solving the system of linear equations WE[β|^{˜}β,D,σ21, . . . , σ2M]=N*^{˜}β·h2g is estimated as described above (see LDpred-inf). D is estimated using validation data, restricting to windows of size 0.15% M+. In principle, it is possible to use banding to define the LD matrices, where LD between distant pairs of SNPs (10 Mb or more) is rounded to zero (Yang, J. et al. Conditional and joint multiple-snp analysis of gwas summary statistics identifies additional variants influencing complex traits. Nat. Genet. 44, 369-375 (2012)), but Applicants elected to use the simpler window-based approach (as in Vilhjilmsson, B. J. et al. Modeling linkage disequilibrium increases accuracy of polygenic risk scores. Am J. Hum Genet. 97, 576-592 (2015)).

### LDpred-funct

Applicants modify LDpred-funct-inf to regularize posterior mean causal effect sizes using cross-validation. Applicants rank the SNPs by their (absolute) posterior mean causal effect sizes, partition the SNPs into K bins (analogous to ref. 56) where each bin has roughly the same sum of squared posterior mean effect sizes, and determine the relative weights of each bin based on the predictive value in the validation data. Intuitively if a bin is dominated by non-causal SNPs, the inferred relative weight will be lower than for a bin with a high proportion of causal SNPs. This non-parametric shrinkage approach can optimize prediction accuracy regardless of the genetic architecture. In detail, let S=ΣiE[βi|\tilde{β}i]2. To define each bin, Applicants first rank the posterior mean effect sizes based on their squared values E[βi|\tilde{β}i]2. Applicants define bin b1 as the smallest set of top SNPs with Σi∈b1E[βi|\tilde{β}βi]2≥SK, and iteratively define bin bk as the smallest set of additional top SNPs with Σi∈b1, . . . , bkE[βi|\tilde{β}i]2≥kSK. Let PRS(k)=Σi∈bkE[βi|\tilde{β}i]gi. Applicants define

PRSLDpred−funct=Σk=1KαkPRS(k), (5)
where the bin-specific weights (E±k are optimized using validation data via 10-fold cross-validation. For each held-out fold in turn, Applicants can optionally split the data so Applicants estimate the weights (E±k using the samples from the other nine folds (90% of the validation) and compute PRS on the held-out fold using these weights (10% of the validation); thus, in each cross-validation fold, the validation samples used to estimate regularization weights are disjoint from the validation samples used to compute predictions. Applicants then compute the average prediction R2 across the 10 held-out folds. To avoid overfitting when K is very close to N, the number of bins (K) can optionally be between 1 and 100, such that it is proportional to h2ghg2 and the number of samples used to estimate the K weights in each fold is at least 100 times larger than K:

K=min(100,┌0.9N*h2g100┐), (6)

where N is the number of validation samples. For highly heritable traits (h2g˜0.5), LDpred-funct reduces to the LDpred-funct-inf method if there are ˜200 validation samples or fewer; for less heritable traits (h2g˜0.1), LDpred-funct reduces to the LDpred-funct-inf method if there are ˜1000 validation samples or fewer. In simulations, Applicants set K to 40 (based on 7,585 validation samples; see below), approximately concordant with Eq. (6). The value of 100 in the denominator of Eq. (6) was coarsely optimized in simulations, but was not optimized using real trait data. Applicants note that functional annotations are not used in the cross-validation step (although they do impact the posterior mean causal effect size provided as input to this step). Thus, it is likely that SNPs from a given functional annotation will fall into different bins (possibly all of the bins).

### EXAMPLE WORKFLOWS

### Example Workflow 1—Whole Genome Sequence

Individual is asked by medical provider to take a genetic test;

Patient provides blood, saliva, or buccal (cheek swab) sample;

Sample is sent to lab for DNA extraction using standard techniques;

DNA sample is sequenced using a whole genome sequencing machine (e.g., from Illumina, or Ultima Genomics, or Oxford Nanopore, or BGI, etc.);

Sequence is analyzed with DeepVariant or GATK and aligned against a reference genome to create a variant call file (VCF) that includes all variants;

VCF is uploaded into the software which annotates variants, including for disease risk, disease protection, and drug response;

Results are sent to provider and/or individual in curated and/or raw format; and/or

Based on results, individual takes another genetic test, or adopts a screening or monitoring regimen, or undertakes a new therapeutic regimen, or adopts a lifestyle change.

### Example Workflow 2—Array

Individual decides to take a genetic test;

Patient provides blood, saliva, or buccal (cheek swab) sample;

Sample is sent to lab for DNA extraction using standard techniques;

DNA sample is genotyped with a microarray (e.g., from Illumina, or Affymetrix);

Genotype is converted into a variant call file (VCF) that includes all variants;

VCF is uploaded into the software which annotates variants, including for disease risk, disease protection, and drug response;

Results are sent to individual in curated and/or raw format; and/or

Based on results, individual takes another genetic test, or adopts a screening or monitoring regimen, or undertakes a new therapeutic regimen, or adopts a lifestyle change (e.g., takes statins at the right dose).

### Example Workflow 3—Companion Diagnostic

Individual decides to take a genetic test;

Patient provides blood, saliva, or buccal (cheek swab) sample;

Sample is sent to lab for DNA extraction using standard techniques;

DNA sample is genotyped with a microarray (e.g., from Illumina, or Affymetrix);

Genotype is converted into a variant call file (VCF) that includes all variants;

VCF is uploaded into the software which annotates variants, including for disease risk, disease protection, and drug response;

Results are sent to individual in curated and/or raw format;

In particular, software identifies that individual has a particular genetic variant that makes them a target candidate for a genetic therapy, e.g., a gene editing treatment for a disease; and/or

Individual signs up for gene editing treatment.

### Specific Example 1

In an example, the method can include a functionally informed whole-genome polygenic risk score model for disease risk. This new polygenic prediction model combines the power of a Bayesian supervised learning method that leverages trait-specific functional prior annotations, LDpred-funct, and an advanced enhancer-gene connection framework, called Activity-by-Contact (ABC) that allows for comprehensive genome-wide functional variant annotations. This model can be fully HIPAA-compliant. In variants, the technology can achieve a better polygenic prediction model by: (1) incorporating comprehensive functional non-coding annotations into disease risk models, and using Bayesian supervised learning methods to improve the performance of those models, (2) using functional data to ensure efficacy across diverse ethnic backgrounds, and (3) annotating functional pathways contributing to an individual's risk level to improve the interpretability of predictive genetic test results. To address the issue of polygenic risk models being hard to interpret, the models can be trained using functional data, and provide functional variant annotations so that clinicians can understand what contributes to a high-risk score. For each variant that has a high weight for an individual, clinicians are shown: (1) the variant's impact on relevant genes, such as a noncoding variant that decreases the transcription and expression of the SORT1 gene, or a coding variant that causes a missense mutation in LDLR; (2) what functional pathway is impacted by this change; (3) and how all these small effect variants come together to contribute to the overall risk score. Moreover, training the models using functional data should significantly improve the ethnic inclusivity of risk prediction, addressing the second obstacle towards clinical adoption. Finally, HIPAA-compliant computational infrastructure can enable the validation of the models. A benefit of these innovations is improving the accuracy, interpretability, and ethnic inclusivity of genomic disease risk prediction in preventative healthcare.

These new polygenic prediction models combine the power of a Bayesian supervised learning method that leverages trait-specific functional prior annotations, LDpred-funct, with an enhancer-gene connection framework, called activity-by-contact (ABC). Incorporating whole-genome mappings of variant function, both coding and non-coding, can significantly improve the accuracy and ethnic generalizability of the polygenic risk score that estimates the effect of many genetic variants on an individual's common disease risk.

LD-pred-funct can be used to incorporate trait-specific functional priors informed from the activity-by-contact maps. For a cardiovascular disease model, ABC data can be used, wherein the ABC data can be tailored to cell lines linked to cardiovascular disease, such as coronary artery, coronary artery smooth muscle cell, and heart ventricle. Functional priors can be fit using a baseline-LD model, which includes coding, conserved, regulatory, and LD-related annotations. LDpred-funct first analytically estimates posterior mean causal effect sizes, accounting for functional priors and LD between variants. LDpred-funct then uses cross-validation within validation samples to regularize causal effect size estimates in bins of different magnitude, improving prediction accuracy for sparse architectures. LDpred-funct can attain higher polygenic prediction accuracy than other methods in simulations with real genotypes, analyses of 21 highly heritable UK Biobank traits, and meta-analyses of height using training data from UK Biobank and 23andMe cohorts. LDpred-funct attained +10% (P<2×10-4) and +4.6% (P=0.04) relative improvements compared to LDpred and SBayesR, two state-of-the-art methods that do not model functional enrichment.

The method can optionally include validating the model. In variants, model validation can include: obtaining patient data from a set of biorepositories, and for each individual in these biorepositories, cardiovascular disease risk can be predicted using the cardiovascular disease model. To benchmark this performance against existing methods simulations can be run and model outcomes can be compared to predictions made from (1) existing single-variant tests, such as clinical coding-genome based tests as documented in ClinVar, and other well-studied monogenic variants in the literature as well as (2) other known polygenic risk scoring methods and similar predictive risk models from the literature, Existing single-variant tests include monogenic familial hypercholesterolemia variants, (e.g., pathogenic variants in genes LDLR, APOB, and PCSK9 which confer an up to 3-fold increased risk for coronary artery disease).

First, each individual's genomic sequence in the test datasets can be analyzed using the activity-by-contact (ABC) informed LDpred-funct model as described in the approach. Performance can be compared against panels that only detect monogenic variants. Panels can be simulated based on other well-studied monogenic variants in the literature, including regions identified in the recent follow-up paper using ABC data for heart disease, which validates functional ABC predictions for whole-genome Coronary Artery Disease loci using CRISPRi-Perturb-Seq and collates against functional experiments in animal models. Finally, the method can be compared against existing polygenic risk methods for cardiovascular disease, including those highlighted by the recent American Heart Association statement on polygenic risk.

The performance of currently available cardiovascular disease tests and other predictive models can be compared against the model using standard metrics from state-of-the-art machine learning approaches: precision, sensitivity (recall), specificity, Area-Under-the-Curve (AUC), and accuracy. Precision-recall plots can be constructed for each model to compare performance. Pairwise and nested model comparisons can be performed to characterize the predictive ability of each model. For calculations, a “case” or “positive” individual refers to an individual in the dataset who develops cardiovascular disease, while a “control” or “negative” individual refers to one who does not develop cardiovascular disease. In variants, (1) Precision: Precision denotes the proportion of correct positive classifications and is calculated as the ratio between correctly classified positives and all positives. (2) Sensitivity (Recall): Also known as the True Positive Rate (TPR), recall denotes the rate of true positives classified correctly, and is calculated as the ratio between correctly classified positive samples and all samples assigned to the positive class. This metric is also regarded as being among the most important for medical studies since it is desired to miss as few positive instances as possible, which translates to a high recall. (3) Specificity: The specificity is the negative class version of sensitivity and denotes the rate of negative samples correctly classified. It is calculated as the ratio between correctly classified negative samples and all samples classified as negative. (4) AUC: The receiver operating characteristic (ROC) curve measures the total 2D area under the curve, which on one axis the true positive rate (recall) and on the other the false positive rate (which is the ratio of false positives to all negative samples). AUC is the metric typically used in polygenic risk scoring literature and machine learning literature to quantify the overall performance of predictive models, with the added benefit of being invariant to the classification threshold chosen for a particular model. (5) Accuracy: Is the ratio between the correctly classified samples and the total number of samples in the evaluation dataset.

The method can optionally include evaluating the ethnic inclusivity of the cardiovascular disease prediction model as compared to currently available genetic prediction models. Historically, those of non-European ancestry (e.g., African, Asian, Latino ancestry) have been underrepresented in genomic analysis and research, and therefore, in clinical relevance of available genetic tests. Disease risk predictions in non-European populations have historically been 53-89% less accurate than in European populations. However, incorporating functional annotations into predictive risk models can significantly improve ethnic inclusivity by picking up on real signal rather than statistical artifacts in biased training data. For example, diverse ethnic populations have differing linkage disequilibrium (LD) and minor allele frequencies (MAF). Given that historically available biorepository samples have been primarily of European ancestry, attempts at training models directly from biorepository data without any functional annotation have been hampered by the ethnic bias in these datasets, with LD and MAF specific to European populations. Instead, these databases can be annotated with functional data, which is more universal across populations. Because this method is the first to incorporate comprehensive functional data across the genome (coding and non-coding), the added benefit of ethnic inclusivity can be achieved from the models. The method can include segmenting the test set by ancestral group (e.g., European, African, Latino, South Asian, and East Asian). For each method, performance metrics can be calculated within each ethnic group, and then quantify differences across ethnicity for that method. Finally, the variance in performance by ethnicity can be plotted for each method to benchmark whether the approach really does improve ethnic inclusivity.

### Specific Example 2

In another example, the outcome of all or parts of the method can include detailed validation for a whole-genome-based predictive genetic test. Predictive genetic tests can be used clinically to assess risk of developing a disease, so tailored prevention strategies can be enacted to minimize risk. The method can optionally include quantifying the potential improvement in accuracy and ethnic inclusivity of the new predictive models for complex disease, starting with cardiovascular disease. This can enable the creation of the first clinical-grade whole-genome-based predictive genetic test. This project addresses the technical challenge of developing a whole-genome-based predictive genetic test. Key technological innovations can include: (1) incorporating comprehensive whole-genome annotations into disease risk models, (2) tailoring ancestry-informed scores to ensure efficacy across diverse ethnic backgrounds, and (3) using machine learning methods to optimize the performance of those models. A key benefit of these innovations can be improving the accuracy and ethnic inclusivity of genetic tests, thereby improving preventative healthcare.

In variants, the systems and methods described herein provide the first functionally informed whole-genome predictive models for disease risk in an accurate and ethnically inclusive manner By adopting the novel approach of incorporating non-coding functional genomic analysis into supervised learning models, predictive accuracy and ethnic generalizability can be improved.

The technology can include a new machine-learning-based disease risk prediction model to analyze whole genome sequences. Key technological innovations can include: (1) incorporating comprehensive functional non-coding annotations into disease risk models for the first time (e.g., analyzing the noncoding genome), (2) tailoring ancestry-informed scores to ensure efficacy across diverse ethnic backgrounds, and (3) using advanced machine learning methods to improve the performance of those models. The technology can optionally include a custom cloud infrastructure to curate disease-relevant databases and enable the validation of the models. This can enable the first clinical-grade whole-genome-based predictive genetic test. A key benefit of these innovations is improving the accuracy and ethnic inclusivity of genetic tests, thereby improving preventative healthcare.

Examples of diseases that risk scores can be predicted for include: cardiovascular disease, breast cancer, colorectal cancer, prostate cancer, and/or other genetically-linked diseases.

In variants, the method can leverage recent advances in providing a functional understanding of the enhancer-gene connections for noncoding gene sequences, which has historically been challenging because the noncoding genes do not create physical proteins. In Objective 1, features can be engineered using ABC maps to functionally annotate the biorepository data, so that the models can train on functionally annotated data across the whole genome, not just the coding genome.

The method can optionally include Objective 1: Creating a dataset to facilitate model validation and testing. The goal of this objective is to establish a dataset to validate the models (Objective 2), as well as to quantitatively benchmark their performance (Objective 3).

Objective 1 can optionally include Task 11: using the backend infrastructure, curating a comprehensive dataset of whole genome samples and associated disease labels. This task can include receiving access to biorepository data, which include hundreds of thousands of de-identified whole genome sequences along with demographic data (sex, age, self-reported ethnicity) and ICD9/10 code labels related to disease development. Standard quality control practices can be conducted over each dataset using AWS Sagemaker, including removing any duplicate data, verifying self-reported ethnicity against genome-calculated ancestry from 1000 genomes principal component analysis, and filtering out samples with inconsistent ICD9/10 label quality. Each sample can be tagged into a case versus control set for cardiovascular disease. Data can be merged from the biorepositories into a master dataset in AWS S3 including genomes, demographic and ancestry information, and disease labels (case or control for cardiovascular disease).

Objective 1 can optionally include Task 12: engineering features based on non-coding and coding annotations. In this task, to facilitate validation of the machine learning models in Objective 2, feature engineering can be conducted to create new columns in the dataset. Genomic sequences can be analyzed for functional elements in both the coding and non-coding genome so that the models can learn from these features. Constructing a model with functionally-informed features can yield benefits in both performance and ethnic inclusivity.

Genome-based ancestry can optionally be determined and included demographic and ancestry information as features in the dataset. Each genomic sequence can be analyzed using an activity-by-contact (ABC) method, and ABC scores for variants in each genome can be included as features. However, other functional annotation methods can be used. For cardiovascular disease as a disease of interest, all or parts of the method can be repeated using ABC data tailored to cell lines linked to cardiovascular disease (e.g., coronary artery, coronary artery smooth muscle cell, heart ventricle, etc.). However, other cell lines can be used. Second, for each genomic sequence we will scan for variants directly perturbed in high-throughput CRISPR experimental data, and include CRISPR functional data as features for relevant cardiovascular disease variants. Next, will incorporate functional experimental data on individual non-coding elements studied in cardiovascular disease. Finally, any functional annotations on the coding genome already collated in existing databases (e.g., ClinVar) can optionally be incorporated. These activities aim to engineer features that allow us to interpret the function of both coding and non-coding genomic variants across the whole genome so the model can learn from these features.

Risks and contingency: in variants, ICD 9/10 codes can include disease diagnosis misclassifications due to clerical errors or self-reported diagnoses. In variants, to address this potential pitfall from electronic health records (EHRs), a random sample of cases classified as positive and negative for each disease using ICD 9/10 codes can be cross-referenced against the entirety of their medical records, and remove samples with inconsistencies. The replication of null signals can optionally be analyzed to determine if there are any patterns of bias. Another risk for machine learning methods is limited data; however, all or portions of the method can optionally access biobanks, and training methods can optionally be adjusted for low sample size. In the training dataset, standard machine learning data augmentation methods can optionally be performed to increase signal as needed, (e.g., up-sampling, Synthetic Minority Oversampling Technique, etc.).

The method can optionally include Objective 2: selecting the best predictive machine learning model for cardiovascular disease by leveraging genome-wide variation from coding and noncoding regions. In this objective, a new predictive model can be validated for cardiovascular disease genetic testing.

Objective 2 can optionally include Task 21: training the new supervised learning predictive model that uses both coding and non-coding annotations and multi-ethnic data as features in the cardiovascular disease dataset. A Bayesian supervised learning model can be used to predict cardiovascular disease risk. In this task, individual-level data from diverse ancestral backgrounds from the training set can be incorporated into this model. The non-coding and coding annotations built in objective 1 can be used as functional priors for variant selection and regularization of weights, while also controlling for linkage-disequilibrium (LD), which represents the correlation between variants. This can be used to construct a multivariate regression model to compute a genomic risk score in the cardiovascular disease dataset.

Classical supervised learning models can be run, including support vector machines (SVMs), k-nearest-neighbors, boosted decision trees, random forests and neural networks, using the training data subset in Objective 1. Model outputs (eg, posterior prediction probabilities) can optionally be combined with the results from the Bayesian supervised learning model (eg, using a simple weighted combination of probabilities) to create a joint model trained on the cardiovascular disease dataset.

Objective 2 can optionally include Task 22: conducting model selection by evaluating the models in the validation biorepository subset. In variants, the best model can be selected based on the performance in the validation subset, measured using Area Under the ROC Curve (AUC), as is best practice in the field. A model threshold can then be set based on the AUC in the validation set, so that in the test phase for Objective 3, the model will output a single set of predictions for any given disease.

Risks and contingency: Machine learning methods run the risk of overfitting the model to the training dataset, limiting generalizability in the real world. In variants, to mitigate this risk, models can be validated in a completely held-out validation set and test set (as created in Task 13) so that the model cannot learn from the same data it is evaluated against. Moreover, by using multiple different biorepositories across differing geography and sequencing methods, the generalizability of the results to a real-world setting can be improved. Another concern is that machine learning models can have limited interpretability or be considered a “black box;” the use of functional data has the added benefit of improving the interpretability of the model because weights assigned to any feature can then be interpreted in terms of the biological function of each variant.

The method can optionally include Objective 3: characterizing the performance of the new model as compared to currently available genetic tests in the coding genome.

Objective 3 can optionally include Task 31: evaluating model performance in the held-out test dataset. The performance of the new model from Objective 2 in the held-out test dataset can be evaluated, and the predictions for each genome for cardiovascular disease risk can be determined. To benchmark this performance against existing methods, simulations of existing genetic tests can optionally be run in the held-out test dataset. In an example, currently available cardiovascular disease tests can be simulated from all services reimbursed by CMS, including Ambry Genetics, Myriad Genetics, and Invitae, using the ClinVar clinical model resource. The method can optionally include simulating previous predictive risk models in the literature, such as additive models (eg, polygenic risk scores), including the LDpred-funct model.

For each of these models, performance in the test set can be quantified using standard metrics from state-of-the-art machine learning approaches: precision, sensitivity (recall), specificity, and overall accuracy. For example, precision-recall plots for each model can be constructed to compare performance. Pairwise and nested model comparisons can be performed to characterize the predictive ability of each model.

Objective 3 can optionally include Task 32: evaluating ethnic inclusivity of models in the held-out test dataset. Model performance can be compared across different ancestral groups to quantify ethnic inclusivity. To do this, the test set can optionally be segmented by ancestral group (e.g., European, African/African American, Latino or Hispanic, South Asian, and East Asian). For each model, performance metrics can be calculated within each ethnic group, and then quantify differences across ethnicity for that model. Finally, the variance in performance can be plotted by ethnicity for each model to benchmark whether the approach really does improve ethnic inclusivity.

Risks and contingency plans: variants of the method can optionally ensure that no held-out test data leak into the training or validation datasets in Objective 2. If this were to occur, it could risk biasing the modeling process and limiting the generalizability of the results. In variants, the test dataset can be stored in a separate AWS S3 bucket and restrict access using the AWS identity access management console until Objective 3, so that even by accident, test data cannot be accessed until the best performing model in the validation set has been selected.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Different subsystems and/or modules discussed above can be operated and controlled by the same or different entities. In the latter variants, different subsystems can communicate via: APIs (e.g., using API requests and responses, API keys, etc.), requests, and/or other communication channels.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

1. A method, comprising, by one or more computing systems:
determining a set of segmented annotation data, comprising a plurality of subsets of annotation data, wherein the annotation data comprises annotations mapped to variable value sets, wherein each variable value set corresponds to a genomic variant, and wherein each subset of annotation data corresponds to a search region in a set of search regions;
receiving unannotated genomic data for a subject;
comparing the unannotated genomic data to a reference genome to identify variant loci;
determining a variable value set for each identified variant locus in the unannotated genomic data;
generating annotated variant loci, comprising, for each variable value set associated with an identified variant locus:
selecting a search region from the set of search regions based on the variable value set;
searching within a subset of annotation data corresponding to the selected search region to identify annotations associated with a matching variable value set; and
annotating the identified variant locus with the identified annotations; and
displaying the annotated variant loci.

2. The method of claim 1, wherein each search region comprises a loci range within a chromosome.

3. The method of claim 2, wherein, for each search region, a length of the loci range is determined based on whether the search region includes a coding sequence.

4. The method of claim 2, wherein, when an annotation is mapped to variable value sets associated with multiple search regions within a chromosome, segmenting the annotation data comprises sorting the annotation into a subset of annotation data for a single search region.

5. The method of claim 1, wherein the set of search regions is determined based on a disease of interest, the method further comprising providing a risk score for the disease of interest based on the annotated variant loci, using a risk model.

6. The method of claim 1, wherein the annotation data comprises annotations mapped to variable value sets for non-coding DNA variants.

7. The method of claim 1, wherein the selected search region corresponds to a first range of loci, wherein generating the annotated variant loci further comprises:
selecting a second search region from the set of search regions based on the variable value set, wherein the second search region corresponds to a second range of loci adjacent to the first range of loci;
searching within a subset of annotation data corresponding to the selected second search region to identify annotations associated with a matching variable value set; and
annotating the identified variant locus with the identified annotations.

8. The method of claim 1, wherein variables comprise at least one of: chromosome number, overall chromosome location, variant start position, variant stop position, variant identification number, variant type, reference allele, present allele, or reference assembly number.

9. The method of claim 1, wherein each subset of annotation data is independently stored in a multi-dimensional array data structure, wherein generating annotated variant loci comprises searching multiple subsets of annotation data in parallel.

10. The method of claim 1, wherein the annotation data are used to determine priors for training a risk model.

11. The method of claim 1, wherein determining a variable value set for each identified variant locus in the unannotated genomic data comprises converting the unannotated genomic data into a standardized format.

12. A method, comprising, by one or more computing system:
receiving annotations from a plurality of data sources comprising different data types;
generating annotation data by mapping the annotations to variable value sets, wherein each variable value set corresponds to a genomic variant, wherein the annotation for at least one variable value set comprises a weighted aggregation of multiple annotations, from different data sources, associated with the respective variable value set;
receiving unannotated genomic data for a subject;
comparing the unannotated genomic data to a reference genome to identify variant loci;
determining a variable value set for each identified variant locus in the unannotated genomic data;
annotating each identified variant locus using annotations, from the annotation data, associated with a variable value set matching the variable value set for the identified variant locus; and
displaying the annotated variant loci.

13. The method of claim 12, wherein the weighted aggregation is performed based on a weight metric for each of the different data sources.

14. The method of claim 13, wherein the weight metric for a data source is determined based on at least one of: a number of published annotations associated with the data source, whether annotations from the data source are clinical grade, or whether annotations from the data source are based on expert panel review presence.

15. The method of claim 13, wherein the weight metric comprises a predictive weight determined using a supervised learning model.

16. The method of claim 12, wherein each annotation of the multiple annotations is associated with a weight metric, wherein performing the weighted aggregation of the multiple annotations comprises identifying conflicting annotations in the multiple annotations and selecting an annotation from the conflicting annotations with a higher weight metric.

17. The method of claim 12, wherein each annotation of the multiple annotations is associated with a weight metric, wherein performing the weighted aggregation of the multiple annotations comprises filtering out annotations with a weight metric below a threshold.

18. The method of claim 12, wherein the annotation data comprises annotations mapped to variable value sets for non-coding DNA variants.

19. The method of claim 18, wherein at least a portion of the annotations mapped to variable value sets for non-coding DNA variants are determined using at least one of: genome-wide association studies (GWAS), CRISPR-based functional screens, or by activity-by-contact models.

20. The method of claim 12, further comprising providing, based on the annotated variant loci, at least one of: a recommendation for further clinical testing, a disease diagnosis, a recommended therapeutic regimen, or a recommended modification to an existing therapeutic regimen.
